SEQUENCE LISTING (51 sequences)

SEQ ID NO: 1 - protein, 8 aa, synthetic construct
  SITE 1: note = MISC_FEATURE - X1 is either G or G-R1
  SITE 8: note = MISC_FEATURE - X8 is either Q or Q-R2
XPQGIWGX

SEQ ID NO: 2 - protein, 7 aa, synthetic construct
  SITE 1: note = MISC_FEATURE - X1 is R1-P
  SITE 7: note = MISC_FEATURE - X7 is Q-R2
XQGIWGX

SEQ ID NO: 3 - protein, 4 aa, synthetic construct
GGGG

SEQ ID NO: 4 - protein, 5 aa, synthetic construct
LPETG

SEQ ID NO: 5 - protein, 8 aa, synthetic construct
LPETGGGG

SEQ ID NO: 6 - protein, 6 aa, synthetic construct
HHHHHH

SEQ ID NO: 7 - protein, 15 aa, synthetic construct
GGSGGSGGSGGSGGS

SEQ ID NO: 8 - protein, 6 aa, synthetic construct
  SITE 1: note = MISC_FEATURE - X1 is Gly or Fmoc-Gly
  SITE 6: note = MISC_FEATURE - X6 is Cys-Resin, Cys-NH2, Cys(Cys)(disulfide), (N3-(CH2)3CONH)C (disulfide)
XGGGRX

SEQ ID NO: 9 - protein, 6 aa, synthetic construct
  SITE 1: note = MISC_FEATURE - X1 is Gly or Fmoc-Gly
  SITE 6: note = MISC_FEATURE - X6 is Lys(mtt)-Resin, Lys-Resin, Lys(N3-oNB)-Resin, or Lys(N3-oNB)-NH2
XGGGRX

SEQ ID NO: 10 - protein, 16 aa, synthetic construct
  SITE 1: note = MISC_FEATURE - X1 is Gly or Fmoc-Gly
  SITE 16: note = MISC_FEATURE - X16 is Cys-Resin, Cys-NH2, Cys(Cys) (disulfide), (HN(CO(CH2)3N3))Cys (disulfide)
XGGGRGPQGIWGQGRX

SEQ ID NO: 11 - protein, 6 aa, synthetic construct
  SITE 1: note = MISC_FEATURE - X1 is Gly or Fmoc-Gly
  SITE 6: note = MISC_FEATURE - X6 is Gly, G-oNB-GlyArgCys-NH2, Gly-oNB-GlyArgCys(Cys) (disulfide), Gly-oNB-GlyArgCys(HN(CO(CH2)3N3))Cys (disulfide)
XGGGRX

SEQ ID NO: 12 - protein, 16 aa, synthetic construct
  SITE 1: note = MISC_FEATURE - X1 is Gly or Fmoc-Gly
  SITE 16: note = MISC_FEATURE - X16 is Lys(mtt)-Resin, Lys-Resin, Lys(N3-oNB)-Resin, and Lys(N3-oNB)-NH2
XGGGRGPQGIWGQGRX

SEQ ID NO: 13 - protein, 22 aa, synthetic construct
  SITE 1: note = MISC_FEATURE - X1 is Gly or Fmoc-Gly
  SITE 7: note = MISC_FEATURE - X7 is Cys or Cys (disulfide)
  SITE 19: note = MISC_FEATURE - X19 is Cys or Cys (disulfide)
  SITE 22: note = MISC_FEATURE - X22 is Lys-Resin, Lys-NH2, or Lys(CO(CH2)3N3)-NH2
XGGGRGXGPQGIWGQGQGXGRX

SEQ ID NO: 14 - skipped

SEQ ID NO: 15 - protein, 9 aa, synthetic construct
  SITE 1: note = MISC_FEATURE - X1 is Gly or Fmoc-Gly
  SITE 7: note = MISC_FEATURE - X7 is Cys or Cys(Cys) disulfide
  SITE 9: note = MISC_FEATURE - X9 is Gly, Gly(O-oNB-GlyGlyCysGlyArgLys-Resin), Gly(O-oNB-GlyGlyCysGlyArgLys-NH2), or Gly(O-oNB-GlyGlyCysGlyArgLys(CO(CH2)3N3)-NH2)
XGGGRGXGX

SEQ ID NO: 16 - skipped

SEQ ID NO: 17 - protein, 17 aa, synthetic construct
  SITE 1: note = MISC_FEATURE - X1 is Fmoc-Gly
  SITE 7: note = MISC_FEATURE - X7 is Lys(mtt)
XGGGRGXGGPQGIWGQG

SEQ ID NO: 18 - protein, 22 aa, synthetic construct
  SITE 1: note = MISC_FEATURE - X1 is Gly or Fmoc-Gly
  SITE 7: note = MISC_FEATURE - X7 is Lys, Lys(mtt), Lys(oNB-N3), or Lys(oNB) (linked)
  SITE 18: note = MISC_FEATURE - X18 is Lys(CO(CH2)2CCH) or Lys(CO(CH2)2 (linked)
  SITE 22: note = MISC_FEATURE - X22 is Lys-Resin, Lys-NH2, or Lys(CO(CH2)3N3)-NH2
XGGGRGXGGPQGIWGQGXGRGX

SEQ ID NO: 19 - protein, 7 aa, synthetic construct
ENLYFQG

SEQ ID NO: 20 - DNA, 20 bp, synthetic construct
  source 1..20: mol_type = other DNA
gtaacgatccagctgtcact

SEQ ID NO: 21 - protein, 6 aa, synthetic construct
  SITE 1: note = MISC_FEATURE - X1 is Lys(Dde)
XGCGRK

SEQ ID NO: 22 - protein, 12 aa, synthetic construct
  SITE 1: note = MISC_FEATURE - X1 is Arg, N3(CH2)3CONH-Arg
  SITE 12: note = MISC_FEATURE - X12 is Lys(Dde)-Resin, Lys(CO(CH2)3N3-Resin, or Lys(CO(CH2)3N3)-NH2
XGPQGIWGQGRX

SEQ ID NO: 23 - skipped

SEQ ID NO: 24 - protein, 13 aa, synthetic construct
  SITE 1: note = MISC_FEATURE - X1 is Arg, or N3(CH2)3CONH-Arg
  SITE 13: note = MISC_FEATURE - X13 is Cys-Resin, Cys-NH2, Cys(H2NCys-OH)-NH2 (disulfide), or Cys(N3(CH2)3CONH-Cys-OH) (disulfide)
XGPQGIWGQGRGX

SEQ ID NO: 25 - protein, 8 aa, synthetic construct
QPQGIWGQ

SEQ ID NO: 26 - skipped

SEQ ID NO: 27 - protein, 16 aa, synthetic construct
  SITE 1: note = MISC_FEATURE - X1 is Arg, N3(CH2)3CONH-Arg or oNB-Arg
  SITE 3: note = MISC_FEATURE - X3 is Cys or Cys(Cys) (disulfide)
  SITE 13: note = MISC_FEATURE - X13 is Cys or Cys(Cys) (disulfide)
  SITE 16: note = MISC_FEATURE - X16 is Lys-Resin, Lys-NH2 or Lys(CO(CH2)3N3)-NH2
XGXGPQGIWGQGXGRX

SEQ ID NO: 28 - protein, 15 aa, synthetic construct
  SITE 1: note = MISC_FEATURE - X1 is Arg, NH(CO(CH2)2CCH)-Arg or NH(CO(CH2)2-Arg (linked)
  SITE 3: note = MISC_FEATURE - X3 is Lys or Lys(CO(CH2)3N3
  SITE 13: note = MISC_FEATURE - X13 is Lys or Lys(oNB) (linked)
  SITE 15: note = MISC_FEATURE - X15 is Lys-Resin, Lys-NH2 or Lys(CO(CH2)3N3)-NH2
XGXGPQGIWGQGXRX

SEQ ID NO: 29-30 - skipped (2 entries)

SEQ ID NO: 31 - protein, 12 aa, synthetic construct
  SITE 1: note = MISC_FEATURE - X1 is Arg, oNB-Arg, AcCysGly-oNB-Arg, AcCys(Cys(disulfide))Gly-oNB-Arg or AcCys(N3(CH2)3CONH-Cys(disulfide)Gly-oNB-Arg
  SITE 12: note = MISC_FEATURE - X12 is Lys-Resin, Lys-NH2, Lys(CO(CH2)3N3-NH2, Lys(N3)-NH2 or Lys(N3)-Resin
XGPQGIWGQGRX

SEQ ID NO: 32-33 - skipped (2 entries)

SEQ ID NO: 34 - protein, 9 aa, synthetic construct
  SITE 1: note = MISC_FEATURE - X1 is Gly or PEG2-Gly
  SITE 9: note = MISC_FEATURE - X9 is Gly or Gly-PEG2
XPQGIWGQX

SEQ ID NO: 35 - protein, 15 aa, synthetic construct
  SITE 1: note = MISC_FEATURE - X1 is Gly or NH-oNB-Gly
  SITE 2: note = MISC_FEATURE - X2 is Lys or Lys(CO(CH2)3N3
  SITE 12: note = MISC_FEATURE - X12 is Cys or Cys (disulfide)
  SITE 15: note = MISC_FEATURE - X15 is Lys-Resin, Lys-NH2 or Lys(CO(CH2)3N3)-NH2
XXGPQGIWGQGXGRX

SEQ ID NO: 36 - protein, 13 aa, synthetic construct
  SITE 1: note = MISC_FEATURE - X1 is Gly, NH-oNB-Gly, or ArgGlyCysGly-NH-oNB-Gly
  SITE 10: note = MISC_FEATURE - X10 is Cys or Cys (disulfide)
  SITE 13: note = MISC_FEATURE - X13 is Lys-Resin, Lys-NH2 or Lys(CO(CH2)3N3)-NH2
XPQGIWGQGXGRX

SEQ ID NO: 37 - protein, 14 aa, synthetic construct
  SITE 1: note = MISC_FEATURE - X1 is Gly, NH-oNB-Gly, or AcArgGlyCysGlyLysGly-oNB-Gly
  SITE 2: note = MISC_FEATURE - X2 is Lys or Lys(CO(CH2)3N3
  SITE 12: note = MISC_FEATURE - X12 is Cys or Cys (disulfide)
  SITE 14: note = MISC_FEATURE - X14 is Arg-Resin or Arg-NH2
XXGPQGIWGQGXGX

SEQ ID NO: 38 - skipped

SEQ ID NO: 39 - protein, 13 aa, synthetic construct
  SITE 1: note = MISC_FEATURE - X1 is Gly, NH-oNB-Gly, or ArgGlyCysGly-oNB-Gly
  SITE 2: note = MISC_FEATURE - X2 is Cys or Cys (disulfide)
  SITE 13: note = MISC_FEATURE - X13 is Lys-Resin, Lys-NH2 or Lys(CO(CH2)3N3)-NH2
XXGPQGIWGQGRX

SEQ ID NO: 40 - skipped

SEQ ID NO: 41 - protein, 13 aa, synthetic construct
  SITE 1: note = MISC_FEATURE - X1 is Ac-Lys
  SITE 4: note = MISC_FEATURE - X4 is Lys(Dde) or Lys-oNB
  SITE 13: note = MISC_FEATURE - X13 is Gly or Gly (linked)
XRGXGPQGIWGQX

SEQ ID NO: 42 - skipped

SEQ ID NO: 43 - protein, 13 aa, synthetic construct
  SITE 1: note = MISC_FEATURE - X1 is Gly or FAM-Gly
  SITE 13: note = MISC_FEATURE - X13 is Lys or Lys(Boc)-Resin
XRGPQGIWGQGRX

SEQ ID NO: 44 - protein, 13 aa, synthetic construct
  SITE 1: note = MISC_FEATURE - X1 is Gly or FAM-Gly
  SITE 13: note = MISC_FEATURE - X13 is Cys-Resin, Cys-NH2, Cys(Cys) (disulfide), or Cys(N3(CH2)3CONH-Cys) (disulfide)
XRGPQGIWGQGRX

SEQ ID NO: 45 - protein, 16 aa, synthetic construct
  SITE 1: note = MISC_FEATURE - X1 is Gly or FAM-Gly
  SITE 16: note = MISC_FEATURE - X16 is Cys-Resin, Cys-NH2, Cys(Cys) disulfide or Cys(N3(CH2)3CONH-Cys) (disulfide)
XRRGPQGIWGQGRGRX

SEQ ID NO: 46 - protein, 17 aa, synthetic construct
  SITE 1: note = MISC_FEATURE - X1 is Gly or FAM-Gly
  SITE 4: note = MISC_FEATURE - X4 is Cys or Cys (disulfide)
  SITE 14: note = MISC_FEATURE - X14 is Cys or Cys (disulfide)
  SITE 17: note = MISC_FEATURE - X17 is Lys(Boc)-Resin, Lys-NH2, or Lys(CO(CH2)3N3)-NH2
XRGXGPQGIWGQGXGRX

SEQ ID NO: 47-48 - skipped (2 entries)

SEQ ID NO: 49 - protein, 17 aa, synthetic construct
  SITE 1: note = MISC_FEATURE - X1 is Fmoc-Gly or N3(CH2)3CONH-Gly
  SITE 3: note = MISC_FEATURE - X3 is Lys(Dde) or Lys (linked)
  SITE 15: note = MISC_FEATURE - X15 is Lys(Boc), Lys, or Lys(FAM)
  SITE 17: note = MISC_FEATURE - X17 is Lys(CO(CH2)2CCH-Resin, Lys(CO(CH2)2CCH-NH2, or Lys(linked)-NH2
XRXRGPQGIWGQGRXGX

SEQ ID NO: 50 - protein, 17 aa, synthetic construct
  SITE 1: note = MISC_FEATURE - X1 is Gly or FAM-Gly
  SITE 4: note = MISC_FEATURE - X4 is Cys or Cys (disulfide)
  SITE 14: note = MISC_FEATURE - X14 is Cys or Cys (disulfide)
  SITE 17: note = MISC_FEATURE - X17 is Lys(Boc)-Resin, Lys-NH2, or Lys(linked)-NH2
XRGXGPQGIWGQGXGRX

SEQ ID NO: 51 - protein, 18 aa, synthetic construct
  SITE 1: note = MISC_FEATURE - X1 is Gly or Gly-PL-N3
  SITE 5: note = MISC_FEATURE - X5 is Cys or Cys (disulfide)
  SITE 15: note = MISC_FEATURE - X15 is Cys or Cys (disulfide)
  SITE 18: note = MISC_FEATURE - X18 is Glu(tBu)-Resin, Glu-NH2 or Glu(DOX)-NH2
XRGRXGPQGIWGQGXGRX

(19) United States
(12) Patent Application Publication
(10) Pub. No.: US 2025/0135016 A1
(43) Pub. Date: May 1, 2025

(54) MOLECULAR LOGIC GATES FOR CONTROLLED MATERIAL DEGRADATION

(71) Applicant: University of Washington, Seattle, WA (US)

(72) Inventors: Cole DeForest, Seattle, WA (US); Barry Badeau, Seattle, WA (US)

(73) Assignee: University of Washington, Seattle, WA (US)

(21) Appl. No.: 18/935,315

(22) Filed: Nov. 1, 2024

Related U.S. Application Data
(63) Continuation of application No. 16/335,664, filed on Mar. 21, 2019.
(60) Provisional application No. 62/398,424, filed on Sep. 22, 2016.

(51) Int. Cl. A61K 47/65; A61K 47/69

(52) U.S. Cl. CPC A61K 47/65; A61K 47/6903

(57) ABSTRACT

The present disclosure features, inter alia, a cyclic multifunctional linker, including at least two cleavable moieties; at least two connecting chains connected to the at least two cleavable moieties to provide a cyclic structure; and at least two linking groups, each linking group being bonded at one end to a connecting chain and being located between two cleavable moieties, and each linking group having a second end configured to bond to crosslinkable moieties. In the cyclic multifunctional linker, each connecting chain has at least two ends, and at least two of the connecting chains are each connected at each end to a cleavable moiety.

### CROSS-REFERENCES TO RELATED APPLICATIONS

This application is a continuation of application Ser. No. 16/335,664, filed Mar. 21, 2019, which is a National Stage of International Application No. PCT/US2017/053034, filed Sep. 22, 2017, which claims the benefit of U.S. Provisional Application No. 62/398,424, filed Sep. 22, 2016; the disclosures of which are incorporated herein by reference in their entirety.

### STATEMENT REGARDING SEQUENCE LISTING

The Sequence Listing XML associated with this application is provided in XML format and is hereby incorporated by reference into the specification. The name of the XML file containing the sequence listing is 3915-P870US.CON.UW_Seq_List_20241031.xml. The XML file is 70,528 bytes; was created on Oct. 31, 2024; and is being submitted electronically via Patent Center with the filing of the specification.

### BACKGROUND

Recent innovations in therapeutic development have yielded powerful tools to combat an increasing number of debilitating and life-threatening diseases. Despite these advances, several barriers to clinical translation remain, including the significant challenge of limiting therapeutic deployment to sites of disease that can be widespread and unknown. Targeted delivery strategies that exploit disease-related biomarkers improve treatment efficiency and efficacy by reducing dosage requirements and adverse off-target effects. Generally, these methods employ polymer-based vehicles to facilitate delivery while protecting therapeutic cargo from immune recognition, clearance, and non-specific cellular uptake. Cell-based therapies further necessitate that these vehicles recapitulate critical aspects of native tissue to ensure sustained cell viability and function. Hydrogels offer promise in each of these regards, representing robust material platforms whose biochemical and biophysical properties can be tuned to preserve and promote specific cell fates, that are readily formulated into a variety of shapes and stiffnesses to control transport to and within tissues, and that can be engineered to degrade in response to locally-presented cues to facilitate therapeutic release.

Smart materials have been engineered to leverage pathophysiology for targeted delivery by integrating functional groups that cleave or change conformation in response to an external stimulus (e.g., enzyme, pH, temperature, redox conditions), allowing them to dynamically sense and respond to disease-associated biochemical hallmarks. Though materials sensitive to single factors can enrich therapeutic delivery to sites of disease, release specificity is often poor due to the lack of uniqueness for individual biomarkers. For example, cancer microenvironments have been targeted through their extensive matrix metalloproteinase (MMPp) activity, reducing conditions, and sub-physiological pH; however, these conditions are also native to healthy joints, the intracellular milieu, and the stomach, respectively.

For therapeutic protein delivery, hydrogels have emerged as effective vehicles for the controlled delivery of therapeutic proteins. Their tunable mesh size, functionality, and degradability enables tailored biomacromolecule presentation in response to biologically-relevant external cues. While gel-based platforms that swell or degrade in response to physical or chemical signals (e.g., temperature, pH, enzyme, light, redox, other biomolecules) have been extensively investigated for the simultaneous release of several proteins, strategies that permit independent and differentially-triggered release of many proteins from a common material remain elusive. Moreover, the ability to regulate release without sacrificing protein stability or bioactivity represents an open challenge that is of continued interest to the biomaterials community.

As for small molecule drug delivery, there has been a broad array of sensitive materials designed and synthesized to target specific tissues or characteristic biochemical responses to disease, focusing on simple physical or chemical changes in those tissues. The traditional approach has been designing a system to capitalize on a biochemical response by encapsulating a therapeutic in a biomaterial which will degrade and release the therapeutic when introduced to that biochemical change. Researchers have found ways to elicit these types of degradation or release including thermal expansion, pH sensitivity, reductive, enzyme responsive, photodegradation, polysaccharides, oligonucleotides, ultrasound, glucose concentrations, and many more. A primary limitation in the design of encapsulation systems is the size exclusion of the network related to the therapeutic being encapsulated. As such, care must be taken to carefully design encapsulation systems appropriately for the size or hydrodynamic radius of the chosen therapeutic, often proteins or proteoglycans, which conversely restricts the ability to apply these systems to smaller therapeutics. Moreover, the environmental stimuli required to elicit the desired response in these biomaterials are often far from unique to one place in the body. This leads to off target release with the potential of causing extremely deleterious effects, especially when attempting to deliver cytotoxic compounds.

There is a need for a versatile approach to create multi-stimuli responsive hydrogel platforms that are (1) able to perform biocomputation, (2) modular in design, and (3) fully cytocompatible. There is also a need for delivery systems that permit independent and differentially-triggered release of therapeutic agents (e.g., proteins and/or small molecules) from a common material that allows for regulated release without sacrificing therapeutic agent stability or bioactivity. The present disclosure seeks to fulfill these needs and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features cyclic multifunctional linker, including at least two cleavable moieties; at least two connecting chains connected to the at least two cleavable moieties to provide a cyclic structure, wherein each connecting chain has at least two ends, and at least two of the connecting chains are each connected at each end to a cleavable moiety; and at least two linking groups, each linking group being bonded at one end to a connecting chain and being located between two cleavable moieties, and each linking group having a second end configured to bond to crosslinkable moieties.

In another aspect the present disclosure features a cyclic multifunctional linker having Formula (I):

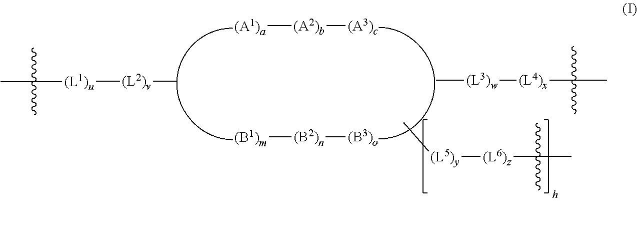

wherein
A¹, A², A³, B¹, B², B³, L¹, L², L³, L⁴, L⁵, and L⁶ are each independently a cleavable moiety configured to cleave when exposed to a stimulus;
a and m are each independently selected from 1, 2, and 3;
b, c, n, and o are each independently selected from 0, 1, 2, and 3;
v and w are each independently selected from 0, 1, and 2;
u and x are each independently selected from 0, 1, and 2;
y is selected from 0, 1, and 2;
z is selected from 0, 1, and 2; and
h is selected from 0, 1, 2, or 3.

In another aspect, the present disclosure features a cyclic multifunctional linker having Formula (II):

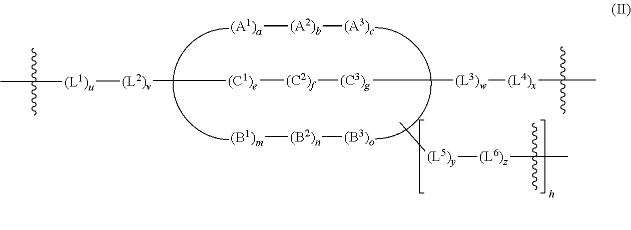

wherein:
A¹, A², A³, B¹, B², B³, C¹, C², C³, L¹, L², L³, L⁴, L⁵, and L⁶ are each independently a cleavable moiety configured to cleave when exposed to a stimulus;
a, e, and m are each independently selected from 1, 2, and 3;
b, c, f, g, n, and o are each independently selected from 0, 1, 2, and 3;
v and w are each independently selected from 0, 1, and 2;
u and x are each independently selected from 0, 1, and 2;
y is selected from 0, 1, and 2;
z is selected from 0, 1, and 2; and
h is selected from 0, 1, 2, or 3.

In yet another aspect, the present disclosure features a cyclic multifunctional linker having Formula (III):

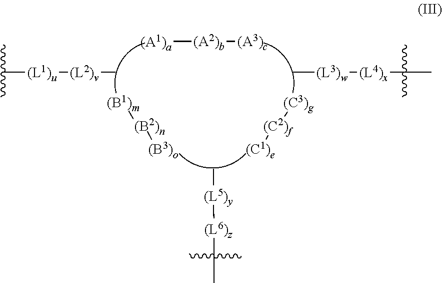

wherein:
A¹, A², A³, B¹, B², B³, C¹, C², C³, L¹, L², L³, L⁴, L⁵, and L⁶ are each independently a cleavable moiety configured to cleave when exposed to a stimulus;
a, e, and m are each independently selected from 1, 2, and 3;
b, c, f, g, n, and o are each independently selected from 0, 1, 2, and 3;
v, w, and y are each independently selected from 0, 1, and 2; and
u, x, and z are each independently selected from 0, 1, and 2.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A a scheme showing a YES-gate crosslinker containing a stimuli-labile moiety (middle shaded region). Presence of the corresponding chemical stimuli (i.e., input) cleaves the stimuli-labile moiety, breaking the covalent linkage between molecular endpoints, resulting in material degradation. Each region of the Venn diagram corresponds to a unique combination of inputs and indicates whether the material is expected to degrade (shaded) or remain intact (not shaded).

FIG. 1B is a scheme showing an OR-gate crosslinker containing two different stimuli-labile moieties (shaded regions) connected in series. The presence of either relevant stimulus (i.e., input) covalently cleaves the crosslinker resulting in material degradation.

FIG. 1C is a scheme showing an AND-gate crosslinker containing two different stimuli-labile moieties (shaded regions) connected in parallel. The presence of a single programmed stimulus covalently cleaves a single arm of the linker but does not sever the crosslink, and therefore does not change the crosslinking density or mechanical properties of the material. Both stimuli are required for material degradation.

FIG. 1D is a scheme showing that logic gates can be hierarchically combined to generate higher-order logical responses. For example, seventeen unique materials can be generated by combining three logic gates (YES, OR, AND) with three stimuli.

FIG. 1E shows embodiments of example reactions that can provide cleavage of the stimuli-labile groups: disulfide bonds are reduced into free thiols, proteolytically-sensitive peptide sequence GPQG↓IWGQ (SEQ ID NO. 1) is enzymatically cleaved by MMP, and the ortho-nitrobenzyl (oNB) moiety undergoes photoscission in the presence of near-UV light (λ=365 nm) to produce amino acids 1-4 of (SEQ ID NO. 1) and amino acids 5-8 of (SEQ ID NO. 1).

FIG. 2A is a drawing depicting the chemical structure of an embodiment of a crosslinker of the present disclosure, specifically an E∧P crosslinker that includes an enzyme-labile sequence, a photo-labile group, and two flanking azides for SPAAC-based hydrogel crosslinking, (SEQ ID NO. 28).

FIG. 2B is a graph of an in situ oscillatory rheology of crosslinked hydrogels of the present disclosure, specifically hydrogels crosslinked using treated E∧P, and demonstrate that that AND-gate materials require treatment by both relevant stimuli to provide changes in bulk material properties.

FIG. 3A is a graph showing response profiles of embodiments of crosslinked materials of the present disclosure, specifically single-stimulus YES-gate materials. Plot titles correspond to crosslinker identity, with x-axis labels indicating material treatment conditions (N is no treatment, E is MMP enzyme, R is a chemical reductant, P is light). Shaded bars signify conditions expected to result in material degradation; clear bars indicate conditions expected not to yield material degradation.

FIG. 3B is a graph showing response profiles of embodiments of crosslinked materials of the present disclosure, specifically the two-stimuli OR-gate materials. Plot titles correspond to crosslinker identity, with x-axis labels indicating material treatment conditions (N is no treatment, E is MMP enzyme, R is a chemical reductant, P is light). Shaded bars signify conditions expected to result in material degradation; clear bars indicate conditions expected not to yield material degradation.

FIG. 3C is a graph showing response profiles of embodiments of crosslinked materials of the present disclosure, specifically two-stimuli AND gate materials. Plot titles correspond to crosslinker identity, with x-axis labels indicating material treatment conditions (N is no treatment, E is MMP enzyme, R is a chemical reductant, and P is light). Shaded bars signify conditions expected to result in material degradation; clear bars indicate conditions expected not to yield material degradation.

FIG. 3D is a graph showing response profiles of embodiments of crosslinked materials of the present disclosure, specifically higher-order, three-input OR/AND materials. Plot titles correspond to crosslinker identity, with x-axis labels indicating material treatment conditions (N is no treatment, E is MMP enzyme, R is a chemical reductant, and P is light). Shaded bars signify conditions expected to result in material degradation; clear bars indicate conditions expected not to yield material degradation.

FIG. 3E is a graph showing response profiles of embodiments of crosslinked materials of the present disclosure, specifically higher-order, three-input AND/OR materials. Plot titles correspond to crosslinker identity, with x-axis labels indicating material treatment conditions (N is no treatment, E is MMP enzyme, R is a chemical reductant, and P is light). Shaded bars signify conditions expected to result in material degradation; clear bars indicate conditions expected not to yield material degradation.

FIG. 3F is a graph showing response profiles of embodiments of crosslinked materials of the present disclosure, specifically higher-order, three-input OR/OR materials. Plot titles correspond to crosslinker identity, with x-axis labels indicating material treatment conditions (N is no treatment, E is MMP enzyme, R is a chemical reductant, and P is light). Shaded bars signify conditions expected to result in material degradation; clear bars indicate conditions expected not to yield material degradation.

FIG. 3G is a graph showing response profiles of embodiments of crosslinked materials of the present disclosure, specifically higher-order, three-input AND/AND materials. Plot titles correspond to crosslinker identity, with x-axis labels indicating material treatment conditions (N is no treatment, E is MMP enzyme, R is a chemical reductant, and P is light). Shaded bars signify conditions expected to result in material degradation; clear bars indicate conditions expected not to yield material degradation.

FIG. 4A is a drawing depicting the chemical structure of doxorubicin (DOX) functionalized at the amino group with bicyclononyne (BCN).

FIG. 4B is a schematic showing that degradation of an embodiment of a hydrogel of the present disclosure, specifically R∧E hydrogel degradation, is triggered in the presence of pathophysiological cues associated with tumor microenvironments: reducing conditions and MMPs. Liberated DOX induces apoptosis in cervical cancer-derived HeLa cells.

FIG. 4C is a graph showing a dose-response curve of HeLa cells following treatment with R∧E-DOX conjugate.

FIG. 4D is a graph showing normalized dsDNA content after culturing HeLa with released hydrogel components following varying treatments. X-axis label indicates material treatment conditions (N is no treatment, R is a chemical reductant, E is MMP enzyme). Shaded bars signify conditions expected to result in DOX release though material degradation; clear bars indicate conditions expected not to yield material degradation.

FIG. 5A is a series of micrographs showing spatially-segregated regions of fluorescently-labeled R∧P P, and R∨P hydrogels, formulated and imaged via confocal microscopy following sequential treatments of photomasked light and reducing conditions. Each region responded to the cues as engineered, degrading only in the presence of the proper set of stimulus conditions.

FIG. 5B is a series of micrographs showing hydrogels subjected to the experimental conditions of FIG. 5A, but repeated with encapsulated fluorescent cells (hS5 stably transfected to produce mCherry, GFP, and BFP, respectively) replacing the small molecule fluorophores.

FIG. 5C is a series of graphs showing cells released from the hydrogels of FIG. 5B, quantified using flow cytometry following sequential exposure to photomasked light, reducing conditions, and flood light exposure, respectively. Histograms present fluorescence intensity for channels corresponding to each cell line with the shaded regions indicating positively gated cells.

FIG. 6 is a flow chart describing degradation conditions for embodiments of the crosslinked hydrogels of the present disclosure.

FIG. 7 is a flow chart describing degradation conditions for embodiments of the crosslinked hydrogels of the present disclosure.

FIG. 8 is a series of photographs showing embodiments of the crosslinked hydrogels following degradation conditions.

FIG. 9A is a graph showing degradation of embodiments of bulk crosslinked hydrogels of the present disclosure.

FIG. 9B is a graph showing degradation of embodiments of thin films of crosslinked hydrogels of the present disclosure.

FIG. 10A is a graph showing degradation of embodiments of bulk crosslinked hydrogels of the present disclosure.

FIG. 10B is a graph showing degradation of embodiments of thin films of crosslinked hydrogels of the present disclosure.

FIG. 11A is a graph showing degradation of embodiments of bulk crosslinked hydrogels of the present disclosure.

FIG. 11B is a graph showing degradation of embodiments of thin films of crosslinked hydrogels of the present disclosure.

FIG. 12 is flow chart describing degradation conditions for embodiments of the crosslinked hydrogels of the present disclosure.

FIG. 13A is a graph showing biological response of cells incubated with embodiments of the crosslinked hydrogels lacking doxorubicin of the present disclosure, upon exposure to various stimuli.

FIG. 13B is a graph showing biological response of cells incubated with embodiments of the crosslinked hydrogels including doxorubicin of the present disclosure, upon exposure to various stimuli.

FIG. 14 is a graph showing dose-response curve of cells incubated with doxorubicin.

FIG. 15A is a schematic representation of programmed YES logic therapeutic release from a biomaterial matrix of the present disclosure.

FIG. 15B is a drawing of an embodiment of an enzyme cleavable sequence, PQGIWGQ (SEQ ID NO. 2) to produce amino acids 1-4 of (SEQ ID NO. 2) and amino acids 5-7 of (SEQ ID NO. 2).

FIG. 15C is a drawing of an embodiment of a sequence that is cleavable with a reducing agent.

FIG. 15D is a drawing of an embodiment of a photolabile sequence.

FIG. 16 is a schematic representation of a release of a therapeutic agent from an embodiment of a hydrogel of the present disclosure, where the therapeutic agent releases only in response to two required stimuli.

FIG. 17A is a photograph an embodiment of a photolabile-AND-reducible multifunctional linker, tested for selectivity and spatial control in 4-25 μL gels on azide functionalized glass slides. A hatched square photomask with 100 μm squares was used. The gels were exposed to PBS only.

FIG. 17B is a photograph an embodiment of a photolabile-AND-reducible multifunctional linker, tested for selectivity and spatial control in 4-25 μL gels on azide functionalized glass slides. A hatched square photomask with 100 μm squares was used. The gels were exposed to dithiothreitol (DTT) only.

FIG. 17C is a photograph an embodiment of a photolabile-AND-reducible multifunctional linker, tested for selectivity and spatial control in 4-25 μL gels on azide functionalized glass slides. A hatched square photomask with 100 μm squares was used. The gels were exposed to photomasked light only.

FIG. 17D is a photograph an embodiment of a photolabile-AND-reducible multifunctional linker, tested for selectivity and spatial control in 4-25 μL gels on azide functionalized glass slides. A hatched square photomask with 100 μm squares was used. The gels were exposed to photomasked light and DTT treatments.

FIG. 18 is a schematic representation of a release of a fluorescent reporter from an embodiment of a biomaterial of the present disclosure using a model-AND/OR-responsive multifunctional linker.

FIG. 19 is a schematic representation of a release of a therapeutic agent from an embodiment of a biomaterial of the present disclosure using a model-OR-responsive multifunctional linker.

FIG. 20A is a schematic of a synthesis and logic-based release of site-specifically-modified proteins from embodiments of hydrogels of the present disclosure. The sortase-mediated transpeptidation reaction, employed by STEPL methodologies, enables degradable polyglycine (GGGG (SEQ ID NO. 3)) probes to be quantitatively affixed to the C-termini of proteins of interest, such as LPETG (SEQ ID NO. 4), to produce the C-Functionalized Releasable Protein with LPETGGGG (SEQ ID NO. 5), and a HHHHHH (SEQ ID NO. 6) tagged Sortase.

FIG. 20B is a drawing of degradable moieties that are responsive to MMP, such as GPQGIWGQ (SEQ ID NO. 1) to produce amino acids 1-4 of (SEQ ID NO. 1) and amino acids 5-8 of (SEQ ID NO. 1).

FIG. 20C is a drawing of degradable moieties that are responsive to reducing agents.

FIG. 20D is a drawing of degradable moieties that are responsive to light.

FIG. 20E is a schematic showing hydrogel-bound proteins that are independently released from hydrogels when materials are exposed to different stimuli combinations corresponding to the degradable moieties. By coupling proteins to the gel through a cyclic peptide, multiple stimuli are required to trigger release.

FIG. 21A is a graph showing quantification of logic-based protein release from hydrogels for C-modified EGFP variants, specifically YES-based EGFP-E-N₃, following logical release of EGFP upon no treatment (N) or by MMP enzyme (E), reductive treatment (R), light (P), reductive and enzyme (RE), enzyme and light (EP), reductive and light (RP), reductive, enzyme and light (REP). In all cases, solid back bars indicate the conditions where release was expected and crosshatched bars represent those where no release was expected. Released EGFP was quantified by supernatant fluorescent analysis and was plotted normalized to the condition yielding the highest release.

FIG. 21B is a graph showing quantification of logic-based protein release from hydrogels for C-modified EGFP variants, specifically YES-based EGFP-R—N₃, following logical release of EGFP upon no treatment (N) or by MMP enzyme (E), reductive treatment (R), light (P), reductive and enzyme (RE), enzyme and light (EP), reductive and light (RP), reductive, enzyme and light (REP). In all cases, solid back bars indicate the conditions where release was expected and crosshatched bars represent those where no release was expected. Released EGFP was quantified by supernatant fluorescent analysis and was plotted normalized to the condition yielding the highest release.

FIG. 21C is a graph showing quantification of logic-based protein release from hydrogels for C-modified EGFP variants, specifically YES-based EGFP-P—N₃, following logical release of upon no treatment (N) or by MMP enzyme (E), reductive treatment (R), light (P), reductive and enzyme (RE), enzyme and light (EP), reductive and light (RP), reductive, enzyme and light (REP). In all cases, solid back bars indicate the conditions where release was expected and crosshatched bars represent those where no release was expected. Released EGFP was quantified by supernatant fluorescent analysis and was plotted normalized to the condition yielding the highest release.

FIG. 21D is a graph showing quantification of logic-based protein release from hydrogels for C-modified EGFP variants, specifically OR-based EGFP-E∨R—N₃, following logical release of EGFP upon no treatment (N) or by MMP enzyme (E), reductive treatment (R), light (P), reductive and enzyme (RE), enzyme and light (EP), reductive and light (RP), reductive, enzyme and light (REP). In all cases, solid back bars indicate the conditions where release was expected and crosshatched bars represent those where no release was expected. Released EGFP was quantified by supernatant fluorescent analysis and was plotted normalized to the condition yielding the highest release.

FIG. 21E is a graph showing quantification of logic-based protein release from hydrogels for C-modified EGFP variants, specifically OR-based EGFP-R∨P—N₃, following logical release of EGFP upon no treatment (N) or by MMP enzyme (E), reductive treatment (R), light (P), reductive and enzyme (RE), enzyme and light (EP), reductive and light (RP), reductive, enzyme and light (REP). In all cases, solid back bars indicate the conditions where release was expected and crosshatched bars represent those where no release was expected. Released EGFP was quantified by supernatant fluorescent analysis and was plotted normalized to the condition yielding the highest release.

FIG. 21F is a graph showing quantification of logic-based protein release from hydrogels for C-modified EGFP variants, specifically OR-based EGFP-E∨P—N₃, following logical release of EGFP upon no treatment (N) or by MMP enzyme (E), reductive treatment (R), light (P), reductive and enzyme (RE), enzyme and light (EP), reductive and light (RP), reductive, enzyme and light (REP). In all cases, solid back bars indicate the conditions where release was expected and crosshatched bars represent those where no release was expected. Released EGFP was quantified by supernatant fluorescent analysis and was plotted normalized to the condition yielding the highest release.

FIG. 21G is a graph showing quantification of logic-based protein release from hydrogels for C-modified EGFP variants, specifically AND-based EGFP-E∧R—N₃, following logical release of EGFP upon no treatment (N) or by MMP enzyme (E), reductive treatment (R), light (P), reductive and enzyme (RE), enzyme and light (EP), reductive and light (RP), reductive, enzyme and light (REP). In all cases, solid back bars indicate the conditions where release was expected and crosshatched bars represent those where no release was expected. Released EGFP was quantified by supernatant fluorescent analysis and was plotted normalized to the condition yielding the highest release.

FIG. 21H is a graph showing quantification of logic-based protein release from hydrogels for C-modified EGFP variants, specifically AND-based EGFP-R∧P—N₃, following logical release of EGFP upon no treatment (N) or by MMP enzyme (E), reductive treatment (R), light (P), reductive and enzyme (RE), enzyme and light (EP), reductive and light (RP), reductive, enzyme and light (REP). In all cases, solid back bars indicate the conditions where release was expected and crosshatched bars represent those where no release was expected. Released EGFP was quantified by supernatant fluorescent analysis and was plotted normalized to the condition yielding the highest release

FIG. 21I is a graph showing quantification of logic-based protein release from hydrogels for C-modified EGFP variants, specifically AND-based EGFP-E∧P—N₃, following logical release of EGFP upon no treatment (N) or by MMP enzyme (E), reductive treatment (R), light (P), reductive and enzyme (RE), enzyme and light (EP), reductive and light (RP), reductive, enzyme and light (REP). In all cases, solid back bars indicate the conditions where release was expected and crosshatched bars represent those where no release was expected. Released EGFP was quantified by supernatant fluorescent analysis and was plotted normalized to the condition yielding the highest release.

FIG. 22 is a series of micrographs showing independent release of EGFP and mCherry fluorescent proteins from the same gel via different logic-based responses. A-D: YES-based logical release of EGFP-P—N₃ and mCherry-E-N₃; E-H: OR-based logical release of EGFP-P∨R—N₃ (and mCherry-E∨R—N₃; I-L: AND-based logical release of EGFP-E∧P—N₃ and mCherry-R∧P—N₃ upon sequential enzyme, masked light exposure, and reductive treatments. All gels were visualized by fluorescent confocal microscopy. Channels 1 and 2 correspond to EGFP (green) and mCherry (red), respectively. Scale bars=400 μm.

FIG. 23A is a schematic showing independently-triggered release of three distinct proteins from a crosslinked gel of the present disclosure.

FIG. 23B is a graph showing quantification of release of EGFP-R—N₃, mCherry-P—N₃, and mCerulean-E-N₃ from a crosslinked hydrogel of the present disclosure upon no treatment (N) or treatment by combinations of MMP enzyme (E), reductive treatment (R), and light (P). In all cases, solid bars indicate conditions where release was expected, while checkered bars represent those in which no release was expected.

FIG. 24 is a synthetic scheme of a reduction-degradable, sortaggable peptide H-GGGGRC(N₃(CH₂)₃CONH-Cys)-NH₂ (SEQ ID NO. 8).

FIG. 25 is a synthetic scheme of a photo-degradable, sortaggable peptide H-GGGGRK(oNB-N₃)—NH₂ (SEQ ID NO. 9).

FIG. 26 is a synthetic scheme of an enzyme-OR-reductive-degradable, sortaggable peptide H-GGGGRGPQGIWGQGRC(N₃(CH₂)₃CONH-Cys)-NH₂ (SEQ ID NO. 10).

FIG. 27 is a synthetic scheme of a reductive-OR-photo-degradable, sortaggable peptide H-GGGGRG-oNB-GRC(N₃(CH₂)₃CONH-Cys)-NH₂ (SEQ ID NO. 11).

FIG. 28 is a synthetic scheme of an enzyme-OR-photo-degradable, sortaggable peptide H-GGGGRGPQGIWGQGRK(N₃-oNB)—NH₂ (SEQ ID NO. 12).

FIG. 29 is a synthetic scheme of an enzyme-AND-reductive-degradable, sortaggable peptide H-GGGGRGCGPQGIWGQGQGCGRK(CO(CH₂)₃N₃)—NH₂ (SEQ ID NO. 13).

FIG. 30 is a synthetic scheme of a reductive-AND-photo-degradable, sortaggable peptide (SEQ ID NO. 15 bound to (SEQ ID NO. 14) through oNB.

FIG. 31 is a synthetic scheme of an enzyme-AND-photo-degradable, sortaggable peptide H-GGGGRGK(oNB)GGPQGIWGQGK(CO(CH₂)₂)GRGK(CO(CH₂)₃N₃)—NH₂ stapled intramolecularly via triazole (SEQ ID NO. 18).

### DETAILED DESCRIPTION

In one aspect, the present disclosure features a cyclic multifunctional linker, including at least two cleavable moieties; at least two connecting chains connected to the at least two cleavable moieties to provide a cyclic structure; and at least two linking groups, each linking group being bonded at one end to a connecting chain and being located between two cleavable moieties, and each linking group having a second end configured to bond to crosslinkable moieties. In the cyclic multifunctional linker, each connecting chain has at least two ends, and at least two of the connecting chains are each connected at each end to a cleavable moiety.

Without wishing to be bound by theory, the transport of drug- and cell-based therapeutics to diseased sites is believed to be a major barrier to clinical translation. Targeted delivery can be mediated through biomaterial vehicles that utilize environmental cues characteristic of diseased-states to trigger payload release. The present disclosure features a modular framework for imparting materials with degradability in response to multiple external cues governed by user-programmable Boolean logic, including YES/OR/AND gates and combinations thereof. Thus, the present disclosure makes use of biocomputation, which is the ability to simultaneously sense multiple biologically-presented inputs and follow a user-programmed Boolean logic-based algorithm to provide a functionally-useful output, in the form of material degradation and delivery of biologically active agents. System modularity allows both the inputs and the logical functions to be changed to generate any number of materials, each with unique and user-specified release characteristics. In some embodiments, the use of cytocompatible bioorthogonal chemistries allows responsive material platforms to be formed and degraded on demand in the presence of live cells.

### Definitions

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

As used herein, the term “substituted” or “substitution” refers to the replacing of a hydrogen atom with a substituent other than H. For example, an “N-substituted piperidin-4-yl” refers to replacement of the H atom from the NH of the piperidinyl with a non-hydrogen substituent such as, for example, alkyl.

As used herein, the term “alkyl” refers to a saturated hydrocarbon group which is straight-chained (e.g., linear) or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 30, from 1 to about 24, from 2 to about 24, from 1 to about 20, from 2 to about 20, from 1 to about 10, from 1 to about 8, from 1 to about 6, from 1 to about 4, or from 1 to about 3 carbon atoms.

As used herein, the term “aryl” refers to monocyclic or polycyclic (e.g., having 2, 3, or 4 fused rings) aromatic hydrocarbons such as, for example, phenyl, naphthyl, anthracenyl, phenanthrenyl, indanyl, and indenyl. In some embodiments, aryl groups have from 6 to about 20 carbon atoms.

As used herein, the term “halo” or “halogen” includes fluoro, chloro, bromo, and iodo.

As used herein, the term “alkylene” refers to a linking alkyl group.

As used herein, “alkenyl” refers to an alkyl group having one or more double carbon-carbon bonds. The alkenyl group can be linear or branched. Example alkenyl groups include ethenyl, propenyl, and the like. An alkenyl group can contain from 2 to about 30, from 2 to about 24, from 2 to about 20, from 2 to about 10, from 2 to about 8, from 2 to about 6, or from 2 to about 4 carbon atoms.

As used herein, “alkenylene” refers to a linking alkenyl group.

As used herein, “alkynyl” refers to an alkyl group having one or more triple carbon-carbon bonds. The alkynyl group can be linear or branched. Example alkynyl groups include ethynyl, propynyl, and the like. An alkynyl group can contain from 2 to about 30, from 2 to about 24, from 2 to about 20, from 2 to about 10, from 2 to about 8, from 2 to about 6, or from 2 to about 4 carbon atoms.

As used herein, “alkynylene” refers to a linking alkynyl group.

As used herein, “alkoxy” refers to an —O-alkyl group. Example alkoxy groups include methoxy, ethoxy, propoxy (e.g., n-propoxy and isopropoxy), t-butoxy, and the like.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “haloalkenyl” refers to an alkenyl group having one or more halogen substituents.

As used herein, “haloalkynyl” refers to an alkynyl group having one or more halogen substituents.

As used herein, “haloalkoxy” refers to an —O-(haloalkyl) group.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “arylene” refers to a linking aryl group.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of pentane, pentene, hexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbomyl, norpinyl, norcamyl, adamantyl, and the like.

As used herein, “cycloalkylene” refers to a linking cycloalkyl group.

As used herein, “heteroalkyl” refers to an alkyl group having at least one heteroatom such as sulfur, oxygen, or nitrogen.

As used herein, “heteroalkylene” refers to a linking heteroalkyl group.

As used herein, a “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heteroarylene” refers to a linking heteroaryl group.

As used herein, “amino” refers to NH₂.

As used herein, “alkylamino” refers to an amino group substituted by an alkyl group.

As used herein, “dialkylamino” refers to an amino group substituted by two alkyl groups.

As used herein, the term “random copolymer” is a copolymer having an uncontrolled mixture of two or more constitutional units. The distribution of the constitutional units throughout a polymer backbone can be a statistical distribution, or approach a statistical distribution, of the constitutional units. In some embodiments, the distribution of one or more of the constitutional units is favored. For a polymer made via a controlled polymerization (e.g., RAFT, ATRP, ionic polymerization), a gradient can occur in the polymer chain, where the beginning of the polymer chain (in the direction of growth) can be relatively rich in a constitutional unit formed from a more reactive monomer while the later part of the polymer can be relatively rich in a constitutional unit formed from a less reactive monomer, as the more reactive monomer is depleted. To decrease differences in distribution of the constitutional units, comonomers in the same family (e.g., methacrylate-methacrylate, acrylamide-acrylamido) can be used in the polymerization process, such that the monomer reactivity ratios are similar.

As used herein, the term “constitutional unit” of a polymer refers to an atom or group of atoms in a polymer, comprising a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O— corresponding to a repeat unit, or —CH₂CH₂OH corresponding to an end group.

As used herein, the term “repeat unit” corresponds to the smallest constitutional unit, the repetition of which constitutes a regular macromolecule (or oligomer molecule or block).

As used herein, the term “end group” refers to a constitutional unit with only one attachment to a polymer chain, located at the end of a polymer. For example, the end group can be derived from a monomer unit at the end of the polymer, once the monomer unit has been polymerized. As another example, the end group can be a part of a chain transfer agent or initiating agent that was used to synthesize the polymer.

As used herein, the term “terminus” of a polymer refers to a constitutional unit of the polymer that is positioned at the end of a polymer backbone.

As used herein, the term “biodegradable” refers to a process that degrades a material via hydrolysis and/or a catalytic degradation process, such as enzyme-mediated hydrolysis and/or oxidation. For example, polymer side chains can be cleaved from the polymer backbone via either hydrolysis or a catalytic process (e.g., enzyme-mediated hydrolysis and/or oxidation).

As used herein, “biocompatible” refers to a property of a molecule characterized by it, or its in vivo degradation products, being not, or at least minimally and/or reparably, injurious to living tissue; and/or not, or at least minimally and controllably, causing an immunological reaction in living tissue. As used herein, “physiologically acceptable” is interchangeable with biocompatible.

As used herein, the term “hydrophobic” refers to a moiety that is not attracted to water with significant apolar surface area at physiological pH and/or salt conditions. This phase separation can be observed via a combination of dynamic light scattering and aqueous NMR measurements. Hydrophobic constitutional units tend to be non-polar in aqueous conditions. Examples of hydrophobic moieties include alkyl groups, aryl groups, etc.

As used herein, the term “hydrophilic” refers to a moiety that is attracted to and tends to be dissolved by water. The hydrophilic moiety is miscible with an aqueous phase. Hydrophilic constitutional units can be polar and/or ionizable in aqueous conditions. Hydrophilic constitutional units can be ionizable under aqueous conditions and/or contain polar functional groups such as amides, hydroxyl groups, or ethylene glycol residues. Examples of hydrophilic moieties include carboxylic acid groups, amino groups, hydroxyl groups, etc.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “peptide” refers to natural biological or artificially manufactured short chains of amino acid monomers linked by peptide (amide) bonds. As used herein, a peptide has at least 2 amino acid repeating units.

As used herein, the term “oligomer” refers to a macromolecule having 10 or less repeating units.

As used herein, the term “polymer” refers to a macromolecule having more than 10 repeating units.

As used herein, the term “polysaccharide” refers to a carbohydrate that can be decomposed by hydrolysis into two or more molecules of monosaccharides.

As used herein, the term “hydrogel” refers to a water-swollen, and cross-linked polymeric network produced by the reaction of one or more monomers. The polymeric material exhibits the ability to swell and retain a significant fraction of water within its structure, but does not dissolve in water.

As used herein, the term “protein” refers to any of various naturally occurring extremely complex substances that consist of amino-acid residues joined by peptide bonds, contain the elements carbon, hydrogen, nitrogen, oxygen, usually sulfur, and occasionally other elements (such as phosphorus or iron), and include many essential biological compounds (such as enzymes, hormones, or antibodies).

As used herein, the term “tissue” refers to an aggregate of similar cells and cell products forming a definite kind of structural material with a specific function, in a multicellular organism.

As used herein, the term “organs” refers to a group of tissues in a living organism that have been adapted to perform a specific function.

As used herein, the term “therapeutic agent” refers to a substance capable of producing a curative effect in a disease state.

As used herein, the term “small molecule” refers to a low molecular weight (<2000 daltons) organic compound that may help regulate a biological process, with a size on the order of 1 nm. Most drugs are small molecules.

As used herein, the term “biomaterial” refers to a natural or synthetic material (such as a metal or polymer) that is suitable for introduction into living tissue, for example, as part of a medical device (such as an artificial joint).

As used herein, the term “ceramic” refers to an inorganic, non-metallic, solid material comprising metal, non-metal or metalloid atoms primarily held in ionic and covalent bonds.

As used herein, the term “composite” refers to a composition material, a material made from two or more constituent materials with significantly different physical or chemical properties that, when combined, produce a material with characteristics different from the individual components. The individual components remain separate and distinct within the finished structure.

As used herein, the term “chelating agent” refers to a ligand that forms two or more separate coordinate bonds to a single central metal ion.

One letter codes for amino acids are used herein. For example, alanine is A, arginine is R, asparagine is N, aspartic acid is D, asparagine or aspartic acid is B, cysteine is C, glutamic acid is E, glutamine is Q, glutamine or glutamic acid is Z, glycine is G, histidine is H, isoleucine is I, leucine is L, lysine is K, methionine is M, phenylalanine is F, proline is P, serine is S, threonine is T, tryptophan is W, tyrosine is Y, valine is V.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:
(1) preventing the disease; for example, preventing a disease, condition or disorder in an individual who may be predisposed to the disease, condition or disorder but does not yet experience or display the pathology or symptomatology of the disease;
(2) inhibiting the disease; for example, inhibiting a disease, condition or disorder in an individual who is experiencing or displaying the pathology or symptomatology of the disease, condition or disorder; and
(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Cyclic Multifunctional Linkers

Incorporation of stimuli-cleavable units within crosslinkers can provide materials that degrade in response to external triggers. The present disclosure features a modular approach to engineering materials with tailored, user-defined, logic-based responsiveness to predetermined stimuli. Without wishing to be bound by theory, it is believed that by controlling the molecular architecture and connectivity of multiple stimuli-labile moieties, materials with computational capacity through hierarchical combinations of Boolean YES/OR/AND gates can be obtained. The simplest Boolean logical function—the YES gate—can implemented when a single stimuli-labile moiety is included in the crosslinker. Materials can be programmed with increased complexity through incorporation of a second stimuli-labile group: when degradable units are connected in series, cleavage of either group can cause material dissolution, forming an OR gate (denoted with logic symbol v); when degradable units are connected in parallel, cleavage of both groups is required to cause material dissolution, forming an AND gate (indicated by logic symbol A). These concepts can be extended hierarchically, combining multiple gates into a logical circuit to engineer complex material responses.

The present disclosure features a cyclic multifunctional linker, including at least two cleavable moieties; at least two connecting chains connected to the at least two cleavable moieties to provide a cyclic structure, and at least two linking groups, each linking group being bonded at one end to a connecting chain and being located between two cleavable moieties, and each linking group having a second end configured to bond to crosslinkable moieties. In the cyclic multifunctional linker, each connecting chain has at least two ends, and at least two of the connecting chains are each connected at each end to a cleavable moiety.

In some embodiments, the cyclic multifunctional linker includes a first connecting chain connecting a first cleavable moiety and a second cleavable moiety, a second connecting chain connecting the second cleavable moiety (or a third cleavable moiety, when the cyclic multifunctional linker has more than 3 cleavable moieties) to a fourth cleavable moiety, and a third connecting chain connecting the fourth cleavable moiety (or a fifth cleavable moiety, when the cyclic multifunctional linker has more than 4 cleavable moieties) to the first cleavable moiety (or a sixth cleavable moiety, when the cyclic multifunctional linker has more than 5 cleavable moieties) to provide a cyclic structure; and at least two linking groups, each linking group being bonded at one end to a connecting chain and being located between two cleavable moieties, and each linking group having a second end configured to bond to crosslinkable moieties. In some embodiments, the second cleavable moiety is connected to the third cleavable moiety, the fourth cleavable moiety is connected to the fifth cleavable moiety, and/or the sixth cleavable moiety is connected to the first cleavable moiety. In some embodiments, the cyclic multifunctional linker includes additional cleavable moieties in the cyclic structure. In some embodiments, the cyclic multifunctional linker has a bicyclic structure, where cleavable moieties are located at each arc in the bicyclic structure, and the cleavable moieties are connected to one another via connecting chains.

The connecting chains in the cyclic multifunctional linkers above can each be linear chains or branched chains. When the connecting chain is branched, it can connect to three or more cleavable moieties, one at each terminus of the connecting chain. The connecting chains can be independently a peptide (linear or branched), a DNA strand (linear or branched), a RNA strand (linear or branched), a polymer (linear or branched), an oligomer (linear or branched), a polysaccharide (linear or branched), and/or any combination thereof.

In some embodiments, the cleavable moieties (e.g., the degradable moieties) are independently selected from a photo-cleavable moiety, an enzyme-cleavable moiety (e.g., a matrix metalloproteinase (MMP)-cleavable moiety), a ribozyme-cleavable moiety, a redox-cleavable moiety, an acid-cleavable moiety, a base-cleavable moiety, a nucleophile-cleavable moiety, an electrophile-cleavable moiety, an organometallic moiety having one or more chelating agents, a double-stranded DNA, a temperature-cleavable moiety, and a hydrolyzable moiety. Examples of cleavable moieties are described, for example, in Leriche et al., Cleavable linkers in chemical biology, Bioorganic & Medicinal Chemistry 20 (2012), 571-582; and in Yang et al., Cleavable Linkers in Chemical Proteomics Applications, Activity-Based Proteomics, Methods in Molecular Biology, volume 149 (2017) Edited by Herman S. Overkleeft and Bogdan I. Florea, Chapter 14, 185-203, herein incorporated by reference in their entireties.

In some embodiments, the multifunctional linker is a small molecule having a molecular weight of 100 Da or more (e.g., 200 Da or more, 500 Da or more, 1000 Da or more) and/or 2000 Da or less (e.g., 1000 Da or less, 500 Da or less, or 200 or less).

The multifunctional linker can be configured to crosslink crosslinkable moieties such as oligomers, DNA, polymers, hydrogels, proteins, peptides, cells, tissues, organs, therapeutic agents, small molecules, particles (e.g., nanoparticles, microparticles), surfaces, biomaterials, ceramics, composites, glass, metals, and any combinations thereof. The multifunctional linker can be bonded to the crosslinkable moieties to provide crosslinked moieties. Each multifunctional linker can independently be connected to the crosslinkable moieties via a non-covalent bond (e.g., (strept)avidin-biotin, Lys donor peptide (KDP)-Gln acceptor peptide (QAP) or -enzyme sensitive peptide (ESP), a covalent bond, an ester, an amide, a triazole, an oxime, and/or an alkyl sulfide. Examples of linkages between the multifunctional linker and the crosslinkable moieties are described, for example, in Zhu Junmin, Bioactive modification of poly(ethylene glycol) hydrogels for tissue engineering, Biomaterials, (2010), 31(17), 4639-4656 and Jiang et al., Click hydrogels, microgels and nanogels: Emerging platforms for drug delivery and tissue engineering, Biomaterials, (2014), 35(18), 4969-4985, each herein incorporated by reference in its entirety.

When the cyclic multifunctional linker is bonded to crosslinkable moieties, the crosslinked moieties provide a material that includes crosslinks formed of the multifunctional linkers interspersed throughout the material, or interspersed in a portion of the material. When exposed to two or more stimuli that are configured to cleave two or more of the cleavable moieties, the multifunctional linker can degrade and thereby release the crosslinked moieties from the material. Degradation of the material can proceed as soon as the two or more cleavable moieties are cleaved when exposed to the two or more stimuli. In some embodiments, exposure to only one stimulus cannot release the crosslinked moieties from the material.

In some embodiments, the two or more stimuli are different. For example, the two or more stimuli can be independently selected from light of a predetermined wavelength (see, e.g., Leriche et al., Cleavable linkers in chemical biology, Bioorganic & Medicinal Chemistry 20 (2012), 571-582, herein incorporated by reference in its entirety), an enzyme (id), a reductant (id), an oxidant (id), a nucleophile (id), an electrophile (id), a chelating agent (see, e.g., Chueh et al., Patterning alginate hydrogels using light-directed release of caged calcium in a microfluidic device, (2010) 12(1), 145-151, herein incorporated by reference in its entirety), a DNA (see, e.g., Frezza et al., Modular Multi-Level Circuits from Immobilized DNA-Based Logic Gates, (2007), 129(48), 14875-14879, herein incorporated by reference in its entirety), pH (see, e.g., Leriche et al., Cleavable linkers in chemical biology, Bioorganic & Medicinal Chemistry 20 (2012), 571-582, herein incorporated by reference in its entirety), water (see, e.g., Zustiak S. P. and Leech J. B., Hydrolytically degradable poly(ethylene glycol) hydrogel scaffolds with tunable degradation and mechanical properties, (2010), 11(5), 1348-1357; Jo et al., Tailoring hydrogel degradation and drug release via neighboring amino acid controlled ester hydrolysis, Soft Matter, (2009), 5, 440-446, each herein incorporated by reference in its entirety), a predetermined temperature, and any combination thereof. In some embodiment, when the stimulus is DNA, a complementary DNA strand can competitively bind, thereby displacing an existing linkage and effecting cleavage (e.g., toe-hold mediated strand displacement). For example, the two or more stimuli can be light having different wavelengths, enzymes that recognize different substrates, reductants and/or oxidants having different strengths, nucleophile and/or electrophile having different strengths, exposure to different pH, water, and/or exposure to different temperatures.

In some embodiments, the cyclic multifunctional linker of the present disclosure has Formula (I):

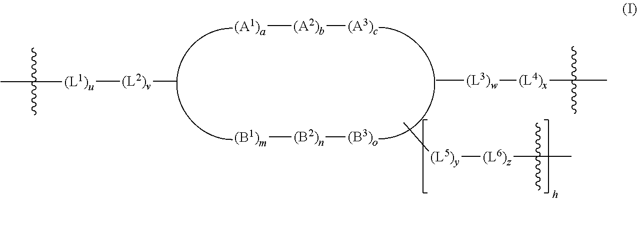

wherein
A¹, A², A³, B¹, B², B³, L¹, L², L³, L⁴, L⁵, and L⁶ are each independently a cleavable moiety configured to cleave when exposed to a stimulus;
a and m are each independently selected from 1, 2, and 3;
b, c, n, and o are each independently selected from 0, 1, 2, and 3;
v and w are each independently selected from 0, 1, and 2;
u and x are each independently selected from 0, 1, and 2;
y is selected from 0, 1, and 2;
z is selected from 0, 1, and 2; and
h is selected from 0, 1, 2, or 3.

In embodiments when a third or additional linking group -(L₅)_{y}-(L₆)_{z}- is present, it is understood that each of L₅ and L₆ can be, at each occurrence, the same or different for each of the linking groups.

It is understood that the -(L₅)_{y}-(L₆)_{z}- can be connected at anywhere on the connecting chain (symbolized by a solid arc in the Formulas of the present disclosure).

In some embodiments, the cyclic multifunctional linker of Formula (I) has Formula (Ia):

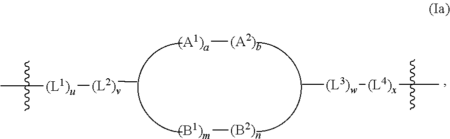

where each of variables A¹, A², B¹, B², L¹, L², L³, L⁴, a, b, m, n, u, v, w, and x are as defined for Formula (I).

In some embodiments, the cyclic multifunctional linker of Formula (I) has Formula (Ib):

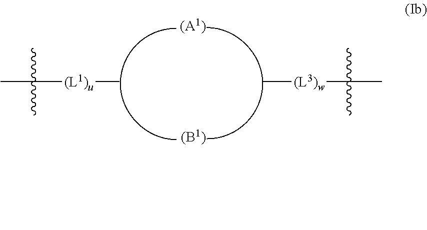

where each of variables A¹, A², u, and w are as defined for Formula (I).

In some embodiments, In-at least one of A¹, A², A³, B¹, B², and B³, when present,

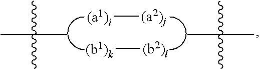

wherein a¹, a², b¹, and b² are each independently a cleavable moiety configured to cleave when exposed to a stimulus;
i and k are each independently 1;
j and 1 are each independently selected from 0 or 1.

In some embodiments, in Formulas (I), (Ia), and (Ib), A¹, A², A³, B¹, B², B³, L¹, L², L³, L⁴, L⁵, L⁶, a¹, a², b¹, and b², when present, are each independently selected from a photo-cleavable moiety, an enzyme-cleavable moiety (e.g., a MMP-cleavable moiety), a ribozyme-cleavable moiety, a redox-cleavable moiety, an acid-cleavable moiety, a base-cleavable moiety, a nucleophile-cleavable moiety, an electrophile-cleavable moiety, an organometallic moiety having one or more chelating agents, a double-stranded DNA, a temperature-cleavable moiety, and a hydrolyzable moiety.

In some embodiments, in Formulas (I), (Ia), and (Ib), A¹, A², A³, B¹, B², B³, L¹, L², L³, L⁴, L⁵, L⁶, a¹, a², b¹, and b², when present, are each independently selected from a photo-cleavable moiety, an enzyme-cleavable moiety (e.g., a MMP-cleavable moiety), a redox-cleavable moiety, an acid-cleavable moiety, a base-cleavable moiety, a nucleophile-cleavable moiety, an electrophile-cleavable moiety, and a hydrolyzable moiety.

In some embodiments, in Formulas (I), (Ia), and (Ib), A¹, A², A³, B¹, B², B³, L¹, L², L³, L⁴, L⁵, L⁶, a¹, a², b¹, and b², when present, are each independently selected from a photo-cleavable moiety, an enzyme-cleavable moiety (e.g., a MMP-cleavable moiety), and a redox-cleavable moiety.

In some embodiments, in Formulas (I), (Ia), and (Ib), A¹, A², A³, B¹, B², B³, L¹, L², L³, L⁴, L⁵, L⁶, a¹, a², b¹, and b², when present, are each independently selected from:

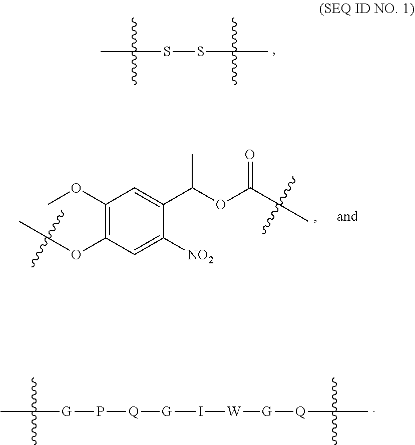

In some embodiments, in Formulas (I), (Ia), and (Ib), A¹, A², A³, B¹, B², B³, L¹, L², L³, L⁴, L⁵, L⁶, a¹, a², b¹, and b², when present, are each independently selected from a MMP-cleavable sequence; a cathepsin-cleavable sequence; an elastase-cleavable sequence; a disulfide moiety; a thioketal moiety; an ortho-nitrobenzyl moiety; a coumarin moiety; a hydrazone moiety; an oxime moiety; an acetal moiety; a silyl ether moiety; and an ester moiety. It is understood that each of A¹, A², A³, B¹, B², B³, L¹, L², L³, L⁴, L⁵, and L⁶ are linking moieties, and as such, are bound at two sites to the cyclic multifunctional linker.

In any of the above-mentioned embodiments, at least A¹ and B¹ can be different (e.g., at least A¹ and B¹ are different). In some embodiments, when a¹ and b¹ are present, a¹ and b¹ are the same. In some embodiments, when a¹ and b¹ are present, a¹ and b¹ are different.

In some embodiments, when present, a and m are each independently selected from 1 and 2 (e.g., a and m are each independently 1); b, c, n, and o are each independently selected from 0, 1, and 2 (e.g., b, c, n, and o are each independently selected from 0 and 1, or are each 0); v and w are each independently selected from 0 and 1 (e.g., v and w are each 0); u and x are each independently selected from 0 and 1 (e.g., u and x are each 0); y is selected from 0 and 1 (e.g., y is 0); z is selected from 0 and 1 (e.g., z is 0); h is selected from 0, 1, and 2 (e.g., h is 0 or 1; or h is 0); and/or j and 1 are each independently 0.

In some embodiments, the cyclic multifunctional linker has Formula (II):

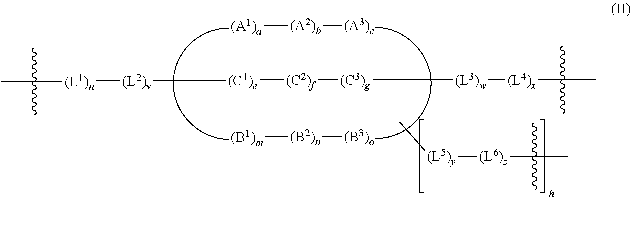

wherein:
A¹, A², A³, B¹, B², B³, C¹, C², C³, L¹, L², L³, L⁴, L⁵, and L⁶ are each independently a cleavable moiety configured to cleave when exposed to a stimulus;
a, e, and m are each independently selected from 1, 2, and 3;
b, c, f, g, n, and o are each independently selected from 0, 1, 2, and 3;
v and w are each independently selected from 0, 1, and 2;
u and x are each independently selected from 0, 1, and 2;
y is selected from 0, 1, and 2;
z is selected from 0, 1, and 2; and
h is selected from 0, 1, 2, or 3.

In embodiments, for cyclic multifunctional linkers of Formula (II), when a third (h is 1) or additional linking group -(L₅)_{y}-(L₆)_{z}- (h is greater than 1) is present, it is understood that each of L₅ and L₆ can be, at each occurrence, the same or different for each of the linking groups. It is also understood that the -(L₅)_{y}-(L₆)_{z}- can be connected at anywhere on the connecting chain (symbolized by a solid arc in the Formulas of the present disclosure).

In some embodiments, the cyclic multifunctional linker has Formula (III):

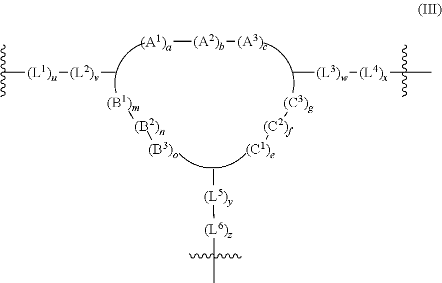

wherein:
A¹, A², A³, B¹, B², B³, C¹, C², C³, L¹, L², L³, L⁴, L⁵, and L⁶ are each independently a cleavable moiety configured to cleave when exposed to a stimulus;
a, e, and m are each independently selected from 1, 2, and 3;
b, c, f, g, n, and o are each independently selected from 0, 1, 2, and 3;
v, w, and y are each independently selected from 0, 1, and 2; and
u, x, and z are each independently selected from 0, 1, and 2.

In some embodiments, exemplary cyclic multifunctional linkers have any one of the Formulas below:

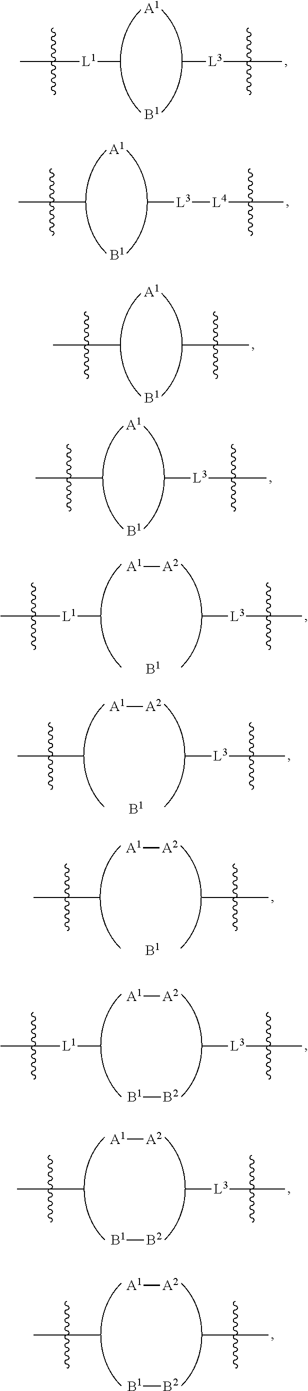

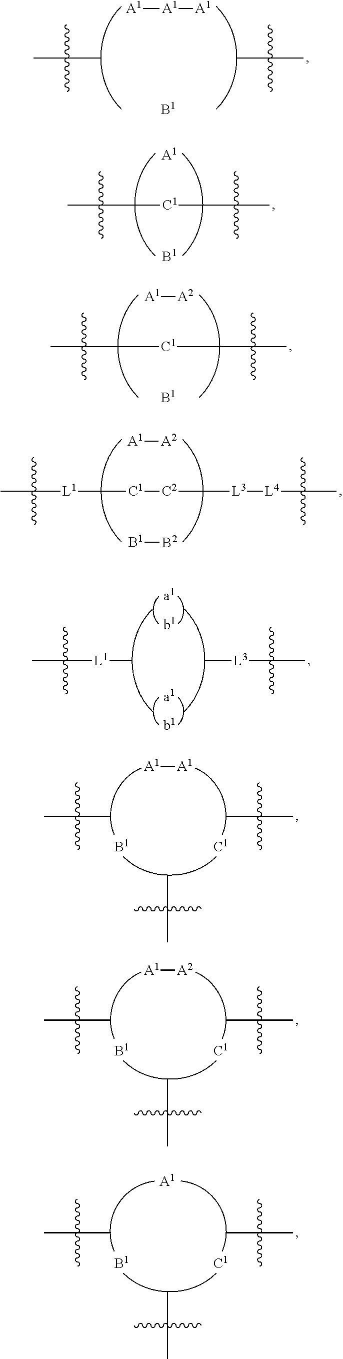

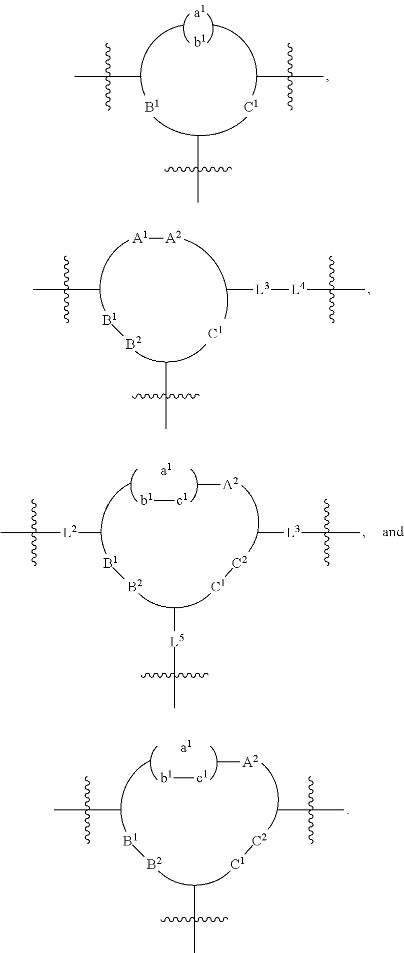

In some embodiments, at least one of A¹, A², A³, B¹, B², B³, C¹, C², C³ when present, is

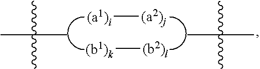

wherein a¹, a², b¹, and b² are each independently a cleavable moiety configured to cleave when exposed to a stimulus;
i and k are each independently 1; and
j and l are each independently selected from 0 or 1.

In some embodiments, a¹, a², b¹, and b² are each independently selected from a photo-cleavable moiety, an enzyme-cleavable moiety (e.g., a MMP-cleavable moiety), a ribozyme-cleavable moiety, a redox-cleavable moiety, an acid-cleavable moiety, a base-cleavable moiety, a nucleophile-cleavable moiety, an electrophile-cleavable moiety, an organometallic moiety having one or more chelating agents, a double-stranded DNA, a temperature-cleavable moiety, and a hydrolyzable moiety.

In some embodiments, for cyclic multifunctional linkers of Formulas (II) and (III), A¹, A², A³, B¹, B², B³, C¹, C², C³, L¹, L², L³, L⁴, L⁵, L⁶, a¹, a², b¹, and b², when present, are each independently selected from a photo-cleavable moiety, an enzyme-cleavable moiety (e.g., a MMP-cleavable moiety), a ribozyme-cleavable moiety, a redox-cleavable moiety, an acid-cleavable moiety, a base-cleavable moiety, a nucleophile-cleavable moiety, an electrophile-cleavable moiety, an organometallic moiety having one or more chelating agents, a double-stranded DNA, a temperature-cleavable moiety, and a hydrolyzable moiety. At least A¹ and B¹ can be different. In some embodiments, at least A¹, B¹, and C¹ are different.

In some embodiments, for cyclic multifunctional linkers of Formulas (II) and (III), A¹, A², A³, B¹, B², B³, C¹, C², C³, L¹, L², L³, L⁴, L⁵, L⁶, a¹, a², b¹, and b², when present, are each independently selected from a photo-cleavable moiety, an enzyme-cleavable moiety (e.g., a MMP-cleavable moiety), a redox-cleavable moiety, an acid-cleavable moiety, a base-cleavable moiety, a nucleophile-cleavable moiety, an electrophile-cleavable moiety, and a hydrolyzable moiety.

In some embodiments, for cyclic multifunctional linkers of Formulas (II) and (III), A¹, A², A³, B¹, B², B³, C¹, C², C³, L¹, L², L³, L⁴, L⁵, L⁶, a¹, a², b¹, and b², when present, are each independently selected from a photo-cleavable moiety, an enzyme-cleavable moiety (e.g., a MMP-cleavable moiety), and a redox-cleavable moiety.

In some embodiments, for cyclic multifunctional linkers of Formulas (II) and (III), A¹, A², A³, B¹, B², B³, C¹, C², C³, L¹, L², L³, L⁴, L⁵, L⁶, a¹, a², b¹, and b², when present, are each independently selected from a MMP-cleavable sequence; a cathepsin-cleavable sequence; an elastase-cleavable sequence; a disulfide moiety; a thioketal moiety; an ortho-nitrobenzyl moiety; a coumarin moiety; a hydrazone moiety; an oxime moiety; an acetal moiety; a silyl ether moiety; and an ester moiety. It is understood that each of A¹, A², A³, B¹, B², B³, L¹, L², L³, L⁴, L⁵, and L⁶ are linking moieties, and as such, are bound at two sites to the cyclic multifunctional linker.

In some embodiments, for cyclic multifunctional linkers of Formulas (II) and (III), A¹, A², A³, B¹, B², B³, C¹, C², C³, L¹, L², L³, L⁴, L⁵, L⁶, a¹, a², b¹, and b², when present, are each independently selected from:

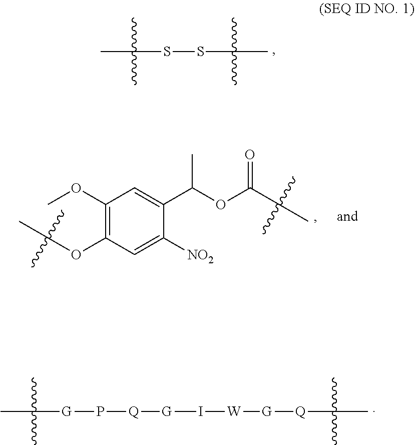

In some embodiments, for cyclic multifunctional linkers of any one of Formulas (I), (Ia), (Ib), (II), and (III), A¹, A², A³, B¹, B², B³, C¹, C², C³, L¹, L², L³, L⁴, L⁵, L⁶, a¹, a², b¹, and b², when present, is selected from

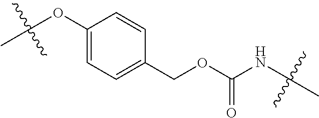 [SMILES: *NC(=O)OCc1ccc(O*)cc1]

(cleavable using trifluoroacetic acid (TFA)),

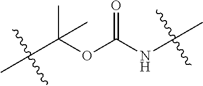 [SMILES: *NC(=O)OC(*)(C)C]

(cleavable using TFA),

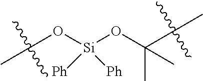 [SMILES: *O[Si](OC(*)(C)C)(c1ccccc1)c1ccccc1]

(Ph is phenyl, cleavable using HCOOH, for example 10% HCOOH),

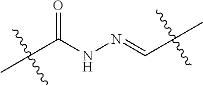 [SMILES: */C=N/NC(*)=O]

(cleavable at pH 4-5, acyl hydrazine),

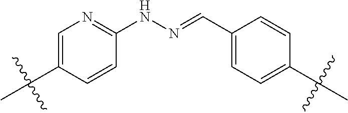 [SMILES: *c1ccc(/C=N/Nc2ccc(*)cn2)cc1]

(cleavable using NH₂OH, aniline at pH 4-5),

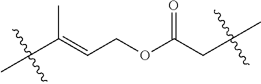 [SMILES: *CC(=O)OC/C=C(/*)C]

(cleavable at pH 11),

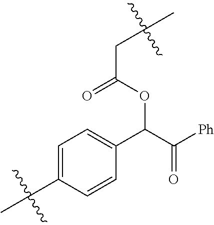 [SMILES: *CC(=O)OC(C(=O)c1ccccc1)c1ccc(*)cc1]

(cleavable at pH 8),

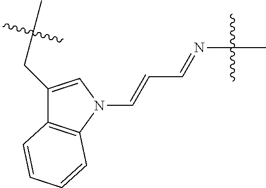 [SMILES: *Cc1cn(/C=C/C=N/*)c2ccccc12]

(cleavable with 100 mN pyrrolidine),

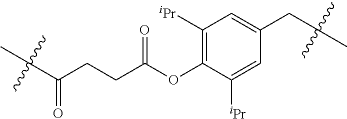 [SMILES: *Cc1cc(C(C)C)c(OC(=O)CCC(*)=O)c(C(C)C)c1]

(cleavable using 100 mM NH₂NH₂),

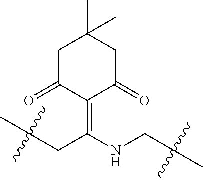 [SMILES: *CNC(C*)=C1C(=O)CC(C)(C)CC1=O]

(cleavable using 2% NH₂NH₂),

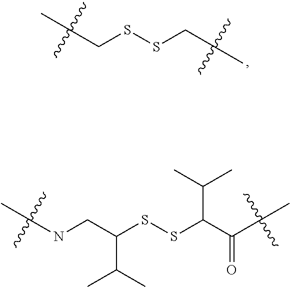 [SMILES: *CSSC* ; *NCC(SSC(C(*)=O)C(C)C)C(C)C]

(cleavable using reducing agents such as dithiothreitol (DTT), (tris(2-carboxyethyl)phosphine (TCEP), or 2-mercaptoethanol (BME)),

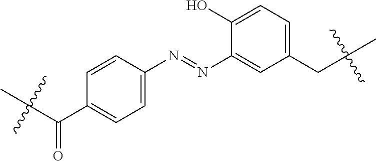 [SMILES: *Cc1ccc(O)c(/N=N/c2ccc(C(*)=O)cc2)c1]

(cleavable using reducing agents such as 10 mM TCEP),

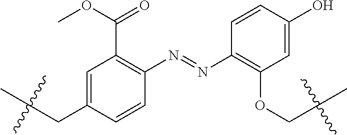 [SMILES: *COc1cc(O)ccc1/N=N/c1ccc(C*)cc1C(=O)OC]

(cleavable using 1 mM Na₂S₂O₄),

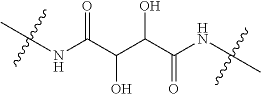 [SMILES: *NC(=O)C(O)C(O)C(=O)N*]

(cleavable using 10 mM NaIO₄),

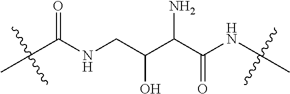 [SMILES: *NC(=O)C(N)C(O)CNC(*)=O]

(cleavable using 10 mM NaIO₄),

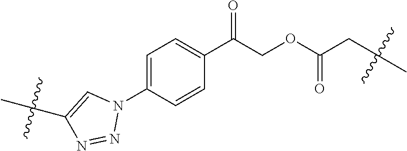 [SMILES: *CC(=O)OCC(=O)c1ccc(-n2cc(*)nn2)cc1]

(cleavable using 254 nm light),

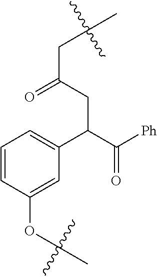 [SMILES: *CC(=O)CC(C(=O)c1ccccc1)c1cccc(O*)c1]

(cleavable using 365 nm light),

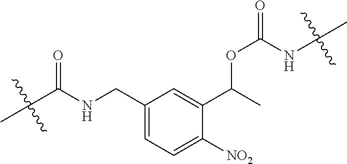 [SMILES: *NC(=O)OC(C)c1cc(CNC(*)=O)ccc1[N+](=O)[O-]]

(cleavable using 365 nm light),

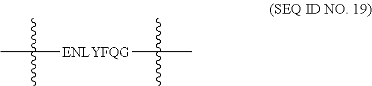

(cleavable using TEV protease),
-5′-GTAACGATCCAGCTGTCACT-3′- (SEQ ID NO. 20) (GTAC are guanine, thymine, adenine, and cytosine, respectively; cleavable using endonuclease),

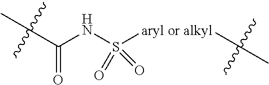

(cleavable using ICH₂CN, pH 9, NH₄OH, or pH 5, ICH₂COO⁻, and

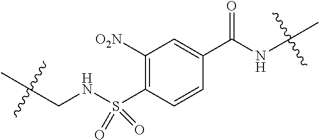 [SMILES: *CNS(=O)(=O)c1ccc(C(=O)N*)cc1[N+](=O)[O-]]

(cleavable using thiols at pH 9.2).

In some embodiments, for cyclic multifunctional linkers of any one of Formulas (I), (Ia), (Ib), (II), and (III), A¹, A², A³, B¹, B², B³, C¹, C², C³, L¹, L², L³, L⁴, L⁵, L⁶, a¹, a², b¹, and b², when present, is selected from enzyme cleavable groups such as TEV, trypsin, thrombin, cathepsin B, cathepsin D, cathepsin K, caspase 1, matrix metalloproteinase sequences, phosphodiester, phospholipid, ester, and b-galactose; nucleophile or base-cleavable groups such as dialkyl dialkoxysilane, cyanoethyl group, sulfone, ethylene glycolyl disuccinate, 2-N-acyl nitrobenzenesulfonamide, α-thiophenylester, unsaturated vinyl sulfide, sulfonamide after activation, malondialdehyde (MDA)-indole derivative, levulinoyl ester, hydrazone, acylhydrazone, alkyl thioester; reducing agent-cleavable groups such as disulfide bridges, azo compounds; photo-irradiation cleavable groups such as 2-nitrobenzyl derivatives, phenacyl ester, 8-quinolinyl benzenesulfonate, coumarin, phosphotriester, bis-arylhydrazone, bimane bi-thiopropionic acid derivative; electrophile or acid-cleavable groups such as paramethoxybenzyl derivative, tert-butylcarbamate analogue, dialkyl or diaryl dialkoxysilane, orthoester, acetal, aconityl, hydrazone, b-thiopropionate, phosphoramidate, imine, trityl, vinyl ether, polyketal, alkyl 2-(diphenylphosphino)benzoate derivatives; organometallic and metal catalyst cleavable groups such as allyl ester, 8-hydroxyquinoline ester, picolinate ester; and oxidizing reagent-cleavable groups such as vicinal diols, and selenium compounds.

In some embodiments, for cyclic multifunctional linkers of Formulas (II) and (III), at least A¹, B¹, and C¹ can be different (e.g., at least A¹, B¹, and C¹ are different). In some embodiments, when a¹ and b¹ are present, a¹ and b¹ are the same. In some embodiments, when a¹ and b¹ are present, a¹ and b¹ are different.

As will be shown in Example 1 below, three distinct inputs (e.g., enzyme, redox, and light) were used to exhaustively generate seventeen unique stimuli-responsive materials, which exhibited degradation in accordance with their environment and logical function, demonstrating high-fidelity biocomputation. Sequential and spatiotemporally controlled delivery of both small molecules and cells was achieved from these materials. Example 2 describes the programmable logic-based delivery of small molecule therapeutics from gels. Example 3 describes independent logic-based delivery of site-specifically-modified proteins from gels.

### EXAMPLES

### Example 1. Engineering Modular Biomaterial Logic Gates for Environmentally-Triggered Therapeutic Delivery

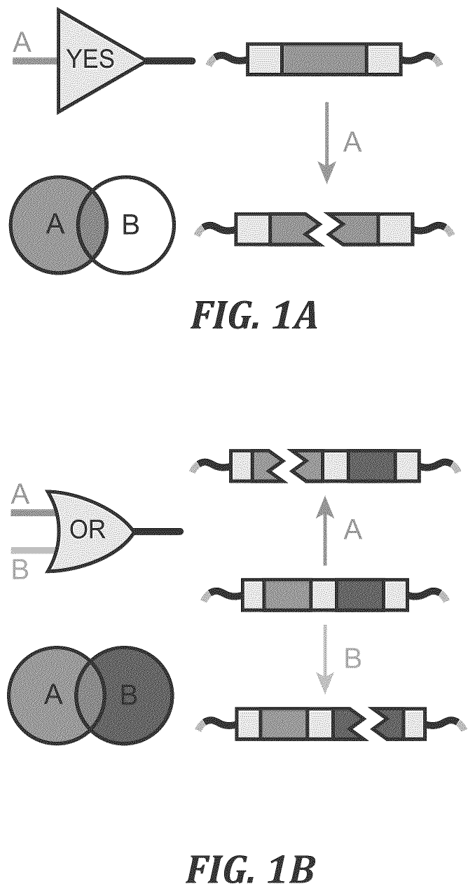

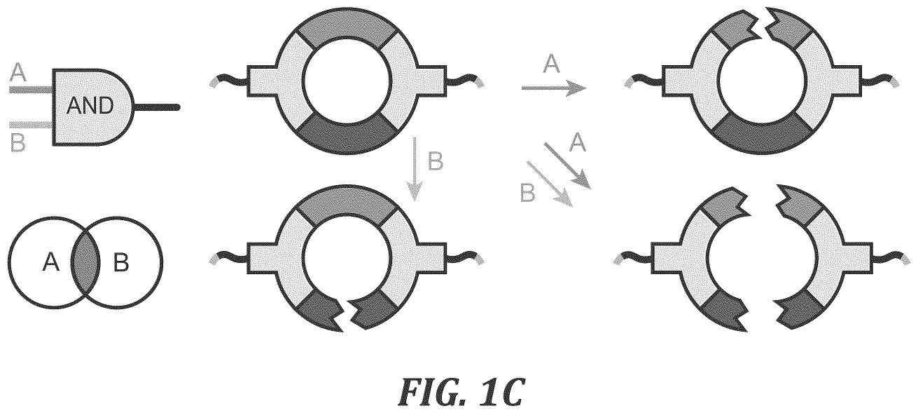

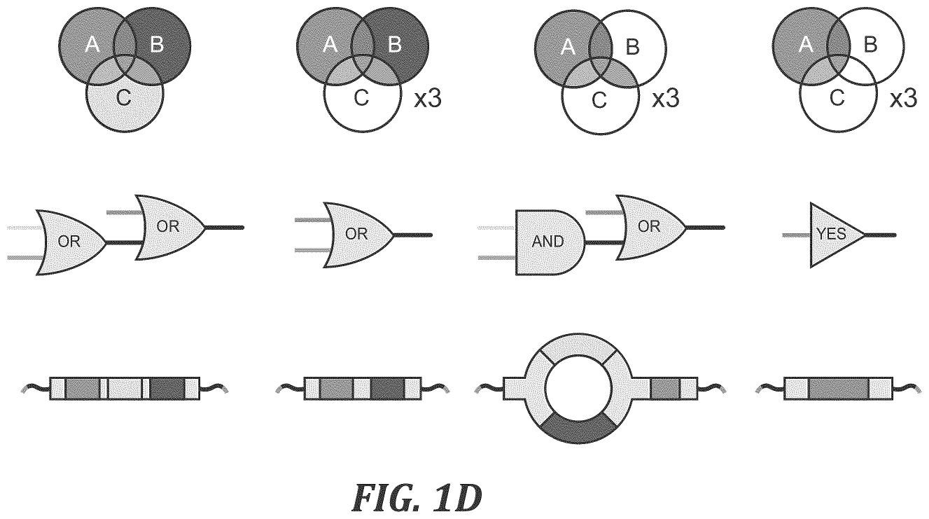

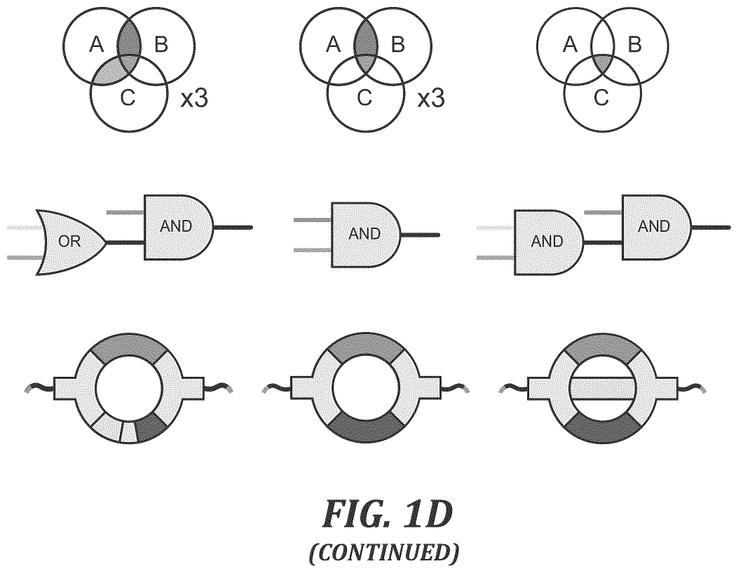

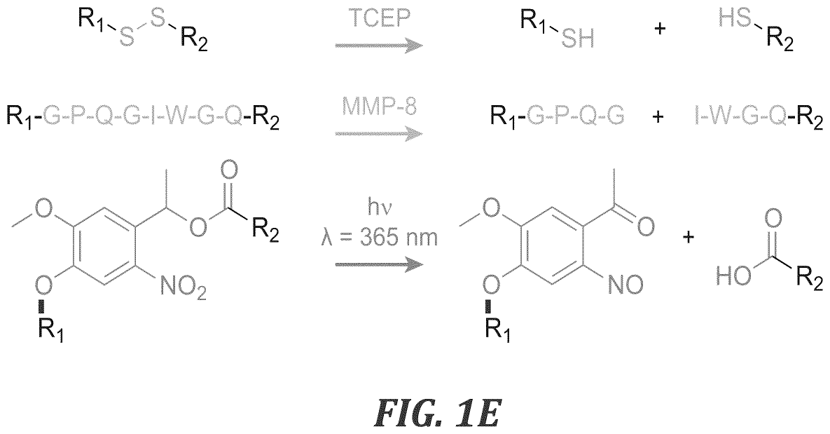

In a rational design-based approach, stimuli-sensitive components are incorporated into discrete, monodisperse, synthetic crosslinkers that, upon reaction with polymer macromers, form hydrogels of well-defined molecular architecture. Information governing the environmental-responsiveness of the resulting material is embedded within the crosslinker domain; when the linker is covalently cleaved, the material degrades and simultaneously releases any encapsulated or tethered payload. The simplest Boolean logical function, the YES-gate, is implemented when a single stimuli-labile moiety is included in the linker. More advanced logical operations could be built through the controlled connectivity of additional cleavable groups within a crosslinker. When two degradable units are connected in series, the cleavage of either moiety causes material dissolution, forming an OR-gate (denoted with logic symbol v); when two degradable units are connected in parallel, the cleavage of both moieties is required for material dissolution, forming an AND-gate (denoted by logic symbol A). These concepts can be expanded hierarchically, combining multiple gates into a logical circuit to engineer complex responses to additional dynamic stimuli (FIGS. 1A-1E). Formalizing the relationship between crosslinker architecture and hydrogel degradability provides a template for creating materials that are structurally simple yet functionally complex.

### Synthesis of Logic-Based Responsive Crosslinkers

Implementation of the outlined biocomputational strategy requires precise control over crosslinker functionality and architecture. Peptide-based crosslinkers were used as a proof of principle due to the efficiency of solid-phase peptide synthesis in generating monodisperse macromolecules that contain a range of functional groups with sequence-defined order and connectivity. Peptides, which possess intrinsic biocompatibility, can be chemically modified to introduce non-canonical functionality, connectivity (e.g., branching, cyclization, intramolecular stapling), and degradability. As a demonstration of this logic-based approach, three chemically orthogonal stimuli-labile moieties from different reaction classes were employed: (i) the enzymatically degradable oligopeptide sequence, GPQG↓IWGQ (SEQ ID NO. 1), which cleaves in the presence of MMPs and allows for cell- and disease-triggered response; (ii) disulfide bonds, which degrade under reducing conditions present both intracellularly and in disease states; and (iii) an ortho-nitrobenzyl ester (oNB), which undergoes photoscission upon cytocompatible near-UV light exposure (λ=365 nm), thereby facilitating user-defined spatiotemporal control over material properties (FIG. 1E). Exhaustively spanning all hierarchical YES/OR/AND combinations of these three stimuli-labile moieties, seventeen distinct crosslinkers that each exhibited a unique logical output (Methods S1-S21, below) were synthesized. Each crosslinker was flanked with two reactive azide moieties to enable formation of nearly ideal step-growth hydrogel networks via a strain-promoted, azide-alkyne cycloaddition (SPAAC) reaction with four-arm poly(ethylene glycol) tetrabicyclononyne (PEG-tetraBCN, Method S22). SPAAC click chemistry rapidly produces homogenous hydrogels in a bioorthogonal fashion, thereby permitting encapsulation of bioactive therapeutics and living cells. Moreover, an extensive toolbox of SPAAC-compatible modifications allows for uniform network functionalization with moieties ranging from small molecules to full-length proteins. Such tunability further enables the design of complex delivery vehicles, for example, through the inclusion of targeting moieties, instructive cues to guide encapsulated cell fate and function, or tethered therapeutics to be released upon material dissolution.

### Assessing Solution-Based Crosslinker Degradation in Response to Environmental Stimuli

To demonstrate that crosslinkers degrade as engineered in response to environmental cues and that stimuli-responsive reactions are chemically orthogonal, each of the one- and two-input linkers were treated with every possible combination of MMP enzyme (_{E}), reducing components (_{R}), and light (_{P}) (Methods S23 and S24). Reaction products were characterized using matrix-assisted laser desorption/ionization time-of-flight mass spectrometry (MALDI-TOF). Detected masses were in excellent agreement with those of the expected reaction products, indicating that the linkers respond as designed on a molecular level. To further investigate, the enzyme AND photo linker (E∧P) was pretreated with different combinations of enzyme and light, added to a stoichiometrically defined amount of PEG-tetraBCN, and characterized by in situ oscillatory rheology to monitor evolution of material properties. Untreated E∧P yielded robust gels, demonstrating the first successful use of a cyclic or stapled peptide for material crosslinking. Final storage moduli of samples containing the untreated linker (G′=1660±170 Pa) were similar to that of the linkers subjected to either enzyme or light (G′=1580±130 Pa and 1540±110 Pa, respectively), while the linker treated with both enzyme and light did not form a gel (G′=200±30). All samples had a final loss modulus (G″) of ˜50 Pa. Consistent with rubber elasticity theory where shear moduli scales with crosslinking density and calculations that distances between network branch points increase ≤3% upon cleavage of a single arm of AND-gated linkers (Method S26), this data suggests that the mechanical properties of these materials depend only on the final logical state of the Boolean linker.

### Logic-Based Hydrogel Degradation in Response to Environmental Stimuli

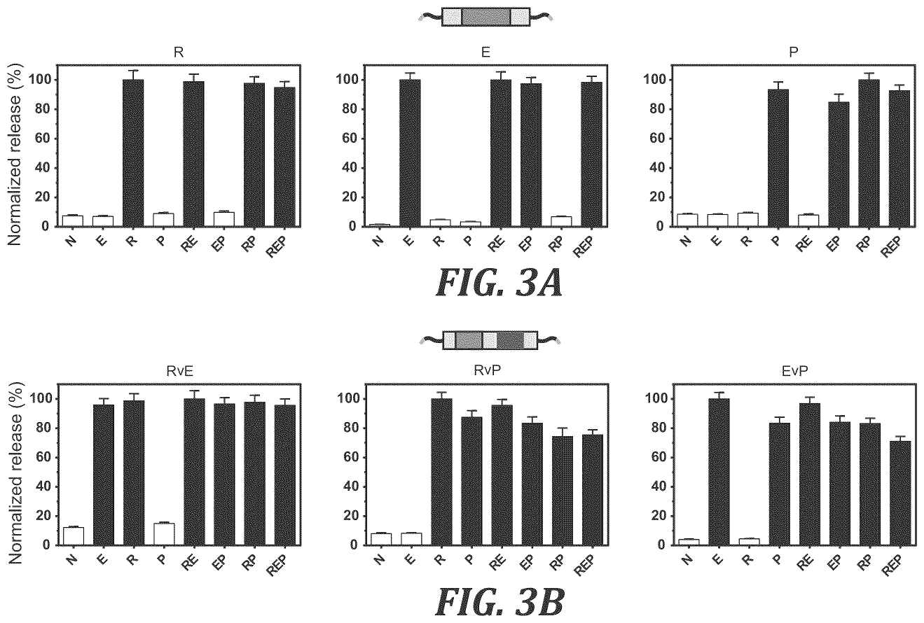

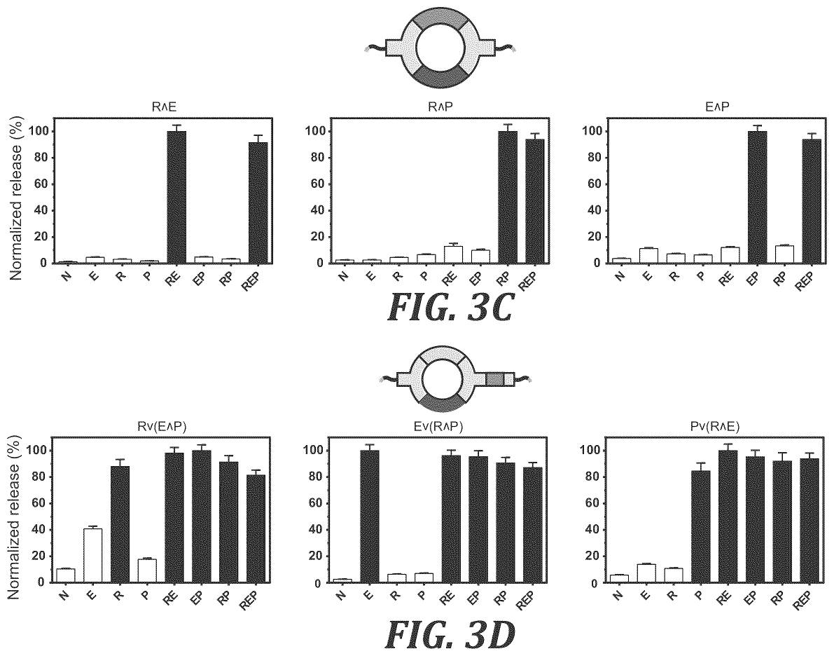

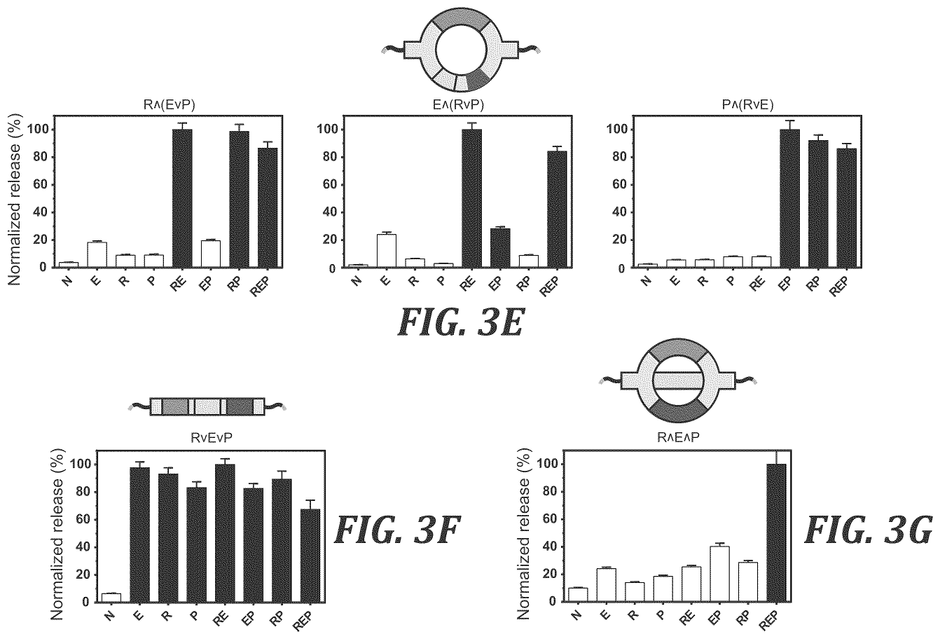

After validating linker behavior on the molecular level, we sought to characterize the logic-based stimuli-responsiveness of bulk materials. Each crosslinker was reacted independently with Alexa568®-labeled PEG-tetraBCN to form seventeen different types of fluorescent hydrogels. For each type, responsiveness to all eight input combinations involving reducing agents, light, and enzyme were evaluated. Hydrogel degradation was quantified by measuring supernatant fluorescence at non-kinetically limited endpoints following treatment (FIGS. 3A-3F, Methods S27 and S28). Each of the YES-gated materials (E, R, and P) behaved as expected, degrading only when the programmed cue was present. The high selectivity (>10-fold over non-specific release) again demonstrates the orthogonality of the employed stimuli-labile chemistries. The OR-gated materials (R∨E, E∨P, R∨P) also responded as expected, degrading fully when either of the relevant cues was present. The AND-gated materials (R∧E, E∧P, R∧P) also functioned properly, fully degrading only when both programmed cues were present. The observed release selectivity (>7-fold) is as or more specific than the most successful dual-input degradable materials previously reported. Of the three-input materials containing two logic gates, six of eight [i.e., E∨(R∧P), P∨(R∧E), R∧(E∨P), P∧(R∨E), R∨E∨P, R∨(E∧P)] behaved fully as designed, degrading with high selectivity only when the respective cues were present. The conditions [E∧(R∨P)]_{EP} and (R∧E∧P)_{REP} did not fully degrade, which were attributed to known decreased proteolytic cleavage kinetics for strained MMP-degradable substrates, in this case due to internal ring strain. These higher-order, three-input crosslinkers are the most complex logical operators ever used to control material degradation. This generalizable approach proves robust as 132 of the 136 treatment conditions yielded engineered degradation (defined as either complete degradation or <30% nonspecific release). The exhaustive synthesis and testing of each possible material demonstrates that complex biomaterial computation can be achieved with high fidelity through the hierarchical combination of simple YES/OR/AND logic gates. Given the initial success of this modular framework, the chosen stimuli-labile groups can be substituted with any number of other chemically orthogonal moieties sensitive to pH, additional proteases, visible light, temperature, or ultrasound.

### Disease-Associated Delivery of Doxorubicin to an In Vitro Cancer Model

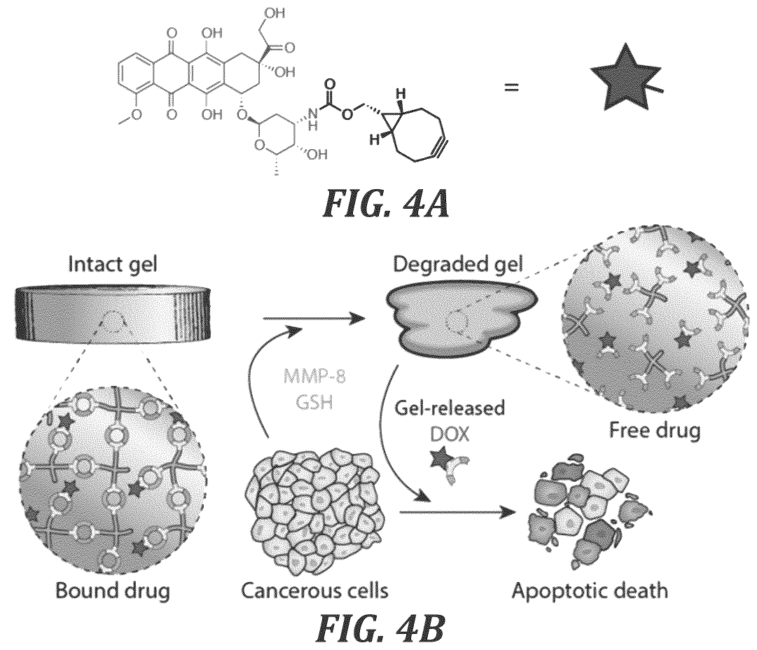

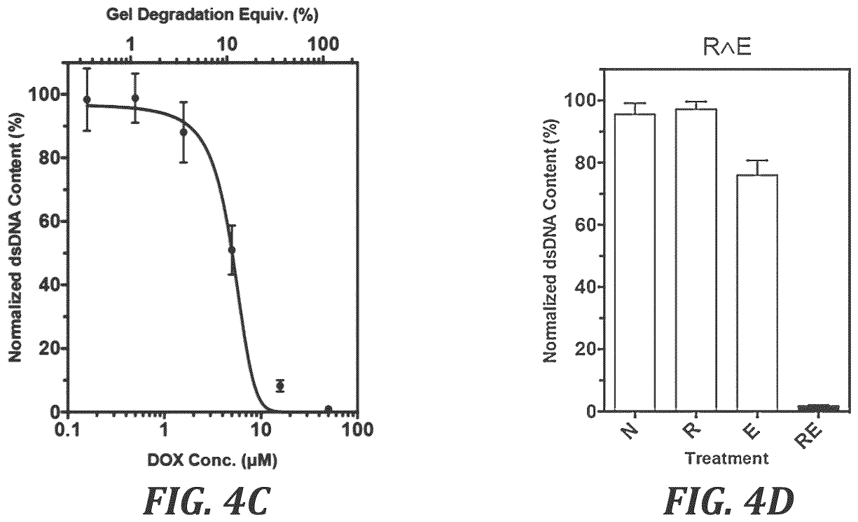

To demonstrate the ability to deliver functional therapeutics in response to precise combinations of pathophysiological stimuli, a BCN-tagged doxorubicin (DOX) chemotherapeutic was tethered into R∧E gels that degrade with high specificity to cancer microenvironmental cues (FIGS. 4A and 4B, Method S29). Extent of hydrogel functionalization was chosen such that solution DOX concentration following full material degradation (44 μM) would yield population-wide apoptotic death of plated cervical cancer-derived HeLa cells (FIG. 4C). Following treatment by each relevant input combination (i.e., _{N}, _{E}, _{R}, _{RE}), cells were incubated in hydrogel supernatants for 48 hours prior to quantitative analysis of double-stranded DNA (dsDNA) content, indicative of the total number of viable cells. In the absence of treatment, or that with just reductant or MMP, normal proliferation was observed (95±3%, 97±2%, and 76±5%, respectively, relative to non-treated controls lacking gels). The slight decrease in total dsDNA content following enzymatic treatment is attributed to secondary effects of the MMP treatment, rather than to non-specific DOX release (Method S29). In stark contrast to treatments with a single input, treatment with both inputs resulted in complete cell eradication (1.8±0.2% dsDNA content relative to controls), as designed. These results highlight the unique capacity of this approach to control release of functional small molecule therapeutics through logic-based gel degradation, enabling precise regulation of cell fate in response to disease-defined combinations of external cues.

Logic-Based Delivery of Live Cells from Stimuli-Responsive Hydrogels

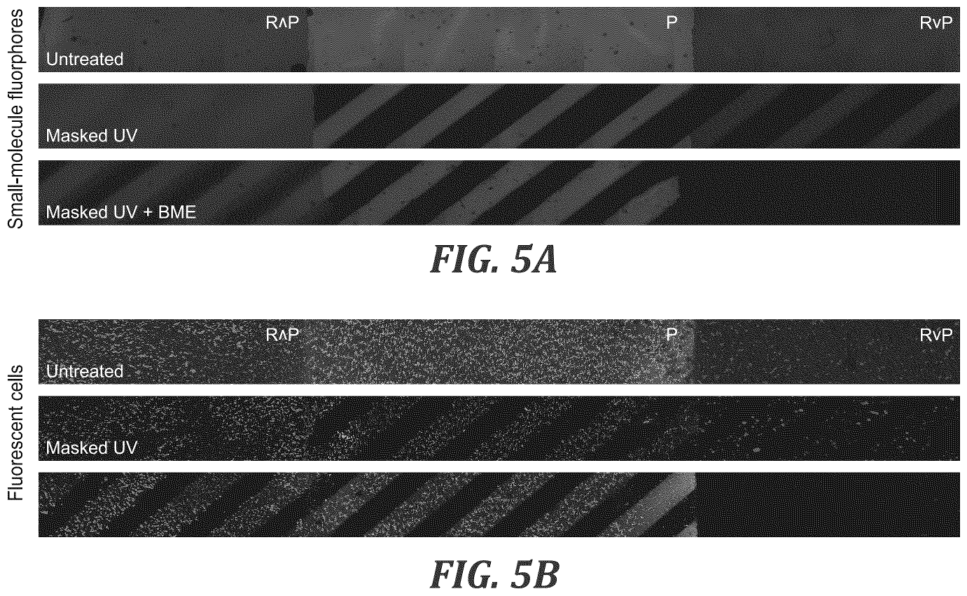

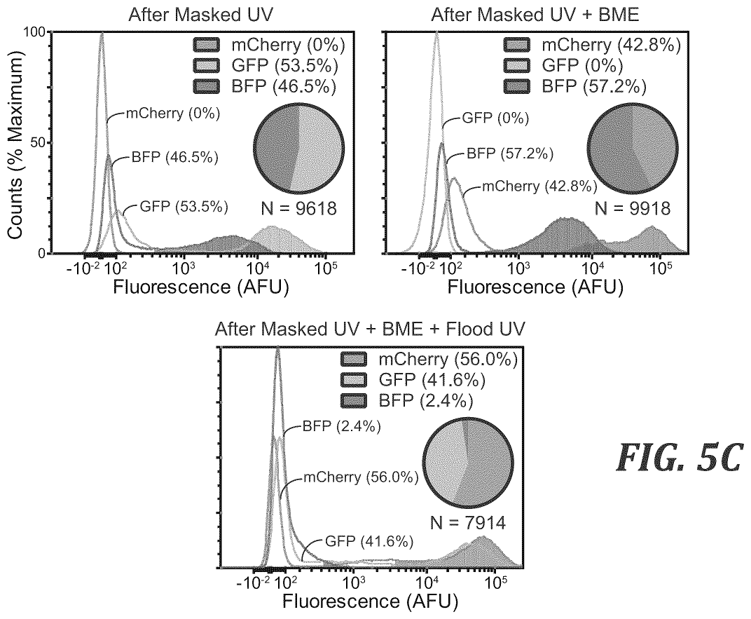

To illustrate the biocomputational response of these engineered materials to a combination of spatially defined as well as environmental cues, a multifunctional hydrogel comprised of three distinct logical regions (R∧P, P, R∨P), each labeled with a different fluorophore (FIGS. 5A-5C) was formulated. These hydrogels were sequentially exposed to masked UV light and reducing conditions, and imaged via fluorescent confocal microscopy (Method S30). Each region responded to external cues as engineered, degrading only when the proper set of input conditions had been presented. To demonstrate cytocompatible gelation and multi-stimuli-responsive degradation, an analogous experiment was performed with each region containing encapsulated hS5 bone marrow-derived stromal cells that constitutively express a different fluorescent protein. Cells were released from gels following sequential masked light exposure, reducing conditions, and flood illumination, harvested after each treatment, and analyzed by flow cytometry. Each treatment yielded a distinct cell collection matching the expected color composition (Method S31). Encapsulated cells were also shown to be viable when released through each stimulus, demonstrating whole process cytocompatibility. This material system, which yields sequential and environmentally triggered release of multiple cell lines in well-defined combinations, represents the most advanced live-cell delivery platform to date.

### DISCUSSION

Although a logic-gated approach to control biomaterial degradation using SPAAC-based PEG hydrogels that respond to reductant, enzyme, and light inputs is implemented above, these general methodologies are readily extendable to different stimuli-labile moieties, polymer compositions, and gelation chemistries. For example, these logic-based strategies can be extended to covalently tether other small molecules, peptides, proteins, polysaccharides, and nucleic acids to a non-degradable hydrogel via a stimuli-responsive linker, affording precise biochemical presentation through environmentally triggered controlled release of bioactive species.

Another potential benefit of the present approach stems from the ability to tailor the “propagation delay”—the time required to transduce input signals into the appropriate functional output—of the Boolean operator for different therapeutic applications. For these logic-based materials, gate delay is governed by the susceptibility of each labile region to its relevant input, overall construct size/geometry, and the concentrations of the environmental cues triggering degradation. The degradation rate of a linkage to a given input may be tuned over several orders of magnitude, for example by modifying substituents on photodegradable groups or substituting single amino acids within enzyme-labile peptide sequences. The propagation delay can be further decreased by formulating materials into geometries where response is reaction-limited rather than diffusion-limited. Careful choice of construct geometry and stimuli-labile group identity enables user-specified control over material response rates.

Here a modular approach was introduced to engineer materials with tailored, user-specified, logic-based responsiveness to environmental cues. By controlling the molecular architecture and connectivity of multiple stimuli-labile moieties within discrete peptide-based crosslinkers, biomaterials have been endowed with unprecedented computational capacity through hierarchical combinations of Boolean YES/OR/AND gates. Having exhaustively synthesized crosslinkers that are each uniquely sensitive to combinations of three orthogonal inputs (i.e., enzyme, reduction, light), constructs that exhibit expected behavior spanning molecular and macroscopic scales are demonstrated. These platforms demonstrate sequential and spatiotemporally varied delivery of multiple cell lines from a single gel, as well as the controlled release of a functional chemotherapeutic in response to disease-associated cues. These platforms can find great utility in targeted drug delivery, where release of therapeutics, proteins, and cells can be confined to sites of disease with high selectivity, as well for applications in diagnostics, tissue engineering, and regenerative medicine.

### Methods

### General Synthetic Information

Chemical reagents and solvents were purchased from either Sigma-Aldrich or Fisher Scientific and used as received. Peptide synthesis reagents were purchased from either ChemPep or Chem-Impex and used as received. Deionized water (dH₂O) was generated by a U.S. Filter Corporation Reverse Osmosis System with a Desal membrane. Synthetic chemical reactions were performed under a nitrogen atmosphere in oven-dried glassware and stirred with a Teflon-coated magnetic stir bar unless otherwise noted. Solvents were removed in vacuo with a Büchi Rotovapor R-3 equipped with a V-700 vacuum pump and V-855 vacuum controller and a Welch 1400 DuoSeal Belt-Drive high vacuum pump. ¹H and ¹³C nuclear magnetic resonance (NMR) data was collected at 298 K on Bruker instruments and chemical shifts are reported relative to tetramethylsilane (TMS, δ=0). Microwave-assisted peptide synthesis was performed on a CEM Liberty 1. Semi-preparative reversed-phase high-pressure liquid chromatography (RP-HPLC) was performed on a Dionex Ultimate 3000 equipped with a variable multiple wavelength detector, automated fraction collector, and Thermo 5 μm Synchronis silica 250×21.2 mm C18 column. Lyophilization was performed on a LABCONCO FreeZone 2.5 Plus freeze-dryer equipped with a LABCONCO rotary vane 117 vacuum pump. Matrix-assisted laser desorption/ionization time of flight (MALDI-TOF) mass spectrometry was performed in reflectron positive ion mode on a Bruker AutoFlex II using a matrix of α-cyano-4-hydroxycinnamic acid:2,5-dihydroxy benzoic acid (2:1). High-resolution mass spectrometry (HRMS) was performed on a Thermo Linear Trap Quadrupole Orbitrap Xclaibur 2.0 DS. The light source for the photochemical cleavage was a Lumen Dynamics OmniCure S1500 Spot UV Curing system with an internal 365 nm filter and an external 360 nm cut-on longpass filter. Light intensity was measured using a Cole-Parmer Radiometer (Series 9811-50, λ=365 nm). Fluorescence readings were acquired on a SpectraMax M5 spectrometer using Thermo Scientific Nunc black polypropylene 96-well plates. Rheological measurements were performed on an Anton Paar MCR301 equipped with a C-PTD200 Peltier plate and a CP25-1 cone and plate geometry. Fluorescent microscopy was performed on a Nikon Eclipse TE2000-U. Confocal microscopy was performed at the University of Washington Keck Microscopy Center on a Leica SP8X. Polymerase chain reaction (PCR) was performed in a Bioer LifeECO thermal cycler. Protein expression was performed in a Thermo Scientific MaxQ 4000 shaker incubator. Cells were lysed using a Fisher Scientific Model 505 Sonic Dismembrator. Mammalian cell culture was performed in a NuAire LabGard ES NU-437 Class II Type A2 Biosafety Cabinet. Cells were maintained in a Sanyo inCu saFe® MCO-17AC incubator at 37° C. and 5% CO₂. Flow cytometry was performed at the University of Washington Pathology Flow Cytometry Core Facility on a BD Biosciences LSR II Flow Cytometer.

### Method S1. Synthesis of 2,5-dioxopyrrolidin-1-yl 4-azidobutanoate (N₃—OSu)

4-azidobutanoic acid (N₃—COOH) was synthesized as previously described and used as a synthetic precursor for 2,5-dioxopyrrolidin-1-yl 4-azidobutanoate (N₃—OSu).

### Synthesis of ethyl 4-azidobutanoate

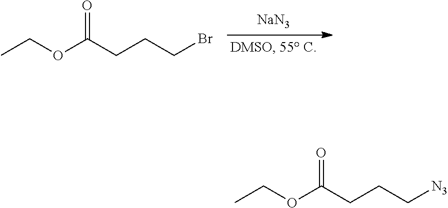

Ethyl-4-bromobutyrate (49.6 g, 254 mmol) and sodium azide (24.7 g, 378 mmol, 1.5×) were dissolved in dimethyl sulfoxide (DMSO, 375 mL) and reacted at 55° C. overnight under a nitrogen atmosphere. The reaction mixture was diluted with water (250 mL) and extracted into diethyl ether (3×250 mL). The organic layer was washed with water (250 mL) and brine (250 mL), dried over MgSO₄, filtered, and concentrated in vacuo to give the intermediate ethyl-4-azidobutanoate (37.7 g, 240 mmol, 94.5% yield) as a clear liquid. ¹H NMR (300 MHz, CDCl₃) δ 4.17 (q, J=7.1 Hz, 2H), 3.38 (t, J=6.7 Hz, 2H), 2.43 (t, J=7.2 Hz, 2H), 1.94 (p, J=7.1 Hz, 2H), 1.29 (t, J=7.1 Hz, 3H).

### Synthesis of 4-azidobutanoic acid (N₃—COOH)

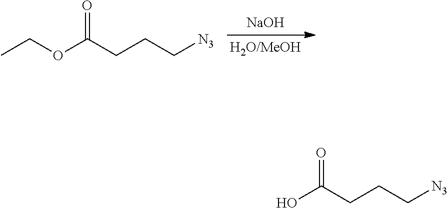

Ethyl-4-azidobutanoate (37.66 g, 240 mmol) was dissolved in aqueous sodium hydroxide (1 M, 250 mL). Methanol was added to homogenize the solution (175 mL) and the reaction mixture was stirred at room temperature for 3 hr. The methanol was removed in vacuo and aqueous hydrochloric acid was added dropwise to the reaction mixture until the pH became 1. The product was extracted into diethyl ether (3×250 mL), dried over magnesium sulfate (MgSO₄), filtered, and concentrated on a rotary evaporator to give 4-azidobutanoic acid (denoted N₃—COOH) as a faint yellow liquid (30.7 g, 237 mmol, 98.8% yield). ¹H NMR (300 MHz, CDCl₃) δ 8.64 (br s, 1H), 3.41 (t, J=6.7 Hz, 2H), 2.50 (t, J=7.2 Hz, 2H), 2.01-1.88 (m, 2H).

### Synthesis of 2,5-dioxopyrrolidin-1-yl 4-azidobutanoate (N₃—OSu)

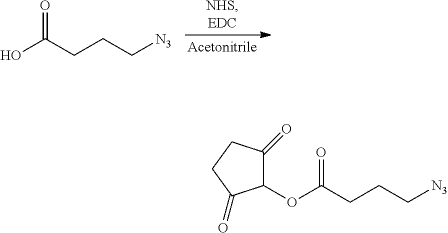 [SMILES: [N-]=[N+]=NCCCC(=O)O ; "NHS,EDC" ; [N-]=[N+]=NCCCC(=O)OC1C(=O)CCC1=O]

N₃—COOH (5.00 g, 38.7 mmol), N-hydroxysuccinimide (5.79 g, 50.3 mmol, 1.3×), and 1-ethyl-3-(3-dimethylaminopropyl)carbodiimide hydrochloride (EDC HCl 9.65 g, 50.3 mmol, 1.3×) were combined in a flame-dried flask and purged with nitrogen. Dry acetonitrile (50 mL) added via syringe to solubilize the reagents. The reaction mixture was stirred overnight at room temperature. The acetonitrile was removed in vacuo and the reaction products were dissolved in dichloromethane (DCM, 100 mL), washed with water (3×100 mL), dried over MgSO₄, filtered, and concentrated via rotary evaporation to give the pure 2,5-dioxopyrrolidin-1-yl 4-azidobutanoate product (denoted N₃—OSu) as a white solid (7.98 g, 35.3 mmol, 91% yield). ¹H NMR (300 MHz, CDCl₃) δ 3.45 (t, J=6.6 Hz, 2H), 2.85 (s, 4H), 2.74 (t, J=7.2 Hz, 2H), 2.09-1.96 (m, 2H).

### Method S2. Synthesis of 2,5-dioxopyrrolidin-1-yl 4-(4-(1-((4-azidobutanoyl)oxy)ethyl)-2-methoxy-5-nitrophenoxy)butanoate (N₃-oNB—OSu)

The synthesis of N₃-oNB—OSu involved the reaction of 4-(4-(1-hydroxyethyl)-2-methoxy-5-nitrophenoxy)butanoic acid and N₃—COOH (Method S1).

### Synthesis of ethyl 4-(4-acetyl-2-methoxyphenoxy)butanoate

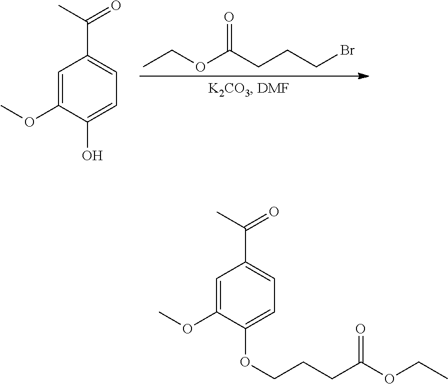

Acetovanillone (30.0 g, 180 mmol) and ethyl-4-bromobutyrate (42.3 g, 217 mmol, 1.2×) were dissolved in dimethylformamide (DMF, 150 mL) in a flame-dried flask. Potassium carbonate (37.4 g, 271 mmol, 1.5×) was added, and the reaction mixture was stirred overnight at room temperature under a nitrogen atmosphere. The reaction mixture was slowly poured into stirring water (1.5 L), and stirring was continued at room temperature for 2 hours. The mixture was stored overnight at 4° C. to precipitate the product. The product was vacuum filtered and lyophilized to give the intermediate, ethyl 4-(4-acetyl-2-methoxyphenoxy)butanoate, as a yellow solid (49.29 g, 175.8 mmol, 97.4% yield). ¹H NMR (300 MHz, CDCl₃) δ 7.54 (m, 2H), 6.89 (d, J=8.2 Hz, 1H), 4.20-4.09 (m, 4H), 3.91 (s, 3H), 2.60-2.48 (m, 5H), 2.18 (p, J=6.8 Hz, 2H), 1.26 (t, J=7.1 Hz, 3H).

### Synthesis of ethyl 4-(4-acetyl-2-methoxy-5-nitrophenoxy)butanoate

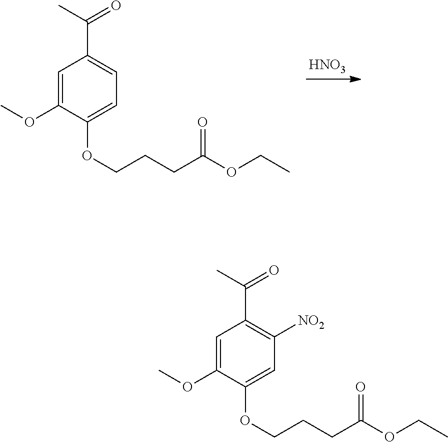

Nitric acid (140 mL) was cooled in an ice bath in a round bottom flask (1 L). Ethyl 4-(4-acetyl-2-methoxyphenoxy)butanoate (48.65 g, 173.6 mmol) was added in small portions to the continually stirred solution, allowing for full dissolution before subsequent additions (approximately 40 minutes to dissolve the product). The reaction mixture was stirred while in the ice bath, and progress was monitored via thin-layer chromatography (TLC, hexanes/ethyl acetate/acetic acid, 50:50:1, product R_{f}˜0.5). Upon reaction completion (˜40 minutes), the reaction mixture was added dropwise to continually stirred ice-cold water (1.5 L). This mixture was further agitated at room temperature for 1 hour, after which the crude product was precipitated overnight at 4° C. The precipitate was filtered, rinsed with ice-cold water, and lyophilized to give a yellow-brown solid. The solid was dissolved in 65° C. ethanol (700 mL), cooled to room temperature, and recrystallized overnight at 4° C. The product was vacuum filtered, rinsed with −20° C. ethanol, and dried to give the intermediate, ethyl 4-(4-acetyl-2-methoxy-5-nitrophenoxy)butanoate, as a yellow solid (33.90 g, 104.2 mmol, 60.0% yield). ¹H NMR (300 MHz, CDCl₃) δ 7.60 (s, 1H), 6.74 (s, 1H), 4.20-4.11 (m, 4H), 3.95 (s, 3H), 2.54 (t, J=7.2 Hz, 2H), 2.49 (s, 3H), 2.19 (p, J=6.7 Hz, 2H), 1.26 (t, J=7.1 Hz, 3H).

### Synthesis of ethyl 4-(4-(1-hydroxyethyl)-2-methoxy-5-nitrophenoxy)butanoate

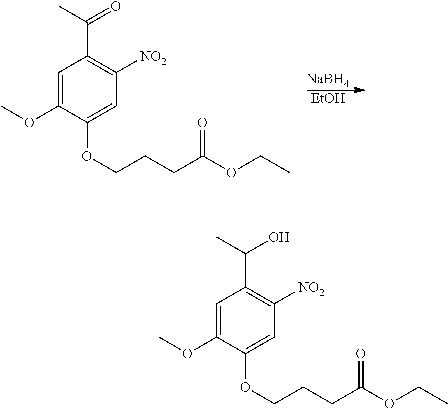

Ethyl 4-(4-acetyl-2-methoxy-5-nitrophenoxy)butanoate (47.12 g, 144 mmol) was dissolved in anhydrous ethanol (700 mL), purged with nitrogen, and heated to 38° C. Sodium borohydride (5.33 g, 141 mmol, 0.97×) was added to the reaction mixture in equal portions every 5 minutes over a 40 minute period. The mixture was purged with nitrogen and stirred overnight at 38° C. The reaction mixture was added to water (7 L), stirred 1 hour at room temperature, and the product was precipitated overnight at 4° C. The suspension was filtered, rinsed with ice-cold water, and lyophilized to yield the intermediate, ethyl 4-(4-(1-hydroxyethyl)-2-methoxy-5-nitrophenoxy)butanoate, as a dark yellow solid (37.84 g, 115.6 mmol, 80% yield). ¹H NMR (300 MHz, DMSO) δ 7.53 (s, 1H), 7.36 (s, 1H), 5.48 (d, J=4.4 Hz, 1H), 5.33-5.19 (m, 1H), 4.17-3.99 (m, 4H), 3.91 (s, 3H), 2.48 (m, 2H), 1.99 (p, J=6.8 Hz, 2H), 1.37 (d, J=6.2 Hz, 3H), 1.19 (t, J=7.1 Hz, 3H).

### Synthesis of 4-(4-(1-hydroxyethyl)-2-methoxy-5-nitrophenoxy)butanoic acid

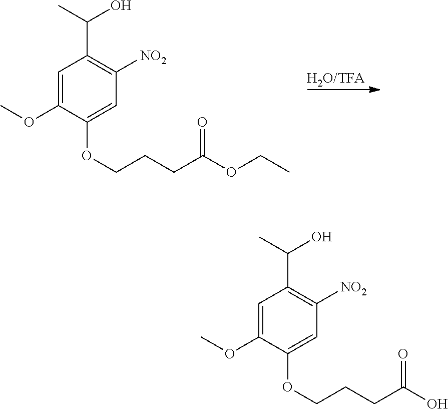

Ethyl 4-(4-(1-hydroxyethyl)-2-methoxy-5-nitrophenoxy)butanoate (37.84 g, 115.6 mmol) was added to a solution of water (1 L) and trifluoroacetic acid (TFA, 96 mL) in a round-bottomed flask (2 L). The reaction mixture was stirred and heated to 90° C. After 6 hours, additional TFA (48 mL) was added to the reaction mixture. After another 18 hours, additional TFA (48 mL) was added to the reaction mixture. After another 6 hours, the reaction mixture was filtered to remove the black solid. The filtrate was cooled to room temperature, upon which the precipitate was dissolved in minimal aqueous sodium hydroxide (1 M, 150 mL) and acidified to a pH of 1 via dropwise addition of hydrochloric acid. The precipitated product was vacuum filtered, washed, and lyophilized to give 4-(4-(1-hydroxyethyl)-2-methoxy-5-nitrophenoxy)butanoic acid as a yellow solid (20.08 g, 93.6 mmol, 81.0% yield). ¹H NMR (300 MHz, DMSO) δ 12.18 (s, 1H), 7.55 (s, 1H), 7.37 (s, 1H), 5.50 (m, 1H), 5.27 (m, 1H), 4.07 (t, J=6.5 Hz, 2H), 3.92 (s, 3H), 2.40 (t, J=7.3 Hz, 2H), 1.96 (p, J=6.8 Hz, 2H), 1.37 (d, J=6.2 Hz, 3H).

### Synthesis of 4-(4-(1-((4-azidobutanoyl)oxy)ethyl)-2-methoxy-5-nitrophenoxy)butanoic acid (N₃-oNB—COOH)

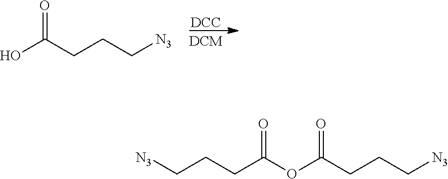

N₃—COOH (Method S1, 64.6 g, 500 mmol) and N,N′-Dicyclohexylcarbodiimide (33.0 g, 160 mmol) were combined in a flame-dried flask, purged with nitrogen, dissolved in anhydrous DCM (400 mL), and reacted at room temperature for 60 minutes. The reaction mixture was filtered to remove the dicyclohexylurea byproduct, concentrated in vacuo, and filtered. The crude product was repeatedly redissolved in anhydrous DCM (˜90 mL), concentrated, and filtered until urea formation ceased.

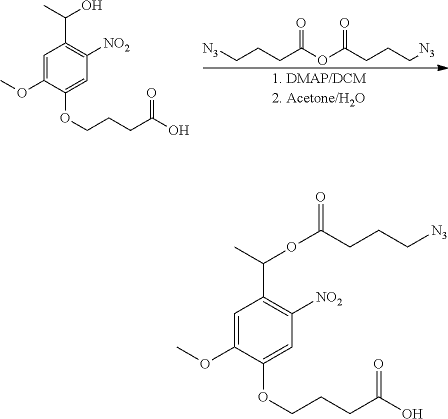 [SMILES: COc1cc(C(C)O)c([N+](=O)[O-])cc1OCCCC(=O)O ; [N-]=[N+]=NCCCC(=O)OC(=O)CCCN=[N+]=[N-] ; COc1cc(C(C)OC(=O)CCCN=[N+]=[N-])c([N+](=O)[O-])cc1OCCCC(=O)O]

4-(4-(1-hydroxyethyl)-2-methoxy-5-nitrophenoxy)butanoic acid (10.0 g, 33.4 mmol), 4-dimethylaminopyridine (DMAP, 205 mg, 1.67 mmol) were added to the crude anhydride and dissolved in minimal DCM (250 mL). Pyridine (2.69 mL, 33.4 mmol) was added to the reaction and stirred at room temperature overnight under a nitrogen atmosphere. The reaction mixture was washed with saturated aqueous sodium carbonate (250 mL), 1 M hydrochloric acid (250 mL), and concentrated in vacuo. The intermediate was dissolved in water/acetone (50:50, 1400 mL) and stirred at room temperature overnight. The acetone was removed in vacuo and the product was extracted into DCM (3×300 mL). The organic layer was washed with 1 M hydrochloric acid, brine, dried over MgSO₄, filtered, and concentrated in vacuo. The crude mixture was purified on a silica flash column (20-40% Ethyl acetate in hexanes with 1% acetic acid) and concentrated to give 4-(4-(1-((4-azidobutanoyl)oxy)ethyl)-2-methoxy-5-nitrophenoxy)butanoic acid (denoted N₃-oNB—COOH) as a yellow solid (11.59 g, 28.24 mmol, 85% yield). ¹H NMR (500 MHz, DMSO) δ 12.08 (s, 1H), 7.57 (s, 1H), 7.10 (s, 1H), 6.21 (q, J=6.4 Hz, 1H), 4.07 (t, J=6.4 Hz, 2H), 3.93 (s, 3H), 3.32 (t, J=6.8 Hz, 2H), 2.43 (t, J=7.3 Hz, 2H), 2.38 (t, J=7.3 Hz, 2H), 1.95 (p, J=6.8 Hz, 2H), 1.76 (p, J=7.1 Hz, 2H), 1.58 (d, J=6.5 Hz, 3H).

### Synthesis of 2,5-dioxopyrrolidin-1-yl 4-(4-(1-((4-azidobutanoyl)oxy)ethyl)-2-methoxy-5-nitrophenoxy)butanoate (N₃-oNB—OSu)

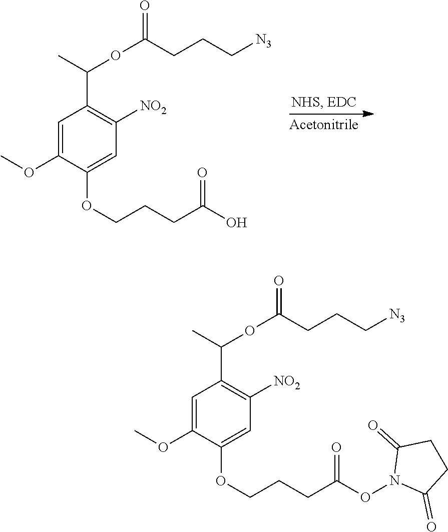 [SMILES: COc1cc(C(C)OC(=O)CCCN=[N+]=[N-])c([N+](=O)[O-])cc1OCCCC(=O)O ; COc1cc(C(C)OC(=O)CCCN=[N+]=[N-])c([N+](=O)[O-])cc1OCCCC(=O)ON1C(=O)CCC1=O]

N₃-oNB—COOH (5.51 g, 13.4 mmol), N-hydroxysuccinimide (2.01 g, 17.5 mmol, 1.3×), and 1-ethyl-3-(3-dimethylaminopropyl)carbodiimide hydrochloride (EDC HCl 3.35 g, 17.5 mmol, 1.3×) were combined in a flame-dried flask and purged with nitrogen. Dry acetonitrile (50 mL) added via syringe to solubilize the reagents. The reaction mixture was stirred overnight at room temperature. The acetonitrile was removed in vacuo and the reaction mixture was dissolved in DCM (100 mL), washed with water (3×100 mL), dried over MgSO₄, filtered, and concentrated in vacuo to give the product (denoted N₃-oNB—OSu) as a yellow solid (6.69 g, 13.2 mmol, 98% yield). ¹H NMR (500 MHz, CDCl₃) δ 7.62 (s, 1H), 7.03 (s, 1H), 6.52 (q, J=6.4 Hz, 1H), 4.20 (t, J=6.0 Hz, 2H), 4.00 (s, 3H), 3.42-3.31 (m, 2H), 2.91 (t, J=7.3 Hz, 2H), 2.88 (s, 4H), 2.55-2.42 (m, 2H), 2.32 (p, J=6.7 Hz, 2H), 1.93 (p, J=6.9 Hz, 2H), 1.65 (d, J=6.4 Hz, 3H).

### Method S3. Synthesis of (R)-2-((((9H-fluoren-9-yl)methoxy)carbonyl)amino)-6-azidohexanoic acid (Fmoc-Lys(N₃)—OH)

Imidazole-1-sulfonyl azide hydrochloride (Stick's reagent) and (R)-2-((((9H-fluoren-9-yl)methoxy)carbonyl)amino)-6-azidohexanoic acid (Fmoc-Lys(N₃)—OH) were synthesized following known synthetic routes.

### Synthesis of imidazole-1-sulfonyl azide hydrochloride

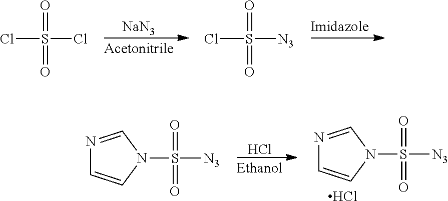

Sulfuryl chloride (27.0 g, 16.2 mL, 200 mmol) was added dropwise to a suspension of sodium azide (13.0 g, 200 mmol) in acetonitrile (200 mL, 0° C.), and the reaction was stirred overnight at room temperature. The mixture was cooled in an ice bath, and imidazole (25.9 g, 380 mmol) was added in small portions to the stirred reaction mixture. The reaction mixture was brought to room temperature and stirred for 3 hours. The reaction mixture was diluted with ethyl acetate (400 mL), washed with water (2×400 mL), washed with saturated aqueous sodium carbonate (2×400 mL), dried over MgSO₄, and filtered. Hydrochloric acid in ethanol [formulated under a nitrogen atmosphere by dropwise addition of acetyl chloride (23.5 g, 300 mmol) into ice-cold anhydrous ethanol (75 mL)] was added dropwise to the stirring filtrate. The product was precipitated in an ice bath and filtered. The filter cake was washed with ice-cold ethyl acetate (3×100 mL) to yield the pure product, imidazole-1-sulfonyl azide hydrochloride [Stick's reagent], as a white powder (25.86 g, 123.4 mmol, 61.7% yield). ¹H NMR (300 MHz, D₂O) δ 9.45-9.42 (m, 1H), 8.06-8.01 (m, 1H), 7.65-7.60 (m, 1H); ¹³C-NMR (75 MHz, D₂O) δ=137.7, 123.3, 120.1.

### Synthesis of (R)-2-((((9H-fluoren-9-yl)methoxy)carbonyl)amino)-6-azidohexanoic acid (Fmoc-Lys(N₃)—OH)

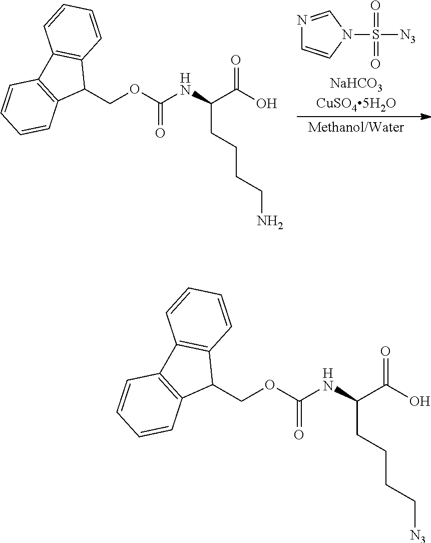 [SMILES: NCCCC[C@@H](NC(=O)OCC1c2ccccc2-c2ccccc21)C(=O)O ; [N-]=[N+]=NS(=O)(=O)n1ccnc1 ; [N-]=[N+]=NCCCC[C@@H](NC(=O)OCC1c2ccccc2-c2ccccc21)C(=O)O]

Fmoc-Lys-OH (11.00 g, 29.86 mmol) was dissolved in water (30 mL) with hydrochloric acid (aqueous 37%, 2.47 mL, 1×). A solution of water/methanol (1:2, 130 mL), imidazole-1-sulfonyl azide hydrochloride (7.50 g, 35.8 mmol, 1.2×), sodium bicarbonate (16.22 g, 193.1 mmol, 6.5×), and aqueous CuSO₄·5H₂O (74.6 mg in 2 mL dH₂O, 0.299 mmol, 0.01×) were added, in order, to the reaction mixture and stirred for 17 hours at room temperature. The methanol was removed in vacuo, and the mixture was diluted with water (300 mL) and adjusted to a pH of 2 with hydrochloric acid. The crude product was extracted into ethyl acetate (3×150 mL), dried over MgSO₄, filtered, and concentrated on a rotary evaporator. The crude product was purified on a silica column with a mobile phase of toluene/ethyl acetate/acetic acid (85:10:5) and concentrated to give the pure product [(R)-2-((((9H-fluoren-9-yl)methoxy)carbonyl)amino)-6-azidohexanoic acid, denoted Fmoc-Lys(N₃)—OH], as a light-yellow solid (10.82 g, 27.4 mmol, 92% yield). ¹H NMR (300 MHz, CDCl₃) δ 9.93 (s, 1H), 7.79 (d, J=7.5 Hz, 2H), 7.63 (d, J=7.0 Hz, 2H), 7.39 (dt, J=26.7, 7.2 Hz, 4H), 5.43 (d, J=8.2 Hz, 1H), 4.65-4.34 (m, 3H), 4.25 (t, J=6.8 Hz, 1H), 3.38-3.14 (m, 2H), 2.04-1.35 (m, 6H).

### Method S4. Fmoc Solid-Phase Peptide Synthesis

Automated microwave-assisted Fmoc solid-phase peptide synthesis was performed on a CEM Liberty 1 (0.5 mmol scale). Fmoc deprotections were performed in 20% piperidine (v/v) in DMF with 0.1 M 1-hydroxybenzotriazole (HOBt) at 90° C. for 90 seconds. Amino acids (except arginine and cysteine) were coupled to resin-bound peptides upon treatment (75° C. for 5 minutes) with Fmoc-protected amino acid (2 mmol, 4×), 2-(1H-benzotriazol-1-yl)-1,1,3,3-tetramethyluronium hexafluorophosphate (HBTU, 2 mmol, 4×), and N,N-diisopropylethylamine (DIEA, 2 mmol, 4×) in a mixture of DMF (9 mL) and N-Methyl-2-pyrrolidone (NMP, 2 mL). Cysteine couplings were performed using the same reagents as above but the reaction was performed at 50° C. for 30 minutes. Arginine couplings were performed using the same reagents as above but the reaction was performed at 25° C. for 45 minutes, drained, and repeated at 75° C. for 5 minutes.

### Method S5. Enzymatic “YES” Degradable Crosslinker Synthesis (E)

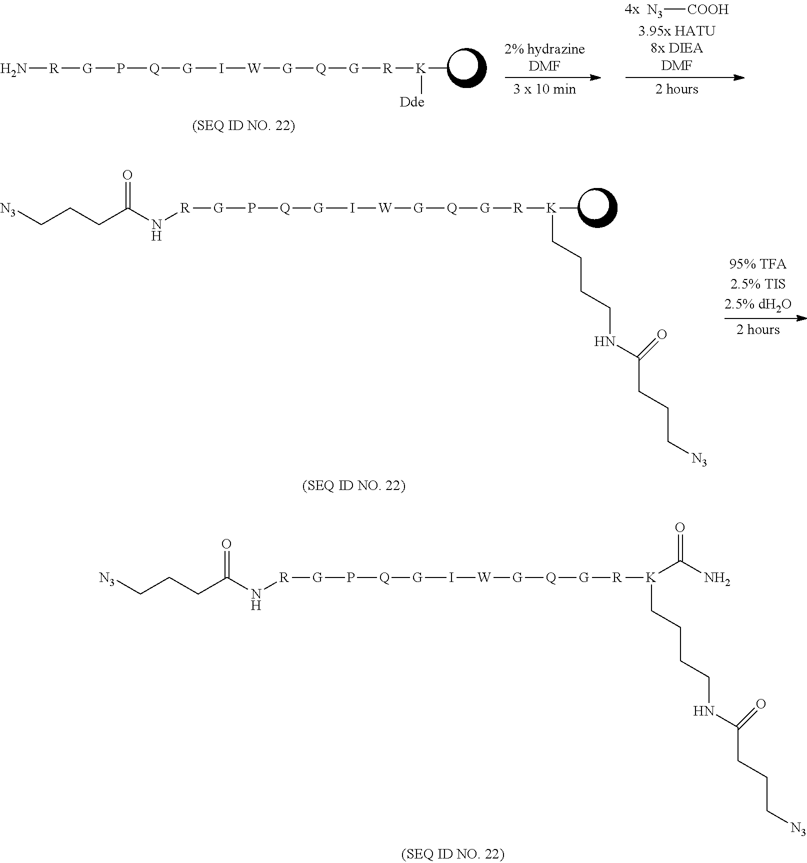

The resin-bound peptide H-RGPQGIWGQGRK(Dde) (SEQ ID NO. 22) was synthesized by microwave-assisted Fmoc solid-phase peptide synthesis (SPPS, Method S4) on Rink amide resin (0.5 mmol scale). The resin was treated with hydrazine monohydrate (2%) in DMF (3×10 min) to remove the N-(1-(4,4-dimethyl-2,6-dioxocyclohexylidene)ethyl) (Dde) protecting group. N₃—COOH (0.517 g, 4.0 mmol, 4×, Method S1) was pre-activated upon reaction with 1-[Bis(dimethylamino)methylene]-1H-1,2,3-triazolo[4,5-b]pyridinium 3-oxide hexafluorophosphate (HATU, 1.502 g, 3.95 mmol, 3.95×) and DIEA (1.034 g, 8.0 mmol, 8×) in minimal DMF for 5 minutes and then reacted with the resin for 90 minutes to functionalize the N-terminus and the ε-amino group of the lysine with an azide. The peptide was simultaneously deprotected and cleaved from resin upon treatment with TFA/triisopropylsilane (TIS)/dH₂O (95:2.5:2.5, 30 mL) for 2 hours, and the crude peptide was precipitated in and washed with ice-cold diethyl ether (2×). The crude peptide was purified using RP-HPLC operating with a 55 minute linear gradient (5-100%) of acetonitrile in water containing TFA (0.1%); lyophilization yielded the final product (N₃—(CH₂)₃CONH-RGPQGIWGQGRK(CO(CH₂)₃N₃))—NH₂, denoted E) (SEQ ID. NO. 22) as a yellow solid (222.0 mg, 0.142 mmol, 28.4% overall yield). Peptide purity was confirmed using MALDI-TOF: calculated for C₆₆H₁₀₆N₂₉O₁₆⁺ [M+¹H]⁺, 1560.8; observed 1560.9.

### Method S6. Reductive “YES” Degradable Crosslinker Synthesis (R)

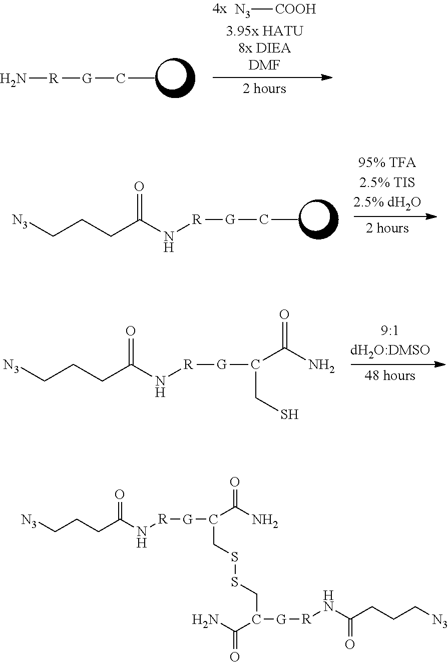

The resin-bound peptide H-RGC-NH₂ was synthesized by microwave-assisted Fmoc SPPS (Method S4) on Rink amide resin (1.0 mmol scale). N₃—COOH (Method S1, 0.517 g, 4.0 mmol, 4×) was pre-activated upon reaction with HATU (1.502 g, 3.95 mmol, 3.95×) and DIEA (1.034 g, 8.0 mmol, 8×) in minimal DMF for 5 minutes and then reacted with the resin for 90 minutes to functionalize the N-terminus with an azide. The peptide was simultaneously deprotected and cleaved from resin upon treatment with TFA/TIS/dH₂O (95:2.5:2.5, 30 mL) for 2 hours, and the crude peptide was precipitated in and washed with ice-cold diethyl ether (2×). The crude peptide was dissolved in a dH₂O/DMSO (90:10) solution (100 mL) and reacted at room temperature for 48 hours. The peptide was concentrated and purified using RP-HPLC operating with a 55 minute linear gradient (5-100%) of acetonitrile in water containing TFA (0.1%); lyophilization yielded the final product (N₃—(CH₂)₃CONH-RGC(N₃—(CH₂)₃CONH-RGC-NH₂)—NH₂ dimerized via disulfide bond through the cysteine side-chain thiols, denoted R) as a yellow solid (139.5 mg, 0.1572 mol, 31.5% overall yield). Peptide purity was confirmed using MALDI-TOF: calculated for C₃₀H₅₅N₂₀O₈S₂⁺ [M+¹H]⁺, 887.4; observed 887.4.

### Method S7. Photo “YES” Degradable Crosslinker Synthesis (P)

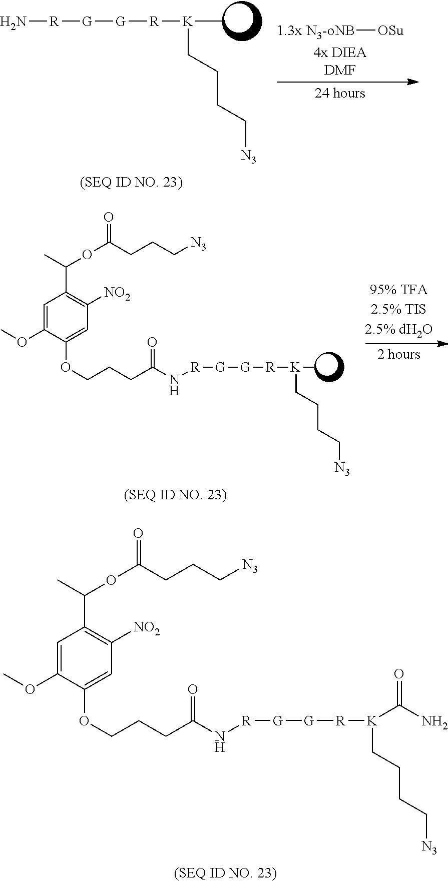

The resin-bound peptide H₂N-RGGRK(N₃) (SEQ ID NO. 23) was synthesized by microwave-assisted Fmoc SPPS (Method S4) on Rink amide resin (0.5 mmol scale) using a C-terminal Fmoc-Lys(N₃)—OH (Method S3). Resin was subsequently treated with N₃-oNB—OSu (Method S2, 0.65 mmol, 330 mg) and DIEA (2.0 mmol, 258 mg) in minimal DMF to introduce oNB functionality to the N-terminus. The peptide was simultaneously deprotected and cleaved from resin upon treatment with TFA/TIS/dH₂O (95:2.5:2.5, 30 mL) for 2 hours, and the crude peptide was precipitated in and washed with ice-cold diethyl ether (2×). The crude peptide was purified using RP-HPLC operating with a 43.4 minute linear gradient (20-100%) of acetonitrile in water containing TFA (0.1%); lyophilization yielded the final product (N₃-oNB-RGGRK(N₃)—NH₂, denoted P) (SEQ ID NO. 23) as a yellow solid (147.4 mg, 0.1488 mol, 29.8% overall yield). Peptide purity was confirmed using MALDI-TOF: calculated for C₃₉H₆₄N₁₉O₁₂⁺ [M+¹H]⁺, 990.5; observed 990.4.

### Method S8. Reductive “OR” Enzymatic Degradable Crosslinker Synthesis (R∨E)

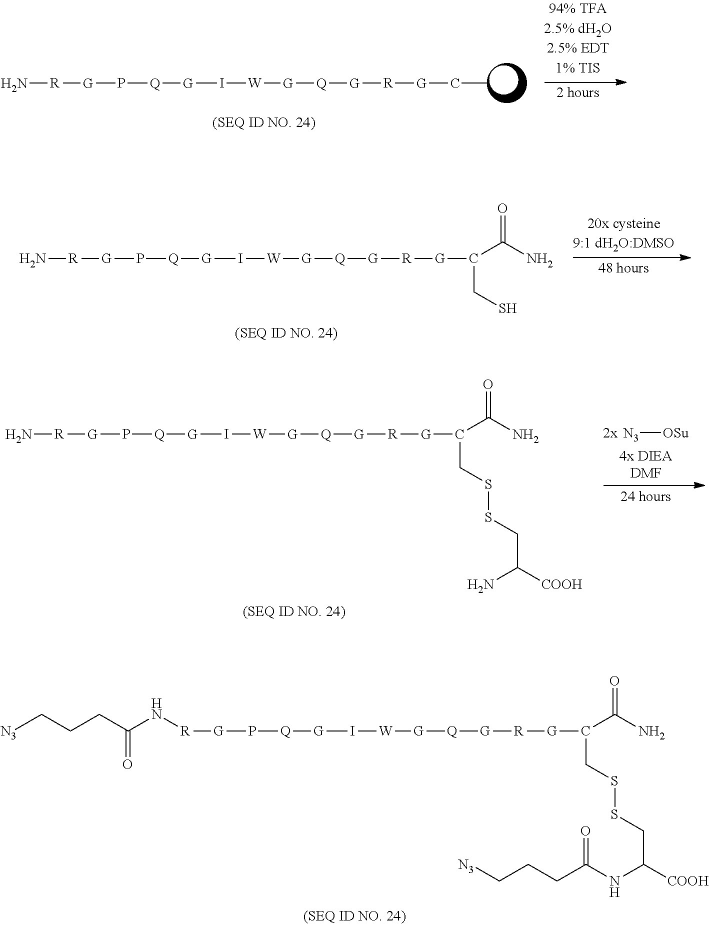

The resin-bound peptide H-RGPQGIWGQGRGC (SEQ ID NO. 24) was synthesized by microwave-assisted Fmoc SPPS (Method S4) on Rink amide resin (0.5 mmol scale). The peptide was simultaneously deprotected and cleaved from resin upon treatment with TFA/dH₂O/1,2-ethanedithiol (EDT)/TIS (94:2.5:2.5:1, 30 mL) for 2 hours, and the crude peptide was precipitated in and washed with ice-cold diethyl ether (2×). The crude peptide was purified using RP-HPLC operating with a 55 minute linear gradient (5-100%) of acetonitrile in water containing TFA (0.1%); lyophilization yielded the pure intermediate (H-RGPQGIWGQGRGC-NH₂) (SEQ ID NO. 24) as a white solid (417.4 mg, 0.305 mmol). The purified peptide and cysteine (0.74 g, 6.1 mmol, 20×) were dissolved in a dH₂O/DMSO (90:10) solution (50 mL) and stirred at room temperature for 48 hours. Product was filtered, concentrated in vacuo, and purified using RP-HPLC operating with a 55 minute linear gradient (5-100%) of acetonitrile in water containing TFA (0.1%); lyophilization yielded the pure intermediate (H-RGPQGIWGQGRGC(H—C—OH)—NH₂ (SEQ ID NO. 24) with cysteines linked via disulfide bond) as a white solid (241.4 mg, 0.162 mmol). To introduce azide functionalities requisite for hydrogel crosslinking, N₃-OSu (Method S1, 146.6 mg, 0.648 mmol, 2×) was dissolved in minimal DMF with DIEA (167.5 mg, 1.30 mmol, 4×) and reacted with the peptide overnight at room temperature. The peptide was purified using RP-HPLC operating with a 55 minute linear gradient (5-100%) of acetonitrile in water containing TFA (0.1%); lyophilization yielded the final product (N₃-RGPQGIWGQGRGC(N₃(CH₂)₃CONH—C—OH)—NH₂ (SEQ ID NO. 24) with cysteines linked via disulfide bridge, denoted R∨E) as a white solid (89.8 mg, 0.0525 mmol, 10.5% overall yield). Peptide purity was confirmed using MALDI-TOF: calculated for C₆₈H₁₀₇N₃₀O₁₉S₂⁺ [M+¹H]⁺, 1711.7; observed 1711.5.

### Method S9. Enzymatic “OR” Photo Degradable Crosslinker Synthesis (E∨P)

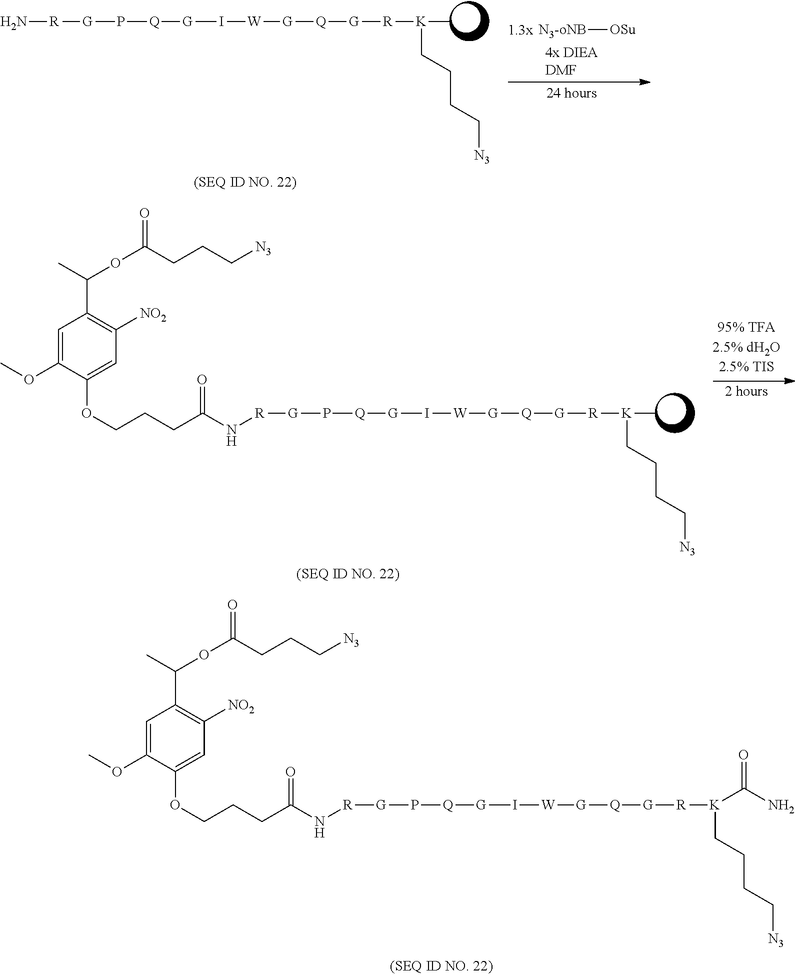

The resin-bound peptide H-RGPQGIWGQGRK(N₃)—NH₂ (SEQ ID NO. 22) was synthesized by microwave-assisted Fmoc SPPS (Method S4) on Rink amide resin (0.5 mmol scale) using a C-terminal Fmoc-Lys(N₃)—OH (Method S3). Resin was subsequently treated with N₃-oNB—OSu (Method S2, 0.65 mmol, 330 mg) and DIEA (2.0 mmol, 258 mg) in minimal DMF to introduce oNB functionality to the N-terminus. The peptide was simultaneously deprotected and cleaved from resin upon treatment with TFA/TIS/dH₂O (95:2.5:2.5, 30 mL) for 2 hours, and the crude peptide was precipitated in and washed with ice-cold diethyl ether (2×). The crude peptide was purified using RP-HPLC operating with a 43.4 minute linear gradient (20-100%) of acetonitrile in water containing TFA (0.1%); lyophilization yielded the final product (N₃-oNB-RGPQGIWGQGRK(N₃)—NH₂ (SEQ ID NO. 22), denoted E∨P) as a yellow solid (159.9 mg, 0.0910 mmol, 18.2% overall yield). Peptide purity was confirmed using MALDI-TOF: calculated for C₇₅H₁₁₄N₂₉O₂₁⁺ [M+¹H]⁺, 1756.9; observed 1756.9.

### Method S10. Reductive “OR” Photo Degradable Crosslinker Synthesis (R∨P)

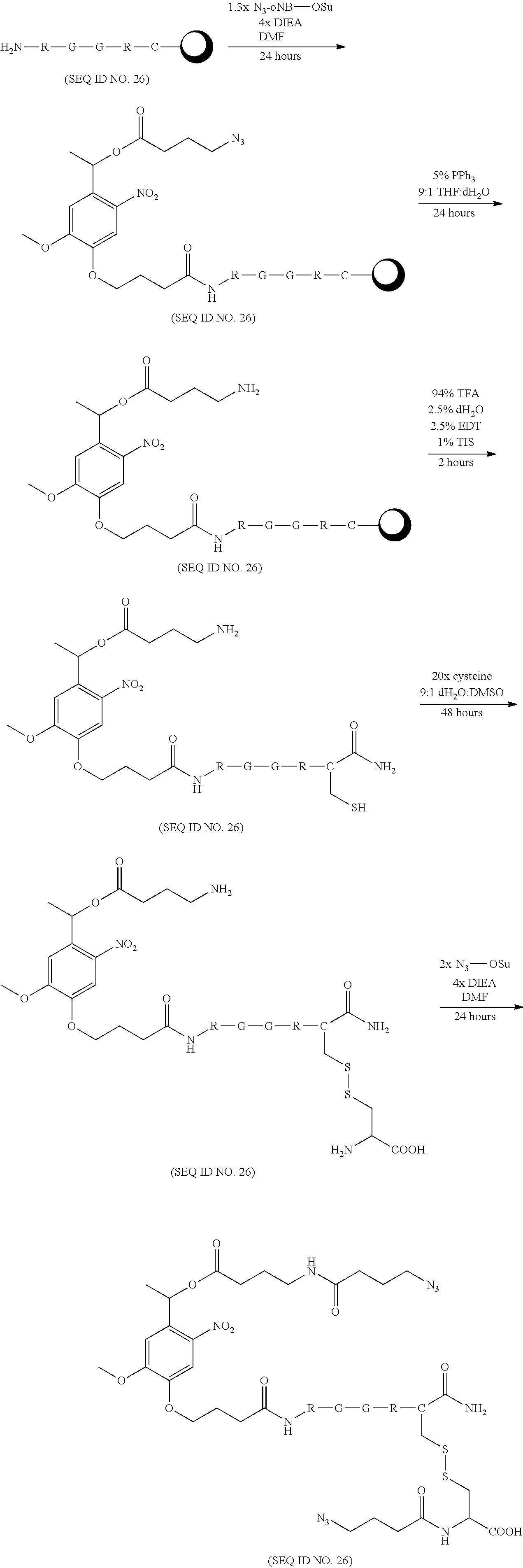

The resin-bound peptide H-RGGRC-NH₂ (SEQ ID NO. 26) was synthesized by microwave-assisted Fmoc SPPS (Method S4) on Rink amide resin (0.5 mmol scale). Resin was subsequently treated with N₃-oNB—OSu (Method S2, 0.65 mmol, 330 mg) and DIEA (2.0 mmol, 258 mg) in minimal DMF to introduce oNB functionality to the N-terminus. The N-terminal azide was reduced to an amine by a Staudinger reduction; resin was washed with tetrahydrofuran (THF)/H₂O (90:10, 3×20 mL), followed by reaction with 5 wt % triphenylphosphine in THF/H₂O (90:10, 30 mL) for 18 hours. The peptide was simultaneously deprotected and cleaved from resin upon treatment with TFA/dH₂O/EDT/TIS (94:2.5:2.5:1, 30 mL) for 2 hours, and the crude peptide was precipitated in and washed with ice-cold diethyl ether (2×). The crude peptide was purified using RP-HPLC operating with a 43.4 minute linear gradient (20-100%) of acetonitrile in water containing TFA (0.1%); lyophilization yielded the pure intermediate (H-oNB-RGGRC-NH₂) (SEQ ID NO. 26) as a yellow solid (146.4 mg, 0.160 mmol). The purified peptide and cysteine (0.39 g, 3.2 mmol, 20×) were dissolved in a dH₂O/DMSO (90:10) solution (50 mL) and stirred at room temperature for 48 hours. The peptide was filtered, concentrated in vacuo, and purified using RP-HPLC operating with a 43.4 minute linear gradient (20-100%) of acetonitrile in water containing TFA (0.1%); lyophilization yielded the pure intermediate (H-oNB-RGGRC(H—C—OH)—NH₂ (SEQ ID NO. 26) with cysteines linked via disulfide bond) as a yellow solid (120.8 mg, 0.117 mmol). N₃—OSu (Method S1, 121 mg, 0.468 mmol, 2×) was dissolved in minimal DMF with DIEA (167.5 mg, 0.963 mmol, 4×) and reacted with the peptide overnight at room temperature. The peptide was purified using RP-HPLC operating with a 43.4 minute linear gradient (20-100%) of acetonitrile in water containing TFA (0.10%); lyophilization yielded the final product (N₃-oNB-RGGRC(N₃(CH₂)₃CONH—C—OH)—NH₂ (SEQ ID NO. 26) with cysteines linked via disulfide bond, denoted R∨P) as a yellow solid (32.2 mg, 0.0257 mmol, 5.1% overall yield). Peptide purity was confirmed using MALDI-TOF: calculated for C₄₇H₇₆N₂₁O₁₆S₂⁺ [M+¹H]⁺, 1254.5; observed 1254.6.

### Method S11. Reductive “AND” Enzymatic Degradable Crosslinker Synthesis (R∧E)

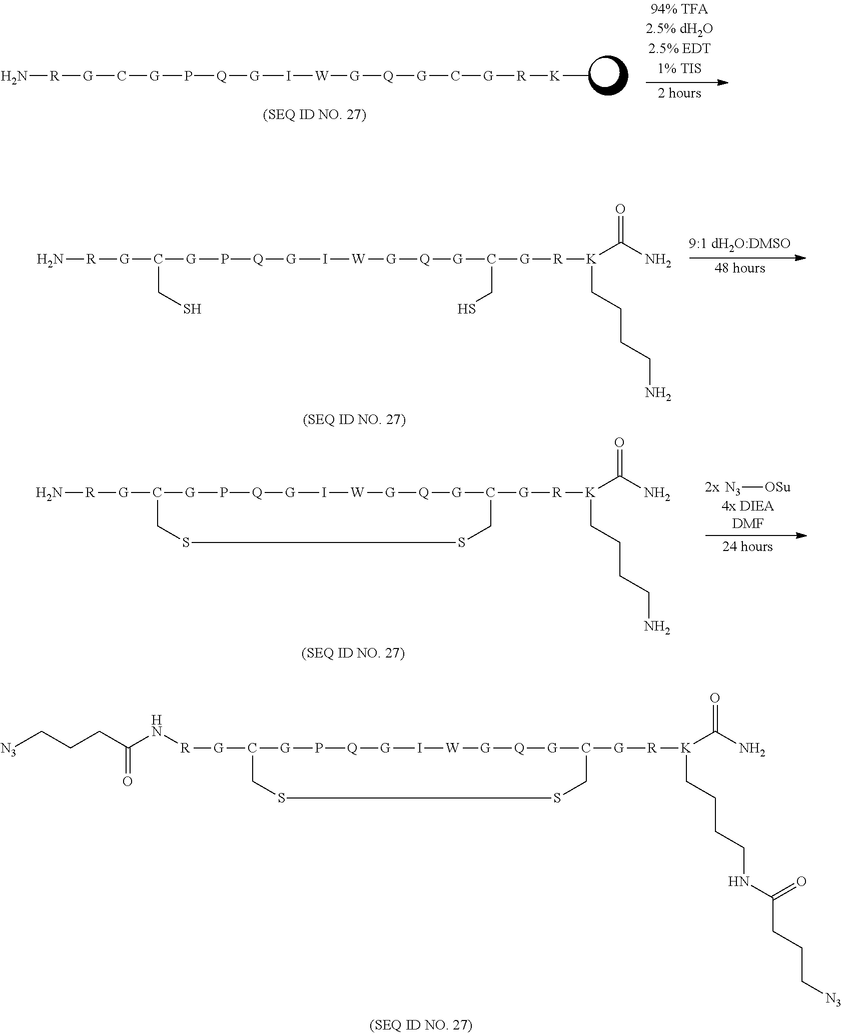

The resin-bound peptide H-RGCGPQGIWGQGCGRK (SEQ ID NO. 27) was synthesized by microwave-assisted Fmoc SPPS (Method S4) on Rink amide resin (1.0 mmol scale). The peptide was simultaneously deprotected and cleaved from resin upon treatment with TFA/dH₂O/EDT/TIS (94:2.5:2.5:1) for 3 hours and the crude peptide was precipitated in and washed with ice-cold diethyl ether (2×). The crude peptide was purified using RP-HPLC operating with a 55 minute linear gradient (5-100%) of acetonitrile in water containing TFA (0.1%); lyophilization yielded the pure intermediate (H-RGCGPQGIWGQGCGRK-NH₂) (SEQ ID NO. 27) as a white solid (531.0 mg, 0.320 mmol). To promote disulfide-mediated intramolecular stapling, the purified peptide was dissolved at 0.5 mM in a dH₂O/DMSO (90:10) solution (700 mL) and reacted at room temperature with no agitation for 48 hours. Stapled product was concentrated in vacuo and purified using RP-HPLC operating with a 55 minute linear gradient (5-100%) of acetonitrile in water containing TFA (0.1%); lyophilization yielded the pure intermediate (H-RGCGPQGIWGQGCGRK-NH₂ (SEQ ID NO. 27) with intramolecular stapling via cysteine-cysteine disulfide bond) as a white solid (489 mg, 0.295 mmol). To introduce azide functionalities requisite for hydrogel crosslinking, N₃—OSu (Method S1, 267 mg, 1.18 mmol, 2×) was dissolved in minimal DMF with DIEA (305 mg, 2.36 mmol, 4×) and reacted with the peptide overnight at room temperature. Stapled peptide was purified using RP-HPLC operating with a 55 minute linear gradient (5-100%) of acetonitrile in water containing TFA (0.1%); lyophilization yielded the final product (N₃-RGCGPQGIWGQGCGRK(CO(CH₂)₃N₃)—NH₂ (SEQ ID NO. 27) with intramolecular stapling via cysteine-cysteine disulfide bond, denoted R∧E) as a white solid (197 mg, 0.105 mmol, 10.5% overall yield). Peptide purity was confirmed using MALDI-TOF: calculated for C₇₆H₁₂₀N₃₃O₂₀S₂⁺ [M+¹H]⁺, 1878.9; observed 1879.0.

### Method S12. Enzymatic “AND” Photo Degradable Crosslinker Synthesis (E∧P)

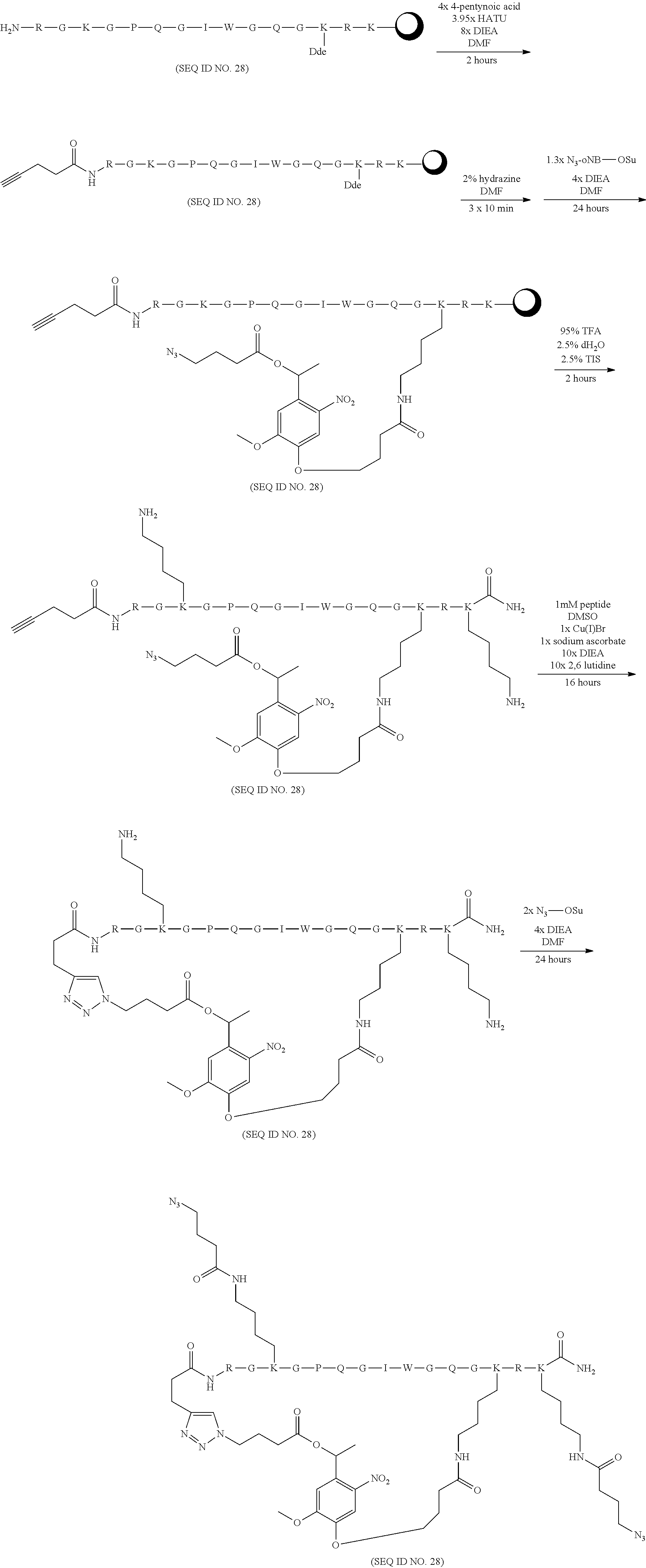

The resin-bound peptide H-RGKGPQGIWGQGK(Dde)RK (SEQ ID NO. 28) was synthesized by microwave-assisted Fmoc SPPS (Method S4) on Rink amide resin (0.5 mmol scale). 4-pentynoic acid (0.196 g, 2.0 mmol, 4×) was pre-activated upon reaction with HATU (0.751 g, 1.975 mmol, 3.95×) and DIEA (0.517 g, 4.0 mmol, 8×) in minimal DMF for 5 minutes and then reacted with the resin for 90 minutes to functionalize the N-terminus with an alkyne. The resin was treated with hydrazine monohydrate (2%) in DMF (3×10 min) to remove the Dde protecting group. Resin was subsequently treated with N₃-oNB—OSu (Method S2, 0.65 mmol, 330 mg) and DIEA (2.0 mmol, 258 mg) in minimal DMF to introduce oNB functionality to the 8-amino group of the unprotected lysine. The peptide was simultaneously deprotected and cleaved from resin upon treatment with TFA/TIS/dH₂O (95:2.5:2.5, 30 mL) for 2 hours, and the crude peptide was precipitated in and washed with ice-cold diethyl ether (2×). The crude peptide was purified using RP-HPLC operating with a 43.4 minute linear gradient (20-100%) of acetonitrile in water containing TFA (0.10%); lyophilization yielded the pure intermediate (HCC(CH₂)₂CO-RGKGPQGIWGQGK(oNB-N₃)RK—NH₂) (SEQ ID NO. 28) as a yellow solid (116 mg, 0.0546 mmol). To induce CuAAC-mediated intramolecular stapling (copper(I)-catalyzed azide-alkyne cycloaddition), pure linear peptide (1 mM) was dissolved in nitrogen-purged DMSO (55 mL) containing copper(I) bromide (7.8 mg, 0.055 mmol, 1×), sodium ascorbate (10.8 mg, 0.055 mmol, 1×) in water (550 μL), 2,6 lutidine (55.8 mg, 0.546 mmol, 10×), and DIEA (70.6 mg, 0.546 mmol, 10×). This mixture was allowed to react under nitrogen at room temperature for 16 hours. The mixture was concentrated in vacuo, redissolved in dH₂O, passed through an ion exchange column (Dowex M4195 resin, 5 grams), and lyophilized. Stapled product was purified using RP-HPLC operating with a 43.4 minute linear gradient (20-100%) of acetonitrile in water containing TFA (0.1%); lyophilization yielded the pure intermediate HCC(CH₂)₂CO-RGKGPQGIWGQGK(oNB-N₃)RK—NH₂ (SEQ ID NO. 28) with an intramolecular staple via a triazole linkage between the alkyne and oNB-N₃ side chains) as a yellow solid (36 mg, 0.017 mmol). To introduce azide functionalities requisite for hydrogel crosslinking, N₃—OSu (Method S1, 15.4 mg, 0.068 mmol, 2×) was dissolved in minimal DMF with DIEA (17.6 mg, 0.69 mmol, 4×) and reacted with the peptide overnight at room temperature. The stapled peptide was purified using RP-HPLC operating with a 43.4 minute linear gradient (20-100%) of acetonitrile in water containing TFA (0.1%); lyophilization yielded the final product (HCC(CH₂)₂CO-RGK(CO(CH₂)₃N₃)GPQGIWGQGK(oNB-N₃)RK(CO(CH₂)₃N₃)—NH₂ (SEQ ID NO. 28) with an intramolecular staple via a triazole linkage between the alkyne and oNB-N₃ side chains, denoted E∧P) as a yellow solid (11.2 mg, 0.00477 mmol, 0.95% overall yield). Peptide purity was confirmed using MALDI-TOF: calculated for C₁₀₈H₁₆₆N₄₁O₃₀⁺ [M+¹H]⁺, 2346.2; observed 2346.2.

### Method S13. Reductive “AND” Photo Degradable Crosslinker Synthesis (R∧P)

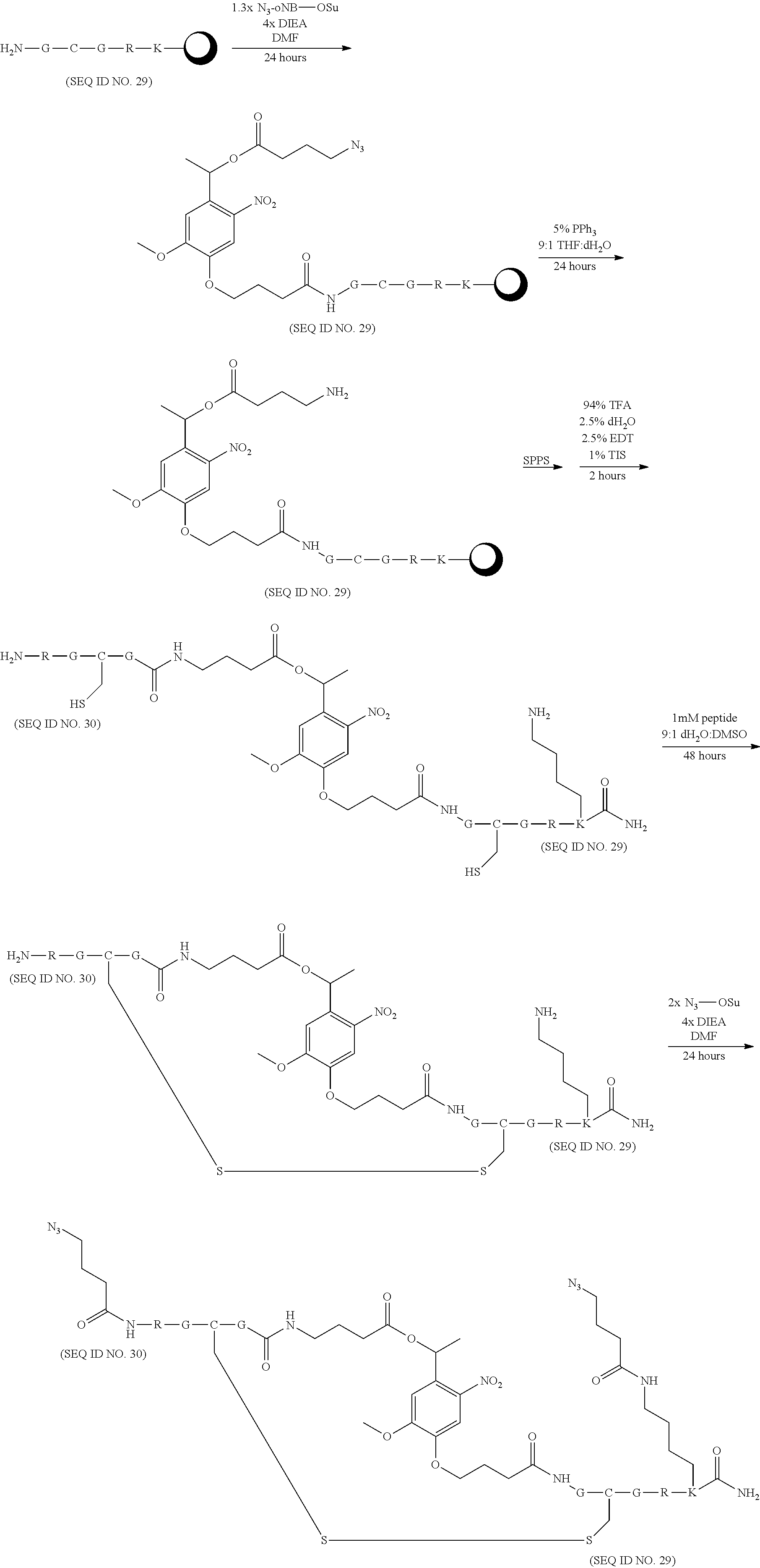

The resin-bound peptide H-GCGRK (SEQ ID NO. 29) was synthesized by microwave-assisted Fmoc SPPS (Method S4) on Rink amide resin (0.5 mmol scale). Resin was subsequently treated with N₃-oNB—OSu (Method S2, 0.65 mmol, 330 mg) and DIEA (2.0 mmol, 258 mg) in minimal DMF to introduce oNB functionality to the N-terminus. The N-terminal azide was reduced to an amine by a Staudinger reduction; resin was washed with THF/H₂O (90:10, 3×20 mL), followed by reaction with 5 wt % triphenylphosphine in THF/H₂O (90:10, 30 mL) for 18 hours. The peptide sequence H-RGCG (SEQ ID NO. 30) was appended to the N-terminus by standard microwave-assisted Fmoc SPPS (Method S4). The peptide was simultaneously deprotected and cleaved from resin upon treatment with TFA/dH₂O/EDT/TIS (94:2.5:2.5:1, 30 mL) for 2 hours, and the crude peptide was precipitated in and washed with ice-cold diethyl ether (2×). The crude peptide was purified using RP-HPLC operating with a 55 minute linear gradient (5-100%) of acetonitrile in water containing TFA (0.10%) lyophilization yielded the pure intermediate (H-RGCG-oNB-GCGRK-NH₂) (SEQ ID NO. 30) connected to (SEQ ID NO. 29) through oNB as a yellow solid (119 mg, 0.0946 mmol). To promote disulfide-mediated intramolecular stapling, the purified peptide was dissolved at 0.5 mM in a dH₂O/DMSO (90:10) solution (200 mL) and reacted at room temperature with no agitation for 48 hours. Stapled product was concentrated in vacuo and purified using RP-HPLC operating with a 55 minute linear gradient (5-100%) of acetonitrile in water containing TFA (0.1%); lyophilization yielded the intermediate (H-RGCG-oNB-GCGRK-NH₂ (SEQ ID NO. 30) connected to (SEQ ID NO. 29) through oNB with intramolecular stapling via cysteine-cysteine disulfide bond) as a yellow solid (101 mg, 0.0807 mmol). To introduce azide functionalities requisite for hydrogel crosslinking, N₃—OSu (Method S1, 73.0 mg, 0.323 mmol, 2×) was dissolved in minimal DMF with DIEA (83.4 mg, 0.646 mmol, 4×) and reacted with the peptide overnight at room temperature. The stapled peptide was purified using RP-HPLC operating with a 55 minute linear gradient (5-100%) of acetonitrile in water containing TFA (0.1%); lyophilization yielded the final product (N₃(CH₂)₃CONH-RGCG-oNB-GCGRK(CO(CH₂)₃N₃)—NH₂ (SEQ ID NO. 30) connected to (SEQ ID NO. 29) through oNB with intramolecular stapling via cysteine-cysteine disulfide bond, denoted R∧P) as a yellow solid (20.0 mg, 0.0135 mmol, 2.7% overall yield). Peptide purity was confirmed using MALDI-TOF: calculated for C₅₇H₉₂N₂₅O₁₈S₂⁺ [M+¹H]⁺, 1478.6; observed 1478.8.

### Method S14. Enzymatic “OR” Reductive “OR” Photo Degradable Crosslinker Synthesis [R∨E∨P]

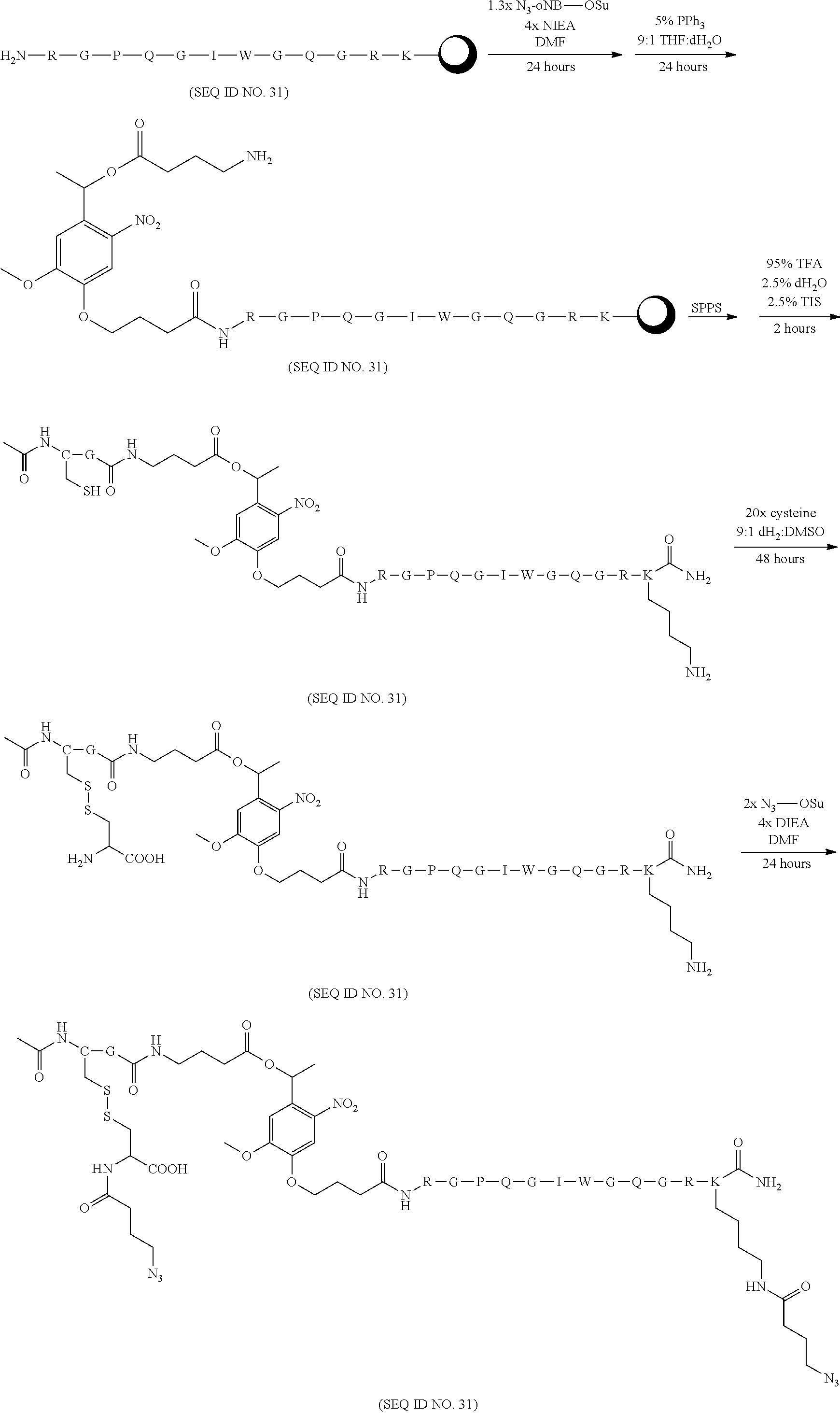

The resin-bound peptide H-RGPQGIWGQGRK (SEQ ID NO. 31) was synthesized by microwave-assisted Fmoc SPPS (Method S4) on Rink amide resin (0.5 mmol scale). Resin was subsequently treated with N₃-oNB—OSu (Method S2, 0.65 mmol, 330 mg) and DIEA (2.0 mmol, 258 mg) in minimal DMF to introduce oNB functionality to the N-terminus. The azide was reduced to an amine by a Staudinger reduction; resin was washed with THF/H₂O (90.10, 3×20 mL), followed by reaction with 5 wt % triphenylphosphine in THF/H₂O (90:10, 30 mL) for 18 hours. The peptide sequence Ac-CG was appended to the N-terminus by standard microwave-assisted Fmoc SPPS. The peptide was simultaneously deprotected and cleaved from resin upon treatment with TFA/TIS/dH₂O (95:2.5:2.5, 30 mL) for 2 hours, and the crude peptide was precipitated in and washed with ice-cold diethyl ether (2×). The crude peptide was purified using RP-HPLC operating with a 43.4 minute linear gradient (20-100%) of acetonitrile in water containing TFA (0.1%); lyophilization yielded the pure intermediate (Ac-CG-oNB-RGPQGIWGQGRK-NH₂) (SEQ ID NO. 31) as a yellow solid (81 mg, 0.0425 mmol). The purified peptide and cysteine (103 mg, 0.850 mmol, 20×) were dissolved in a dH₂O/DMSO (90:10) solution (100 mL) and stirred at room temperature for 48 hours. The peptide was filtered, concentrated in vacuo, and purified using RP-HPLC operating with a 43.4 minute linear gradient (20-100%) of acetonitrile in water containing TFA (0.1%); lyophilization yielded the pure intermediate (Ac—C(H—C—OH)G-oNB-RGPQGIWGQGRK-NH₂ (SEQ ID NO. 31) with cysteines linked via disulfide bond) as a yellow solid (30.9 mg, 0.0152 mmol). To introduce azide functionalities requisite for hydrogel crosslinking, N₃—OSu (Method S1, 13.8 mg, 0.0608 mmol, 2×) was dissolved in minimal DMF with DIEA (15.7 mg, 0.122 mmol, 4×) and reacted with the peptide overnight at room temperature. The peptide was purified using RP-HPLC operating with a 43.4 minute linear gradient (20-100%) of acetonitrile in water containing TFA (0.1%); lyophilization yielded the final product (Ac—C(N₃—C—OH)G-oNB-RGPQGIWGQGRK(N₃)—NH₂ (SEQ ID NO. 31) with cysteines linked via disulfide bond, denoted RV[E∨P]) as a yellow solid (32.2 mg, 0.0257 mmol, 5.1% overall yield). Peptide purity was confirmed using MALDI-TOF: calculated for C₉₃H₁₄₃N₃₄O₂₈S₂⁺ [M+¹H]⁺, 2248.0; observed 2248.6.

### Method S15. Enzymatic “AND” Reductive “AND” Photo Degradable Crosslinker Synthesis [R∧E∧P]

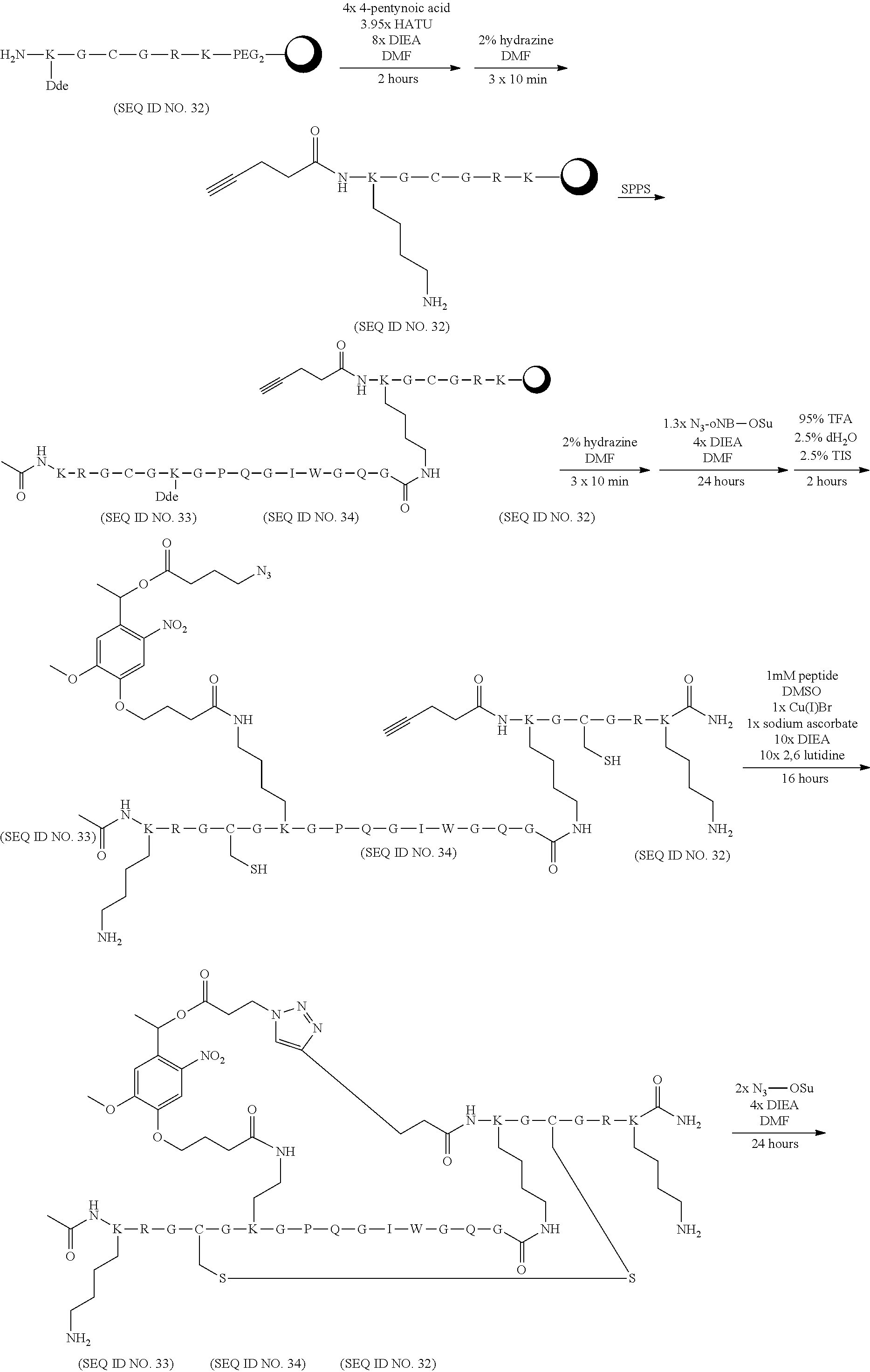

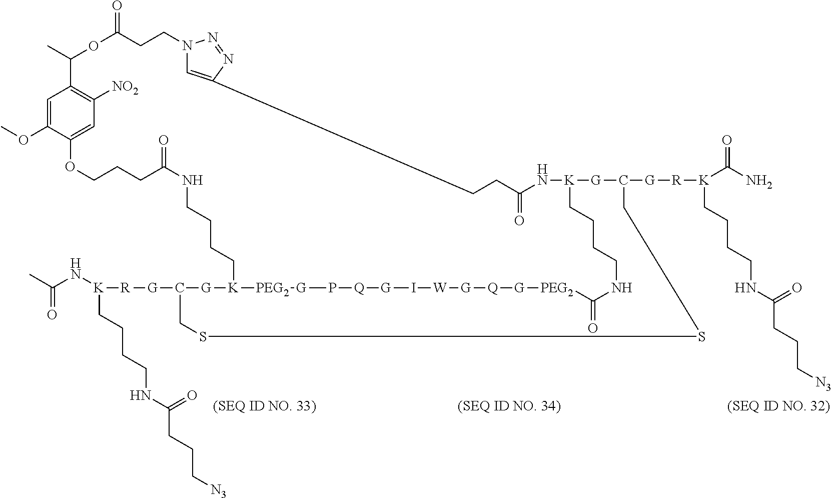

The resin-bound peptide H—K(Dde)GCGRK (SEQ ID NO. 32) was synthesized by microwave-assisted Fmoc SPPS (Method S4) on Rink amide resin (0.5 mmol scale). 4-pentynoic acid (0.196 g, 2.0 mmol, 4×) was pre-activated upon reaction with HATU (0.751 g, 1.975 mmol, 3.95×) and DIEA (0.517 g, 4.0 mmol, 8×) in minimal DMF for 5 minutes and then reacted with the resin for 90 minutes to functionalize the N-terminus with an alkyne. The resin was treated with hydrazine monohydrate (2%) in DMF (3×10 min) to remove the Dde protecting group. Fmoc-8-amino-3,6-dioxaoctanoic acid (residue denoted PEG₂) was incorporated into the peptide to produce sequence Ac-KRGCGK(Dde)-PEG₂-GPQGIWGQG-PEG₂ ((SEQ ID NO. 33) connected to (SEQ ID NO. 34) through PEG₂), which was appended to the ε-amino group of the unprotected lysine by standard microwave-assisted Fmoc SPPS. The resin was again treated with hydrazine monohydrate (2%) in DMF (3×10 min) to remove the second Dde protecting group. Resin was subsequently treated with N₃-oNB—OSu (Method S2, 0.65 mmol, 330 mg) and DIEA (2.0 mmol, 258 mg) in minimal DMF to introduce oNB functionality to the ε-amino group of the unprotected lysine. The peptide was simultaneously deprotected and cleaved from resin upon treatment with TFA/TIS/dH₂O (95:2.5:2.5, 30 mL) for 2 hours, and the crude peptide was precipitated in and washed with ice-cold diethyl ether (2×). The crude peptide was purified using RP-HPLC operating with a 43.4 minute linear gradient (20-100%) of acetonitrile in water containing TFA (0.1%); lyophilization yielded the pure intermediate (Ac-KRGCGK(oNB-N₃)-PEG₂-GPQGIWGQG-PEG₂-K(CO(CH₂)₂CCH)GCGRK-NH₂) (SEQ ID NO. 33) connected to (SEQ ID NO. 34) through PEG₂, and (SEQ ID NO. 34) connected to (SEQ ID NO. 32) by PEG₂, as a yellow solid (88 mg, 0.030 mmol). To induce CuAAC-mediated intramolecular stapling, pure linear peptide (1 mM) was dissolved in nitrogen-purged DMSO (30 mL) containing copper(I) bromide (4.3 mg, 0.030 mmol, 1×), sodium ascorbate (5.8 mg, 0.030 mmol, 1×) in water (300 μL), 2,6 lutidine (31.4 mg, 0.35 mmol, 10×), and DIEA (38.3 mg, 0.35 mmol, 10×). This mixture was allowed to react under nitrogen at room temperature for 16 hours. The mixture was concentrated in vacuo, redissolved in dH₂O, passed through an ion exchange column (Dowex M4195 resin, 5 grams), and lyophilized. Double-stapled product was purified using RP-HPLC operating with a 43.4 minute linear gradient (20-100%) of acetonitrile in water containing TFA (0.1%); lyophilization yielded the pure intermediate (Ac-KRGCGK(oNB-N₃)-PEG₂-GPQGIWGQG-PEG₂-K((CO(CH₂)₂CCH)GCGRK-NH₂ (SEQ ID NO. 33) connected to (SEQ ID NO. 34) through PEG₂, and (SEQ ID NO. 34) connected to (SEQ ID NO. 32) by PEG₂, with an intramolecular staple via a triazole linkage between the alkyne and oNB-N₃ side chains and a second intramolecular staple via cysteine-cysteine disulfide bond) as a yellow solid (20.0 mg, 0.0068 mmol). To introduce azide functionalities requisite for hydrogel crosslinking, N₃—OSu (Method S1, 6.2 mg, 0.027 mmol, 2×) was dissolved in minimal DMF with DIEA (7.1 mg, 0.54 mmol, 4×) and reacted with the peptide overnight at room temperature. The double-stapled product was purified using RP-HPLC operating with a 43.4 minute linear gradient (20-100%) of acetonitrile in water containing TFA (0.1%); lyophilization yielded the final product [Ac—K(N₃)RGCGK(oNB-N₃)-PEG₂-GPQGIWGQG-PEG₂-K((CO(CH₂)₂CCH)GCGRK(N₃)—NH₂ (SEQ ID NO. 33) connected to (SEQ ID NO. 34) through PEG₂, and (SEQ ID NO. 34) connected to (SEQ ID NO. 32) by PEG₂, with an intramolecular staple via a triazole linkage between the alkyne and oNB-N₃ side chains and a second intramolecular staple via cysteine-cysteine disulfide bond, denoted R∧(E∧P)] as a yellow solid (4.2 mg, 0.0013 mmol, 0.26% overall yield). Peptide purity was confirmed using HRMS: calculated for C₁₃₄H₂₁₀N₄₇O₄₀S₂⁺ [M+3 ¹H]³⁺ 1061.181; observed 1061.185.

### Method S16. Enzymatic “AND” (Reductive “OR” Photo) Degradable Crosslinker Synthesis [E∧(R∨P)]

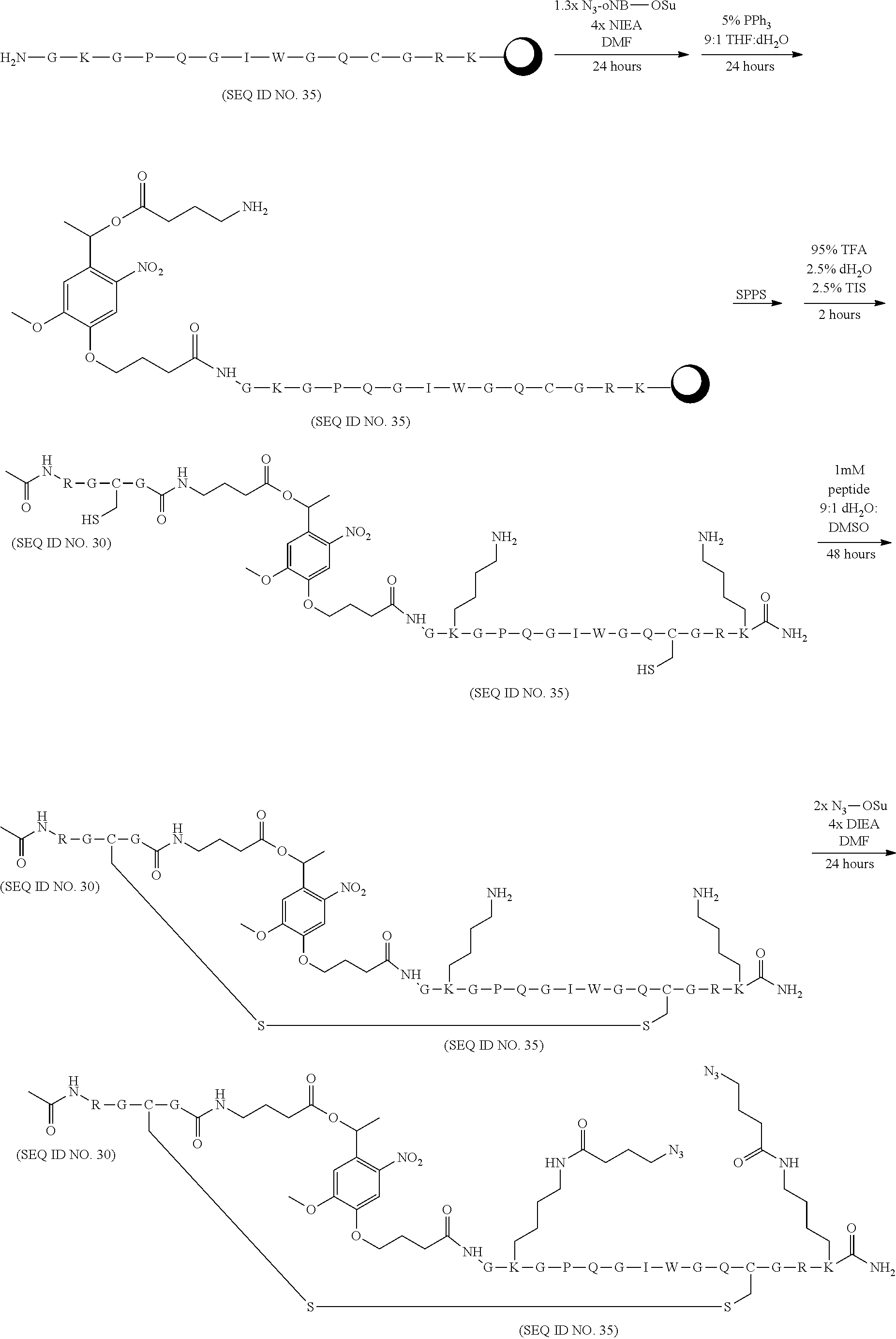

The resin-bound peptide H-GKGPQGIWGQGCGRK (SEQ ID NO. 35) was synthesized by microwave-assisted Fmoc SPPS (Method S4) on Rink amide resin (0.5 mmol scale). Resin was subsequently treated with N₃-oNB—OSu (Method S2, 0.65 mmol, 330 mg) and DIEA (2.0 mmol, 258 mg) in minimal DMF to introduce oNB functionality to the N-terminus. The N-terminal azide was reduced to an amine by a Staudinger reduction; resin was washed with THF/H₂O (90:10, 3×20 mL), followed by reaction with 5 wt % triphenylphosphine in THF/H₂O (90:10, 30 mL) for 18 hours. The peptide sequence Ac-RGCG (SEQ ID NO. 30) was appended to the N-terminus by standard microwave-assisted Fmoc SPPS. The peptide was simultaneously deprotected and cleaved from resin upon treatment with TFA/TIS/dH₂O (95:2.5:2.5, 30 mL) for 2 hours, and the crude peptide was precipitated in and washed with ice-cold diethyl ether (2×). The crude peptide was purified using RP-HPLC operating with a 55 minute linear gradient (5-100%) of acetonitrile in water containing TFA (0.1%); lyophilization yielded the pure intermediate (Ac-RGCG-oNB-GKGPQGIWGQGCGRK-NH₂) (SEQ ID NO. 30) connected to (SEQ ID NO. 35) through oNB, as a yellow solid (57.9 mg, 0.0251 mmol). To promote disulfide-mediated intramolecular stapling, the purified peptide was dissolved at 0.5 mM in a dH₂O/DMSO (90:10) solution (50 mL) and reacted at room temperature with no agitation for 48 hours. Stapled product was concentrated in vacuo and purified using RP-HPLC operating with a 55 minute linear gradient (5-100%) of acetonitrile in water containing TFA (0.1%); lyophilization yielded the pure intermediate (Ac-RGCG-oNB-GKGPQGIWGQGCGRK-NH₂ (SEQ ID NO. 30) connected to (SEQ ID NO. 35) through oNB, with intramolecular stapling via cysteine-cysteine disulfide bond) as a yellow solid (38.5 mg, 0.0167 mmol). To introduce azide functionalities requisite for hydrogel crosslinking, N₃—OSu (Method S1, 15.1 mg, 0.0668 mmol, 2×) was dissolved in minimal DMF with DIEA (17.3 mg, 0.134 mmol, 4×) and reacted with the peptide overnight at room temperature. The stapled peptide was purified using RP-HPLC operating with a 55 minute linear gradient (5-100%) of acetonitrile in water containing TFA (0.1%); lyophilization yielded the final product [Ac-RGCG-oNB-GK(N₃)GPQGIWGQGCGRK(N₃)—NH₂ (SEQ ID NO. 30) connected to (SEQ ID NO. 35) through oNB, with intramolecular stapling via cysteine-cysteine disulfide bond, denoted E∧(R∨P)] as a yellow solid (14.0 mg, 0.0055 mmol, 1.1% overall yield). Peptide purity was confirmed using MALDI-TOF: calculated for C₁₀₅H₁₆₂N₃₉O₃₁S₂⁺ [M+¹H]⁺, 2529.2; observed 2529.4.

### Method S17. Reductive “AND” (Enzymatic “OR” Photo) Degradable Crosslinker Synthesis [R∧(E∨P)]

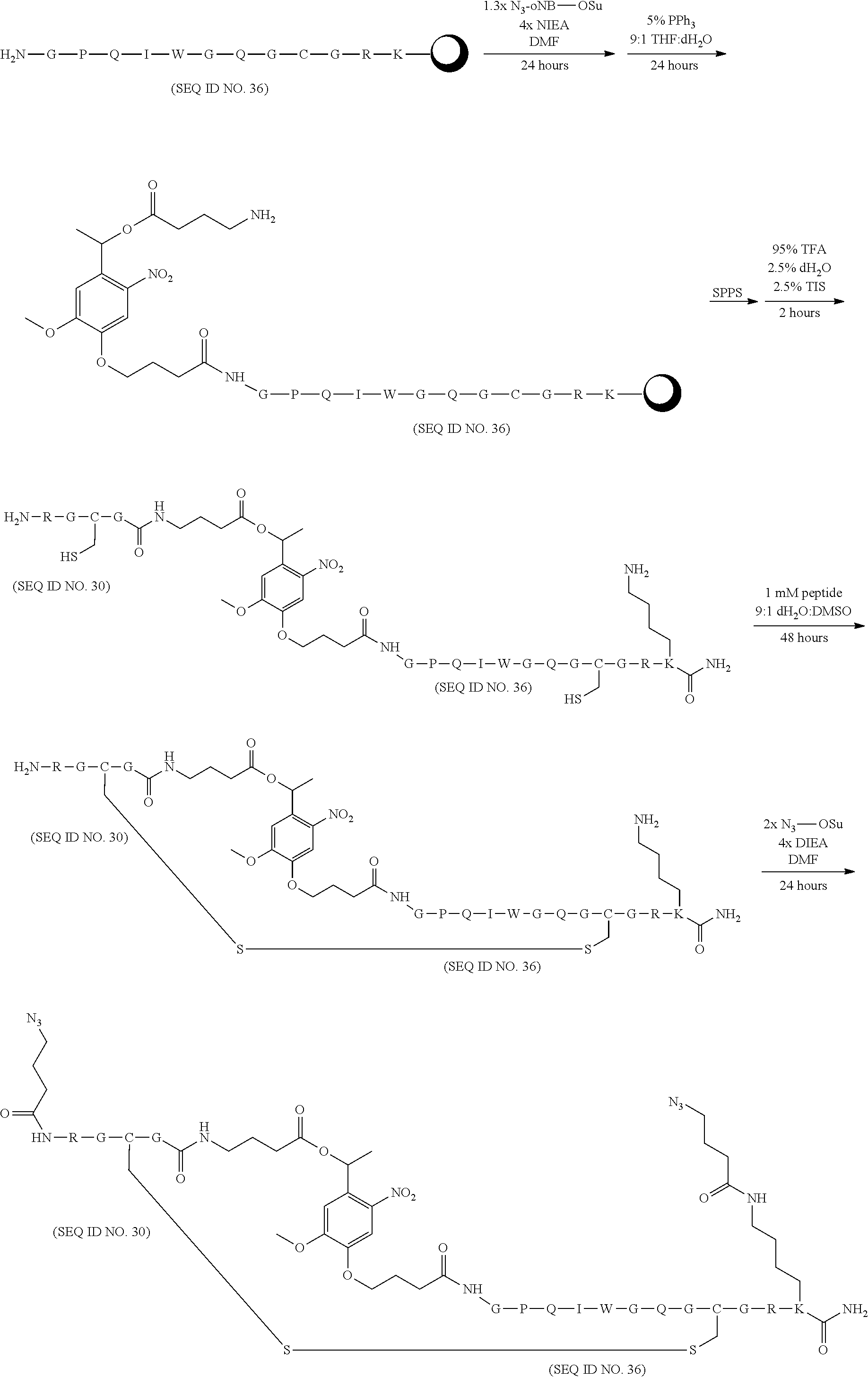

The resin-bound peptide H-GPQGIWGQGCGRK (SEQ ID NO. 36) was synthesized by microwave-assisted Fmoc SPPS (Method S4) on Rink amide resin (0.5 mmol scale). Resin was subsequently treated with N₃-oNB—OSu (Method S2, 0.65 mmol, 330 mg) and DIEA (2.0 mmol, 258 mg) in minimal DMF to introduce oNB functionality to the N-terminus. The N-terminal azide was reduced to an amine by a Staudinger reduction; resin was washed with THF/H₂O (90:10, 3×20 mL), followed by reaction with 5 wt % triphenylphosphine in THF/H₂O (90:10, 30 mL) for 18 hours. The peptide sequence H-RGCG (SEQ ID NO. 30) was appended to the N-terminus by standard microwave-assisted Fmoc SPPS. The peptide was simultaneously deprotected and cleaved from resin upon treatment with TFA/TIS/dH₂O (95:2.5:2.5, 30 mL) for 2 hours, and the crude peptide was precipitated in and washed with ice-cold diethyl ether (2×). The crude peptide was purified using RP-HPLC operating with a 55 minute linear gradient (5-100%) of acetonitrile in water containing TFA (0.1%); lyophilization yielded the pure intermediate (H-RGCG-oNB-GPQGIWGQGCGRK-NH₂) (SEQ ID NO. 30) connected to (SEQ ID NO. 36) through oNB, as a yellow solid (58 mg, 0.028 mmol). To promote disulfide-mediated intramolecular stapling, the purified peptide was dissolved at 0.5 mM in a dH₂O/DMSO (90:10) solution (55 mL) and reacted at room temperature with no agitation for 48 hours. Stapled product was concentrated in vacuo and purified using RP-HPLC operating with a 55 minute linear gradient (5-100%) of acetonitrile in water containing TFA (0.1%); lyophilization yielded the pure intermediate (H-RGCG-oNB-GPQGIWGQGCGRK-NH₂ (SEQ ID NO. 30) connected to (SEQ ID NO. 36) through oNB, with intramolecular stapling via cysteine-cysteine disulfide bond) as a yellow solid (23.5 mg, 0.0113 mmol). To introduce azide functionalities requisite for hydrogel crosslinking, N₃—OSu (Method 51, 10.2 mg, 0.0452 mmol, 2×) was dissolved in minimal DMF with DIEA (11.7 mg, 0.0904 mmol, 4×) and reacted with the peptide overnight at room temperature. The stapled peptide was purified using RP-HPLC operating with a 55 minute linear gradient (5-100%) of acetonitrile in water containing TFA (0.10%); lyophilization yielded the final product [N₃-RGCG-oNB-GPQGIWGQGCGRK(CO(CH₂)₃N₃)—NH₂ (SEQ ID NO. 30) connected to (SEQ ID NO. 36) through oNB, with intramolecular stapling via cysteine-cysteine disulfide bond, denoted R∧(E∨P)] as a yellow solid (11.2 mg, 0.0049 mmol, 1.0% overall yield). Peptide purity was confirmed using MALDI-TOF: calculated for C₉₅H₁₄₅N₃₆O₂₈S₂⁺ [M+¹H]⁺, 2302.0; observed 2302.5.

### Method S18. Photo “AND” (Reductive “OR” Enzymatic) Degradable Crosslinker Synthesis [P∧(R∨E)]

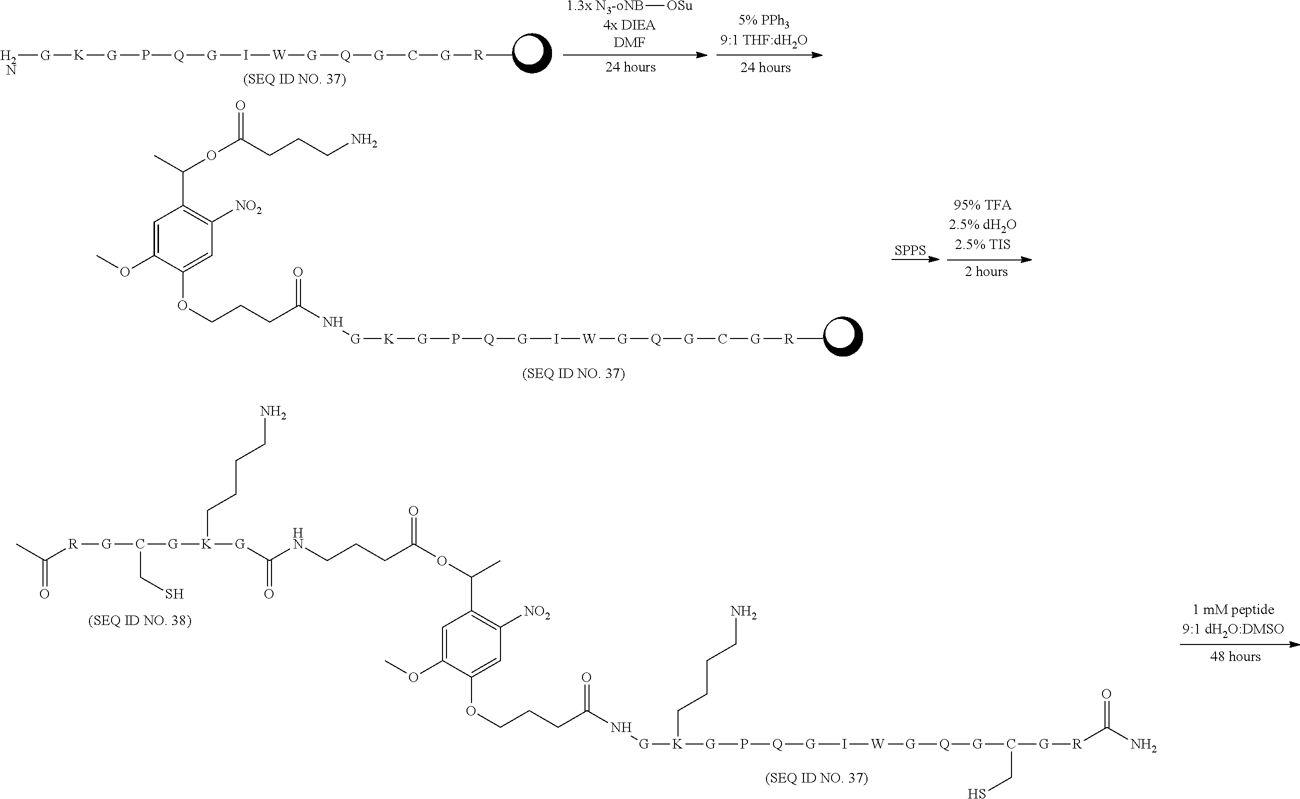

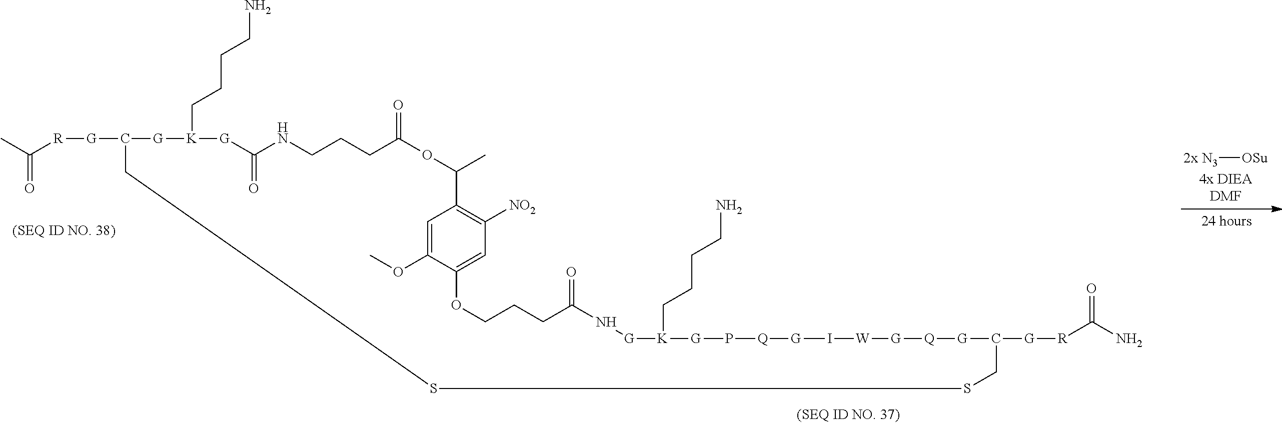

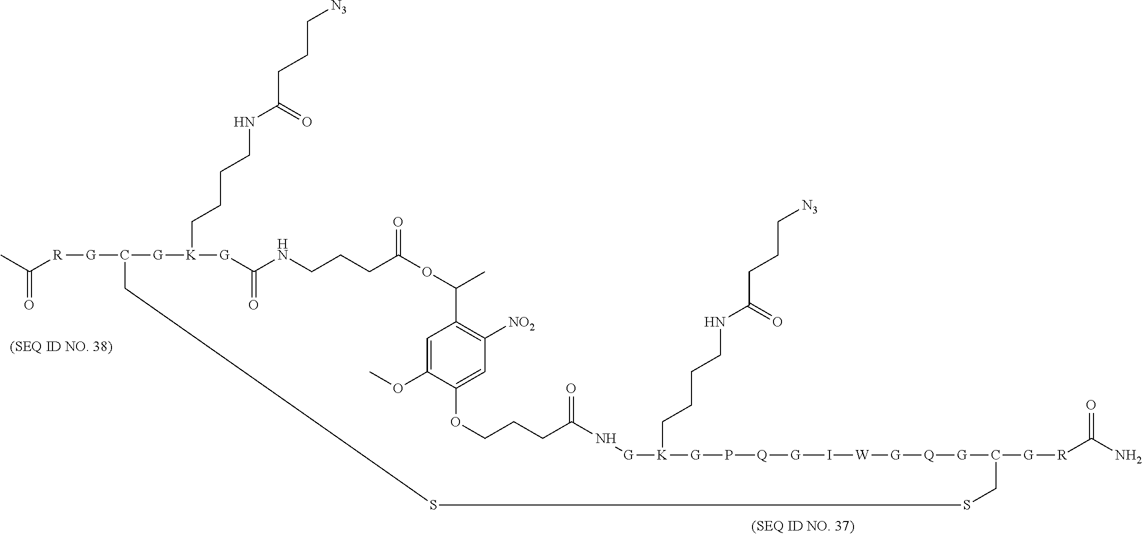

The resin-bound peptide H-GKGPQGIWGQGCGR (SEQ ID NO. 37) was synthesized by microwave-assisted Fmoc SPPS (Method S4) on Rink amide resin (0.5 mmol scale). Resin was subsequently treated with N₃-oNB—OSu (Method S2, 0.65 mmol, 330 mg) and DIEA (2.0 mmol, 258 mg) in minimal DMF to introduce oNB functionality to the N-terminus. The N-terminal azide was reduced to an amine by a Staudinger reduction; resin was washed with THF/H₂O (90:10, 3×20 mL), followed by reaction with 5 wt % triphenylphosphine in THF/H₂O (90:10, 30 mL) for 18 hours. The peptide sequence Ac-RGCGKG (SEQ ID NO. 38) was appended to the N-terminus by standard microwave-assisted Fmoc SPPS. The peptide was simultaneously deprotected and cleaved from resin upon treatment with TFA/TIS/dH₂O (95:2.5:2.5, 30 mL) for 2 hours, and the crude peptide was precipitated in and washed with ice-cold diethyl ether (2×). The crude peptide was purified using RP-HPLC operating with a 55 minute linear gradient (5-100%) of acetonitrile in water containing TFA (0.1%); lyophilization yielded the pure intermediate (Ac-RGCGKG-oNB-GKGPQGIWGQGCGR-NH₂) (SEQ ID NO. 38) connected to (SEQ ID NO. 37) through oNB, as a yellow solid (93.1 mg, 0.0393 mmol). To promote disulfide-mediated intramolecular stapling, the purified peptide was dissolved at 0.5 mM in a dH₂O/DMSO (90:10) solution (80 mL) and reacted at room temperature with no agitation for 48 hours. Stapled product was concentrated in vacuo and purified using RP-HPLC operating with a 55 minute linear gradient (5-100%) of acetonitrile in water containing TFA (0.1%); lyophilization yielded the pure intermediate (Ac-RGCGKG-oNB-GKGPQGIWGQGCGR-NH₂ (SEQ ID NO. 38) connected to (SEQ ID NO. 37) through oNB, with intramolecular stapling via cysteine-cysteine disulfide bond) as a yellow solid (55.3 mg, 0.0234 mmol). To introduce azide functionalities requisite for hydrogel crosslinking, N₃—OSu (Method S1, 21.2 mg, 0.0936 mmol, 2×) was dissolved in minimal DMF with DIEA (24.2 mg, 0.187 mmol, 4×) and reacted with the peptide overnight at room temperature. The stapled peptide was purified using RP-HPLC operating with a 55 minute linear gradient (5-100%) of acetonitrile in water containing TFA (0.1%); lyophilization yielded the final product [Ac-RGCGK(N₃)G-oNB-GK(CO(CH₂)₃N₃)GPQGIWGQGCGR-NH₂ (SEQ ID NO. 38) connected to (SEQ ID NO. 37) through oNB, with intramolecular stapling via cysteine-cysteine disulfide bond, denoted P∧(R∨E)] as a yellow solid (11.0 mg, 0.0043 mmol, 0.9% overall yield). Peptide purity was confirmed using MALDI-TOF: calculated for C₁₀₇H₁₆₅N₄₀O₃₂S₂⁺ [M+¹H]⁺, 2586.2; observed 2586.4.

### Method S₁₉. Enzymatic “OR” (Reductive “AND” Photo) Degradable Crosslinker Synthesis [E∨(R∧P)]

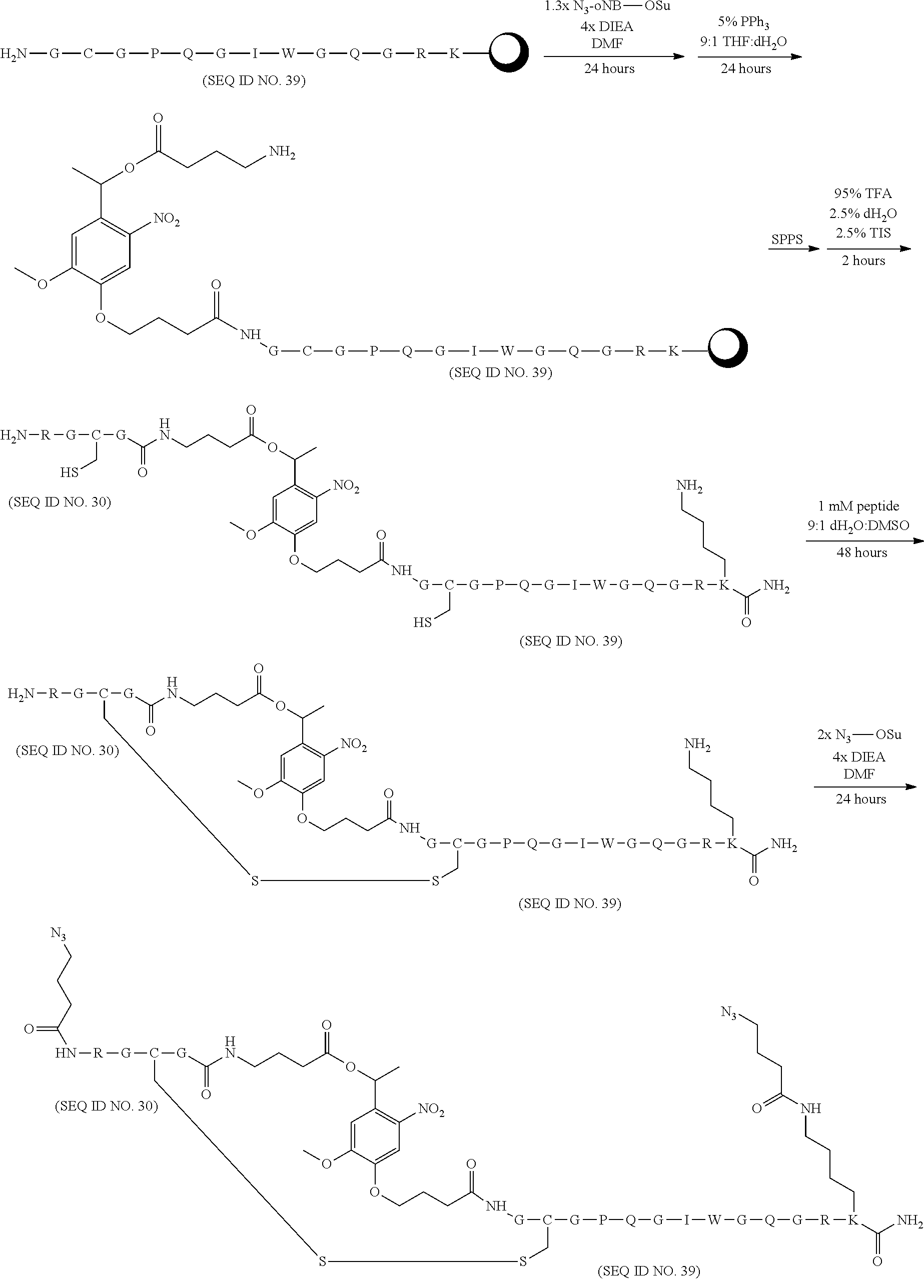

The resin-bound peptide H-GCGPQGIWGQGRK (SEQ ID NO. 39) was synthesized by microwave-assisted Fmoc SPPS (Method S4) on Rink amide resin (0.5 mmol scale). Resin was subsequently treated with N₃-oNB—OSu (Method S2, 0.65 mmol, 330 mg) and DIEA (2.0 mmol, 258 mg) in minimal DMF to introduce oNB functionality to the N-terminus. The N-terminal azide was reduced to an amine by a Staudinger reduction; resin was washed with THF/H₂O (90:10, 3×20 mL), followed by reaction with 5 wt % triphenylphosphine in THF/H₂O (90:10, 30 mL) for 18 hours. The peptide sequence H-RGCG (SEQ ID NO. 30) was appended to the N-terminus by standard microwave-assisted Fmoc SPPS. The peptide was simultaneously deprotected and cleaved from resin upon treatment with TFA/TIS/dH₂O (95:2.5:2.5, 30 mL) for 2 hours, and the crude peptide was precipitated in and washed with ice-cold diethyl ether (2×). The crude peptide was purified using RP-HPLC operating with a 43.4 minute linear gradient (20-100%) of acetonitrile in water containing TFA (0.1%); lyophilization yielded the pure intermediate (H-RGCG-oNB-GCGPQGIWGQGRK-NH₂) (SEQ ID NO. 30) connected to (SEQ ID NO. 39) through oNB, as a yellow solid (85 mg, 0.0408 mmol). To promote disulfide-mediated intramolecular stapling, the purified peptide was dissolved at 0.5 mM in a dH₂O/DMSO (90:10) solution (80 mL) and reacted at room temperature with no agitation for 48 hours. Stapled product was concentrated in vacuo and purified using RP-HPLC operating with a 43.4 minute linear gradient (20-100%) of acetonitrile in water containing TFA (0.1%); lyophilization yielded the pure intermediate (H-RGCG-oNB-GCGPQGIWGQGRK-NH₂ (SEQ ID NO. 30) connected to (SEQ ID NO. 39) through oNB, with intramolecular stapling via cysteine-cysteine disulfide bond) as a yellow solid (33.9 mg, 0.0163 mmol). To introduce azide functionalities requisite for hydrogel crosslinking, N₃—OSu (Method 51, 14.7 mg, 0.0652 mmol, 2×) was dissolved in minimal DMF with DIEA (16.9 mg, 0.130 mmol, 4×) and reacted with the peptide overnight at room temperature. Stapled product was purified using RP-HPLC operating with a 43.4 minute linear gradient (20-100%) of acetonitrile in water containing TFA (0.10%); lyophilization yielded the final product [N₃-RGCG-oNB-GCGPQGIWGQGRK(CO(CH₂)₃N₃)—NH₂ (SEQ ID NO. 30) connected to (SEQ ID NO. 39) through oNB, with intramolecular stapling via cysteine-cysteine disulfide bond, denoted E∨(R∧P)] as a yellow solid (14.9 mg, 0.0065 mmol, 1.3% overall yield). Peptide purity was confirmed using MALDI-TOF: calculated for C₉₅H₁₄₅N₃₆O₂₈S₂⁺ [M+¹H]⁺, 2302.0; observed 2303.0.

### Method S20. Reductive “OR” (Enzymatic “AND” Photo) Degradable Crosslinker Synthesis [R∨(E∧P)]

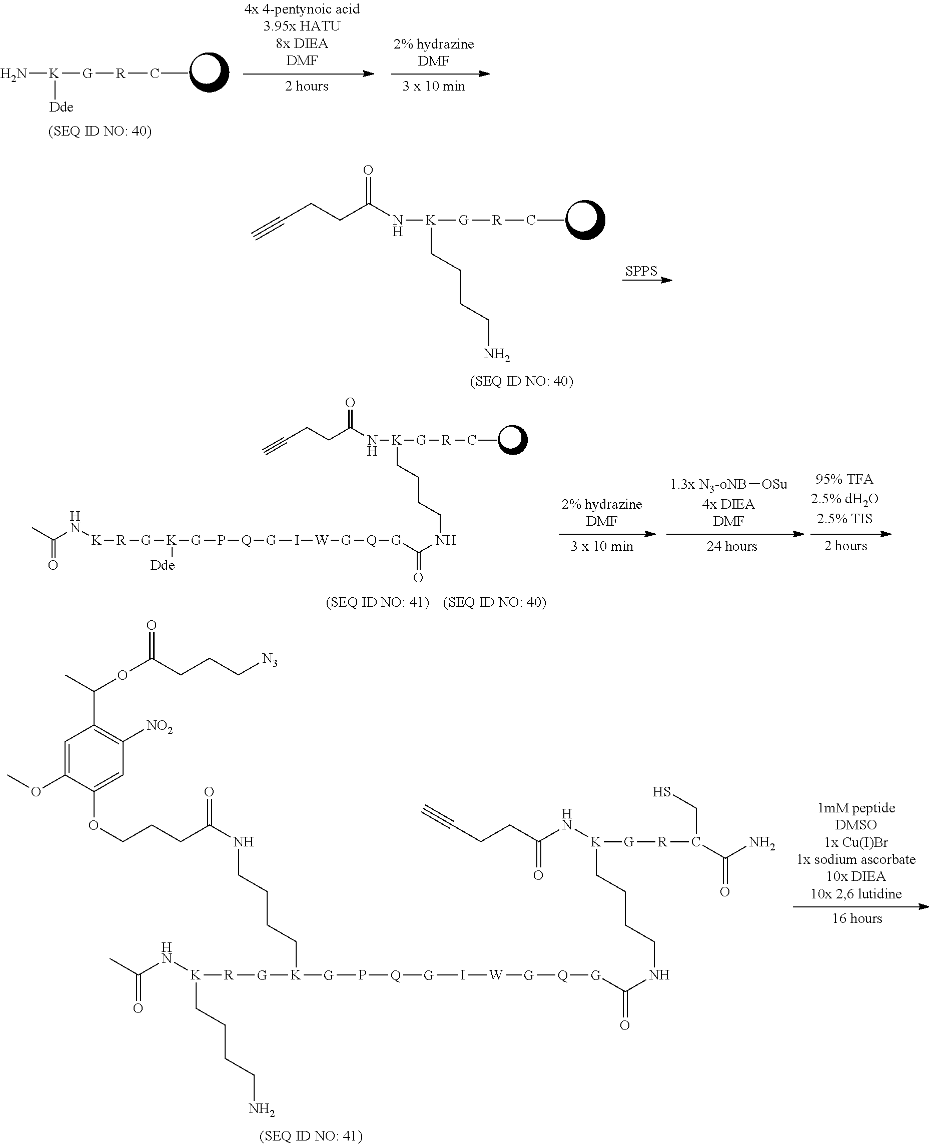

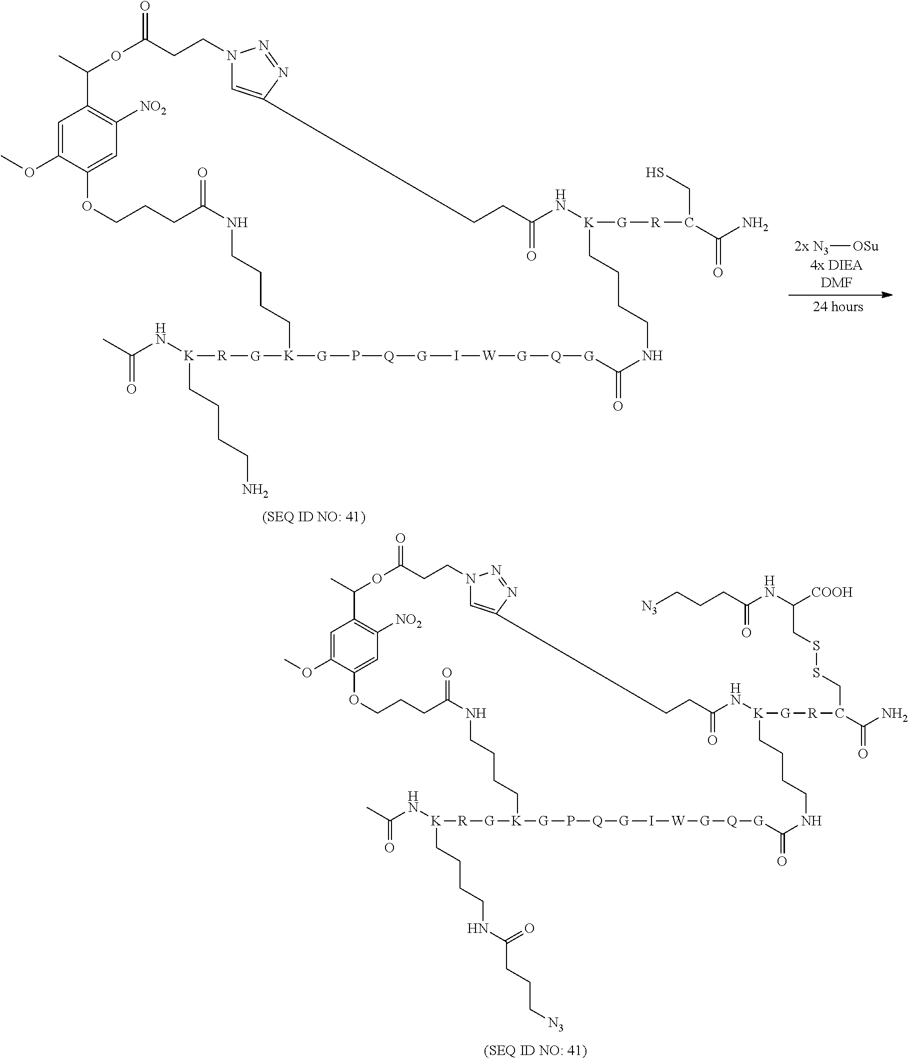

The resin-bound peptide H—K(Dde)GRC (SEQ ID NO. 40) was synthesized by microwave-assisted Fmoc SPPS (Method S4) on Rink amide resin (0.5 mmol scale). 4-pentynoic acid (0.196 g, 2.0 mmol, 4×) was pre-activated upon reaction with HATU (0.751 g, 1.975 mmol, 3.95×) and DIEA (0.517 g, 4.0 mmol, 8×) in minimal DMF for 5 minutes and then reacted with the resin for 90 minutes to functionalize the N-terminus with an alkyne. The resin was treated with hydrazine monohydrate (2%) in DMF (3×10 min) to remove the Dde protecting group. The peptide sequence Ac-KRGK(Dde)GPQGIWGQG (SEQ ID NO. 41) was appended to the ε-amino group of the unprotected lysine by standard microwave-assisted Fmoc. The resin was again treated with hydrazine monohydrate (2%) in DMF (3×10 min) to remove the second Dde protecting group. Resin was subsequently treated with N₃-oNB—OSu (Method S2, 0.65 mmol, 330 mg) and DIEA (2.0 mmol, 258 mg) in minimal DMF to introduce oNB functionality to the 8-amino group of the unprotected lysine. The peptide was simultaneously deprotected and cleaved from resin upon treatment with TFA/TIS/dH₂O (95:2.5:2.5, 30 mL) for 2 hours, and the crude peptide was precipitated in and washed with ice-cold diethyl ether (2×). The crude peptide was purified using RP-HPLC operating with a 43.4 minute linear gradient (20-100%) of acetonitrile in water containing TFA (0.1%); lyophilization yielded the pure intermediate (Ac-KRGK(oNB-N₃)GPQGIWGQG-K(CO(CH₂)₂CCH)GRC-NH₂) (SEQ ID NO. 41) connected with (SEQ ID NO. 40), as a yellow solid (115 mg, 0.0494 mmol). To induce CuAAC-mediated intramolecular stapling, pure linear peptide (1 mM) was dissolved in nitrogen-purged DMSO (50 mL) containing copper(I) bromide (7.1 mg, 0.049 mmol, 1×), sodium ascorbate (9.8 mg, 0.049 mmol, 1×) in water (480 μL), 2,6 lutidine (52.9 mg, 0.494 mmol, 10×), and DIEA (63.8 mg, 0.494 mmol, 10×). This mixture was allowed to react under nitrogen at room temperature for 16 hours. The mixture was concentrated in vacuo, redissolved in dH₂O, passed through an ion exchange column (Dowex M4195 resin, 5 grams), and lyophilized. Stapled product was purified using RP-HPLC operating with a 43.4 minute linear gradient (20-100%) of acetonitrile in water containing TFA (0.1%); lyophilization yielded the pure intermediate (Ac-KRGK(oNB-N₃)GPQGIWGQG-K(CO(CH₂)₂CCH)GRC-NH₂(SEQ ID NO. 41) connected with (SEQ ID NO. 40), with an intramolecular staple via a triazole linkage between the alkyne and oNB-N₃ side chains) as a yellow solid (55 mg, 0.0236 mmol). The purified peptide and cysteine (57 mg, 0.47 mmol, 20×) were dissolved in a dH₂O/DMSO (90:10) solution (20 mL) and stirred at room temperature for 48 hours. The stapled peptide was filtered, concentrated in vacuo, and purified using RP-HPLC operating with a 43.4 minute linear gradient (20-100%) of acetonitrile in water containing TFA (0.1%); lyophilization yielded the pure intermediate (Ac-KRGK(oNB-N₃)GPQGIWGQG-K(CO(CH₂)₂CCH)GRC(H—C—OH)—NH₂ (SEQ ID NO. 41) connected with (SEQ ID NO. 40), with an intramolecular staple via a triazole linkage between the alkyne and oNB-N₃ side chains and cysteines linked via disulfide bond) as a yellow solid (30.0 mg, 0.0123 mmol). To introduce azide functionalities requisite for hydrogel crosslinking, N₃—OSu (Method S1, 11.1 mg, 0.049 mmol, 2×) was dissolved in minimal DMF with DIEA (12.7 mg, 0.098 mmol, 4×) and reacted with the peptide overnight at room temperature. The stapled peptide was purified using RP-HPLC operating with a 43.4 minute linear gradient (20-100%) of acetonitrile in water containing TFA (0.1%); lyophilization yielded the final product [Ac—K(N₃)RGK(oNB-N₃)GPQGIWGQG-K(CO(CH₂)₂CCH)GRC(N₃—C—OH)—NH₂ (SEQ ID NO. 41) connected with (SEQ ID NO. 40), with an intramolecular staple via a triazole linkage between the alkyne and oNB-N₃ side chains and cysteines linked via disulfide bond, denoted R∨(E∧P)] as a yellow solid (27.1 mg, 0.0102 mmol, 2.0% overall yield). Peptide purity was confirmed using MALDI-TOF: calculated for C₁₁₂H₁₇₂N₄₁O₃₂S₂⁺ [M+¹H]⁺, 2667.3; observed 2667.9.

### Method S21. Photo “OR” (Reductive “AND” Enzymatic) Degradable Crosslinker Synthesis [P∨(R∧E)]

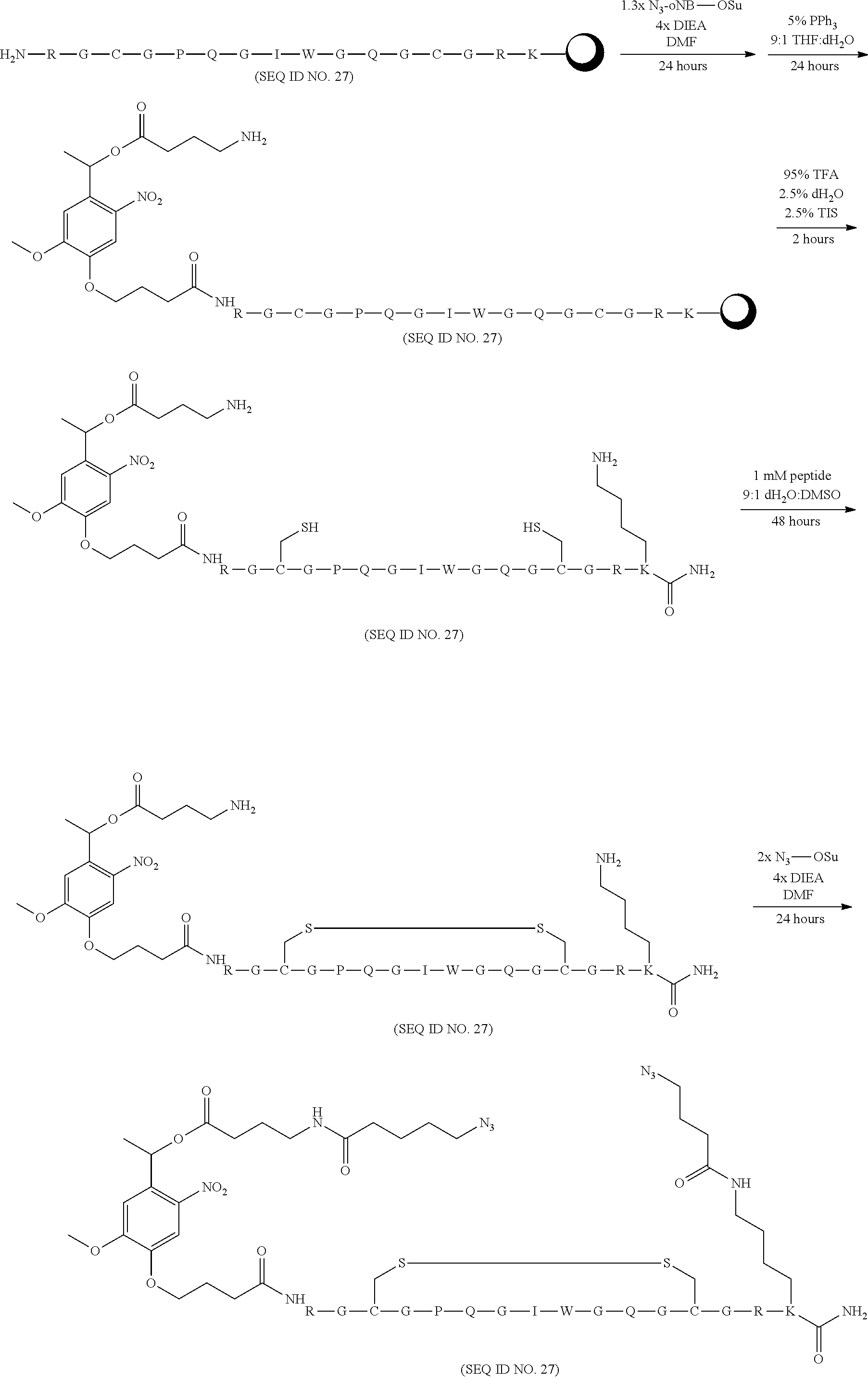

The resin-bound peptide H-RGCGPQGIWGQGCGRK. (SEQ ID NO. 27) was synthesized by microwave-assisted Fmoc SPPS (Method S4) on Rink amide resin (0.5 mmol scale). Resin was subsequently treated with N₃-oNB—OSu (Method S2, 0.65 mmol, 330 mg) and DIEA (2.0 mmol, 258 mg) in minimal DMF to introduce oNB functionality to the N-terminus. The N-terminal azide was reduced to an amine by a Staudinger reduction; resin was washed with THF/H₂O (90:10, 3×20 mL), followed by reaction with 5 wt % triphenylphosphine in THF/H₂O (90:10, 30 mL) for 18 hours. The peptide was simultaneously deprotected and cleaved from resin upon treatment with TFA/TIS/dH₂O (95:2.5:2.5, 30 mL) for 2 hours, and the crude peptide was precipitated in and washed with ice-cold diethyl ether (2×). The crude peptide was purified using RP-HPLC operating with a 43.4 minute linear gradient (20-100%) of acetonitrile in water containing TFA (0.1%); lyophilization yielded the pure intermediate (H₂N-oNB-RGCGPQGIWGQGCGRK-NH₂) (SEQ ID NO. 27) as a yellow solid (99 mg, 0.0489 mmol). To promote disulfide-mediated intramolecular stapling, the purified peptide was dissolved at 0.5 mM in a dH₂O/DMSO (90:10) solution (100 mL) and reacted at room temperature with no agitation for 48 hours. Stapled product was concentrated in vacuo and purified using RP-HPLC operating with a 43.4 minute linear gradient (20-100%) of acetonitrile in water containing TFA (0.1%); lyophilization yielded the pure intermediate (H₂N-oNB-RGCGPQGIWGQGCGRK-NH₂ (SEQ ID NO. 27) with intramolecular stapling via cysteine-cysteine disulfide bond) as a yellow solid (33.7 mg, 0.0167 mmol). To introduce azide functionalities requisite for hydrogel crosslinking, N₃—OSu (Method S1, 15.1 mg, 0.0668 mmol, 2×) was dissolved in minimal DMF with DIEA (17.2 mg, 0.133 mmol, 4×) and reacted with the peptide overnight at room temperature. The stapled peptide was purified using RP-HPLC operating with a 43.4 minute linear gradient (20-100%) of acetonitrile in water containing TFA (0.1%); lyophilization yielded the final product N₃-oNB-RGCGPQGIWGQGCGRK(N₃)—NH₂ (SEQ ID NO. 27) with intramolecular stapling via cysteine-cysteine disulfide bond, denoted P∨(R∧E)] as a yellow solid (10.0 mg, 0.0045 mmol, 0.9% overall yield). Peptide purity was confirmed using MALDI-TOF: calculated for C₉₃H₁₄₂N₃₅O₂₇S₂⁺ [M+¹H]⁺, 2245.0; observed 2245.5.

### Method S22. Synthesis of 4-Arm-PEG_{20 kDa}-Tetrabicyclononyne (PEG-tetraBCN) and Fluorescent Variants (PEG-tetraBCN-AF568, PEG-tetraBCN-FAM, PEG-tetraBCN-Cyanine5)

(1R,8S,9s)-bicyclo[6.1.0]non-4-yn-9-ylmethyl (2,5-dioxopyrrolidin-1-yl) carbonate (BCN-OSu) was synthesized from (1R,8S,9s)-bicyclo[6.1.0]non-4-yn-9-ylmethanol (BCN—OH).

### Synthesis of (1R8S,9s,Z)-ethyl bicyclo[6.1.0]non-4-ene-9-carboxylate (endo)

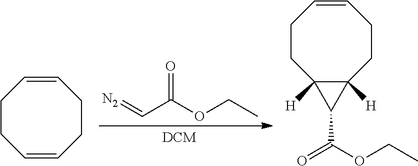

Ethyl diazoacetate (7.56 g, 7.0 mL, 66 mmol) in DCM (35 mL) was added dropwise to a 0° C. solution of 1,5-cyclooctadiene (44.1 g, 50 mL, 408 mmol), rhodium(II) acetate dimer catalyst (1.0 g), and DCM (30 mL). The mixture was reacted at room temperature for 72 hours, filtered to remove the catalyst, and concentrated in vacuo. The product was purified on a silica column (0-1% ethyl acetate in hexanes) and concentrated to give both the exo (5.62 g, 28.9 mmol, 43.8% yield) and desired endo product (3.70 g, 19.0 mmol, 28.9% yield). Endo: ¹H NMR (500 MHz, CDCl₃) δ 5.64 (m, 2H), 4.14 (q, J=7.1 Hz, 2H), 2.53 (m, 2H), 2.23 (m, 2H), 2.08 (m, 2H), 1.86 (m, 2H), 1.73 (t, J=8.8 Hz, 1H), 1.42 (m, 2H), 1.29 (t, J=7.1 Hz, 3H).

### Synthesis of (1R,8S,9s)-bicyclo[6.1.0]non-4-yn-9-ylmethanol

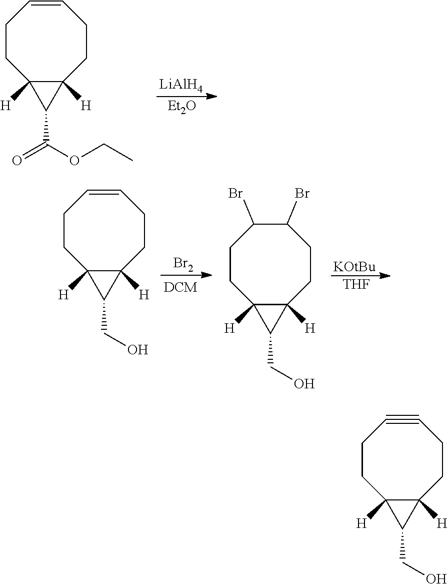

The endo intermediate, (1R,8S,9s,Z)-ethyl bicyclo[6.1.0]non-4-ene-9-carboxylate (3.70 g, 19.0 mmol), was dissolved in 0° C. anhydrous ether (65 mL). Lithium aluminum hydride (750 mg, 19.8 mmol, 1.04×) in 0° C. anhydrous ether (130 mL) was added dropwise to the ester over 15 minutes and the reaction was stirred at room temperature for 20 minutes. Minimal water was added to quench the solution and induced the formation of a grey precipitate. The solution was dried over MgSO₄, filtered, and concentrated in vacuo to yield the crude alcohol intermediate (1R,8S,9s,Z)-bicyclo[6.1.0]non-4-en-9-ylmethanol (2.91 g, 19.1 mmol, quantitative yield).

A solution of bromine (3.1 g, 1.0 mL, 19 mmol) in DCM (13 mL) and added dropwise to a 0° C. solution of the crude hydroxyl intermediate dissolved in anhydrous DCM (140 mL) until a yellow color persisted. The reaction was quenched with aqueous sodium thiosulfate (10 wt %, 50 mL). The product was extracted into DCM (2×70 mL), dried over MgSO₄, filtered, and concentrated in vacuo to yield the crude dibromide intermediate ((1R,8S,9s)-4,5-dibromobicyclo[6.1.0]nonan-9-yl)methanol (5.8 g, 19 mmol, quantitative yield).

The dibromide intermediate was dissolved in 0° C. anhydrous THF (120 mL) to which a solution of potassium tert-butoxide (1 M in THF, 50 mL) was added dropwise. The reaction mixture was stirred while refluxing at 75° C. for 2.5 hours. The solution was cooled to room temperature and quenched with saturated aqueous ammonium chloride (150 mL). THF was removed in vacuo and the product was extracted into DCM (3×70 mL), dried over MgSO₄, filtered, and concentrated. The product was purified on a silica column (0-20% ethyl acetate in hexanes) and concentrated to yield pure (1R,8S,9s)-bicyclo[6.1.0]non-4-yn-9-ylmethanol as a yellow oil (1.48 g, 9.85 mmol, 52% yield over 3 steps). ¹H NMR (500 MHz, CDCl₃) δ 3.62 (d, J=7.7 Hz, 2H), 2.59 (br s, 1H), 2.16 (m, 6H), 1.50 (m, 2H), 1.22 (m, 1H), 0.84 (m, 1H).

### Synthesis of [(1R,8S9S)-bicyclo[6.1.0]non-4-yn-9-yl]methyl 2,5-dioxopyrrolidin-1-yl carbonate (BCN-OSu)

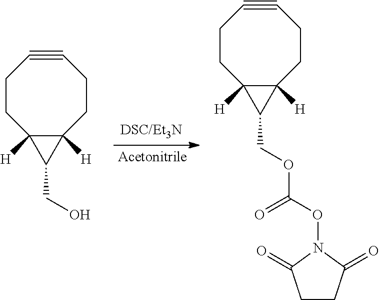 [SMILES: OC[C@H]1[C@H]2CCC#CCC[C@@H]12 ; O=C(OC[C@@H]1[C@@H]2CCC#CCC[C@@H]21)ON1C(=O)CCC1=O]

(1R,8S,9s)-bicyclo[6.1.0]non-4-yn-9-ylmethanol (1.48 g, 9.9 mmol) and N,N′-Disuccinimidyl carbonate (5.1 g, 19.9 mmol, 2×) were dissolved in acetonitrile (75 mL) under a nitrogen atmosphere. Triethylamine (3.3 g, 4.5 mL, 32 mmol) was added and the reaction was stirred overnight at room temperature. The reaction was diluted with 1:1 ethyl acetate:ether (200 mL) and washed with water (6×100 mL) and brine (2×50 mL). The organic layer was dried over MgSO₄, filtered, and concentrated in vacuo. The product was purified on a silica flash column (3:1 hexanes:ethyl acetate) to yield the product [(1R,8S,9S)-bicyclo[6.1.0]non-4-yn-9-yl]methyl 2,5-dioxopyrrolidin-1-yl carbonate (denoted BCN-OSu) as a white solid (1.72 g, 5.9 mmol, 60% yield). ¹H NMR (500 MHz, CDCl₃): δ 4.48 (d, J=8.4 Hz, 2H), 2.87 (s, 4H), 2.30 (m, 6H), 1.57 (m, 3H), 1.09 (m, 2H). These spectral data matched those previously reported.

### Synthesis of 4-arm-PEG_{20 kDa}-tetrabicyclononyne (PEG-tetraBCN)

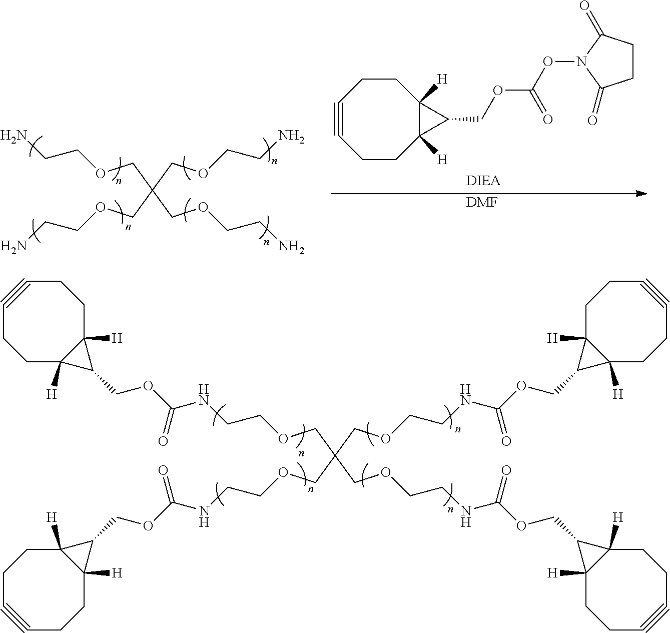

Four-arm poly(ethylene glycol) (PEG) tetraamine (Mₙ˜10 kDa, n˜113, 1.00 g, 0.050 mmol PEG, 0.200 mmol NH₂) and BCN-OSu (87.5 mg, 0.300 mmol, 1.5×) were dissolved in minimal DMF (10 mL) with DIEA (103.4 mg, 0.80 mmol, 4×) and stirred at room temperature overnight. The reaction mixture was diluted with water, dialyzed (molecular weight cut-off (MWCO)˜1 kDa, SpectraPor® 7), and lyophilized to yield the product (denoted PEG-tetraBCN) as a white powder (0.964 g, 0.0466 mmol, 93% yield). ¹H NMR (300 MHz, CDCl₃) δ 5.23 (s, 4H), 4.13 (d, J=8.0 Hz, 8H), 3.76-3.71 (m, 3.3 Hz, 8H), 3.63-3.61 (m, 1818H), 2.34-2.14 (m, 24H), 1.65-1.48 (m, 8H), 1.40-1.27 (m, 4H), 0.99-0.87 (m, 8H). Integral values of the ¹H NMR peaks characteristic of BCN (δ 2.24, 1.57, 1.34, 0.93) were compared to the PEG backbone (δ 3.62) to confirm that functionalization of amines with BCN exceeded 95%.

### Synthesis of Alexa Fluor®568 Functionalized PEG-tetraBCN (PEG-tetraBCN-AF568)

Alexa Fluor® 568 cadavarine (0.963 mg, 1.19 μmol, 1×) and N₃—OSu (Method S1, 0.536 mg, 2.38 μmol, 2×) were dissolved in minimal DMF (1 mL) with DIEA (0.615 mg, 4.76 μmol, 4×) and stirred at room temperature overnight. The reaction mixture was added directly to a solution of 4-arm-PEG_{20 kDa}-tetraBCN (0.964 g, 0.0466 mmol, 40×) in phosphate-buffered saline (PBS, 30 mL) and stirred at room temperature for 3 hours. The reaction mixture was then dialyzed (MWCO˜10 kDa, SnakeSkin™) and lyophilized to give the product (denoted PEG-tetraBCN-AF568) as a pink powder (0.896 g, 0.0433 mmol, 93% yield).

### Synthesis of FAM Functionalized PEG-tetraBCN (PEG-tetraBCN-FAM)

5-FAM azide (AAT Bioquest, 20 μL of 100 mM in DMSO, 0.0020 mmol) and 4-arm-PEG_{20 kDa}-tetraBCN (0.202 g, 0.0101 mmol) was dissolved in PBS (2 mL) and stirred for 2 hours at room temperature. The reaction mixture was diluted with water, dialyzed (MWCO 1 kDa, SpectraPor® 7), and lyophilized to yield the product (denoted PEG-tetraBCN-FAM) as a yellow solid (0.204 g, 0.101 mmol, quantitative yield).

### Synthesis of Cyanine5 Functionalized PEG-tetraBCN (PEG-tetraBCN-Cyanine5)

Cyanine5 azide (Lumiprobe, 20 nmol) in DMSO (2 μL) was added to a solution of PEG-tetraBCN (2 μmol, 100×) in PBS (200 μL). The solution was mixed and reacted at room temperature for 2 hours. The blue product (denoted PEG-tetraBCN-Cyanine5) was used without further purification and kept as a 10 mM stock solution in PBS.

### Method S23. Recombinant Expression and Purification of Matrix Metalloproteinase-8 (MMP-8)

The MMP-8 gene was lifted from pCMV-Tag4A-MMP8 Wt (a gift from Yardena Samuels, Addgene plasmid #29545), adding a C-terminal 6×His purification tag and appropriate restriction enzyme cut sites (NdeI and XhoI) using PCR, placed into pET-28a (+) (EMD Biosciences), and expressed in BL21(DE3)pLysS (Promega Corporation). Cells were lysed via sonication, and the 6×His-tagged protein was solubilized in 6 M urea prior to standard polyhistidine affinity purification using Ni-NTA (Fisher Pierce).

The purified protein was denatured and solubilized in MMP buffer (200 mM sodium chloride, 50 mM tris, 5 mM calcium chloride, 1 μM zinc chloride, pH adjusted to 7.5 with hydrochloric acid) containing 6 M urea and placed into dialysis tubing [molecular weight cut-off (MWCO)˜3.5 kDa, SpectraPor® 7]. The protein was dialyzed against MMP buffer containing progressively lower urea concentrations (4 M→0 M) over the course of 48 hours at 4° C., and then concentrated on a spin column (10 kDa MWCO) to yield the purified, refolded MMP-8.

Protein purity was assessed by SDS-PAGE analysis (12% Bis-Tris gel), and a single band corresponding to the expected molecular weight (43.2 kDa) was observed by Coumassie staining.

### Method 24. Assessing Solution-Based Crosslinker Degradation in Response to External Stimuli

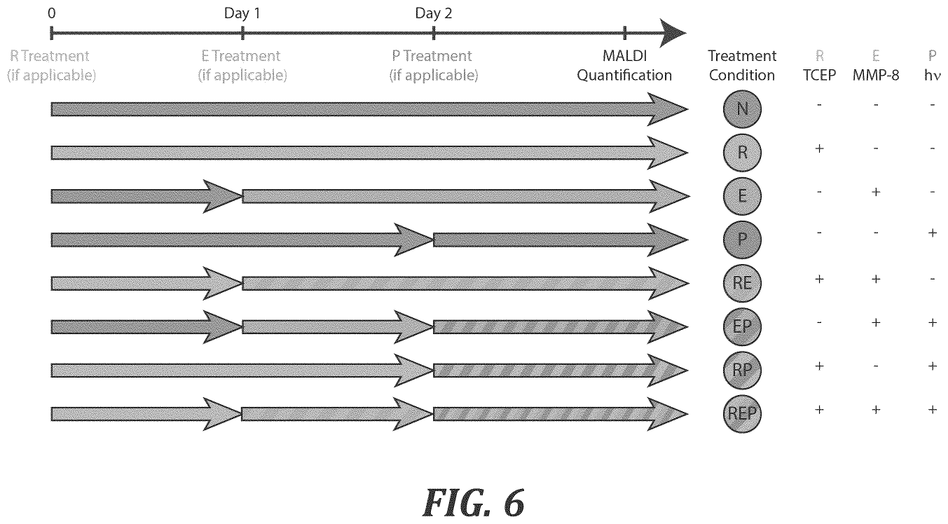

Referring to FIG. 6, to assess crosslinker fragmentation in response to different combinations of external stimuli, a series of solution-based studies were performed on 9 of the synthesized peptides: E, R, P, R∨E, E∨P, R∨P, R∧E, E∧P, and R∧P. Peptide crosslinkers (40 nmol) were dissolved in MMP buffer (110 μL) and exposed to a unique input combination of enzyme, reductive, and light treatments (outlined in further detail below). In each study, degradation products for distinct crosslinker/treatment combinations were identified by MALDI mass spectrometry; observed cleavage products were compared with those expected to assess degradation modalities of the engineered crosslinker species.

Samples receiving the reductive input (_{R}) were treated with tris(2-carboxyethyl)phosphine hydrochloride (TCEP·HCl, 2 μL, 100 mM in MMP buffer) and incubated overnight at 37° C. To quench any unreached TCEP, these samples were further treated with hydroxyethyl disulfide (HEDS, 5 μL, 100 mM in MMP buffer) prior to incubation (4 hr, 37° C.). Samples not receiving reductive input were maintained at 37° C.

Samples receiving the enzyme input (_{E}) were subsequently treated with MMP-8 (Method S23, 5 μL, 0.2 mg mL⁻¹ in MMP buffer) and all samples were incubated (20 hr, 37° C.).

Samples receiving the light input (_{P}) were subsequently exposed to UV light (λ=365 nm, 10 mW cm⁻² incident light, 60 minute exposure).

All samples were diluted with acetonitrile/water (80:20, 100 μL) containing 0.1% TFA and characterized by MALDI-TOF. Treatment conditions are referenced using the crosslinker logical notation with a subscript indicating the inputs. For example, PE corresponds to the “photo YES” crosslinker treated with enzyme; (R∧E)_{REP} corresponds to the “reductive AND enzymatic” crosslinker (R∧E) treated with every cue (_{REP}). Peptide fragments are referenced using the crosslinker logical notation with a subscript indicating the cues required to generate the product. If a crosslinker generates multiple fragments when treated with a set of cues, the products are differentiated by a numerical subscript. For example, (R∧E)_{RE1} and (R∧E)_{RE2} represent the two distinct products generated when the “reductive AND enzymatic” crosslinker (R∧E) is exposed to both reductive and enzymatic treatment.

There are inherent limitations in using MALDI to identify low-mass molecules due to interference of the matrix molecules; only molecules larger than 600 Da are within the MALDI range of detection (ROD). Additionally, molecules analyzed by MALDI that contain an oNB moiety generate the parent molecular ion as well as −16, −30, and −32 Da peaks corresponding to the laser-induced photodecomposition of the nitro (NO₂) group into nitroso (NO), amino (NH₂), and triplet nitrene (N:) groups, respectively. When peptides were treated with reducing conditions, mass shifts of −26 and −52 Da were observed, correspond to a single and double reduction of an azide group (N₃) to an amino group (NH₂), respectively. Free thiols (—SH) oxidized in the presence of reduced HEDS generated mass shifts of +76 Da, corresponding to the disulfide-containing species (—SSCH₂CH₂OH).

The solution degradation products of various crosslikers are shown below:

### Characterization of E Crosslinker Degradation in Solution

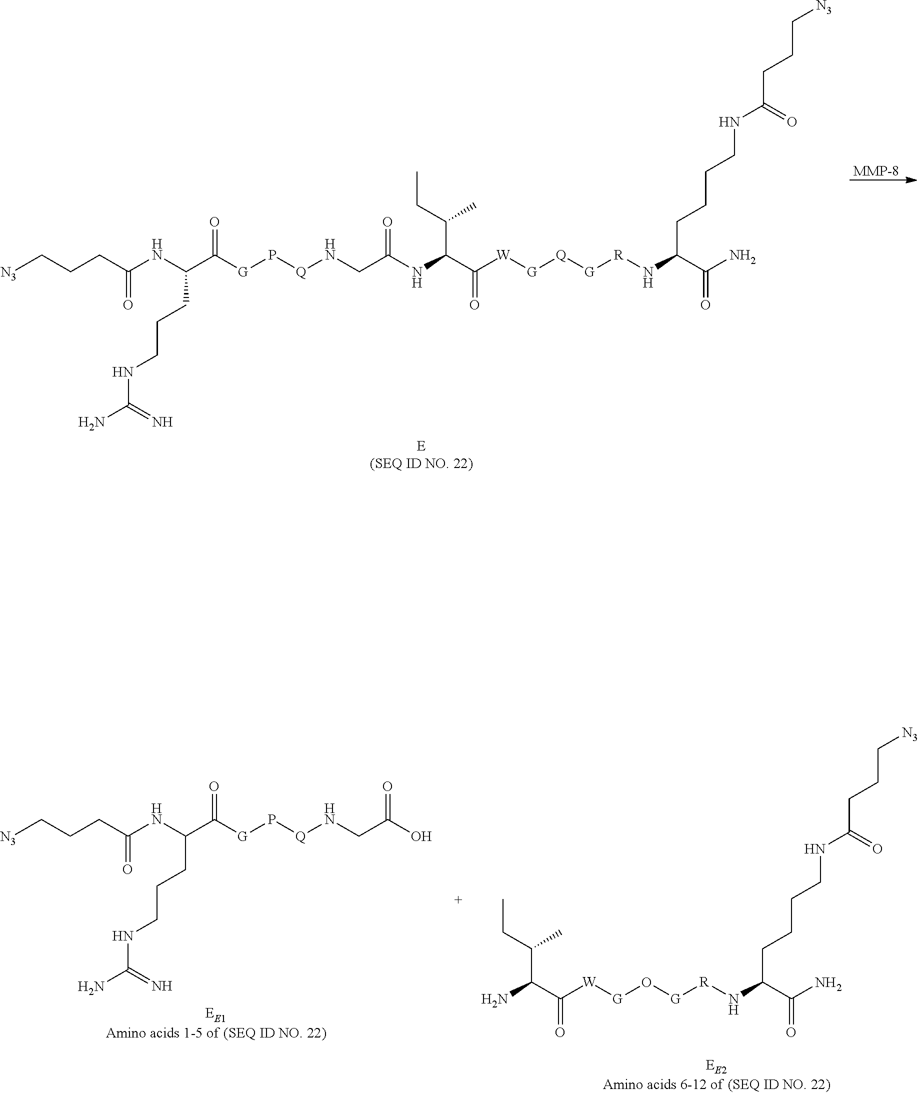

| | Expected | | |
|---|---|---|---|
| Treatment | Peptides | m/z (Da) Calculated | Measured |
| E | E | [M + H]⁺ = 1560.8; | 1560.8; |
| | | [M + H: —N₃ → | 1534.8 |
| | | —NH₂]⁺ = 1534.8 | |
| E_{E} | E_{E1} | [M + H]⁺ = 625.3 | 625.5 |
| | E_{E2} | [M + H]⁺ = 954.5 | 954.6 |
| E_{R} | E | [M + H]⁺ = 1560.8; | 1509.0 |
| | | [M + H: 2 —N₃ → 2 | |
| | | —NH₂] = 1508.8 | |
| E_{P} | E | [M + H]⁺ = 1560.8; | 1560.8; |
| | | [M + H: —N₃ → | 1533.7 |
| | | —NH₂]⁺ = 1534.8 | |
| E_{RE} | E_{E1} | [M + H]⁺ = 625.3; | Below |
| | | [M + H: —N₃ → | ROD |
| | | —NH₂]⁺ = 599.3 | |
| | E_{E2} | [M + H]⁺ = 954.5; | 928.7 |
| | | [M + H: —N₃ → | |
| | | —NH₂]⁺ = 928.5 | |
| E_{EP} | E_{E1} | [M + H]⁺ = 625.3 | 625.4 |
| | E_{E2} | [M + H]⁺ = 954.5 | 954.6 |
| E_{RP} | E | [M + H]⁺ = 1560.8; | 1508.9 |
| | | [M + H: 2 | |
| | | —N₃ → 2 | |
| | | —NH₂]⁺ = 1508.8 | |
| E_{REP} | E_{E1} | [M + H]⁺ = 625.3; | Below |
| | | [M + H: —N₃ → | ROD |
| | | —NH₂]⁺ = 599.3 | |
| | E_{E2} | [M + H]⁺ = 954.5; | 928.6 |
| | | [M + H: —N₃ → | |
| | | —NH₂]⁺ = 928.5 | |

Expected products were identified in all treatment conditions except product E_{E1}, presumably due to being below the MALDI range of detection (m/z>600). Products were of good purity in all cases.

### Characterization of R Crosslinker Degradation in Solution

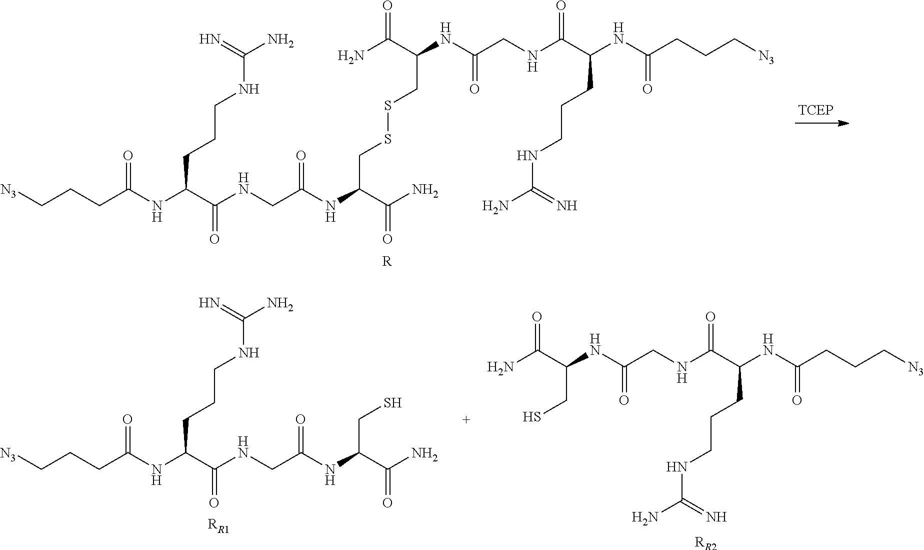

| | Expected | m/z (Da) | |
|---|---|---|---|
| Treatment | Peptides | Calculated | Measured |
| R | R | [M + H]⁺ = 887.4 | 887.4 |
| R_{E} | R | [M + H]⁺ = 887.4 | 887.4 |
| R_{R} | R_{R1} | [M + H]⁺ = 445.2 | Below |
| | | | ROD |
| | R_{R2} | [M + H]⁺ = 445.2 | Below |
| | | | ROD |
| R_{P} | R | [M + H]⁺ = 887.4; | 887.6; |
| | | [M + H: —N₃ → | 861.6 |
| | | —NH₂]⁺ = 861.4 | |
| R_{RE} | R_{R1} | [M + H]⁺ = 445.2 | Below |
| | | | ROD |
| | R_{R2} | [M + H]⁺ = 445.2 | Below |
| | | | ROD |
| R_{EP} | R | [M + H]⁺ = 887.4; | 887.5; |
| | | [M + H: —N₃ → | 860.4 |
| | | —NH₂]⁺ = 861.4 | |
| R_{RP} | R_{R1} | [M + H]⁺ = 445.2 | Below |
| | | | ROD |
| | R_{R2} | [M + H]⁺ = 445.2 | Below |
| | | | ROD |
| R_{REP} | R_{R1} | [M + H]⁺ = 445.2 | Below |
| | | | ROD |
| | R_{R2} | [M + H]⁺ = 445.2 | Below |
| | | | ROD |

Expected products were identified in all treatment conditions except R_{R1} and R_{R2}, presumably due to the products being below the MALDI range of detection (m/z>600). In treatments R_{R}, R_{RE}, R_{RP}, and R_{REP}, the noisy background signal is likely because no expected products were in MALDI ROD.

### Characterization of P Crosslinker Degradation in Solution

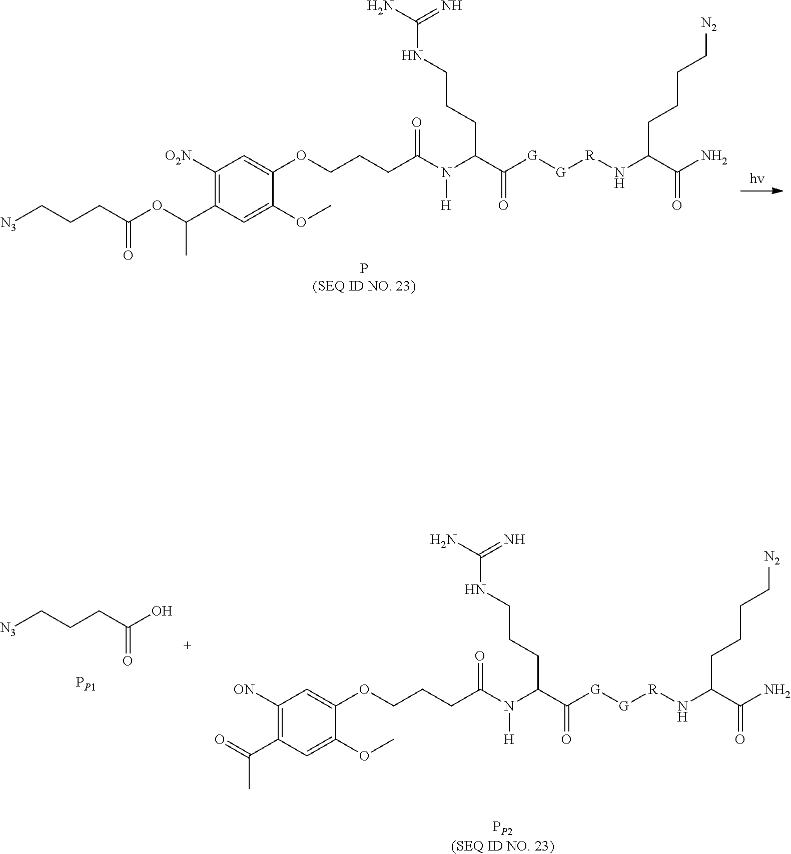

| | Expected | m/z (Da) | |
|---|---|---|---|
| Treatment | Peptides | Calculated | Measured |
| P | P | [M + H]⁺ = 990.5; [M + H: —NO₂ → —NO]⁺ = 974.5 | 990.6; 974.6 |
| P_{E} | P | [M + H]⁺ = 990.5; [M + H: —NO₂ → NO]⁺ = 974.5 | 990.6; 974.6 |
| P_{R} | P | [M + H]⁺ = 990.5; [M + H: 2 —N₃ → 2 —NH₂]⁺ = 938.5; | 938.6; 922.6; |
| | | [M + H: 2 —N₃ → 2 —NH₂ & —NO₂ → —NO]⁺ = 922.5; | 906.5 |
| | | [M + H: 2 —N₃ → 2 —NH₂ & —NO₂ → —N]⁺ = 906.5 | |
| P_{P} | P_{P1} | [M + H]⁺ = 130.1 | Below ROD |
| | P_{P2} | [M + H]⁺ = 861.4; [M + H: —NO₂ → —NH₂]⁺ = 847.4 | 861.6; 847.6 |
| P_{RE} | P | [M + H]⁺ = 990.5; [M + H: 2 —N₃ → 2 —NH₂]⁺ = 938.5; | 938.6; 922.5; |
| | | [M + H: 2 —N₃ → 2 —NH₂ & —NO₂ → —NO]⁺ = 922.5; | 906.5 |
| | | [M + H: 2 —N₃ → 2 —NH₂ & —NO₂ → —N]⁺ = 906.5 | |
| P_{EP} | P_{P1} | [M + H]⁺ = 130.1 | Below ROD |
| | P_{P2} | [M + H]⁺ = 861.4; [M + H: —NO₂ → —NH₂]⁺ = 847.4 | 861.6; 847.5 |
| P_{RP} | P_{P1} | [M + H]⁺ = 130.1 | Below ROD |
| | P_{P2} | [M + H]⁺ = 861.4; [M + H: —N₃ → —NH₂]⁺ = 835.4; | 835.5; 821.5 |
| | | [M + H: —N3 → —NH2 & —NO → —NH2]⁺ = 821.5 | |
| P_{REP} | P_{P1} | [M + H]⁺ = 130.1 | Below ROD |
| | P_{P2} | [M + H]⁺ = 861.4; [M + H: —N₃ → —NH₂]⁺ = 835.4; | 835.6; 821.5 |
| | | [M + H: —N₃ → —NH₂ & —NO → —NH₂]⁺ = 821.5 | |

Expected products were identified in all treatment conditions except P_{P1}, presumably due to being below the MALDI range of detection (m/z>600). Products were of good purity in all cases except for treatments PR and PPE.

### Characterization of R∨E Crosslinker Degradation in Solution

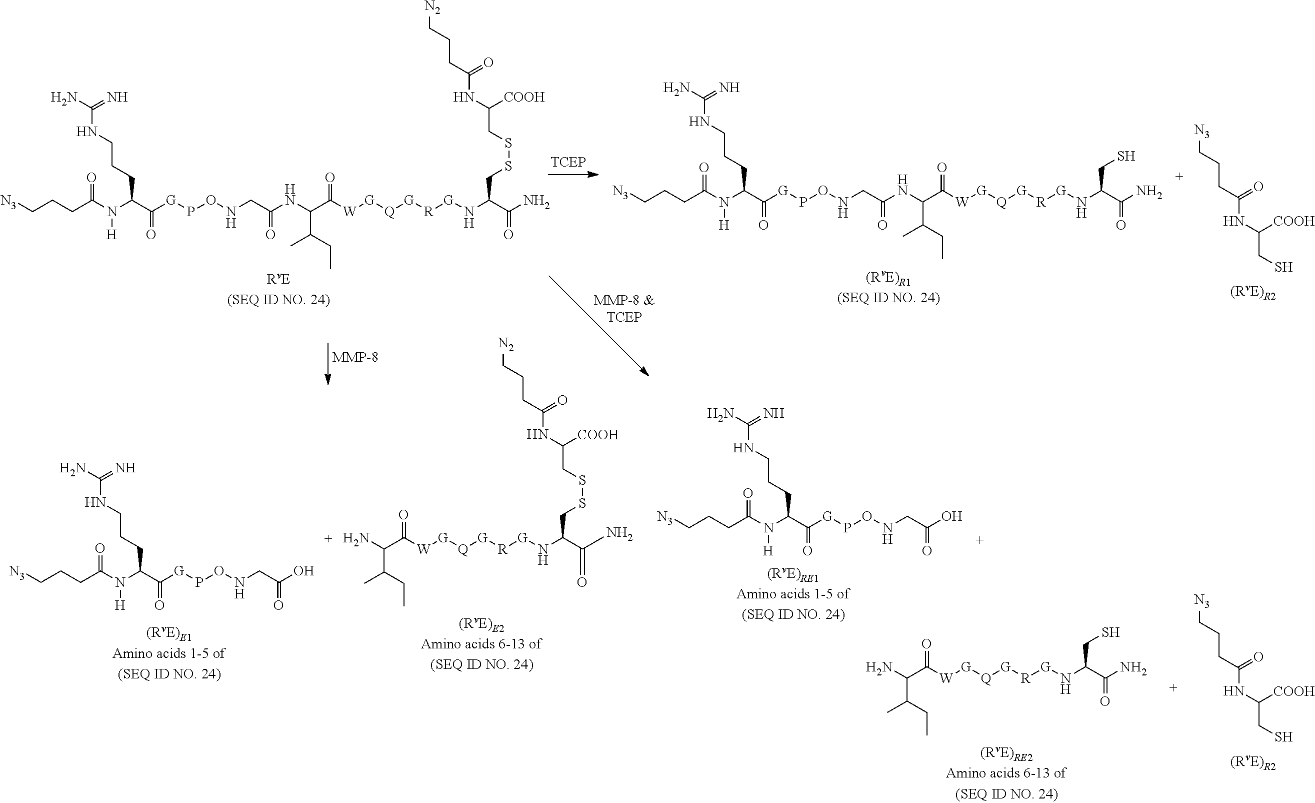

| | Expected | m/z (Da) | |
|---|---|---|---|
| Treatment | Peptides | Calculated | Measured |
| R∨E | R∨E | [M + H]⁺ = 1711.8; [M + H: —N₃ → —NH₂]⁺ = 1685.8 | 1711.5; |
| | | | 1685.4 |
| (R∨E)_{E} | (R∨E)_{E1} | [M + H]⁺ = 625.3; [M + H: —N₃ → —NH₂]⁺ = 599.3 | Below |
| | | | ROD |
| | (R∨E)_{E2} | [M + H]⁺ = 1105.5 | 1105.4 |
| (R∨E)_{R} | (R∨E)_{R1} | [M + H]⁺ = 1481.7; [M + H: —N₃ → —NH₂ & —SH → | 1531.6 |
| | | —SSCH₂CH₂OH]⁺ = 1531.8 | |
| | (R∨E)_{R2} | [M + H]⁺ = 233.1; [M + H: —N₃ → —NH₂ & —SH → | Below |
| | | —SSCH₂CH₂OH]⁺ = 283.2 | ROD |
| (R∨E)_{P} | R∨E | [M + H]⁺ = 1711.8; [M + H: —N₃ → —NH₂]⁺ = 1685.8 | 1711.6; |
| | | | 1685.6 |
| (R∨E)_{RE} | (R∨E)_{RE1} | [M + H]⁺ = 625.3; [M + H: —N₃ → —NH₂]⁺ = 599.3 | Below |
| | | | ROD |
| | (R∨E)_{RE2} | [M + H]⁺ = 875.4; [M + H: —N₃ → —NH₂ & —SH → | 951.4 |
| | | —SSCH₂CH₂OH]⁺ = 950.4 | |
| | (R∨E)_{RE3} | [M + H]⁺ = 233.1; [M + H: —N₃ → —NH₂ & —SH → | Below |
| | | —SSCH₂CH₂OH]⁺ = 283.2 | ROD |
| (R∨E)_{EP} | (R∨E)_{E1} | [M + H]⁺ = 625.3; [M + H: —N₃ → —NH₂]⁺ = 599.3 | Below |
| | | | ROD |
| | (R∨E)_{E2} | [M + H]⁺ = 1105.5 | 1105.4 |
| (R∨E)_{RP} | (R∨E)_{R1} | [M + H]⁺ = 1481.7; [M + H: —N₃ → —NH₂ & —SH → | 1531.7 |
| | | —SSCH₂CH₂OH]⁺ = 1531.8 | |
| | (R∨E)_{R2} | [M + H]⁺ = 233.1 | Below |
| | | | ROD |
| (R∨E)_{REP} | (R∨E)_{RE1} | [M + H]⁺ = 625.3; [M + H: —N₃ → —NH₂]⁺ = 599.3 | Below |
| | | | ROD |
| | (R∨E)_{RE2} | [M + H]⁺ = 875.4; [M + H: —N₃ → —NH₂ & —SH → | 951.4 |
| | | —SSCH₂CH₂OH]⁺ = 950.4 | |
| | (R∨E)_{RE3} | [M + H]⁺ = 233.1; [M + H: —N₃ → —NH₂ & —SH → | Below |
| | | —SSCH₂CH₂OH]⁺ = 283.2 | ROD |

Expected products were identified in all treatment conditions except products (R∨E)_{E1}, (R∨E)_{R2}, (R∨E)_{RE1}, and (R∨E)_{RE3}, presumably due to being below the MALDI range of detection (m/z>600). Products were of good purity in all cases.

### Characterization of E∨P Crosslinker Degradation in Solution

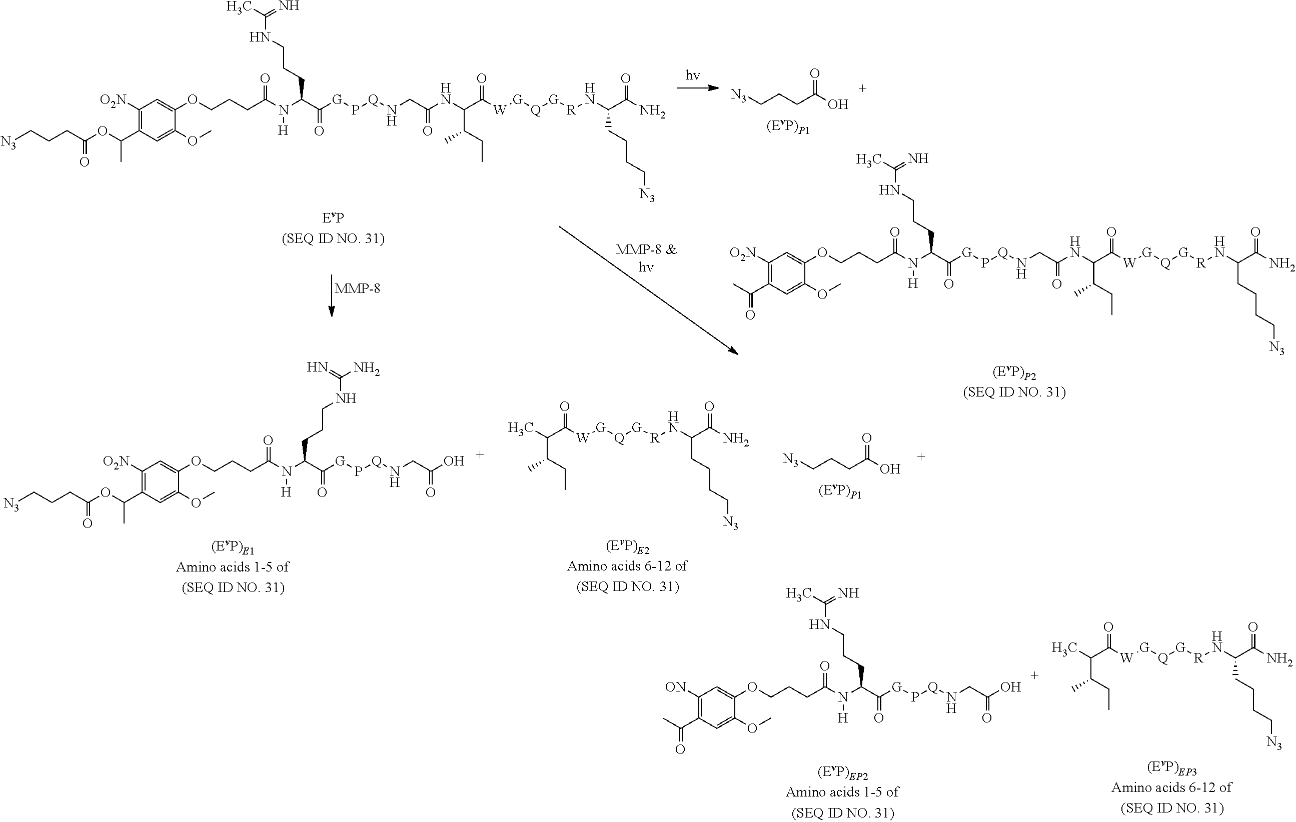

| | Expected | m/z (Da) | |
|---|---|---|---|
| Treatment | Peptides | Calculated | Measured |
| E∨P | E∨P | [M + H]⁺ = 1756.9; [M + H: —NO₂ → —NO]⁺ = 1740.9 | 1756.8; |
| | | | 1740.8 |
| (E∨P)_{E} | (E∨P)_{E1} | [M + H]⁺ = 906.4; [M + H: —NO₂ → —NO]⁺ = 890.4 | 906.5; 860.5 |
| | (E∨P)_{E2} | [M + H]⁺ = 869.5 | 869.5 |
| (E∨P)_{R} | E∨P | [M + H]⁺ = 1756.9; [M + H: 2 —N₃ → 2 —NH₂]⁺ = 1704.9; | 1704.8; |
| | | [M + H: 2 —N₃ → 2 —NH₂ & —NO₂ → —N]⁺ = 1688.9 | 1688.8 |
| (E∨P)_{P} | (E∨P)_{P1} | [M + H]⁺ = 130.1 | Below ROD |
| | (E∨P)_{P2} | [M + H]⁺ = 1627.8; [M + H: —NO → —NH₂]⁺ = 1613.8 | 1627.6; |
| | | | 1613.5 |
| (E∨P)_{RE} | (E∨P)_{E1} | [M + H]⁺ = 906.4; [M + H: —N₃ → —NH₂]⁺ = 880.4; | 880.6; 864.6 |
| | | [M + H: —N₃ → —NH₂ & —NO₂ → —NO]⁺ = 864.4 | |
| | (E∨P)_{E2} | [M + H]⁺ = 869.5; [M + H: —N₃ → —NH₂]⁺ = 843.5 | 843.7 |
| (E∨P)_{EP} | (E∨P)_{EP1} | [M + H]⁺ = 130.1 | Below ROD |
| | (E∨P)_{EP2} | [M + H]⁺ = 777.4; [M + H: —NO → —NH₂]⁺ = 763.4 | 777.5; 763.4 |
| | (E∨P)_{EP3} | [M + H]⁺ = 869.5; [M + H: —N₃ → —NH₂]⁺ = 843.5 | Not observed |
| (E∨P)_{RP} | (E∨P)_{P1} | [M + H]⁺ = 130.1 | Below ROD |
| | (E∨P)_{P2} | [M + H]⁺ = 1627.8; [M + H: —N₃ → —NH₂]⁺ = 1601.8; | 1601.7; |
| | | [M + H: —N₃ → —NH₂ & —NO → —NH₂]⁺ = 1587.8 | 1587.7 |
| (E∨P)_{REP} | (E∨P)_{EP1} | [M + H]⁺ = 130.1 | Below ROD |
| | (E∨P)_{EP2} | [M + H]⁺ = 777.4; [M + H: —NO → —NH₂]⁺ = 763.4 | 777.4; 763.4 |
| | (E∨P)_{EP3} | [M + H]⁺ = 869.5; [M + H: —N₃ → —NH₂]⁺ = 843.5 | Not observed |

Expected products were identified in all treatment conditions except products (E∨P)_{P1}, (E∨P)_{EP1}, (E∨P)_{EP1}, and (E∨P)_{EP3}, presumably due to being below the MALDI range of detection (m/z>600) or relative differences in ionization propensity with other species present. Products were of good purity in all cases except for treatment (E∨P)_{R}.

### Characterization of R∨P Crosslinker Degradation in Solution

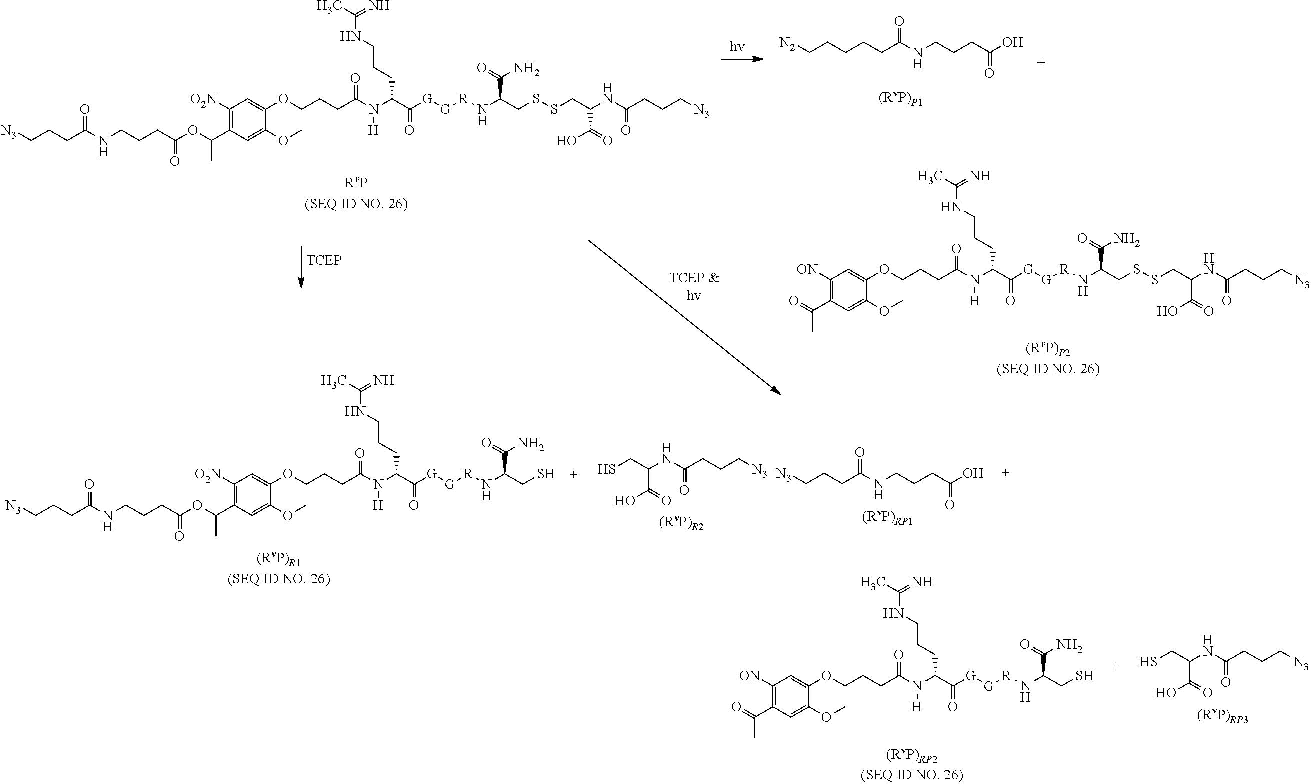

| | Expected | m/z (Da) | |
|---|---|---|---|
| Treatment | Peptides | Calculated | Measured |
| R∨P | R∨P | [M + H]⁺ = 1254.5; [M + H: —NO₂ → —NO]⁺ = 1238.5 | 1254.6; |
| | | | 1238.6 |
| (R∨P)_{E} | R∨P | [M + H]⁺ = 1254.5; [M + H: —NO₂ → —NO]⁺ = 1238.5 | 1254.6; |
| | | | 1238.5 |
| (R∨P)_{R} | (R∨P)_{R1} | [M + H]⁺ = 1024.5; [M + H: —N₃ → —NH₂ & —SH → | 1074.5; |
| | | —SSCH₂CH₂OH]⁺ = 1074.6; | 1058.5 |
| | | [M + H: —N₃ → —NH₂ & —SH → —SSCH₂CH₂OH & | |
| | | —NO₂ → —NO]⁺ = 1058.6 | |
| | (R∨P)_{R2} | [M + H]⁺ = 233.1 | Below |
| | | | ROD |
| (R∨P)_{P} | (R∨P)_{P1} | [M + H]⁺ = 215.1 | Below |
| | | | ROD |
| | (R∨P)_{P2} | [M + H]⁺ = 1040.4; [M + H: —NO₂ → —NH₂]⁺ = 1026.4 | 1026.5 |
| (R∨P)_{RE} | (R∨P)_{R1} | [M + H]⁺ = 1024.5; [M + H: —N₃ → —NH₂ & —SH → | 1074.6; |
| | | —SSCH₂CH₂OH]⁺ = 1074.6; | 1058.5 |
| | | [M + H: —N₃ → —NH₂ & —SH → —SSCH₂CH₂OH & | |
| | | —NO₂ → —NO]⁺ = 1058.6 | |
| | (R∨P)_{R2} | [M + H]⁺ = 233.1 | Below |
| | | | ROD |
| (R∨P)_{EP} | (R∨P)_{P1} | [M + H]⁺ = 215.1 | Below |
| | | | ROD |
| | (R∨P)_{P2} | [M + H]⁺ = 1040.4; [M + H: —NO₂ → —NH₂]⁺ = 1026.4 | 1026.5 |
| (R∨P)_{RP} | (R∨P)_{RP1} | [M + H]⁺ = 215.1 | Below |
| | | | ROD |
| | (RvP)_{RP2} | [M + H]⁺ = 810.4; [M + H: —SH → —SSCH₂CH₂OH]⁺ = 886.5; | 886.5; |
| | | [M + H: —SH → —SSCH₂CH₂OH & —NO₂ → —NH₂]⁺ = 872.5 | 872.5 |
| | (R∨P)_{RP3} | [M + H]⁺ = 233.1 | Below |
| | | | ROD |
| (R∨P)_{REP} | (R∨P)_{RP1} | [M + H]⁺ = 215.1 | Below |
| | | | ROD |
| | (R∨P)_{RP2} | [M + H]⁺ = 810.4; [M + H: —SH → —SSCH₂CH₂OH]⁺ = 886.5; | 886.5; |
| | | [M + H: —SH → —SSCH₂CH₂OH & —NO₂] → —NH₂]⁺ = 872.5 | 872.5 |
| | (R∨P)_{RP3} | [M + H]⁺ = 233.1 | Below |
| | | | ROD |

Expected products were identified in all treatment conditions except products (R∨P)_{R2}, (R∨P)_{P1}, (R∨P)_{RP1}, and (R∨P)_{RP3}, presumably due to being below the MALDI range of detection (m/z>600). Products were of good purity in all cases.

### Characterization of R∧E Crosslinker Degradation in Solution

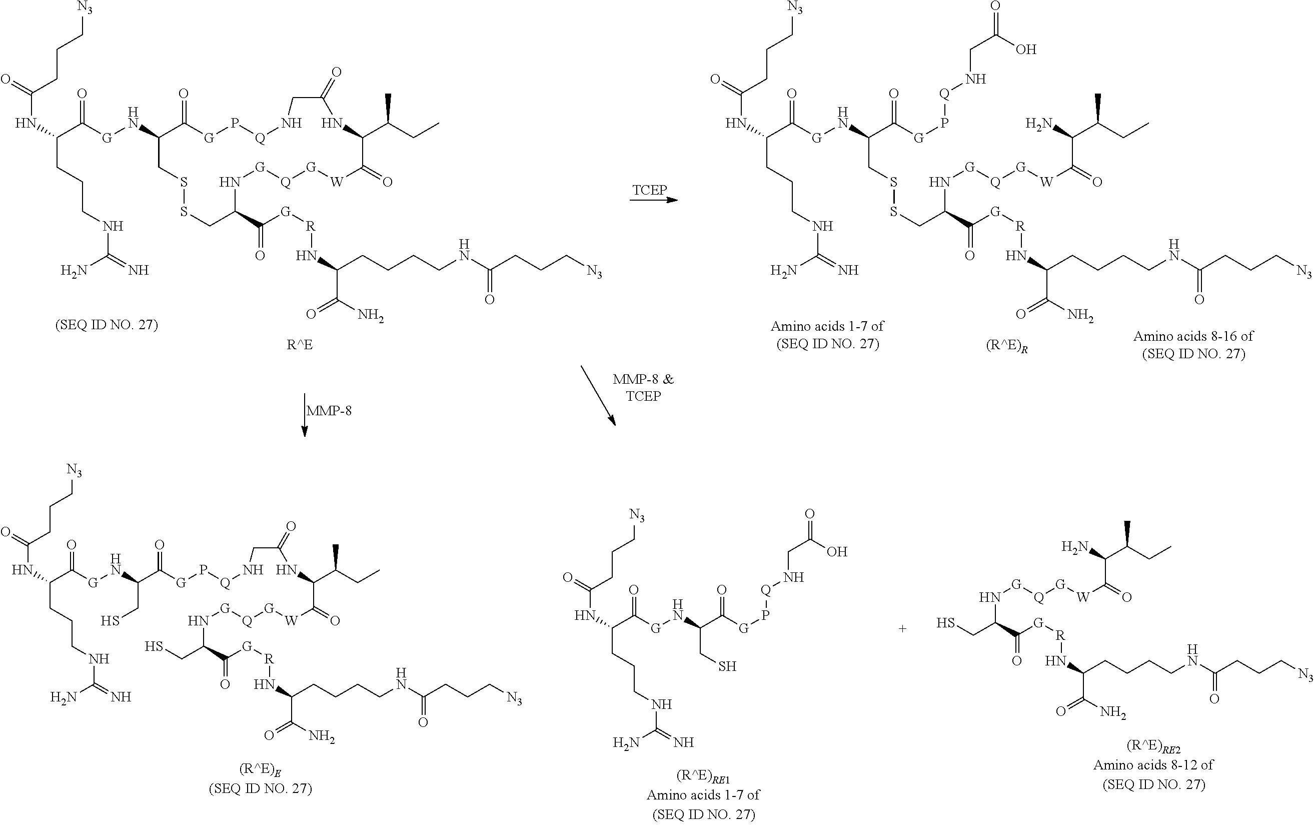

| | Expected | m/z (Da) | |
|---|---|---|---|
| Treatment | Peptides | Calculated | Measured |
| R∧E | R∧E | [M + H]⁺ = 1878.9; [M + H: —N₃ → —NH₂]⁺ = 1852.9 | 1878.6; |
| | | | 1852.6 |
| (R∧E)_{E} | (R∧E)_{E} | [M + H]⁺ = 1896.9; [M + H: —N₃ → —NH₂]⁺ = 1870.9 | Not |
| | | | observed |
| (R∧E)_{R} | (R∧E)_{R} | [M + H]⁺ = 1880.9; [M + H: —N₃ → —NH₂]⁺ = 1854.9; | 1826.6 |
| | | [M + H: 2 —N₃ → 2 —NH₂]⁺ = 1828.9 | |
| (R∧E)_{P} | R∧E | [M + H]⁺ = 1878.9; [M + H: —N₃ → —NH₂]⁺ = 1852.9 | 1878.9; |
| | | | 1852.8 |
| (R∧E)_{RE} | (R∧E)_{RE1} | [M + H]⁺ = 785.3; [M + H: —N₃ → —NH₂ & —SH → | Not |
| | | —SSCH₂CH₂OH]⁺ = 835.4 | observed |
| | (R∧E)_{RE2} | [M + H]⁺ = 1114.6; [M + H: —N₃ → —NH₂ & —SH → | 1164.4 |
| | | —SSCH₂CH₂OH]⁺ = 1164.7 | |
| (R∧E)_{EP} | (R∧E)_{E} | [M + H]⁺ = 1896.9; [M + H: —N₃ → —NH₂]⁺ = 1870.9 | Not |
| | | | observed |
| (R∧E)_{RP} | (R∧E)_{R} | [M + H]⁺ = 1880.9; [M + H: —N₃ → —NH₂]⁺ = 1854.9; | 1826.8 |
| | | [M + H: 2 —N₃ → 2 —NH₂]⁺ = 1828.9 | |
| (R∧E)_{REP} | (R∧E)_{RE1} | [M + H]⁺ = 785.3; [M + H: —N₃ → —NH₂ & —SH → | Not |
| | | —SSCH₂CH₂OH]⁺ = 835.4 | observed |
| | (R∧E)_{RE2} | [M + H]⁺ = 1114.6; [M + H: —N₃ → —NH₂ & —SH → | 1164.5 |
| | | —SSCH₂CH₂OH]⁺ = 1164.7 | |

Expected products were identified in all treatment conditions except product (R/AE)_{RE1}, presumably due relative differences in ionization propensity with other species present. In conditions expected to produce (R∧E)_{E}, only the starting material was observed. Products were of good purity in all cases.

### Characterization of E∧P Crosslinker Degradation in Solution

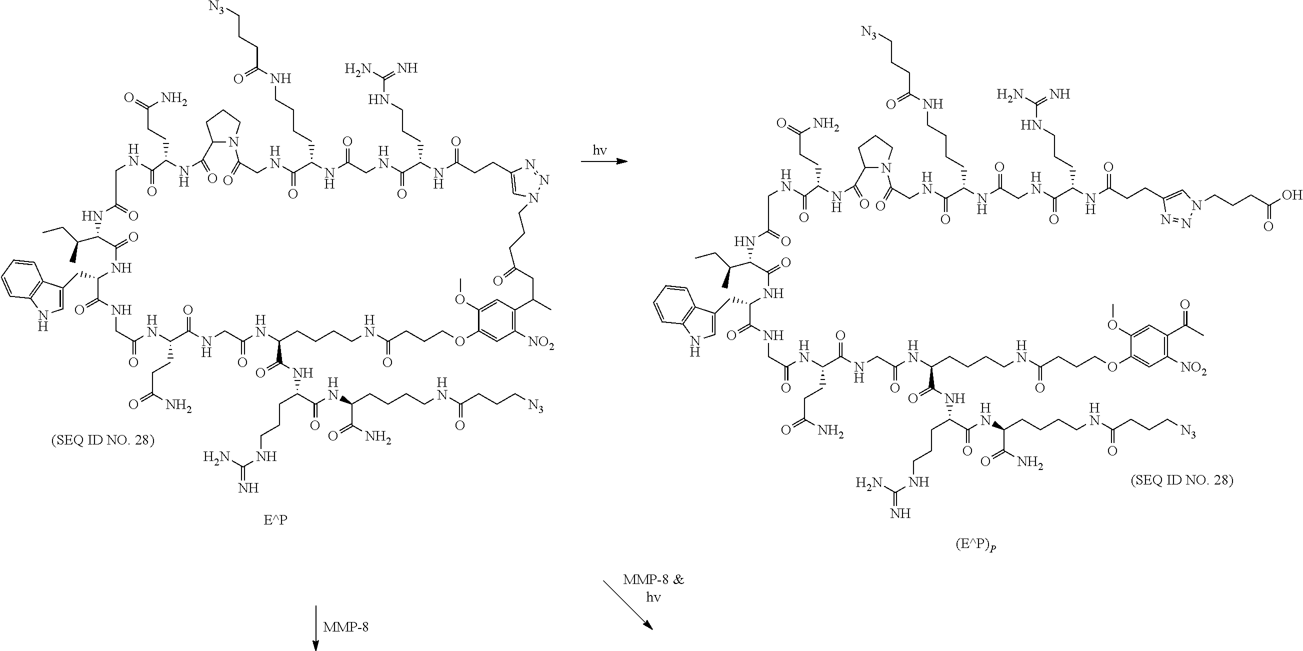

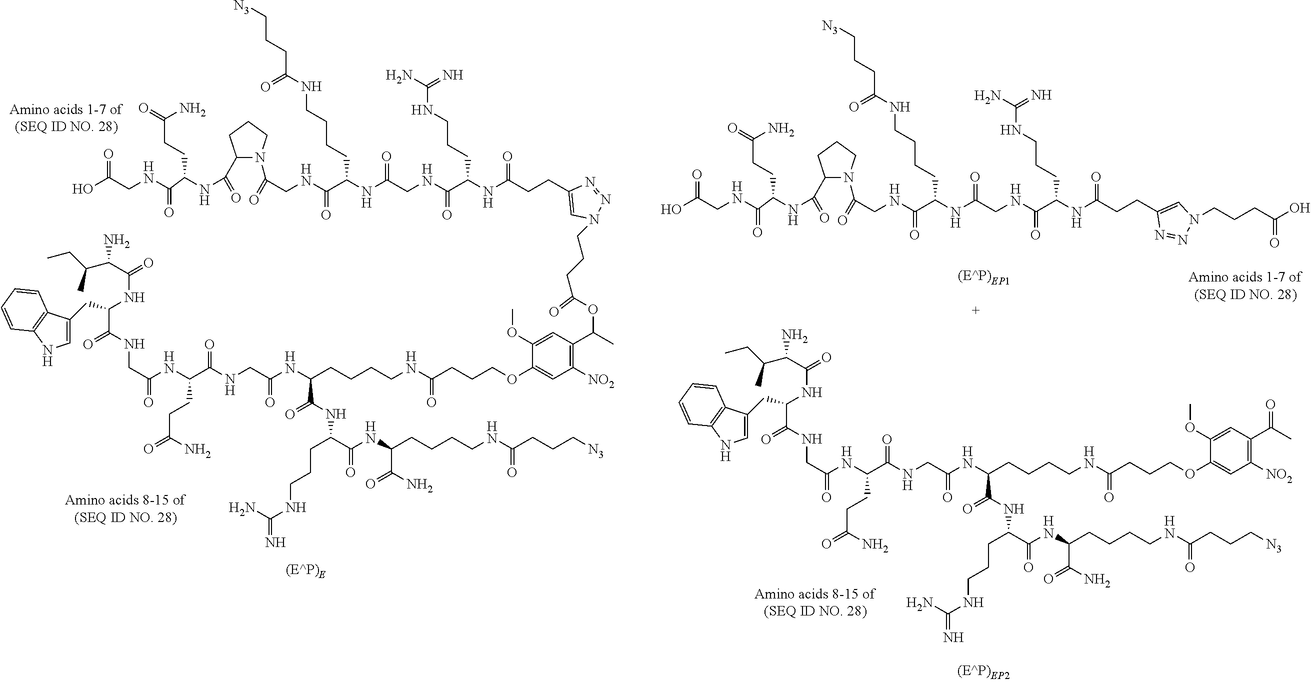

| | Expected | m/z (Da) | |
|---|---|---|---|
| Treatment | Peptides | Calculated | Measured |
| E∧P | E∧P | [M + H]⁺ = 2346.2; [M + H: —NO₂ → —NO]⁺ = 2330.2 | 2346.0; |
| | | | 2330.0 |
| (E∧P)_{E} | (E∧P)_{E} | [M + H]⁺ = 2364.2; [M + H: —NO₂ → —NO]⁺ = 2348.2 | 2364; |
| | | | 2348.1 |
| (E∧P)_{R} | E∧P | [M + H]⁺ = 2346.2; [M + H: 2 —N₃ → 2 —NH₂]⁺ = 2294.2; | 2294.2; |
| | | [M + H: 2 —N₃ → 2 —NH2 & —NO2 → —NO]⁺ = 2278.2 | 2278.1 |
| (E∧P)_{P} | (E∧P)_{P} | [M + H]⁺ = 2346.2; [M + H: —NO— → —N or —NH₂]⁺ = | 2346.2; |
| | | 2330.2/2332.2; | 2332.1; |
| | | [M + H: —N₃ → —NH₂]⁺ = 2320.2; | 2330.2; |
| | | [M + H: —N₃ → —NH₂ & —NO → —N or —NH₂]⁺ = | 2320.1; |
| | | 2304.6/2306.2 | 2306.1; |
| | | | 2304.1 |
| (E∧P)_{RE} | (E∧P)_{E} | [M + H]⁺ = 2364.2; [M + H: 2 —N₃ → 2 —NH₂]⁺ = 2312.2; | 2312.2; |
| | | [M + H: 2 —N₃ → 2 —NH₂ & —NO₂ → —NO]⁺ = 2296.2 | 2296.2 |
| (E∧P)_{EP} | (E∧P)_{EP1} | [M + H]⁺ = 1345.7; [M + H: —NO → —N]⁺ = 1329.7 | 1329.8 |
| | (E∧P)_{EP2} | [M + H]⁺ = 1019.5; [M + H: —N₃ → —NH₂]⁺ = 993.5 | 1019.6 |
| (E∧P)_{RP} | (E∧P)_{P} | [M + H]⁺ = 2346.2; [M + H: 2 —N₃ → 2 —NH₂]⁺ = 2294.2; | 2294.2; |
| | | [M + H: 2 —N₃ → 2 —NH₂ & —NO → —NH₂]⁺ = 2280.2 | 2280.3 |
| (E∧P)_{REP} | (E∧P)_{EP1} | [M + H]⁺ = 1019.5; [M + H: —N₃ → —NH₂]⁺ = 993.5 | 993.6 |
| | (E∧P)_{EP2} | [M + H]⁺ = 1345.7; [M + H: —N₃ → —NH₂]⁺ = 1319.7; | 1319.7; |
| | | [M + H: —N₃ → —NH₂ & —NO → —NH₂]⁺ = 1305.7 | 1305.8 |

Expected products were identified in all treatment. Products were of good purity in all cases.

### Characterization of R∧P Crosslinker Degradation in Solution

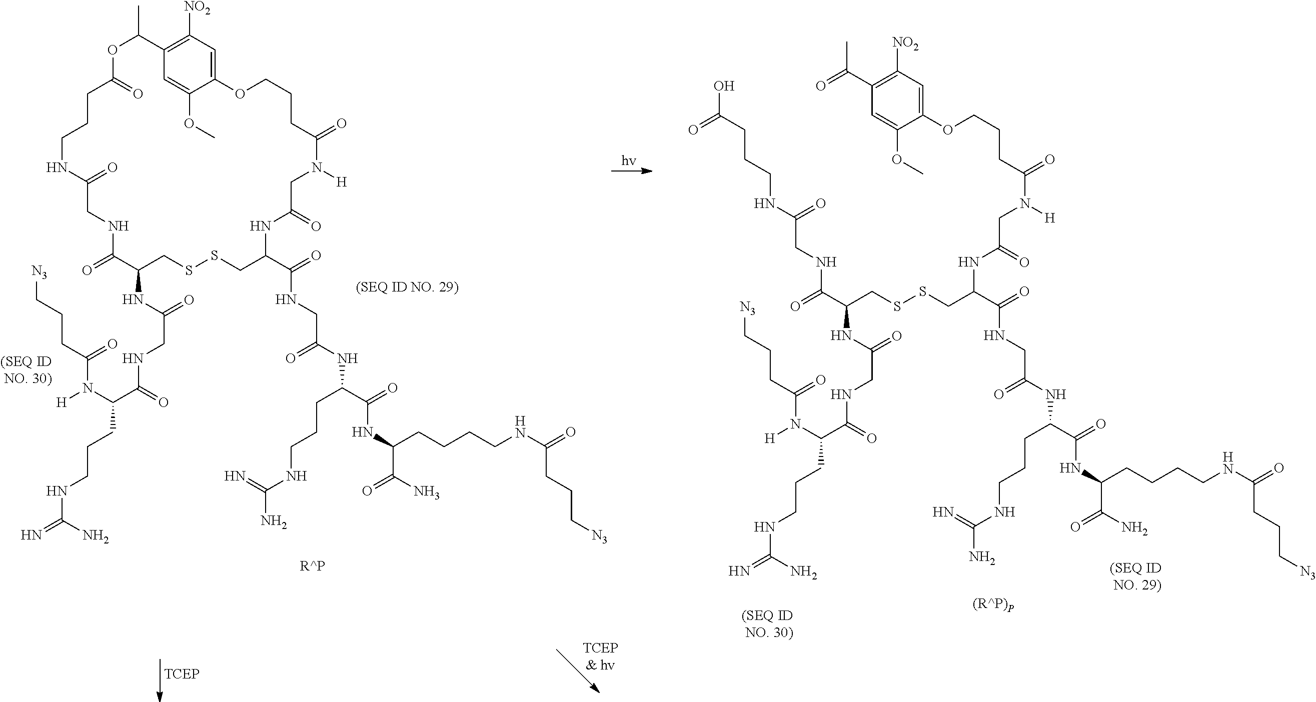 [SMILES: COc1cc2c([N+](=O)[O-])cc1OCCCC(=O)NCC(=O)NC(C(=O)NCC(=O)N[C@@H](CCCNC(=N)N)C(=O)N[C@@H](CCCCNC(=O)CCCN=[N+]=[N-])C(N)=O)CSSC[C@@H](NC(=O)CNC(=O)[C@H](CCCNC(=N)N)NC(=O)CCCN=[N+]=[N-])C(=O)NCC(=O)NCCCC(=O)OC2C ; COc1cc(C(C)=O)c([N+](=O)[O-])cc1OCCCC(=O)NCC(=O)NC(CSSC[C@@H](NC(=O)CNC(=O)[C@H](CCCNC(=N)N)NC(=O)CCCN=[N+]=[N-])C(=O)NCC(=O)NCCCC(=O)O)C(=O)NCC(=O)N[C@@H](CCCNC(=N)N)C(=O)N[C@@H](CCCCNC(=O)CCCN=[N+]=[N-])C(N)=O ; "TCEP"]

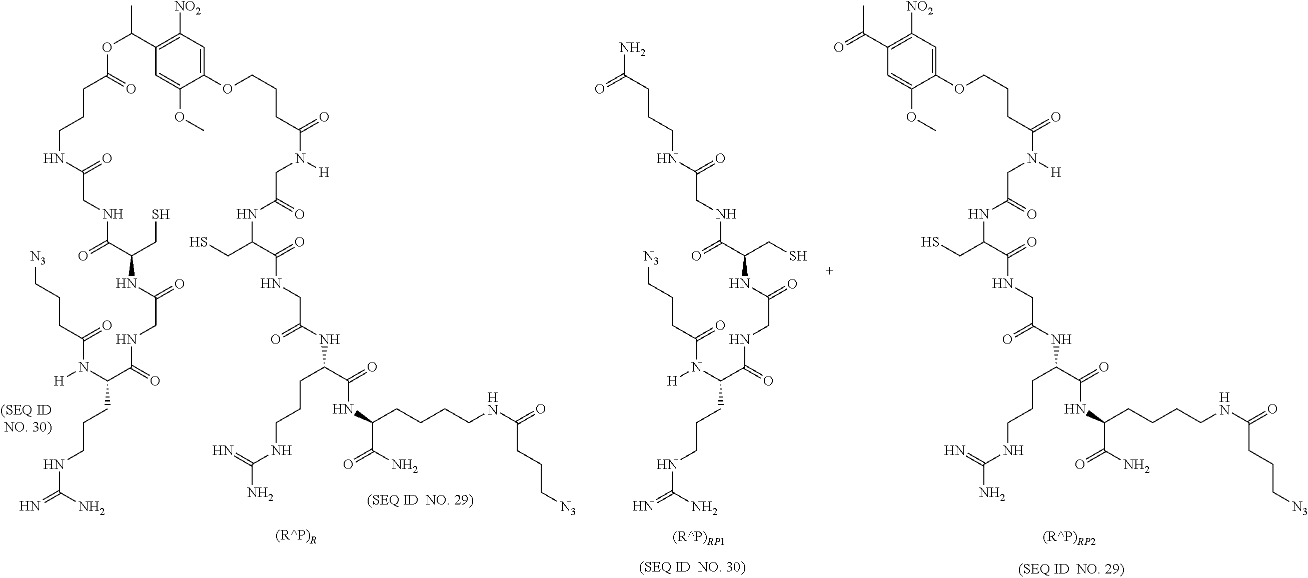 [SMILES: COc1cc(C(C)OC(=O)CCCNC(=O)CNC(=O)[C@@H](CS)NC(=O)CNC(=O)[C@H](CCCNC(=N)N)NC(=O)CCCN=[N+]=[N-])c([N+](=O)[O-])cc1OCCCC(=O)NCC(=O)NC(CS)C(=O)NCC(=O)N[C@@H](CCCNC(=N)N)C(=O)N[C@@H](CCCCNC(=O)CCCN=[N+]=[N-])C(N)=O ; [N-]=[N+]=NCCCC(=O)N[C@@H](CCCNC(=N)N)C(=O)NCC(=O)N[C@H](CS)C(=O)NCC(=O)NCCCC(N)=O ; COc1cc(C(C)=O)c([N+](=O)[O-])cc1OCCCC(=O)NCC(=O)NC(CS)C(=O)NCC(=O)N[C@@H](CCCNC(=N)N)C(=O)N[C@@H](CCCCNC(=O)CCCN=[N+]=[N-])C(N)=O]

| | Expected | m/z (Da) | |
|---|---|---|---|
| Treatment | Peptides | Calculated | Measured |
| R∧P | R∧P | [M + H]⁺ = 1478.6; [M + H: —NO₂ → —NO]⁺ = 1462.6; | 1478.7; |
| | | [M + H: —N₃ → —NH₂]⁺ = 1452.6 | 1462.5; |
| | | | 1452.5 |
| (R∧P)_{E} | R∧P | [M + H]⁺ = 1478.6; [M + H: —NO₂ → —NO]⁺ = 1462.6; | 1478.7; |
| | | [M + H: —N₃ → —NH₂]⁺ = 1452.6 | 1462.6; |
| | | | 1452.6 |
| (R∧P)_{R} | (R∧P)_{R} | [M + H]⁺ = 1480.6; [M + H: 2 —N₃ → 2 —NH₂]⁺ = 1428.6; | 1428.7; |
| | | [M + H: 2 —N₃ → 2 —NH₂ & —NO₂ → —NO]⁺ = 1412.6 | 1412.7 |
| (R∧P)_{P} | (R∧P)_{P} | [M + H]⁺ = 1478.6; [M + H: —NO → —NH₂]⁺ = 1464.6; | 1465.8; |
| | | [M + H: —N₃ → —NH₂ & —NO → —NH₂]⁺ = 1438.6 | 1439.8 |
| (R∧P)_{RE} | (R∧P)_{R} | [M + H]⁺ = 1480.6; [M + H: 2 —N₃ → 2 —NH₂]⁺ = 1428.6; | 1428.0; |
| | | [M + H: 2 —N₃ → 2 —NH₂ & —NO₂ → —NO]⁺ = 1412.6 | 1412.0 |
| (R∧P)_{EP} | (R∧P)_{P} | [M + H]⁺ = 1478.6; [M + H: —NO → —NH₂]⁺ = 1464.6; | 1465.5; |
| | | [M + H: —N₃ → —NH₂ & —NO → —NH₂]⁺ = 1438.6 | 1439.5 |
| (R∧P)_{RP} | (R∧P)_{RP1} | [M + H]⁺ = 587.3; [M + H: —N₃ → —NH₂]⁺ = 561.3 | Below |
| | | | ROD |
| | (R∧P)_{RP2} | [M + H]⁺ = 894.4; | 930.6 |
| | | [M + H: —N₃ → —NH₂ & —SH → —SSCH₂CH₂OH & | |
| | | —NO → —NH₂]⁺ = 944.5 | |
| (R∧P)_{REP} | (R∧P)_{RP1} | [M + H]⁺ = 587.3; [M + H: —N₃ → —NH₂]⁺ = 561.3 | Below |
| | | | ROD |
| | (R∧P)_{RP2} | [M + H]⁺ = 894.4; | 930.5 |
| | | [M + H: —N₃ → —NH₂ & —SH → —SSCH₂CH₂OH & | |
| | | —NO → —NH₂]⁺ = 944.5 | |

Expected products were identified in all treatment conditions except product (R∧E)_{RP1}, presumably due to being below the MALDI range of detection (m/z>600). Products were of good purity in all cases.

### Method S25. In Situ Rheology of Hydrogel Formation

### Strain and Frequency Sweeps:

Oscillatory rheology was performed on an Anton Paar MCR301 rheometer equipped with a cone and plate geometry (25 mm diameter, 1° cone) at 25° C. Strain and frequency sweeps were used to determine the linear viscoelastic region (LVR), the set of imposed conditions from which the elastic modulus is independent.

A solution of PEG-tetraBCN (Method S22, 2 mM) and the R crosslinker (Method S6, 4 mM) in PBS was reacted in situ for 120 minutes to form a hydrogel network. For the strain sweep, the frequency was fixed at 25 Hz, and the storage (G′) and loss (G″) moduli were measured at various strains (0.01-10%). For the frequency sweep, the strain was fixed at 1%, and G′ and G″ were measured at various frequencies (0.1-100 Hz). The sweeps demonstrate that at 25 Hz and 1% strain, the hydrogel networks are in the LVR because a perturbation to either the frequency or strain does not change the value of G′. These conditions were used for future rheological analysis.

### In Situ Hydrogel Rheology:

Oscillatory rheology was performed on an Anton Paar MCR301 rheometer equipped with a cone and plate geometry (25 mm diameter, 1° cone) at 25° C. and 25 Hz with a 1% strain. A solution of PEG-tetraBCN (3 mM, Method S22) and the E∧P crosslinker (6 mM, Method S12) in MMP buffer was reacted in situ, while G′ and G″ were measured as a function of time over the course of two hours. Peptide crosslinkers were pretreated with MMP and/or light as described in Method S24 in experimental triplicate. Hydrogels with similar stiffnesses formed when the E∧P crosslinker was untreated (G′=1660±170), exposed to just light (G′=1580±130), or exposed to just enzyme (G′=1540±110). When the crosslinker was treated with both enzyme and light, a hydrogel did not form (G′=200±30). All samples had a final loss modulus (G″) of ˜50 Pa.

### Method S26. Assessing Network Relaxation Following Partial Cleavage of AND-Gate Crosslinkers

To estimate the effect of partial AND-gate linker cleavage on bulk material properties, the average number of elastically active covalent bonds between network branch points was compared before and after a single degradation event. As these bonds contributed by the 4-arm PEG represent the overwhelming majority of those between crosslinks (>90%), minimal structural relaxation is predicted following the cleavage of a single stimuli-labile moiety (<3% for all AND-gated crosslinkers). This is consistent with rheology data, where AND-gate crosslinker treatment with a single input does not produce a significant change in final material stiffness (Method S25).

### E∧P Crosslinker

Bonds from PEG macromer=702
Bonds shared by both arms of peptide linker=26
Bonds through E-arm=30
Bonds through P-arm=34
758 vs 762 indicates a 0.5% difference

### E∧R Crosslinker

Bonds from PEG macromer=702
Bonds shared by both arms of peptide linker=30
Bonds through E-arm=30
Bonds through R-arm=5
737 vs 762 is a 3.3% difference

### R∧P Crosslinker

Bonds from PEG macromer=702
Bonds shared by both arms of peptide linker=31
Bonds through R-arm=5
Bonds through P-arm=25
738 vs 758 is a 2.6% difference

### Method S27. Logic-Based Hydrogel Degradation in Response to Sequential Stimuli

Fluorescent hydrogels were formulated from a precursor solution of PEG-tetraBCN-AF568 (2 mM) and a diazide peptide crosslinker (4 mM) in MMP buffer. Immediately upon addition of all components (65 μL total), the solution was vortexed and centrifuged. The precursor solution was transferred via a positive displacement pipette into microcentrifuge tubes (6×10 μL), centrifuged, and reacted at room temperature for 1 hour. To remove any fluorescent sol fraction, hydrogels were washed in MMP buffer (1 mL, 2×24 hours, 37° C.) prior to treatment. A total of 24 hydrogels were synthesized from each crosslinker (8 input combinations in experimental triplicate).

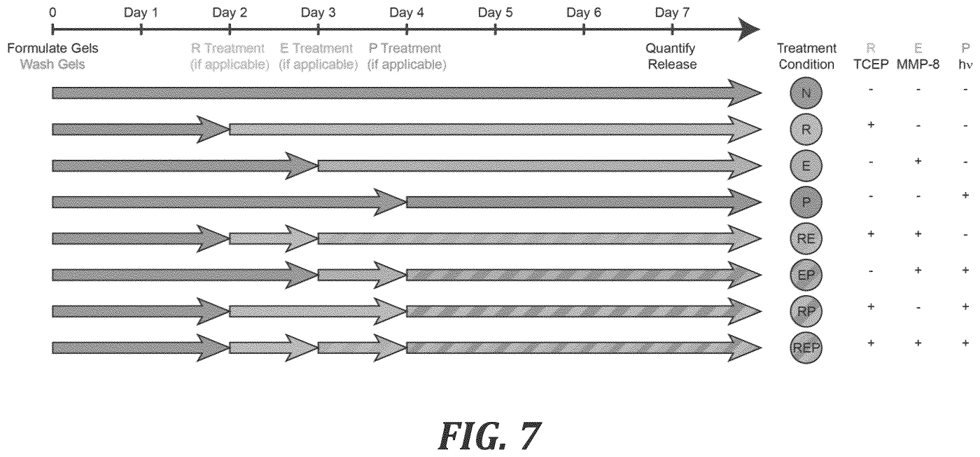

Referring to FIG. 7, Samples receiving the reductive input (_{R}) were treated with tris(2-carboxyethyl)phosphine hydrochloride (TCEP·HCl, 2 μL, 100 mM in MMP buffer) and incubated overnight (22 hr) at 37° C. To quench any unreacted TCEP, these samples were further treated with hydroxyethyl disulfide (5 μL, 100 mM in MMP buffer) prior to incubation (4 hr, 37° C.). Samples not receiving reductive input were maintained at 37° C.

Samples receiving the enzyme input (_{E}) were subsequently treated with MMP-8 (Method S23, 5 μL, 0.2 mg mL⁻¹ in MMP buffer) and all samples were incubated (20 hr, 37° C.).

Samples receiving the light input (_{P}) were subsequently exposed to UV light (λ=365 nm, 10 mW cm⁻² incident light, 60 minute exposure) prior to incubation (72 hr, 37° C.).

After 8 total days of experimental treatment, the extent of gel degradation was assessed by quantifying supernatant fluorescence (excitation: 570 nm, emission: 610 nm, emission cut-off filter: 590 nm).

### Methods S28. Characterization of PEG-tetraBCN-AF568 Fluorescence

### AF568 Photobleaching Study:

To assess the level of AF568 fluorophore photobleaching that occurs during light treatment conditions used in the hydrogel release studies (Method S27), AF568 was dissolved in PBS (10 μM) and exposed to UV light (λ=365 nm, 10 mW cm⁻²) in experimental triplicate. Sample fluorescence (100 μL, 1 μM, excitation wavelength 570 nm, emission wavelength 610 nm) was measured following 10-minute intervals of light exposure. One-way analysis of variance (ANOVA) failed to reject the null hypothesis that fluorescence does not change upon light exposure (p-value=0.82). This suggests that AF568 does not undergo significant photobleaching under light treatment conditions used in the hydrogel degradation studies.

### Quantification of PEG-tetraBCN-AF568 Concentrations Using Fluorescence:

Fluorescence was used to determine the concentration of PEG-tetraBCN-AF568 in solution, and thus to quantify the extent of gel degradation in response to each combination of external stimuli. PEG-tetraBCN-AF568 fluorescence was significantly altered both in the presence of oNB, and in the presence of photocleaved oNB (oNB spectral properties change upon photodecomposition). Therefore, three different calibration curves are required to correlate fluorescent signal with PEG-tetraBCN-AF568 concentration for crosslinkers that: 1) do not contain oNB; 2) contain oNB but have not been exposed to light; 3) contain photocleaved oNB.

A calibration curve was generated using pure PEG-tetraBCN-AF568 in MMP buffer, and was used for constructs that do not contain oNB. A second calibration curve was generated using PEG-tetraBCN-AF568 and P crosslinker (Method S7, 1:2 molar ratio) in MMP buffer, and was used for systems that contain oNB but have not been exposed to light. The final calibration curve was generated using PEG-tetraBCN-AF568 and P crosslinker (1:2 molar ratio) treated by the light condition used in the release studies (λ=365 nm, 10 mW cm², 60 minutes) in MMP buffer, and was used for systems that contain oNB and have been exposed to light.

Sample fluorescence was found to be scale with PEG-AF568 concentration for all cases, enabling the extent of hydrogel degradation to be quantified for each crosslinker system in response to different input combinations.

### Gel Photographs Following Logic-Based Degradation

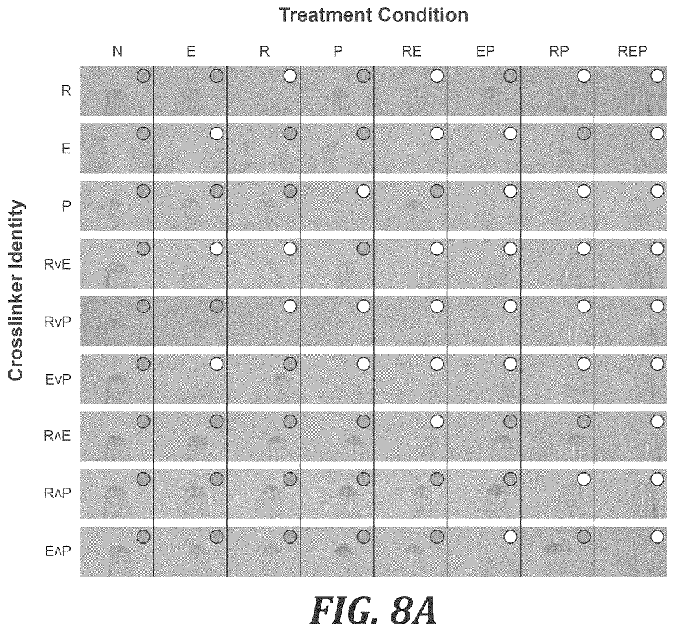

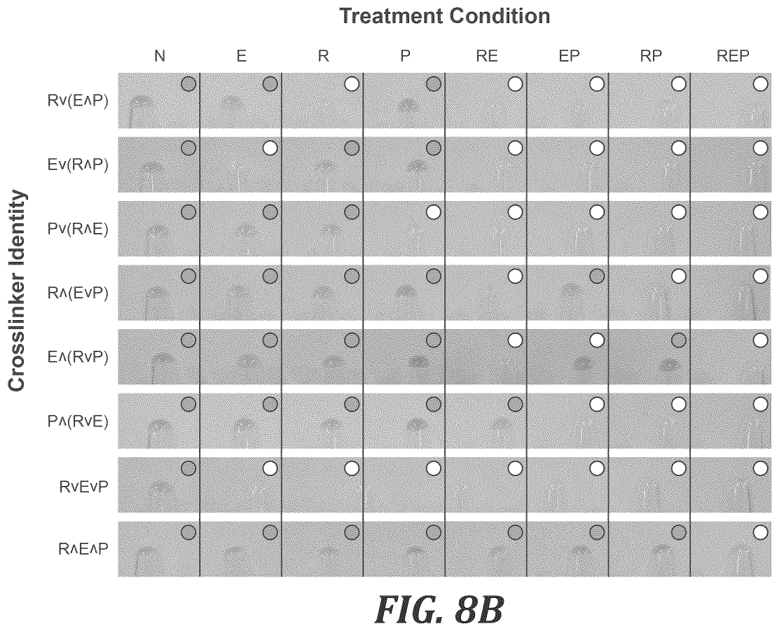

Referring to FIG. 8, fluorescent hydrogels were prepared and treated as described in Method S27. Following complete degradative treatment, samples were photographed with a digital camera. Treatments that did not result in material degradation retained a hydrogel (grey). The expected degradation behavior is indicated by a colored dot in each condition; a grey dot denotes conditions that are expected to keep the material intact while a white dot denotes those expected to yield gel degradation.

### Degradation Kinetics of Single-Input Hydrogels

To assess the kinetics of hydrogel degradation, the influence of input intensity and time on the response of single-input, YES-gated materials (R, E, and P) was examined. Fluorescent hydrogels were formulated as before (10 μL, Method S27) in two different geometries: 1) as a bulk gel in a microcentrifuge tube, and 2) as a thin gel (˜50 μm thickness) in a 24-well plate. Hydrogels were washed with MMP buffer (1 mL, 2×24 hours, 37° C.) prior to treatment. At each timepoint, the extent of gel degradation was assessed by quantifying supernatant fluorescence (excitation: 570 nm, emission: 610 nm, emission cut-off filter: 590 nm), in experimental triplicate.

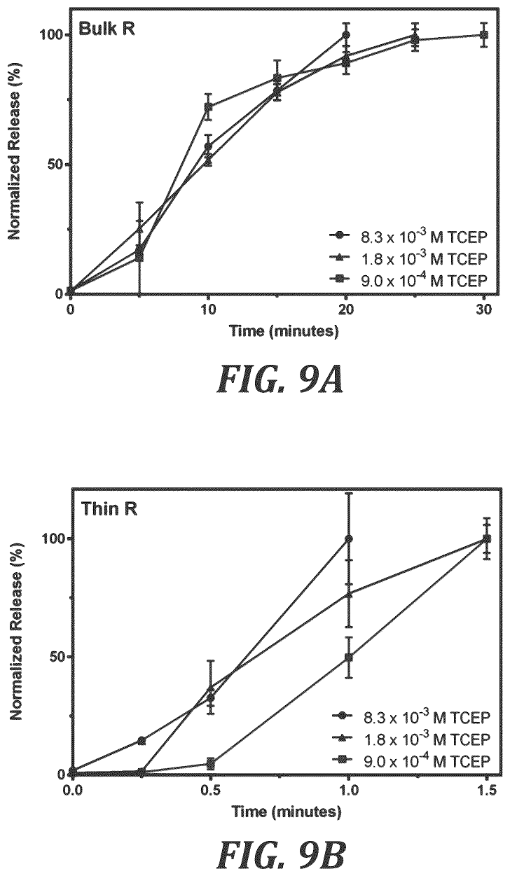

Referring to FIGS. 9A-9B, Reductively degradable hydrogels (R) were treated with TCEP·HCl (8.3 mM, 1.8 mM, or 0.90 mM in MMP buffer) at 37° C.

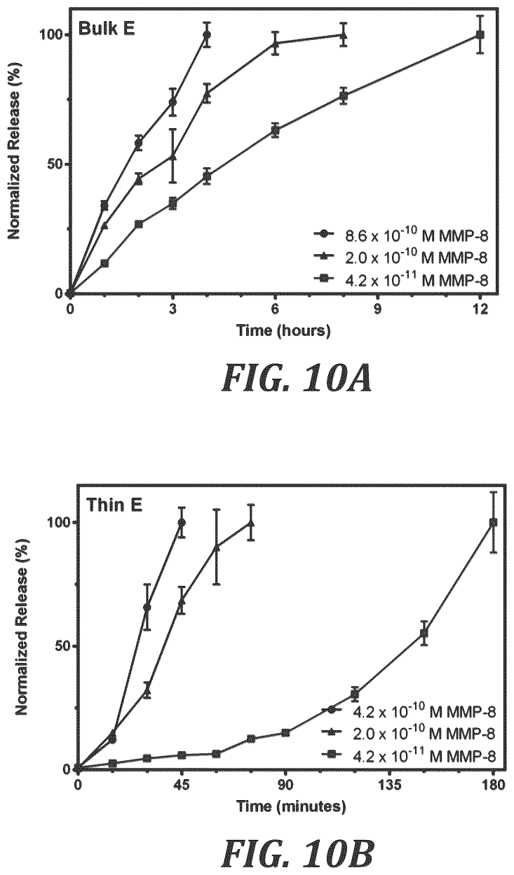

Referring to FIGS. 10A-10B, enzymatically degradable hydrogels (E) were treated with MMP-8 (0.86 nM, 0.42 nM, 0.20 nM, or 0.042 nM in MMP buffer) at 37° C.

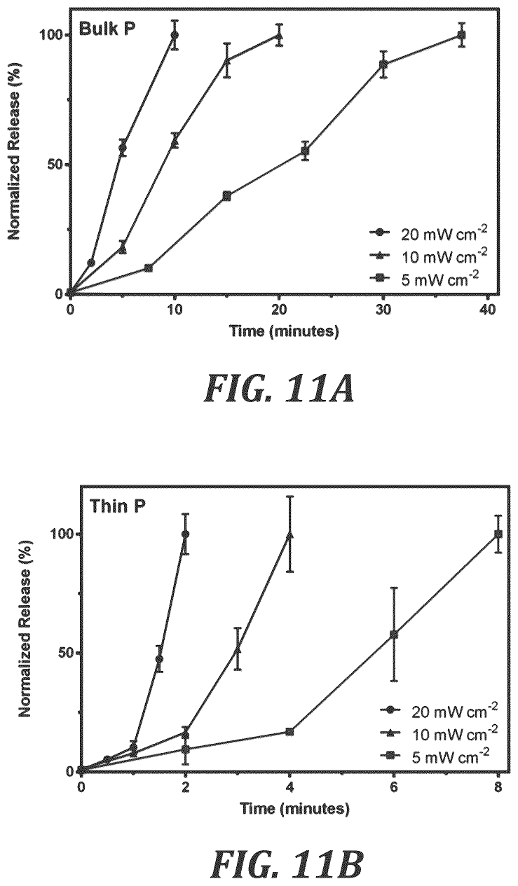

Referring to FIGS. 11A-11B, photolytically degradable hydrogels (P), were treated with UV light (λ=365 nm, 20 mW cm⁻², 10 mW cm⁻², or 5 mW cm⁻²) in MMP buffer.

These experiments highlight that the timescale of logic-based material response is dictated, in part, by construct geometry. As each single-input gel/treatment combination yielded full degradation in <1.5 hours for thin gels or <12 hours for bulk gels, the experimental timeline outlined in Method S27 was deemed appropriate to examine biocomputational responses of higher-ordered logical systems.

### Method S29. Logic-Based Delivery of Functional Doxorubicin from Hydrogels

### Synthesis of (1R,8S,9s)-bicyclo[6.1.0]non-4-yn-9-ylmethyl ((2S,3S,4S,6R)-3-hydroxy-2-methyl-6-(((1S,3S)-3,5,12-trihydroxy-3-(2-hydroxyacetyl)-10-methoxy-6,11-dioxo-1,234,6,11-hexahydrotetracen-1-yl)oxy)tetrahydro-2H-pyran-4-yl)carbamate (DOX-BCN)

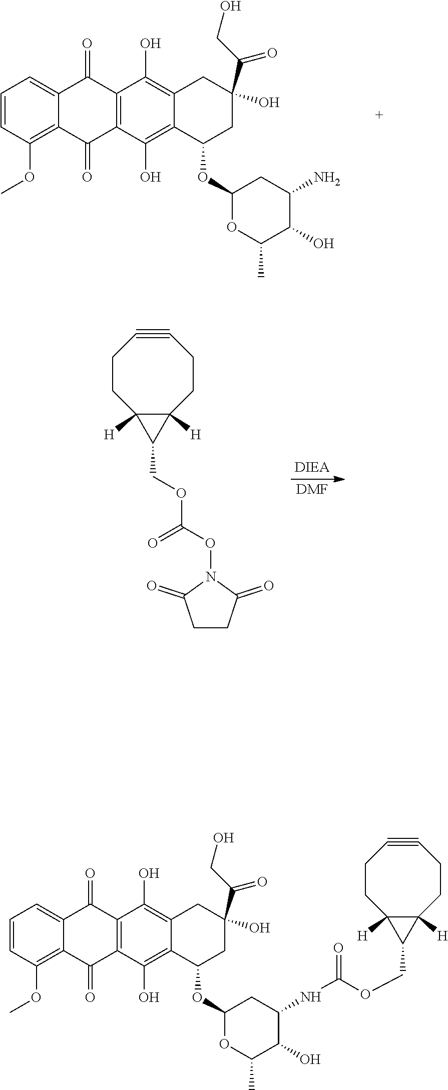

Doxorubicin hydrochloride (DOX, 6.0 mg, 10 μmol), BCN-OSu (Method S22, 6.0 mg, 21 μmol, 2×), and DIEA (5.3 mg, 41 μmol, 4×) were dissolved in minimal DMF and reacted overnight to functionalize doxorubicin with BCN. Complete functionalization of doxorubicin was confirmed via HPLC. The product (DOX-BCN) was used without any further purification.

### Synthesis of R∧E-DOX Crosslinker

DOX-BCN (0.8 μmol) and R∧E crosslinker (4 μmol, 5×) were reacted in a mixture of DMF/PBS (300 μL) for 2 hours, whereby roughly 10% of R∧E-presented azides were modified with DOX. The product (R∧E-DOX) was used without any further purification.

### DOX-Loaded Hydrogel Treatments:

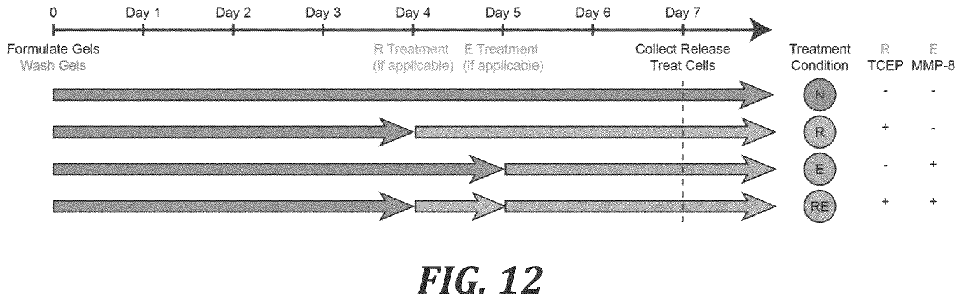

DOX-loaded hydrogels were formulated from a precursor solution of PEG-tetraBCN (2 mM) and either R∧E (4 mM, control group) or R∧E-DOX linker (4 mM, experimental group) in a 4-(2-hydroxyethyl)-1-piperazineethanesulfonic acid) (HEPES) buffer (5 mM HEPES, 3 mM CaCl₂), 5 μM ZnCl₂). Immediately upon addition of all components (95 μL total), the solution was vortexed and centrifuged. The precursor solution was transferred via a positive displacement pipette into microcentrifuge tubes (6×15 μL), centrifuged, and reacted at room temperature for 1 hour. To remove any unconjugated DOX, hydrogels were washed with HEPES buffer (1 mL, 4×24 hours, 37° C.) prior to treatment. Referring to FIG. 12, a total of twelve hydrogels were synthesized (four input combinations in experimental triplicate).

Samples receiving the reductive input (_{R}) were treated with glutathione (GSH, 2 mM) and incubated overnight (24 hr) at 37° C. Samples not receiving reductive input were maintained at 37° C.

Samples receiving the enzyme input (_{E}) were subsequently treated with MMP-8 (Method S23, 10 μL, 0.2 mg mL¹ in MMP buffer) and all samples were incubated (24 hr, 37° C.).

After experimental treatment, hydrogel supernatant (85 μL) was collected and combined with Dulbecco's Modified Eagle's Medium [DMEM, 85 μL, 2× containing 20% fetal bovine serum (FBS, Corning) and 2% Penicillin Streptomycin (PS, ThermoFisher)].

### Biological Response to DOX-Hydrogel Supernatant:

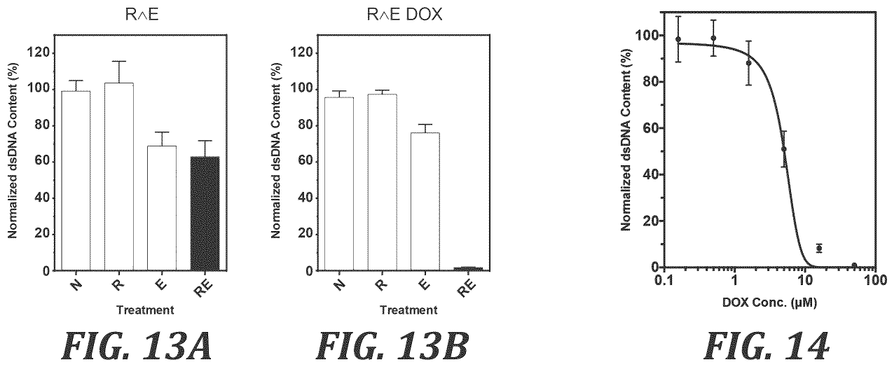

HeLa cells were seeded on a 96-well plate (2×10³ cells per well) and cultured in DMEM supplemented with 10% FBS and 1% PS for 24 hours. The cells were then incubated in the DOX-hydrogel release solution (as described above), or in that from R∧E gels lacking DOX, for an additional 48 hours. Referring to FIGS. 13A-13B, a PicoGreen® dsDNA Assay (ThermoFisher) was performed to quantify the DNA concentration as a proxy for cell density.

### Dose-Response Curve for Doxorubicin Construct:

Referring to FIG. 14, HeLa cells were seeded on a 96-well plate (2×10³ cells per well) and cultured in DMEM supplemented with 10% FBS and 1% PS for 24 hours. The media was replaced with R∧E-DOX in a mixture of HEPES buffer and 2× media (1:1) and incubated for 48 hours prior to quantification of dsDNA by PicoGreen® analysis.

### Method S30. Multi-Logic Hydrogel Treatment and Microscopy

### Synthesis of (3-azidopropyl)trimethoxysilane

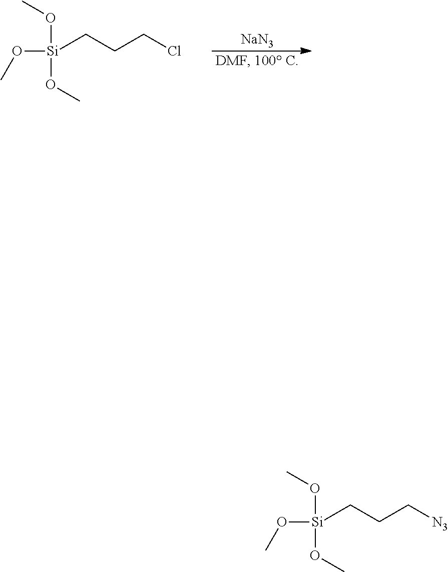

Anhydrous DMF (40 mL) was added to an oven-dried round bottom flask containing 3-chloropropyltrimethoxysilane (12 mL, 65.3 mmol, 1×) and sodium azide (6.38 g, 98.1 mmol, 1.5×) under a nitrogen atmosphere. The reaction was stirred overnight at 100° C., cooled to room temperature, and diluted with diethyl ether:dH₂O (1:1, 150 mL). The organic layer was washed with water (3×) and brine (1×), dried over MgSO₄, and concentrated in vacuo to yield a clear oil (12.74 g, 62.1 mmol, 95% yield). ¹H NMR (500 MHz, CDCl₃) δ 3.57 (s, 9H), 3.26 (t, J=6.9 Hz, 2H), 1.75-1.66 (m, 2H), 0.73-0.66 (m, 2H); ¹³C NMR (126 MHz, CDCl₃) δ 53.86, 50.72, 22.58, 6.46. These spectral data matched those previously reported.

### Treatment of Glass Slides:

Plain glass slides were cleaned in piranha solution (50% sulfuric acid, 35% dH₂O, 15% hydrogen peroxide) for 30 minutes at room temperature. The slides were rinsed with water, acetone (3×) and then dried. The slides were incubated for 90 minutes in a solution of (3-azidopropyl)trimethoxysilane (70 mM) and n-butylamine (70 mM) in toluene. The slides were subsequently rinsed with toluene, wiped dry with a Kimwipe™, and baked overnight at 80° C. Functionalized slides were stable when stored at room temperature under ambient conditions for several weeks.

### Preparation of Three-Region Hydrogels Differently Sensitive to Light and Reductant:

Hydrogels were formed sandwiched between an azide-functional glass slide and a Rain-X®-treated glass slide separated by two 0.005″ thick silicone spacers (McMaster-Carr). A precursor solution containing PEG-tetraBCN-FAM (Method S22, 2 mM) and the P crosslinker (Method S7, 4 mM) was injected between the silicone spacers (0.005 inches) and reacted for one hour. Subsequently, a precursor solution of PEG-tetraBCN-Cyanine5 (Method S22, 2 mM) and the R∨P crosslinker (Method S10, 4 mM) was injected between the FAM functionalized region and a silicone spacer and reacted for one hour. This was repeated on the other side of the FAM functionalized region with PEG-tetraBCN-AF568 (Method S22, 2 mM) and R∧P crosslinker (Method S13, 4 mM). Hydrogels were equilibrated and stored in PBS.

### Treatment of Three-Region Hydrogels Sensitive to Light and Reductant:

Preformed tri-color gels were exposed to UV light (λ=365 nm, 10 mW cm² incident light, 10 minute exposure) through a slitted photomask (200 μm wide lines separated by 200 μm spaces), inducing degradation of the P- and R∨P-based gels in the UV-exposed regions. Following light treatment, gels were soaked in 2-mercaptoethanol (BME, 0.25 mM in 50 mL PBS) for 45 minutes at room temperature, yielding full degradation of the R∨P-based gel portion while leaving the P-based volume fully intact. The R∧P-based material degraded to reveal the original photopatterned exposure, demonstrating that both inputs are required for material degradation (see FIG. 5A).

### Preparation of Four-Region Hydrogels Differently Sensitive to Reductant and Enzyme:

Hydrogels containing four regions, each with a distinct logical crosslinker and fluorophore, were prepared using the method described above. The regions from left to right were: 1) R crosslinker/FAM (Methods S6, S22), 2) R∧E crosslinker/AF568 (Methods S11, S22), 3) E crosslinker/FAM (Methods S5, S22), and 4) R∨E crosslinker/Cyanine5 (Methods S8, S22).

### Treatment of Four-Region Hydrogels Sensitive to Reductant and Enzyme:

Preformed four-color gels were sequentially exposed to reducing conditions (0.25 mM BME, 45 minutes) and MMP-8 (0.20 nM, 2 hours, Method S23), in either order. In response to the initial treatment, two regions of the hydrogel degraded (R∨E and the relevant single-input region). Exposure to the second input induced full degradation on the remainder of the hydrogel.

### Hydrogel Microscopy:

Hydrogels were imaged using fluorescent confocal microscopy. Three channels—one for each fluorophore—were simultaneously monitored. For visualization, fluorescence corresponding to AF568 (excitation 578 nm, emission 594-627 nm), FAM (excitation 515 nm, emission 520-569 nm), and Cyanine5 (excitation 646 nm, emission 663-675 nm) were false colored as red, green, and blue, respectively.

### Method S31. Hydrogel-Encapsulated Cell Release Studies

Four human bone marrow-derived stromal cell lines [hS5], including one non-fluorescent control and three each stably transfected to express a single fluorescent protein [mCherry, green fluorescent protein (GFP), or blue fluorescent protein (BFP)], were cultured at 37° C. in Roswell Park Memorial Institute medium 1640 (RPMI-1640, Corning) supplemented with 10% FBS and 1% PS.

### Cell-Laden Multi-Colored Hydrogel Preparation for Microscopy:

Hydrogels were formed sandwiched between an azide-functional glass slide and a Rain-X®-treated glass slide separated by two 0.005″ thick silicone spacers (McMaster-Carr). A precursor solution containing PEG-tetraBCN (Method S22, 2 mM), the P crosslinker (Method S7, 4 mM), and hS5-GFP⁺ (40×10⁶ cells mL⁻¹) was injected between the silicone spacers and reacted for one hour. Subsequently, a precursor solution of PEG-tetraBCN (2 mM), the R∨P crosslinker (Method S10, 4 mM), and hS5-BFP⁺ (40×10⁶ cells mL⁻¹) was injected between the hS5-GFP⁺ encapsulated region and a silicone spacer. Immediately afterwards, a precursor solution of PEG-tetraBCN (2 mM), R∧P crosslinker (Method S13, 4 mM), and hS5-mCherry⁺ (40×10⁶ cells mL⁻¹) was injected between the other side of the hS5-GFP⁺ encapsulated region and a silicone spacer. The hS5-mCherry⁺ and hS5-BFP⁺ encapsulated regions were simultaneously reacted for one hour. Hydrogels were stored in media overnight.

### Cell-Laden Hydrogel Treatments for Microscopy:

Preformed tri-color cell-laden gels were exposed to UV light (λ=365 nm, 10 mW cm-2 incident light, 10 minute exposure) through a slitted photomask (200 μm wide lines separated by 200 μm spaces), inducing degradation of the P- and R∨P-based gels in the exposed regions and associated release of hS5-GFP⁺ and hS5-BFP⁺ cells. Following light treatment, gels were soaked in 2-mercaptoethanol (BME, 0.25 mM in 50 mL PBS) at 37° C. for 45 minutes, yielding full degradation of the R∨P-based gel portion (including hS5-BFP⁺ release) while leaving the P-based gel fully intact. The R∧P-based gel degraded to match the original photopatterned exposure (causing release of hS5-mCherry⁺ cells), demonstrating that both inputs are required for material degradation. Finally, gels were exposed to unmasked light to induce complete degradation of any remaining material and to release an equal number of hS5-GFP⁺ and hS5-mCherry⁺ cells.

Cell-laden hydrogel microscopy:

Following each degradative step, cells that remained encapsulated in the intact gel were imaged using fluorescent confocal microscopy. Three channels—one for each fluorescent protein—were simultaneously monitored. For visualization, fluorescence corresponding to mCherry (excitation 587 nm, emission 626-703 nm), GFP (excitation 489 nm, emission 499-570 nm), and BFP (excitation 405 nm, emission 413-494 nm) were false colored as red, green, and blue, respectively.

### Quantifying Cell Release by Flow Cytometry:

Following each degradative step, released cells were harvested though successive gel rinsing with PBS and centrifugation. For each treatment condition, released cells were fixed using a solution of 4% formaldehyde in PBS (37° C., 10 minutes), chilled on ice (1 minute), concentrated by centrifugation, and resuspended in PBS. Flow cytometry was performed on released cell populations using a BD Biosciences LSR II Flow Cytometer. Forward scattering (FS), side scattering (SS), and the fluorescence corresponding to BFP (405 nm and 100 mW laser line, 450/50 nm bandpass filter), GFP (488 nm and 100 mW laser line, 530/30 nm bandpass filter), and mCherry (561 nm and 150 mW laser line, 610/20 nm bandpass filter) were measured for each event. Flow cytometry was performed on standards of each cell line (non-fluorescent, mCherry, GFP, and BFP) and cells released from each treatment.

### Analysis of Flow Cytometry Data:

To account for spectral overlap of fluorescent proteins, compensation controls were calculated from the standards using automatic compensation in FACSDiva software. The gating tree was set as follows (shown below for GFP standard). A: FSC/SSC (the distribution of cell size and intracellular complexity, respectively, from light scatter) to B: FSC/pulse width (to isolate events corresponding to single cells) to C: histograms of each fluorescent channel. Gating for A and B was performed using non-fluorescent hS5 cells. Gating for C was performed for red, green, and blue fluorescence using hS5-mCherry⁺, hS5-GFP⁺, hS5-BFP⁺ standards, respectively.

### Cell-Laden Hydrogel Preparation for Viability Studies:

Single-input responsive hydrogels were formed sandwiched between an azide-functional glass slide and a Rain-X®-treated glass slide separated by two 0.005″ thick silicone spacers (McMaster-Carr). A precursor solution containing PEG-tetraBCN (2 mM), a YES crosslinker (either R, E, or P; 4 mM), and hS5 (40×10⁶ cells mL¹) was injected between the silicone spacers and reacted for one hour. Hydrogels were stored in media for one hour prior to treatments. Each material was generated and assayed in experimental triplicate.

### Cell-Laden Hydrogel Degradation for Viability Studies:

Gels were degraded and the viability of the released cells was assessed. To induce gel degradation, R gels were treated with BME (0.25 mM in PBS) at 37° C. for 45 minutes; P gels were exposed to UV light (λ=365 nm, 10 mW cm² incident light) at 25° C. for 10 minutes; E gels were treated with MMP-8 (0.20 nM in RPMI) at 37° C. for 60 minutes. All treatments yielded complete hydrogel degradation. Released cells were collected and isolated via centrifugation prior to Live/Dead® staining (Invitrogen).

### Viability Analysis:

Cells released from the gels and stained with the Live/Dead® assay were imaged on a Nikon Eclipse TE2000-U microscope using filter cubes corresponding to both live (excitation 480/40 nm filter, emission 535/50 nm filter) and dead (excitation 560/20 nm filter, emission 630/60 nm filter) staining. Viability was determined for each treatment by standard image analysis. For each release treatment, representative fluorescent images of live and dead cells are false colored green and red, respectively.

### Example 2. Programmable Logic-Based Delivery of Small Molecule Therapeutics from Gels

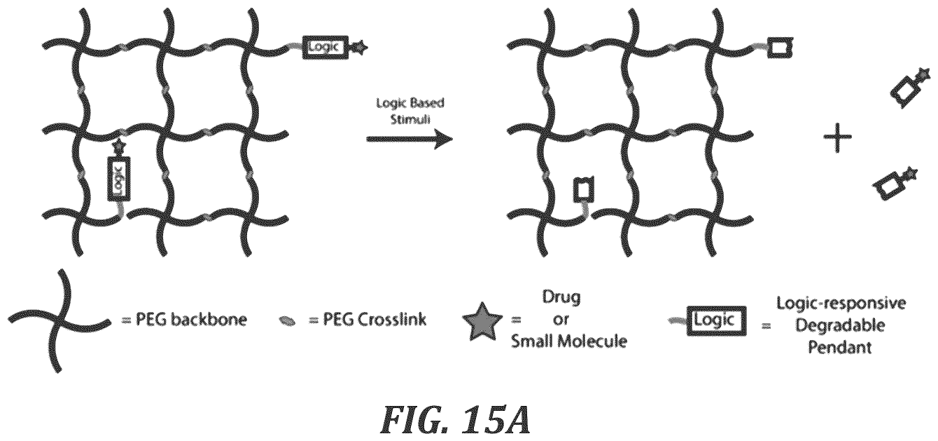

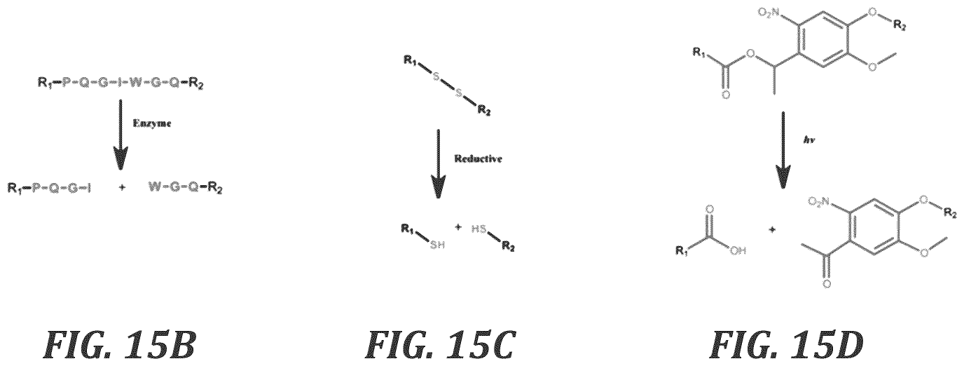

Delivery specificity was obtained by requiring multiple user-defined cues to be programmed into materials to induce a drug release. A peptide-based approach was used to covalently tether therapeutics to a hydrogel biomaterial while employing the utility of linear or cyclic structures to provide the opportunity to program drug release using Boolean logic; YES, AND, OR. Three modes of degradation were chosen to incite release of the therapeutic; an enzyme cleavable sequence, a reducible group, and a photolabile crosslinker. The simplest YES logic pendants incorporate a singular stimuli dependent sequence. It was found that with the incorporation of the amino acid sequence QPQG↓IWGQ (SEQ ID NO. 25) into the backbone of a peptide would be selectively recognized by matrix metalloproteinases (MMPs), including MMP-8, which was identified as an enzyme that is upregulated by cancer cells to promote the digestion of the extracellular matrix. MMP-8 selectively recognizes, and cleaves the sequence between the isoleucine and tryptophan. The induced enzymatic response releases the therapeutic with the remaining peptide sequence. The second logical mode of degradation was accomplished via a reducible disulfide bridge programmed into the tether using cysteines. In the presence of a strongly reductive environment, such as would be seen in the area of damaged or cancerous tissue, the disulfide bridge will readily be cleaved to release the therapeutic. In the case of the model system, dithiothreitol (DTT) was used as a small-molecule redox reagent, which performs a sequential thiol-disulfide exchange to form an oxidized cyclic DTT via a well characterized mechanism. The final logical unit was an orthoxy-nitrobenzenoic ester derivative which undergoes cleavage of the ester bond when with multiphoton excitation. The system utilized in Example 2 is unique in that a known concentration of a therapeutic directly is tethered to a material backbone which then would be released upon degradation of the customized tether. As such, small molecule therapeutics can be delivered to a unique environment in response to targeted environmental cues. This circumvents the issues associated with encapsulation models which suffer from diffusion related losses and thus non-specific systemic releases. The YES encoded peptides were expected to show a selective system (FIG. 15A).

The logical drug release pendants are constitutively designed with three parts; the small molecule or therapeutic for desired delivery, a logic-based degradable sequence, and an azide functional group to conjugate the delivery platform to a biomaterial network. A poly(ethylene glycol) (PEG) was chosen as a model biomaterial for several different reasons. PEG is believed to elicit a nominal immune-response while being relatively bio-inert and biocompatible, thus being one of the few biomaterials with FDA approval. It is optically clear making it useful for photodegradation of the of the ortho-nitroxybenzanoic ester group. PEG is also readily characterized and is hydrophilic at in vivo conditions (37° C., pH=7.4). Furthermore, PEG could be readily functionalized for Strain-Promoted Alkyne-Azide Cycloadditions (SPAAC), thus enabling the use of a diverse set of compounds to be incorporated into the biomaterial at the time of gelation. 5,6-carboxyfluorescein (FAM) was chosen as a fluorescent reporter. FAM was covalently linked to one terminus of each tether. FAM was a good reporter for this system due to its small molecular weight, relatively low hydrodynamic radius, solubility in aqueous solutions, and a linear response curve for the measurable concentrations of pendants we used in this model.

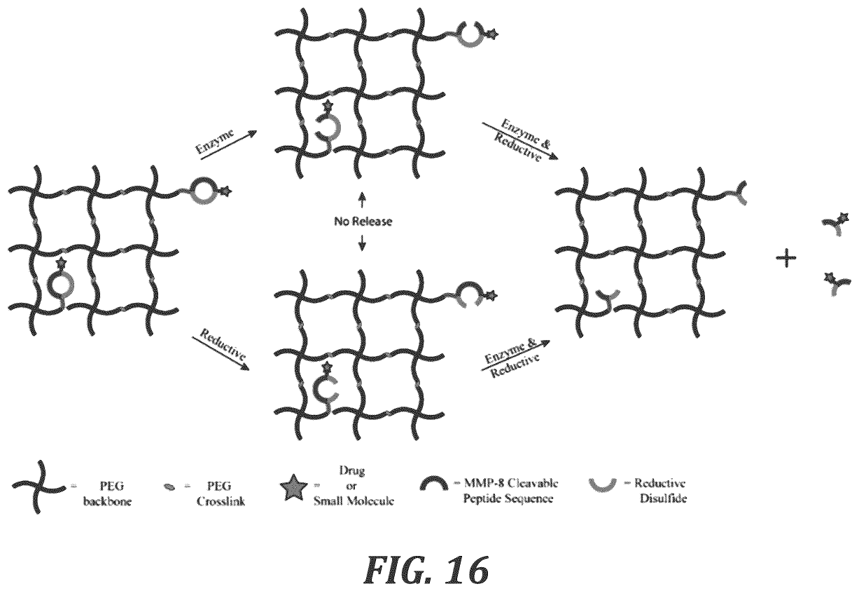

While simple YES logical delivery mechanisms for therapeutics can be useful, the peptide-based design allows the opportunity to modify and cyclize peptides with the different degradable units, thus developing AND logic. When conjugated into a gel based network with a chosen therapeutic, an orthogonal response is expected where the therapeutic would only release when in the presence of both required stimuli (FIG. 16).

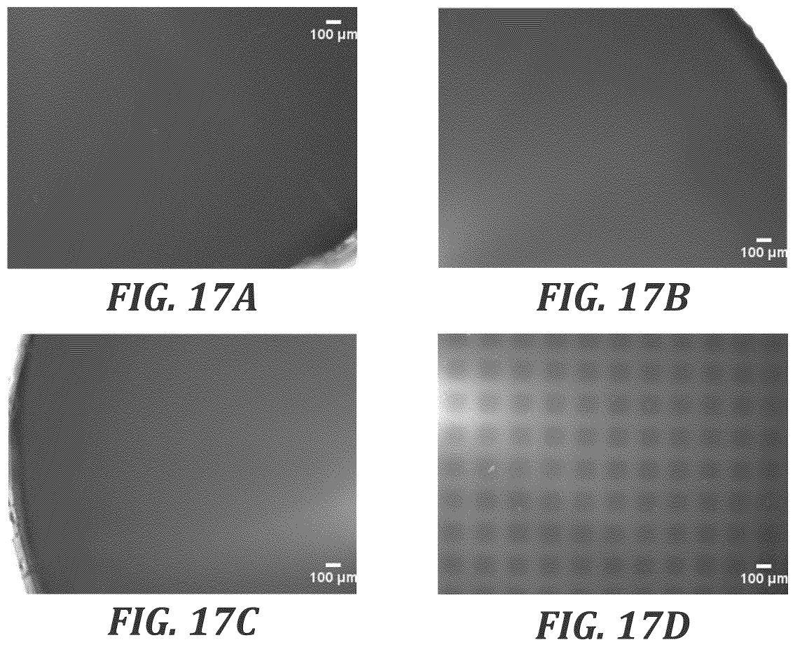

The increased selectivity of the-AND-based logic shows how this system could be applied to tailor a releasable tether for the delivery of therapeutics. Furthermore, our photolabile-AND-systems had the added benefit of spatio-temporal control for multiple applications, especially 3-D cell culture and therapeutic screening assays. This spatio-temporal control was exemplified using a photolabile-AND-reducible pendant with the 5,6-carboxyfluorescein as the model reporter (FIG. 17).

As shown in FIG. 17, the model therapeutic was only released in the immediate areas which were exposed to both light and DTT treatment. This finite spatial control could provide opportunities to better study cell cultures and their responses to small-molecule stimuli in a 3-D cell culture.

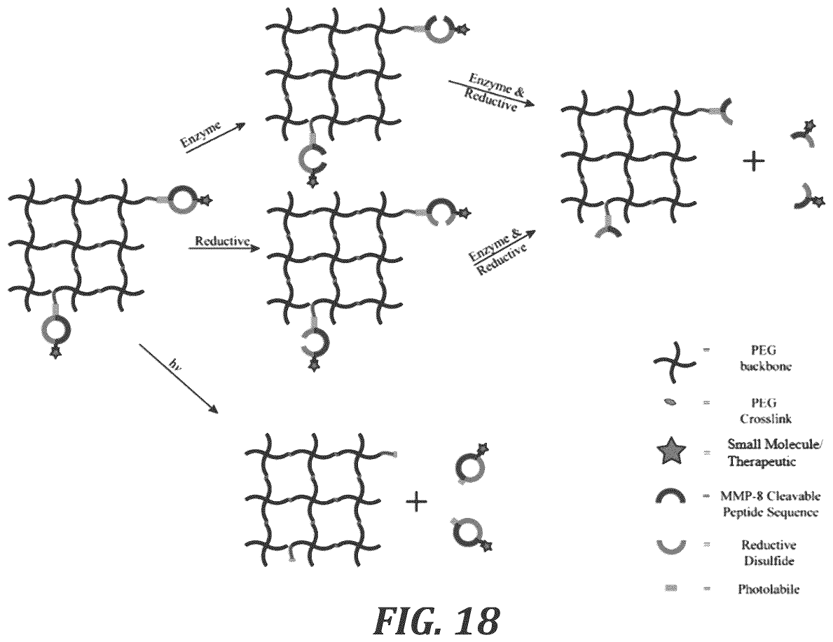

Further control of the system was accomplished by the incorporation of several logical degradation modalities in series. The resulting-OR-stimuli response would release the small-molecule therapeutic as long as only one environmental cue is present, thus maximizing therapeutic delivery as necessary. Conversely, the-OR-logic could be combined with the-AND-responsive subunits to provide a user controlled release system. While Example 1 describes an exhaustive synthesis of the-AND/OR-logical peptides, Example 2 focused on a model-AND/OR-stimuli pendant which would release a therapeutic in the presence of both a reducing environment and the presence of MMP-8, or if the sample was irradiated with light. While the environment around the cells would trigger the release for the-AND-based logic, the user would further have the ability to elicit bulk therapeutic release by irradiating the entire sample or providing masked irradiation for a more finite complete release (FIG. 18).

The syntheses of poly(ethylene glycol) tetra-Bicyclononyne (PEG-tetraBCN), ε-azido-α-Fmoc-L-Lysine-OH, 4-azidobutanoic acid, ethyl 4-azidobutanoate, 4-azidobutanoic acid, hydroxyethyl photolinker, ethyl 4-[4-acetyl-2-methoxyphenoxy]butanoate, ethyl 4-[4-acetyl-2-methoxy-5-nitrophenoxy]butanoate, ethyl 4-[4-(1-hydroxyethyl)-2-methoxy-5-nitrophenoxy]butanoate, ethyl 4-[4-(1-hydroxyethyl)-2-methoxyphenoxy]butanoic acid, photolabile azide acid, 4-azidobutanoic anhydride, photolabile azide, NHS-PL-azide, azido 4-(4-(1-((4-(4-formylbenzamido)butanoyl)oxy)ethyl)-2-methoxy-5-nitrophenoxy)butanoate (NHS-oNB-N₃), and 4-azidobutanoic N-hydroxysuccinimidyl ester are as described above in Example 1.

### Synthesis of 4-azidobutanoic N-Hydroxysuccinimidyl ester

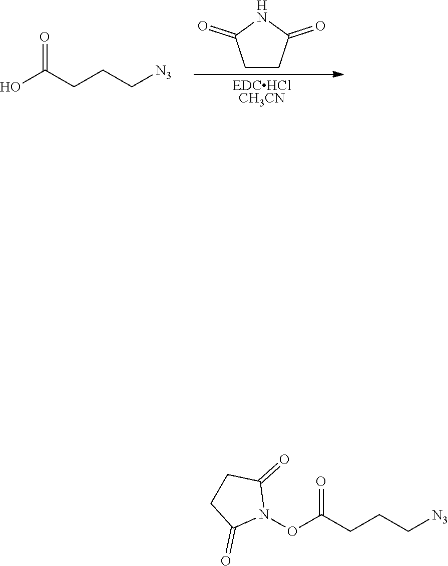

4-Azidobutanoic acid (5.0 g, 38.7 mmol, 1×), N-hydroxysuccinimide (NHS, 5.8 g, 50.3 mmol, 1.3×, Sigma), and 1-(3-Dimethylaminopropyl)-3-ethylcarbodiimide hydrochloride (EDC-HCl, 9.65 g, 50.3 mmol, 1.3×, AK Scientific Inc) were combined and purged under nitrogen. Dry acetonitrile (˜50 mL) was added via cannula transfer. Reaction stirred under nitrogen at room temperature overnight protected from light. The crude product was concentrated via rotary evaporation, then resuspended in DCM (100 mL). The organic phase was washed with dH₂O (3×100 mL), then dried over MgSO₄, filtered, then concentrated via rotary evaporator to yield a clear liquid (7.976 g, 35.3 mmol) with a good yield (91%). ¹H-NMR (500 MHz, CDCl₃) δ=3.42 (t, J=6.6 Hz, 2H), 2.81 (s, 4H), 2.70 (t, J=7.2 Hz, 2H), 2.06-1.93 (m, 2H).

### Synthesis of PEG diazide (N₃—PEG-N₃)

### Synthesis of N₃-PEG N₃

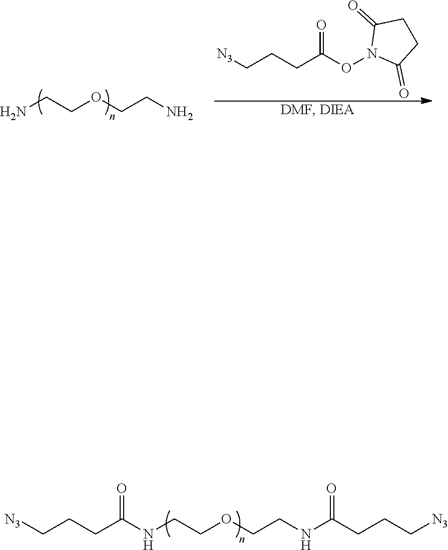

Poly(ethylene glycol) (Mₙ˜3500, 1.0 g, 0.29 mmol, JenKem) and 4-azidobutanoic-N-hydroxysuccinimidyl ester (193.9 mg, 0.86 mmol, 1.5×), were codissolved in DMF (˜3 mL) and DIEA (295.4 mg, 377.8 μL, 4×). The mixture was stirred overnight at room temperature. Resulting mixture was concentrated via rotary evaporator, dissolved in water, and dialyzed for 48 hrs (molecular weight cutoff ˜2 kDa, SpectraPor), and lyophilized to produce white fluffy product (629.8 mg, 0.17 mmol) with a 59.2% yield as a result of glassware breakage during the transfer step. ¹H-NMR (500 MHz, CDCl₃) δ=4.11 (d, J=71.00 Hz, 2H), 3.79-3.74 (m, 4H), 3.66-3.55 (m, 312H), 3.50-3.44 (m, 4H), 3.36 (t, J=6.6 Hz, 4H), 2.38 (t, J=7.3 Hz, 4H), 1.95 (dd, J=7.2 Hz, 4H).

### Synthesis of MMP-8 Single-Input Degradable Pendant

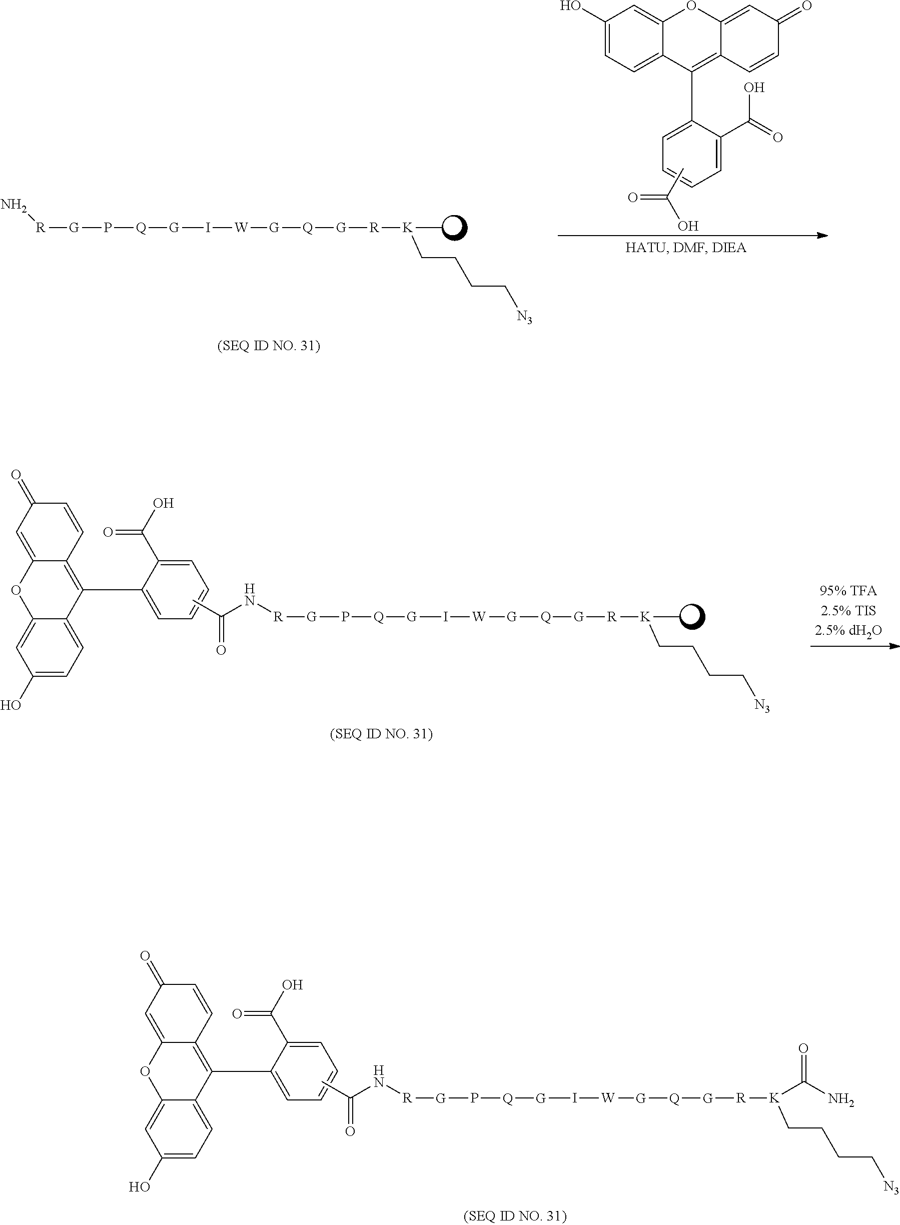

### Synthesis of FAM-RGPQGIWGQGRK(N₃)—NH₂) (SEQ ID NO. 31) Pendant

The base peptide H-RGPQGIWGQGRK(N₃) (SEQ ID NO. 31) was synthesized on rink amide resin (ChemPep, 0.8 meg/mg) via standard microwave-assisted Fmoc solid phase methodology and HBTU activation on a scale of 0.50 mmol (CEM Liberty 1). 5,6-Carboxyfluorescein (4×, 752 mg, Fisher) was coupled at room temperature two times for 2 hours each on resin with HATU (3.9×, 752 mg) dissolved in minimal DMF and activated with DIEA (8×, 661 mg). Resin was treated with trifluoroacetic acid/triisopropylsilane/water (95:2.5:2.5) for 2 hr, and the crude peptide was precipitated in and washed (2×) with ice-cold diethyl ether. The crude peptide was purified using semi-preparative reversed-phase high-performance liquid chromatography (RP-HPLC) using a 55 min linear gradient (5-100% of acetonitrile and 0.1% trifluoroacetic acid) and lyophilized to give the product (FAM-RGPQGIWGQGRK(N₃)—NH₂) (SEQ ID NO. 31) as a fluffy, yellow solid (56.2 mg, 32.6 μmol, 6.52% overall). Peptide purity was confirmed with analytical RP-HPLC and matrix-assisted laser desorption-ionization time-of-flight mass spectrometry using α-cyano-4-hydroxycinnamic acid matrix: MALDI-TOF: calculated for C₇₉H₁₀₃N₂₅O₂₀⁺ [M+¹H]⁺, 1722.8; found 1722.6.

### Synthesis of Reducible Single-Input Pendant

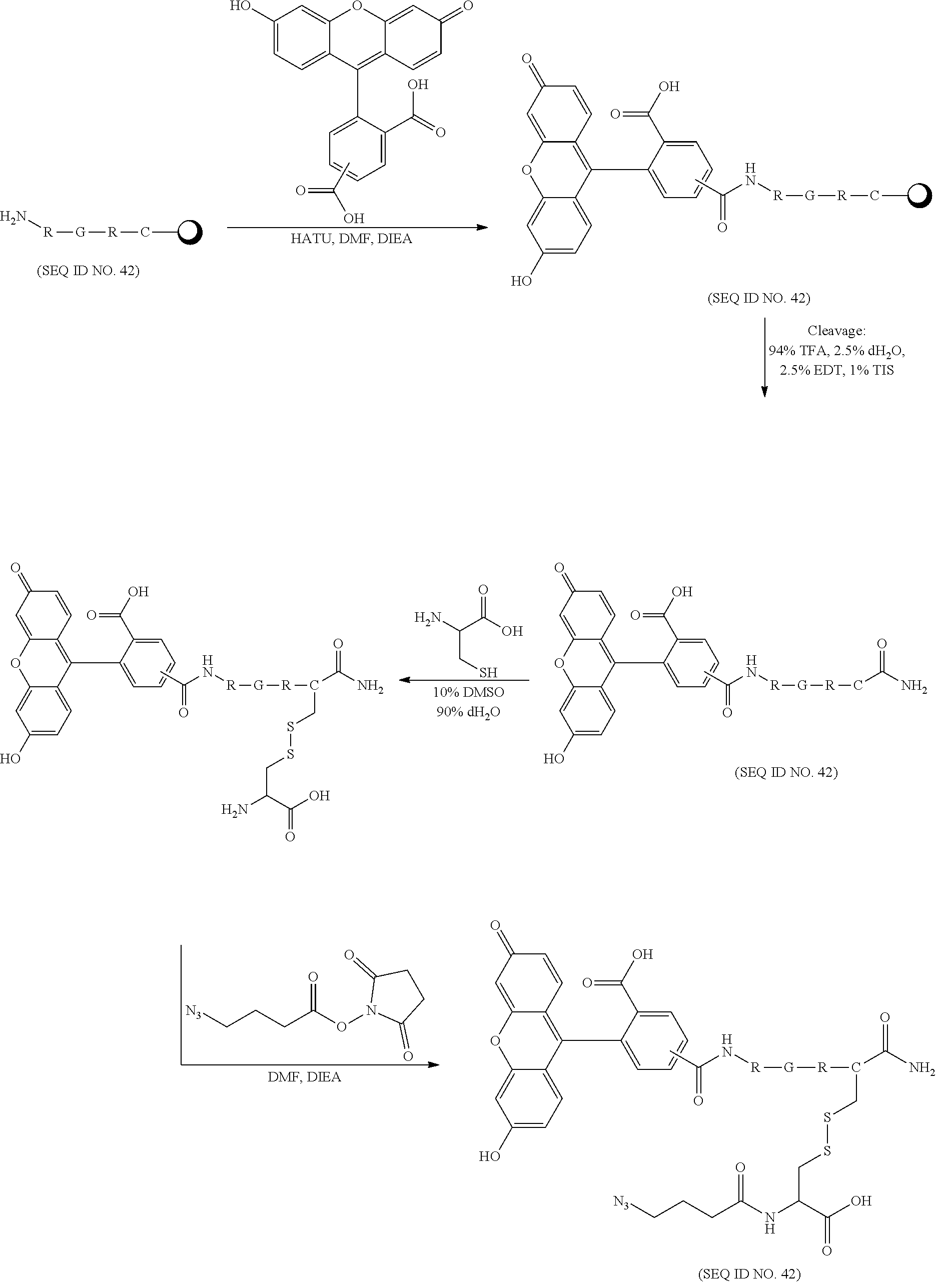

### Synthesis of FAM-RGRC-[S—S]—C—N₃ (SEQ ID NO. 42) Pendant

The base peptide H-RGRC (SEQ ID NO. 42) was synthesized on rink amide resin (ChemPep, 0.8 meg/mg) via standard microwave-assisted Fmoc solid phase methodology and HBTU activation on a scale of 0.25 mmol (CEM Liberty 1). Microwave assisted coupling of 5,6-Carboxyfluorescein (4×, 377 mg, Fisher) was conducted at 60° C. and 25 W for 30 minutes on resin with HATU (3.9×, 371 mg) dissolved in minimal DMF and activated with DIEA (8×, 258.3 mg). Resin was treated with trifluoroacetic acid/1,2-ethanedithiol/water/triisopropylsilane (94:2.5:2.5:1) for 2 hr, and the crude peptide was precipitated in and washed (2×) with ice-cold diethyl ether. The crude peptide was purified using RP-HPLC using a 42 min linear gradient (20-100% of acetonitrile and 0.1% trifluoroacetic acid) and lyophilized to give the intermediate (FAM-RGRC-NH₂) (SEQ ID NO. 42) as a fluffy, yellow solid. Cysteine-OH (605.8 mg, 5 mmol, 20×) was codissolved with intermediate peptide in a dH₂O/DMSO (90:10) solution (50 mL) and rocked for 24 hours. Additional cysteine-OH (605.8 mg, 5 mmol, 20×) was added to solution and allowed to react for 24 hours while rocking at room temperature. Product was vacuum filtered and washed with dH₂O until no color remained in crystals. The filtrate was frozen, lyophilized, and purified using RP-HPLC using a 42 min linear gradient (20-100% of acetonitrile and 0.1% trifluoroacetic acid) and lyophilized to give the intermediate (FAM-RGRC(—S—S—C)—NH₂) (SEQ ID NO. 42) as a fluffy, yellow solid (104.4 mg, 0.10795 mmol). 4-azidobutanoic-OSu (29.32 mg, 0.13 mmol, 1.2×) was coupled overnight with the peptide in minimal DMF and activated with DIEA (55.8 mg, 0.43 mmol, 4×). The peptide was purified using RP-HPLC using a 42 min linear gradient (20-100% of acetonitrile and 0.1% trifluoroacetic acid) and lyophilized to give the product (FAM-RGRC(—S—S—C—CO(CH₂)₃N₃)—NH₂) (SEQ ID NO. 42) as a fluffy, yellow solid (83.2 mg, 0.077 mmol) with a good overall yield (29.6%). Peptide purity was confirmed with analytical RP-HPLC and matrix-assisted laser desorption-ionization time-of-flight mass spectrometry using α-cyano-4-hydroxycinnamic acid matrix: MALDI-TOF: calculated for C₄₅H₅₅N₁₅O₁₃S₂⁺[M+¹H]⁺, 1078.1; found 1079.3.

### Synthesis of Photodegradable Single-Input Pendant

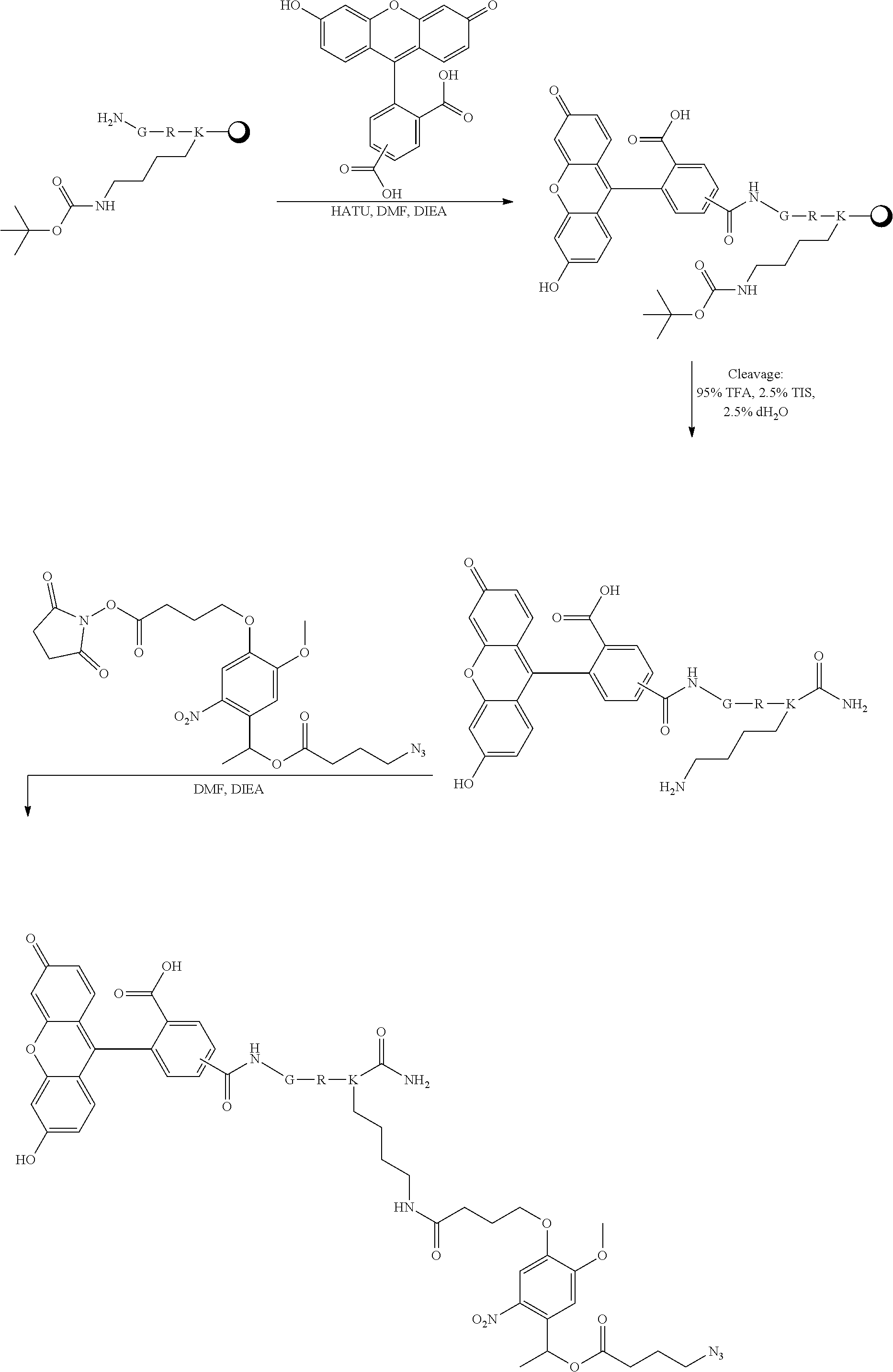

### Synthesis of FAM-RGK(PL-N₃)—NH₂) Pendant

The base peptide H-RGK(boc)-NH₂ was synthesized on rink amide resin (ChemPep, 0.8 meg/mg) via standard microwave-assisted Fmoc solid phase methodology and HBTU activation on a scale of 0.25 mmol (CEM Liberty 1). Microwave assisted coupling of 5,6-Carboxyfluorescein (4×, 377 mg, Fisher) was conducted at 60° C. and 25 W for 30 minutes on resin with HATU (3.9×, 371 mg) dissolved in minimal DMF and activated with DIEA (8×, 258.3 mg). Resin was treated with trifluoroacetic acid/triisopropylsilane/water (95:2.5:2.5) for 2 hr, and the crude peptide was precipitated in and washed (2×) with ice-cold diethyl ether. The crude peptide was purified using RP-HPLC using a 55 min linear gradient (5-100% of acetonitrile and 0.1% trifluoroacetic acid) and lyophilized to give the intermediate (FAM-RGK-NH₂) as a fluffy, yellow solid. OSu-PL-N₃ (53 mg, 0.105 mmol, 1.3×) was dissolved in minimal DMF and activated with DIEA (40 mg, 0.31 mmol, 4×) and added to peptide to react overnight. The peptide was purified using RP-HPLC using a 55 min linear gradient (5-100% of acetonitrile and 0.1% trifluoroacetic acid) and lyophilized to give the product (FAM-RGK(PL-N₃)—NH₂) as a fluffy, yellow solid (11.0 mg, 0.01 mmol) with a yield of 4%. Peptide purity was confirmed with analytical RP-HPLC and matrix-assisted laser desorption-ionization time-of-flight mass spectrometry using α-cyano-4-hydroxycinnamic acid matrix: MALDI-TOF: calculated for C₅₂H₆₀N₁₂O₁₆⁺ [M+¹H]⁺, 1109.1; found 1109.3.

### Synthesis of Reducible-OR-Photolabile Pendant

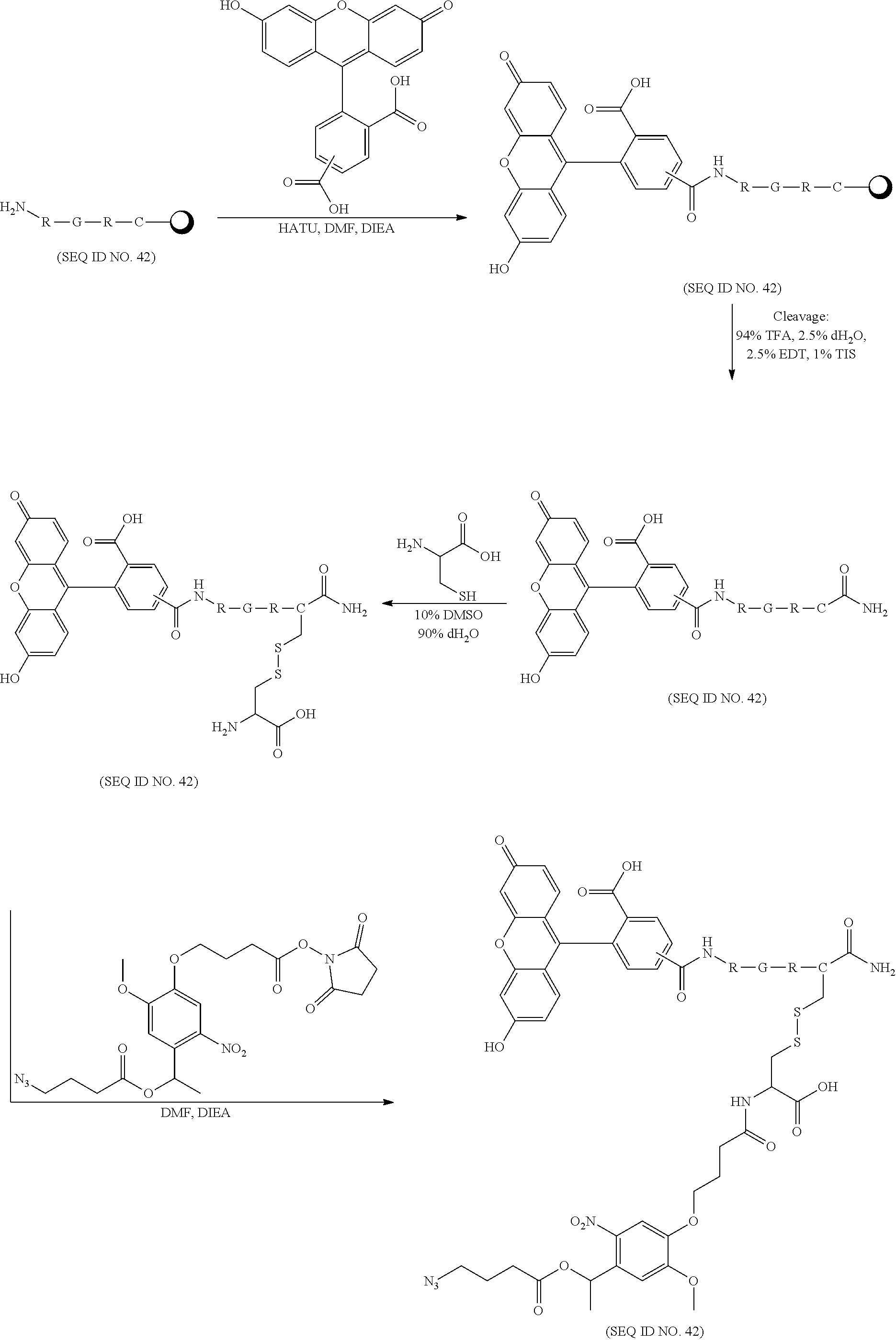

### Synthesis of FAM-RGRC-[S—S]—C-PL-N₃ (SEQ ID NO. 42) Pendant

The base peptide H-RGRC (SEQ ID NO. 42) was synthesized on rink amide resin (ChemPep, 0.8 meg/mg) via standard microwave-assisted Fmoc solid phase methodology and HBTU activation on a scale of 0.25 mmol (CEM Liberty 1). Microwave assisted coupling of 5,6-Carboxyfluorescein (4×, 377 mg, Fisher) was conducted at 60° C. and 25 W for 30 minutes on resin with HATU (3.9×, 371 mg) dissolved in minimal DMF and activated with DIEA (8×, 258.3 mg). Resin was treated with trifluoroacetic acid/1,2-ethanedithiol/water/triisopropylsilane (94:2.5:2.5:1) for 2 hr, and the crude peptide was precipitated in and washed (2×) with ice-cold diethyl ether. The crude peptide was purified using RP-HPLC using a 42 min linear gradient (20-100% of acetonitrile and 0.1% trifluoroacetic acid) and lyophilized to give the intermediate (FAM-RGRC-NH₂) (SEQ ID NO. 42) as a fluffy, yellow solid. Cysteine-OH (605.8 mg, 5 mmol, 20×) was codissolved with intermediate peptide in a dH₂O/DMSO (90:10) solution (50 mL) and rocked for 24 hours. Additional cysteine-OH (605.8 mg, 5 mmol, 20×) was added to solution and allowed to react for 24 hours while rocking at room temperature. Product was vacuum filtered and washed with dH₂O until no color remained in crystals. The filtrate was frozen, lyophilized, and purified using RP-HPLC using a 42 min linear gradient (20-100% of acetonitrile and 0.1% trifluoroacetic acid) and lyophilized to give the intermediate (FAM-RGRC(—S—S—C)—NH₂) (SEQ ID NO. 42) as a fluffy, yellow solid (104.4 mg, 0.10795 mmol). OSu-PL-N₃ (60.5 mg, 0.119 mmol, 1.2×) was dissolved in minimal DMF and activated with DIEA (51.3 mg, 0.40 mmol, 4×) and added to peptide to react overnight. The peptide was purified using RP-HPLC using a 42 min linear gradient (20-100% of acetonitrile and 0.1% trifluoroacetic acid) and lyophilized to give the product (FAM-RGRC(—S—S—C-PL-N₃)—NH₂) (SEQ ID NO. 42) as a fluffy, yellow solid (79.6 mg, 0.054 mmol) with a good overall yield (22.6%). Peptide purity was confirmed with analytical RP-HPLC and matrix-assisted laser desorption-ionization time-of-flight mass spectrometry using α-cyano-4-hydroxycinnamic acid/2,5-dihydroxybenzoic acid (2:1) matrix: MALDI-TOF: calculated for C₅₈H₇₀N₁₆O₁₉S₂⁺ [M+¹H]⁺, 1359.4; found 1359.45.

### Synthesis of MMP-8-OR-Photolabile Pendant

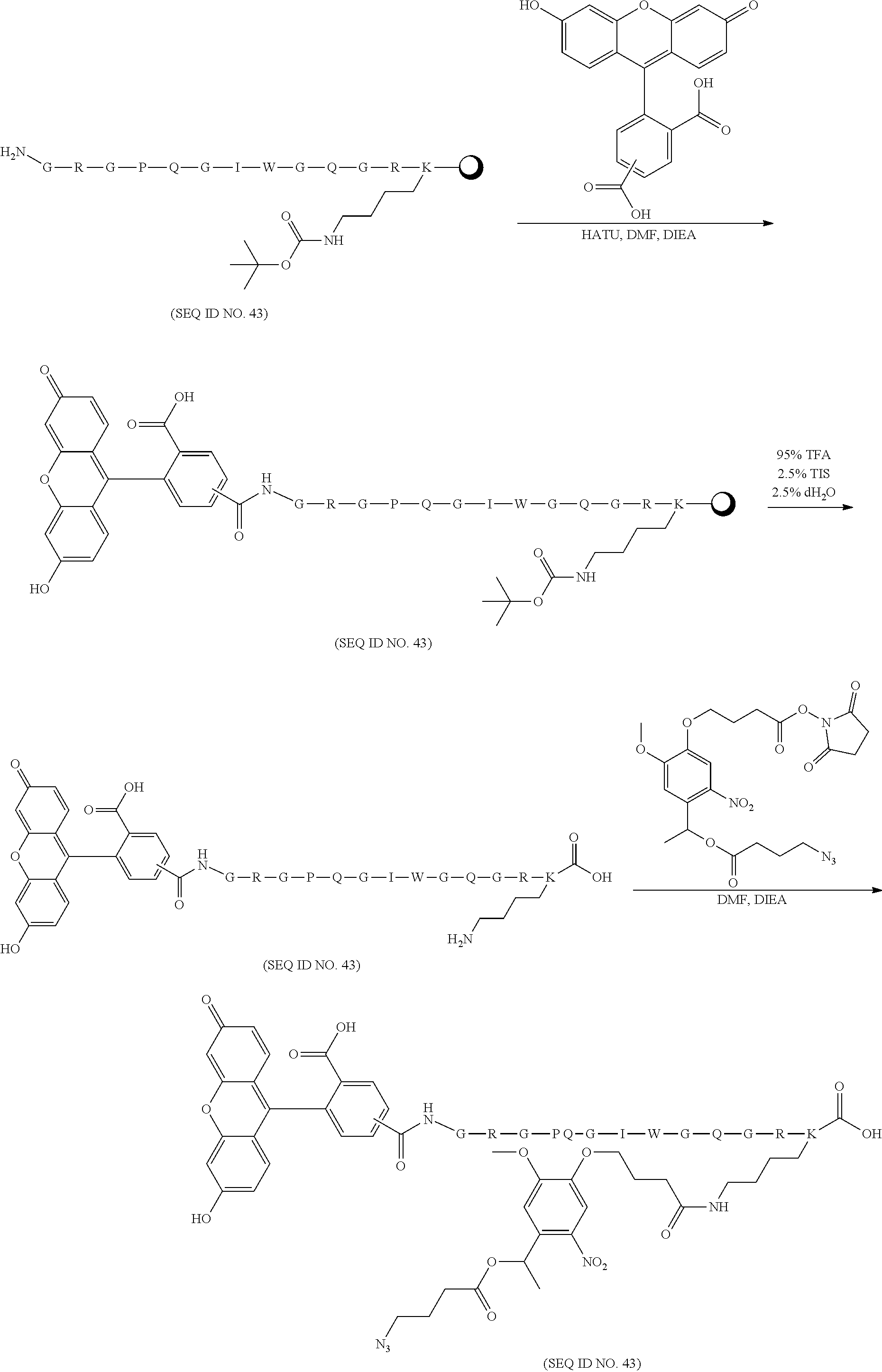

### Synthesis of FAM-GRGPQGIWGQGRK(PL-N₃) NH₂) (SEQ ID NO. 43) Pendant

The base peptide H-GRGPQGIWGQGRK (SEQ ID NO. 43) was synthesized on rink amide resin (ChemPep, 0.8 meg/mg) via standard microwave-assisted Fmoc solid phase methodology and HBTU activation on a scale of 0.25 mmol (CEM Liberty 1). Microwave assisted coupling of 5,6-Carboxyfluorescein (4×, 377 mg, Fisher) was conducted at 60° C. and 25 W for 30 minutes on resin with HATU (3.9×, 371 mg) dissolved in minimal DMF and activated with DIEA (8×, 258.3 mg). Resin was treated with trifluoroacetic acid/triisopropylsilane/water (95:2.5:2.5) for 2 hr, and the crude peptide was precipitated in and washed (2×) with ice-cold diethyl ether. The crude peptide was purified using RP-HPLC using a 55 min linear gradient (5-100% of acetonitrile and 0.1% trifluoroacetic acid) and lyophilized to give the intermediate (FAM-GRGPQGIWGQGRK-NH₂) (SEQ ID NO. 43) as a fluffy, yellow solid. OSu-PL-N₃ (18 mg, 0.036 mmol, 1.3×) was dissolved in minimal DMF and activated with DIEA (10.34 mg, 0.08 mmol, 4×) and added to peptide to react overnight. The peptide was purified using RP-HPLC using a 42 min linear gradient (20-100% of acetonitrile and 0.1% trifluoroacetic acid) and lyophilized to give the product FAM-GRGPQGIWGQGRK(PL-N₃)) (SEQ ID NO. 43) as a fluffy, yellow solid (10.3 mg, 4.8 μmol), an overall yield of 1.9%. Peptide purity was confirmed with analytical RP-HPLC and matrix-assisted laser desorption-ionization time-of-flight mass spectrometry using α-cyano-4-hydroxycinnamic acid/2,5-dihydroxybenzoic acid (2:1) matrix: MALDI-TOF: calculated for C₉₈H₁₂₈N₂₈O₂₈⁺ [M+¹H]⁺, 2146.2; found 2146.7.

### Synthesis of MMP-8-OR-Reducible Pendant

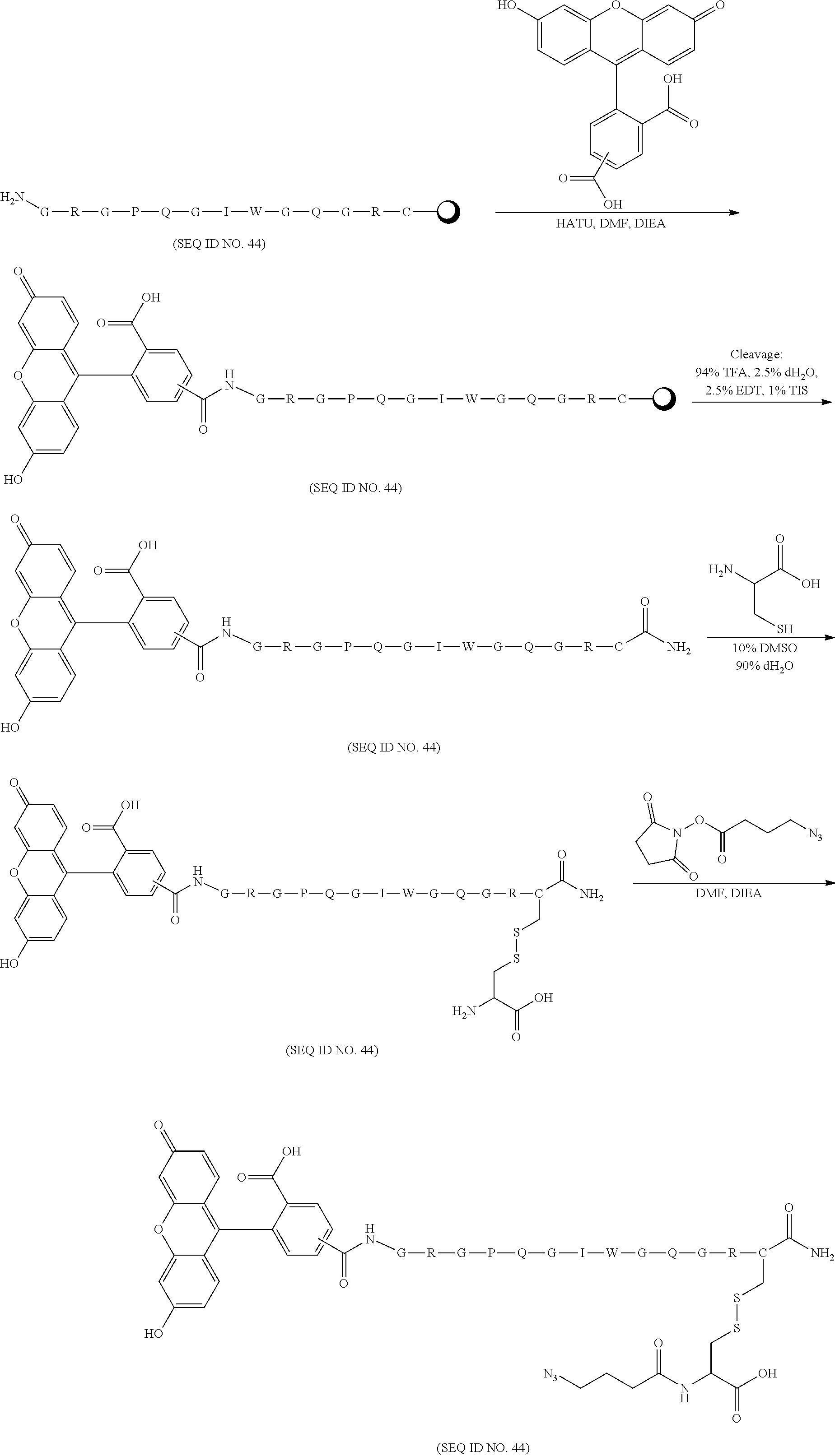

### Synthesis of FAM-GRGPQGIWGQGRC(—S—S—C—N₃)—NH₂) (SEQ ID NO. 44) Pendant

The base peptide H-GRGPQGIWGQGRC (SEQ ID NO. 44) was synthesized on rink amide resin (ChemPep, 0.8 meg/mg) via standard microwave-assisted Fmoc solid phase methodology and HBTU activation on a scale of 0.50 mmol (CEM Liberty 1). Microwave assisted coupling of 5,6-Carboxyfluorescein (4×, 377 mg, Fisher) was conducted stirring at room temperature overnight on resin with HATU (3.9×, 371 mg) dissolved in minimal DMF and activated with DIEA (8×, 258.3 mg). This coupling was performed a second time until a Ninhydrin Colorimetric Assay gave a negative result. Resin was treated with trifluoroacetic acid/1,2-ethanedithiol/water/ triisopropylsilane (94:2.5:2.5:1) for 2 hr, and the crude peptide was precipitated in and washed (2×) with ice-cold diethyl ether. The crude peptide was purified using RP-HPLC using a 55 min linear gradient (5-100% of acetonitrile and 0.1% trifluoroacetic acid) and lyophilized to give the intermediate (FAM-GRGPQGIWGQGRC-NH₂) (SEQ ID NO. 44) as a fluffy, yellow solid. Cysteine-OH (605.8 mg, 5 mmol, 20×) was codissolved with intermediate peptide in a dH₂O/DMSO (90:10) solution (50 mL) and rocked for 24 hours. Product was vacuum filtered and washed with dH₂O until no color remained in crystals. The filtrate was frozen, lyophilized, and purified using RP-HPLC using a 42 min linear gradient (20-100% of acetonitrile and 0.1% trifluoroacetic acid) and lyophilized to give the intermediate (FAM-GRGPQGIWGQGRC(—S—S—C)—NH₂) (SEQ ID NO. 44) as a fluffy, yellow solid. 4-azidobutanoic-OSu (13.6 mg, 0.06 mmol, 2×) was coupled overnight with the peptide in minimal DMF and activated with DIEA (15.5 mg, 0.12 mmol, 4×). The peptide was purified using RP-HPLC using a 42 min linear gradient (20-100% of acetonitrile and 0.1% trifluoroacetic acid) and lyophilized to give the product (FAM-GRGPQGIWGQGRC(—S—S—C—CO(CH₂)₃N₃)—NH₂) (SEQ ID NO. 44) as a fluffy, yellow solid (21.7 mg, 11.1 μmol) with an overall yield of 4.4%. Peptide purity was confirmed with analytical RP-HPLC and matrix-assisted laser desorption-ionization time-of-flight mass spectrometry using α-cyano-4-hydroxycinnamic acid/2,5-dihydroxybenzoic acid (2:1) matrix: MALDI-TOF: calculated for C₈₅H₁₁₁N₂₇O₂₄S₂⁺ [M+¹H]⁺, 1959.1; found 1959.5.

### Synthesis of MMP-8-OR-Reducible-OR-Photolabile Pendant

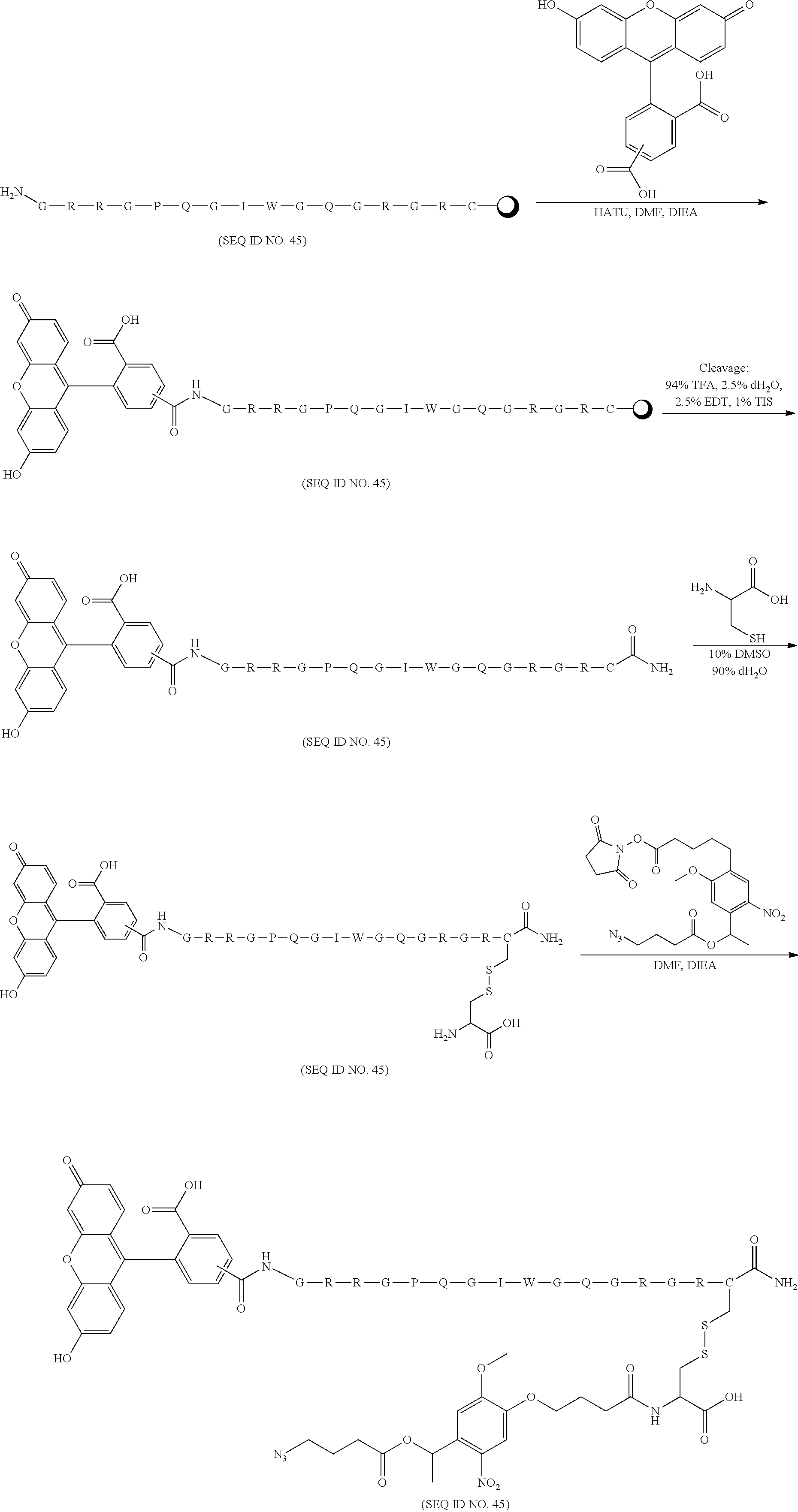

### Synthesis of FAM-GRRGPQGIWGQGRGRC(—S—S—C-PL-N₃)—NH₂) (SEQ ID NO. 45) Pendant

The base peptide H-GRRGPQGIWGQGRGRC (SEQ ID NO. 45) was synthesized on rink amide resin (ChemPep, 0.8 meg/mg) via standard microwave-assisted Fmoc solid phase methodology and HBTU activation on a scale of 0.50 mmol (CEM Liberty 1). Microwave assisted coupling of 5,6-Carboxyfluorescein (4×, 377 mg, Fisher) was conducted at 60° C. and 25 W for 30 minutes on resin with HATU (3.9×, 371 mg) dissolved in minimal DMF and activated with DIEA (8×, 258.3 mg). Resin was treated with trifluoroacetic acid/1,2-ethanedithiol/water/triisopropylsilane (94:2.5:2.5:1) for 2 hr, and the crude peptide was precipitated in and washed (2×) with ice-cold diethyl ether. The crude peptide was purified using RP-HPLC using a 55 min linear gradient (5-100% of acetonitrile and 0.1% trifluoroacetic acid) and lyophilized to give the intermediate (FAM-GRRGPQGIWGQGRC-NH₂) (SEQ ID NO. 45) as a fluffy, yellow solid. Cysteine-OH (605.8 mg, 5 mmol, 20×) was codissolved with intermediate peptide in a dH₂O/DMSO (90:10) solution (50 mL) and rocked for 24 hours. Product was vacuum filtered and washed with dH₂O until no color remained in crystals. The filtrate was frozen, lyophilized, and purified using RP-HPLC using a 42 min linear gradient (20-100% of acetonitrile and 0.1% trifluoroacetic acid) and lyophilized to give the intermediate (FAM-GRRGPQGIWGQGRC(—S—S—C)—NH₂) (SEQ ID NO. 45) as a fluffy, yellow solid. OSu-PL-N₃ (28.4 mg, 0.056 mmol, 1.3×) was dissolved in minimal DMF and activated with DIEA (22.2 mg, 0.172 mmol, 4×) and added to peptide to react overnight. The peptide was purified using RP-HPLC using a 42 min linear gradient (20-100% of acetonitrile and 0.1% trifluoroacetic acid) and lyophilized to give the product (FAM-GRRGPQGIWGQGRGRC(—S—S—C-PL-N₃)—NH₂) (SEQ ID NO. 45) as a fluffy, yellow solid (60.0 mg, 23 μmol) with an overall yield of 9.2%. Peptide purity was confirmed with analytical RP-HPLC and matrix-assisted laser desorption-ionization time-of-flight mass spectrometry using α-cyano-4-hydroxycinnamic acid/2,5-dihydroxybenzoic acid (2:1) matrix: MALDI-TOF: calculated for C₁₁₂H₁₅₃N₃₇O₃₃S₂⁺ [M+¹H]⁺, 2609.8; found 2609.9.

### Synthesis of MMP-8-AND-Reducible Pendant

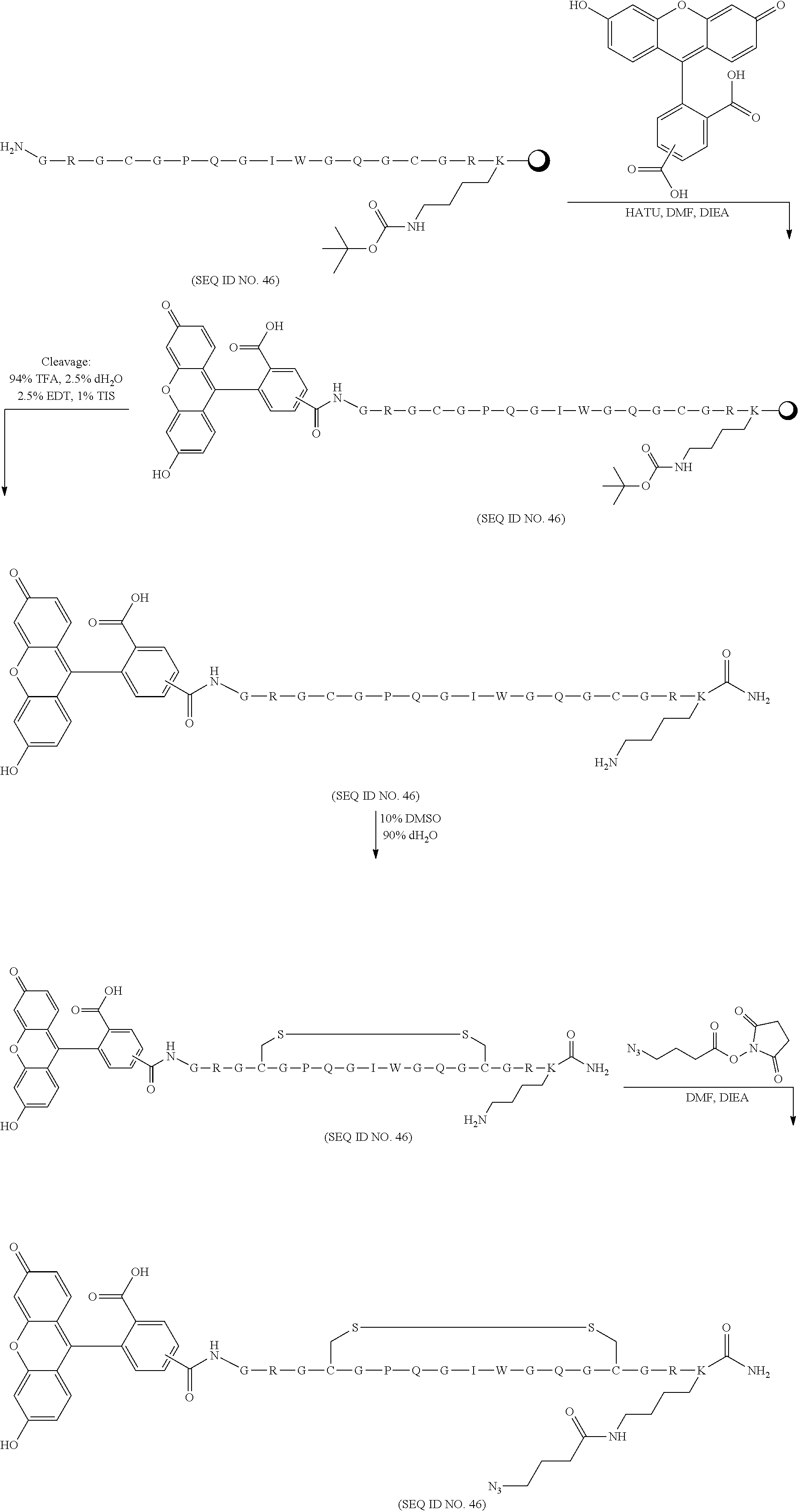

### Synthesis of FAM-GRGCGPQGIWGQGCGRK(N₃)—NH₂ Cyclized) (SEQ ID NO. 46) Pendant

The base peptide H-GRGCGPQGIWGQGCGRK (SEQ ID NO. 46) was synthesized on rink amide resin (ChemPep, 0.8 meg/mg) via standard microwave-assisted Fmoc solid phase methodology and HBTU activation on a scale of 0.25 mmol (CEM Liberty 1). Microwave assisted coupling of 5,6-Carboxyfluorescein (282.2 mg, 0.75 mmol, 4× Fisher) was conducted at 60° C. and 25 W for 30 minutes on resin with HATU (280.9 mg, 0.73 mmol, 2.95×) dissolved in minimal DMF and activated with DIEA (193.9 mg, 1.5 mmol, 6×). Resin was treated with trifluoroacetic acid/1,2-ethanedithiol/water/triisopropylsilane (94:2.5:2.5:1) for 2 hr, and the crude peptide was precipitated in and washed (2×) with ice-cold diethyl ether. The crude peptide was purified using RP-HPLC using a 55 min linear gradient (5-100% of acetonitrile and 0.1% trifluoroacetic acid) and lyophilized to give the intermediate (FAM-GRGPQGIWGQGRK-NH₂) (SEQ ID NO. 46) as a fluffy, yellow solid. The purified peptide (<0.5 nM) was dissolved in a dH₂O/DMSO (90:10) solution (50 mL) and rocked for 24 hours. Product was concentrated, lyophilized and subsequently purified using RP-HPLC using a 55 min linear gradient (5-100% of acetonitrile and 0.1% trifluoroacetic acid) and lyophilized to give the intermediate (FAM-GRGCGPQGIWGQGCGRK-NH₂(SEQ ID NO. 46) cyclized via cysteine-cysteine disulfide bridge) as a fluffy, yellow solid. 4-Azidobutanoic-NHS (2.65 mg, 0.012 mmol, 1.2×) was coupled overnight with the peptide in minimal DMF and activated with DIEA (4.65 mg, 0.04 mmol, 4×). The peptide was purified using RP-HPLC using a 55 min linear gradient (5-100% of acetonitrile and 0.1% trifluoroacetic acid) and lyophilized to give the product (FAM-GRGCGPQGIWGQGCGRK(CO(CH₂)₃N₃)—NH₂ (SEQ ID NO. 46) cyclized via cysteine disulfide bridge) as a fluffy, yellow solid (5.7 mg, 2.6 μmol) with an overall yield of 1.04%. Peptide purity was confirmed with analytical RP-HPLC and matrix-assisted laser desorption-ionization time-of-flight mass spectrometry using α-cyano-4-hydroxycinnamic acid matrix: MALDI-TOF: calculated for C₉₅H₁₂₇N₃₁O₃₃S₂⁺ [M+¹H]⁺, 2183.3; found 2183.2.

### Synthesis of Photolabile-AND-Reducible Pendant

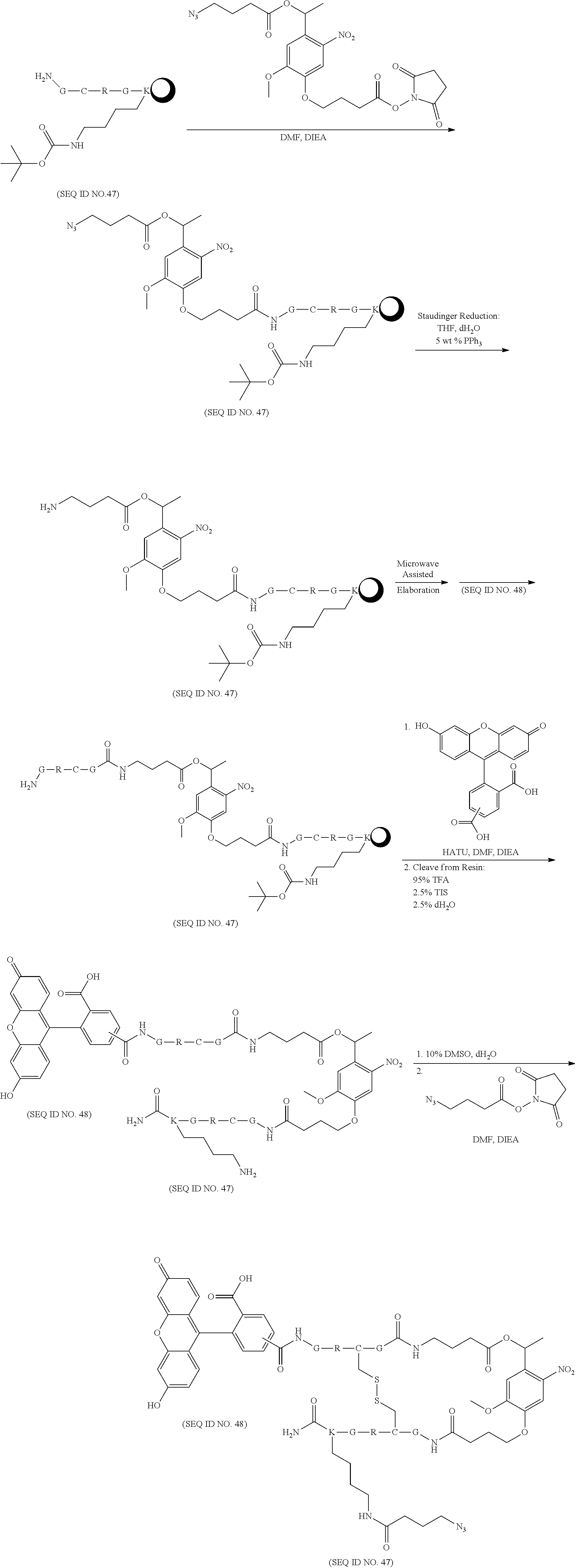

### Synthesis of FAM-GRCG-PL-GCRGK(CO(CH₂)₃N₃)—NH₂ Cyclized) (SEQ ID NO. 47) Pendant

The base peptide H-GCRGK (SEQ ID NO. 47) was synthesized on rink amide resin (ChemPep, 0.8 meg/mg) via standard microwave-assisted Fmoc solid phase methodology and HBTU activation on a scale of 0.25 mmol (CEM Liberty 1). OSu-PL-N3 (329.55 mg, 0.325 mmol, 1.3×) was dissolved in minimal DMF and activated with DIEA (129.2 mg, 1.0 mmol, 4×) and added to peptide on resin to react overnight at room temperature. The azide on the PL was reduced by performing a Staudinger reduction where the resin was first rinsed with a solution of tetrahydrofuran/dH₂O (90:10, 3×20 mL). A solution of 5 wt % triphenylphosphine (1.5 g, Sigma) in tetrahydrafuran/dH₂O (90:10, 30 mL) was added and allowed to react overnight. Peptide was rinsed with DMF (3×10 mL) and DCM (3×10 mL), and standard microwave-assisted Fmoc solid phase methodology and HBTU activation was used to elaborate the peptide to form the peptide H-GRCG-PL-GCRGK-NH₂ (SEQ ID NO. 48) connected to (SEQ ID NO. 47) through PL. 5,6-Carboxyfluorescein (377 mg, 1.0 mmol, 4× Fisher) was conducted at room temperature overnight on resin with HATU (371 mg, 0.98 mmol, 3.9×) dissolved in minimal DMF and activated with DIEA (8×, 258.3 mg). Resin was treated with trifluoroacetic acid/water/triisopropylsilane (95:2.5:2.5) for 2 hr, and the crude peptide was precipitated in and washed (2×) with ice-cold diethyl ether. The crude peptide was purified using RP-HPLC using a 42 min linear gradient (20-100% of acetonitrile and 0.1% trifluoroacetic acid) and lyophilized to give the intermediate (FAM-GRCG-PL-GCRGK-NH₂) (SEQ ID NO. 48) connected to (SEQ ID NO. 47) through PL as a fluffy, yellow solid. The purified peptide (<0.5 mM) was dissolved in a dH₂O/DMSO (90:10) solution (35 mL) and rocked for 24 hours, then frozen and lyophilized. Intermediate product was purified using RP-HPLC using a 42 min linear gradient (20-100% of acetonitrile and 0.1% trifluoroacetic acid) and lyophilized to give the intermediate (FAM-GRCG-PL-GCRGK-NH₂) (SEQ ID NO. 48) connected to (SEQ ID NO. 47) through PL cyclized via cysteine-cysteine disulfide bridge) as a fluffy, yellow solid. 4-Azidobutanoic-NHS (1.2×) was coupled overnight with the peptide in minimal DMF and activated with DIEA (4×). The peptide was purified using RP-HPLC using a 42 min linear gradient (20-100% of acetonitrile and 0.1% trifluoroacetic acid) and lyophilized to give the product (FAM-GRCG-PL-GCGRK(CO(CH₂)₃N₃)—NH₂) (SEQ ID NO. 48) connected to (SEQ ID NO. 47) through PL, cyclized via cysteine-cysteine disulfide bridge) as a fluffy, yellow solid (5.7 mg, 2.6 μmol) with an overall yield of 1.04%. Peptide purity was confirmed with analytical RP-HPLC and matrix-assisted laser desorption-ionization time-of-flight mass spectrometry using α-cyano-4-hydroxycinnamic acid matrix: MALDI-TOF: calculated for C₇₄H₉₆N₂₂O₂₃S₂⁺ [M+¹H]⁺, 1725.8.

### Synthesis of MMP-8-AND-Photolabile Pendant

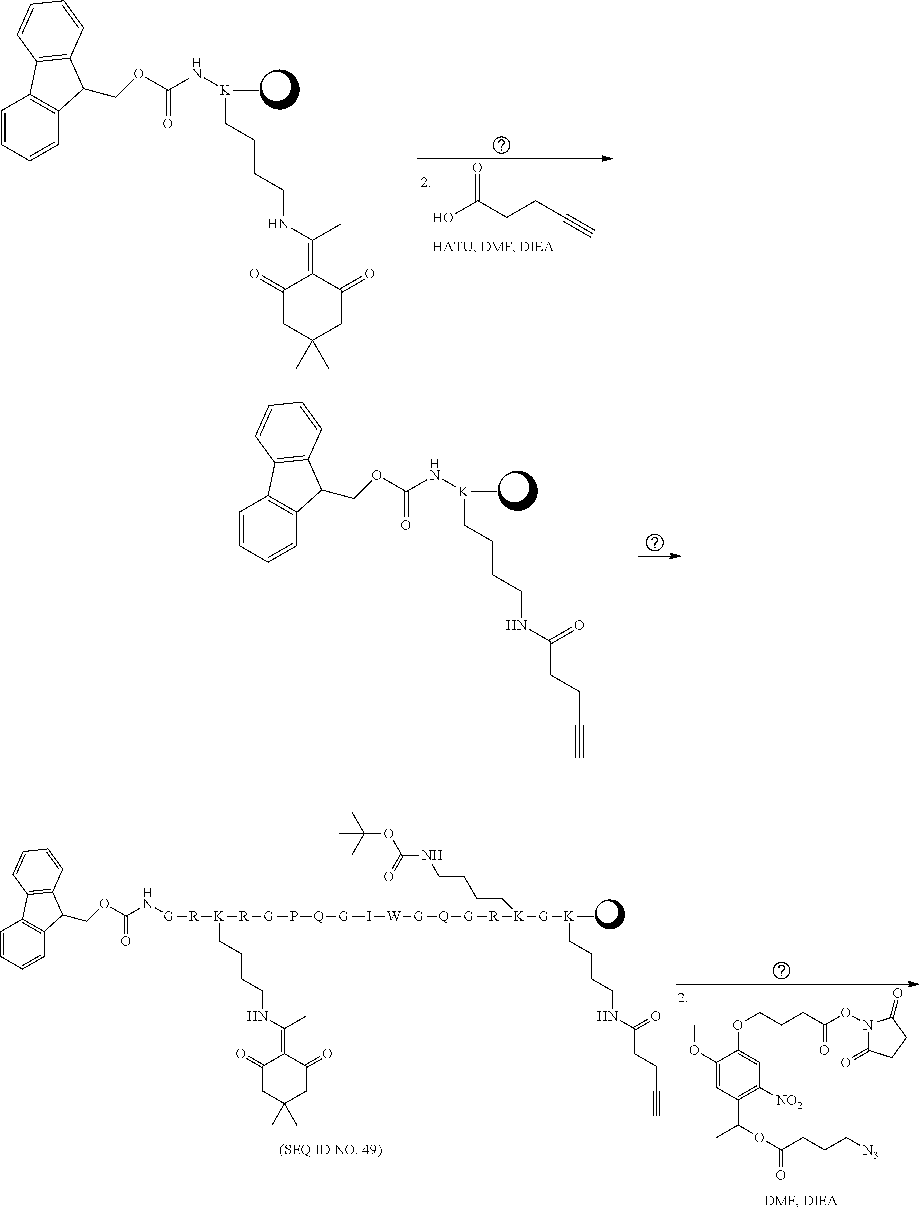

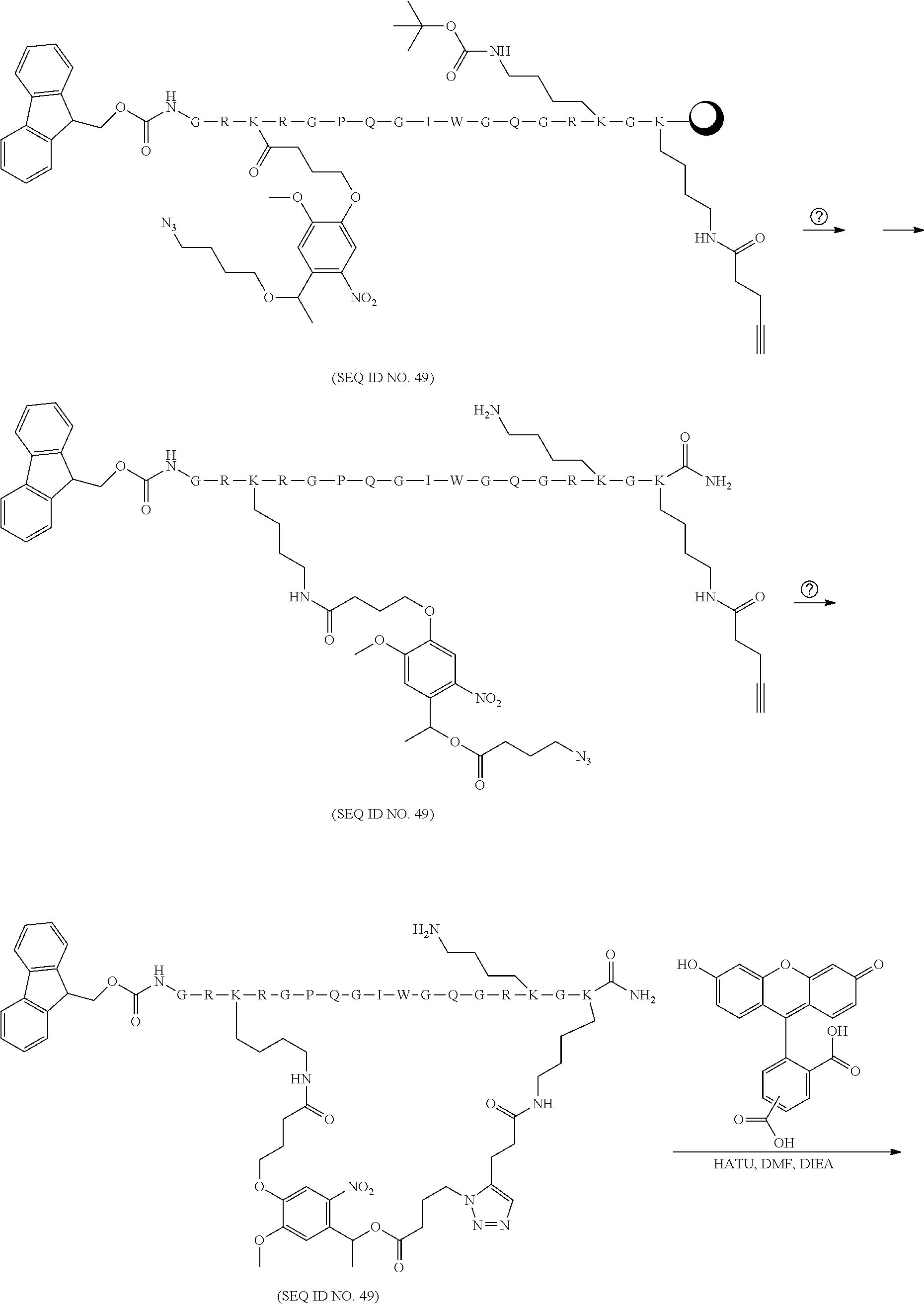

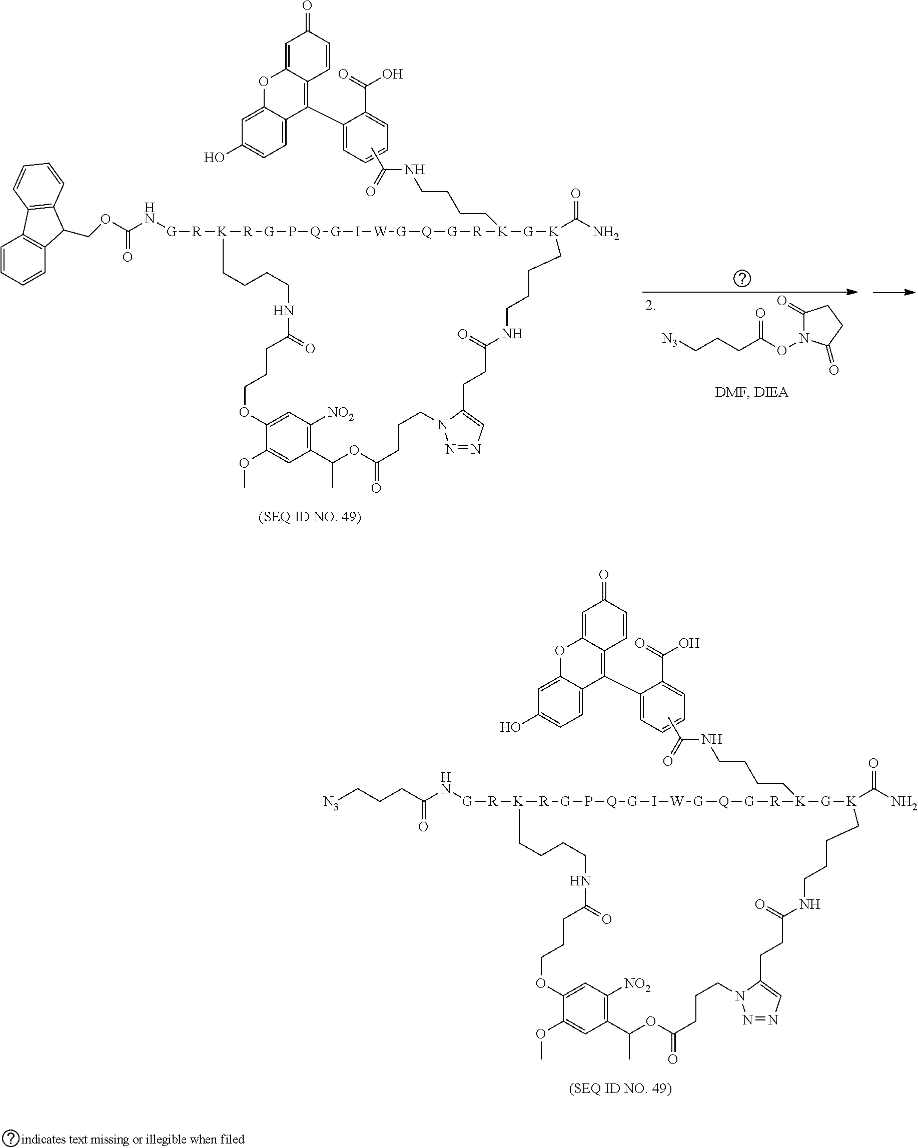

### Synthesis of N₃-GRK(PL)RGPQGIWGQGRK(FAM)GK(pentynoic acid)-NH₂ Cyclized) (SEQ ID NO. 49) Pendant

The first amino acid (Fmoc-K—NH₂) was synthesized on rink amide resin (ChemPep, 0.8 meg/mg) via standard microwave-assisted Fmoc solid phase methodology and HBTU activation on a scale of 0.25 mmol (CEM Liberty 1). The 1-(4,4-dimethyl-2,6-dioxacyclohexylidene)ethyl (dde) group was removed with 1 eq hydrazine HCl (17.4 mg, 0.25 mmol) and 1 eq of imidazole (17.0 mg, 0.25 mmol) in N-methyl-2-pyrrolidone (NMP, 5 mL) and DCM (1 mL), stirred for 3 hours at room temperature, and 4-Pentynoic acid (98.1 mg, 1.0 mmol, 4×) was coupled overnight at room temperature on resin with HATU (371 mg, 0.98 mmol, 3.9×) dissolved in minimal DMF and activated with DIEA (258.3 mg, 2 mmol, 8×). The resin was further elaborated to develop the base peptide (Fmoc-GRK(dde)RGPQGIWGQGRK(boc)GK(pentynoic acid)-NH₂) (SEQ ID NO. 49) via standard microwave-assisted Fmoc solid phase methodology and HBTU activation (CEM Liberty 1). The 1-(4,4-dimethyl-2,6-dioxacyclohexylidene)ethyl (dde) group was removed with 1 eq hydrazine HCl (17.4 mg, 0.25 mmol) and 1 eq of imidazole (17.0 mg, 0.25 mmol) in N-methyl-2-pyrrolidone (NMP, 5 mL) and DCM (1 mL), stirred for 3 hours at room temperature, and OSu-PL-N₃ (165.0 mg, 0.325 mmol, 1.3×) was dissolved in minimal DMF and activated with DIEA (129.4 mg, 1.0 mmol, 4×) and added to peptide to react overnight.

Resin was treated with trifluoroacetic acid/water/triisopropylsilane (95:2.5:2.5) for 2 hr, and the crude peptide was precipitated in and washed (2×) with ice-cold diethyl ether. The crude peptide was purified using RP-HPLC using a 42 min linear gradient (20-100% of acetonitrile and 0.1% trifluoroacetic acid) and lyophilized to give the intermediate (Fmoc-GRK(PL-N₃)RGPQGIWGQGRK(NH₂)GK(pentynoic acid)-NH₂) (SEQ ID NO. 49) as a fluffy, orange solid. Copper-assisted Azide-Alkyne Huisgen Cycloaddition was performed by drying anhydrous DMSO (9.29 mL) with nitrogen for 30 mins, dissolving Cu(I)Br (1.33 mg, 0.009 mmol), then adding the peptide intermediate (0.009 mmol, 1 mM), 1 eq sodium ascorbate (1.85 mg, 0.009 mmol), 10 eq of 2,6-lutidine (10.0 g, 0.093 mmol), 10 eq DIEA (12.0 mg, 0.093 mmol), stirred at room temperature, protected from light and under a nitrogen blanket overnight. Crude product was concentrated, dissolved in dH₂O and the Copper was removed via passage over a M4195 Copper-ion exchange resin (6 g, DOMEX), then frozen, lyophilized. This was purified using RP-HPLC using a 42 min linear gradient (20-100% of acetonitrile and 0.1% trifluoroacetic acid) and lyophilized to give the cyclized intermediate.

(Fmoc-GRK(PL-cyclized)RGPQGIWGQGRK(NH₂)GK(pentynoic acid)-NH₂) (SEQ ID NO. 49) as a fluffy, red solid. 5,6-Carboxyfluorescein (10.6 mg, 6×) was conducted in solution at room temperature with HATU (10.45 mg, 0.027 mmol, 5.85×) dissolved in minimal DMF and activated with DIEA (6.07 mg, 0.047 mmol, 10×). The peptide was purified using RP-HPLC using a 55 min linear gradient (5-100% of acetonitrile and 0.1% trifluoroacetic acid) and lyophilized to give the intermediate product (Fmoc-GRK(PL-cyclized)RGPQGIWGQGRK(FAM)GK(pentynoic acid)-NH₂) (SEQ ID NO. 49). The fluorenylmethyloxycarbonyl (Fmoc) protecting group was deprotected in a 20% by volume solution of piperidine/DMF (5 mL), then precipitated in and washed (1×) with ice-cold diethyl ether. 4-Azidobutanoic-OSu (1.1 mg, 0.0048 mmol) was dissolved in DMF (5 mL), activated with DIEA (1.92 mg, 0.0148 mmol), the peptide was then dissolved and allowed to react overnight. The peptide was purified using RP-HPLC using a 55 min linear gradient (5-100% of acetonitrile and 0.1% trifluoroacetic acid) and lyophilized to give the intermediate product (N₃-GRK(PL-cyclized)RGPQGIWGQGRK(FAM)GK(pentynoic acid)-NH₂) (SEQ ID NO. 49) as a fluffy, orange solid. Peptide purity was confirmed with analytical RP-HPLC and matrix-assisted laser desorption-ionization time-of-flight mass spectrometry using α-cyano-4-hydroxycinnamic acid matrix: MALDI-TOF: calculated for C₁₂₇H₁₇₆N₃₈O₃₄⁺ [M+¹H], 2779.0.

### Synthesis of MMP-8-AND-Reducible-OR-Photolabile Pendant

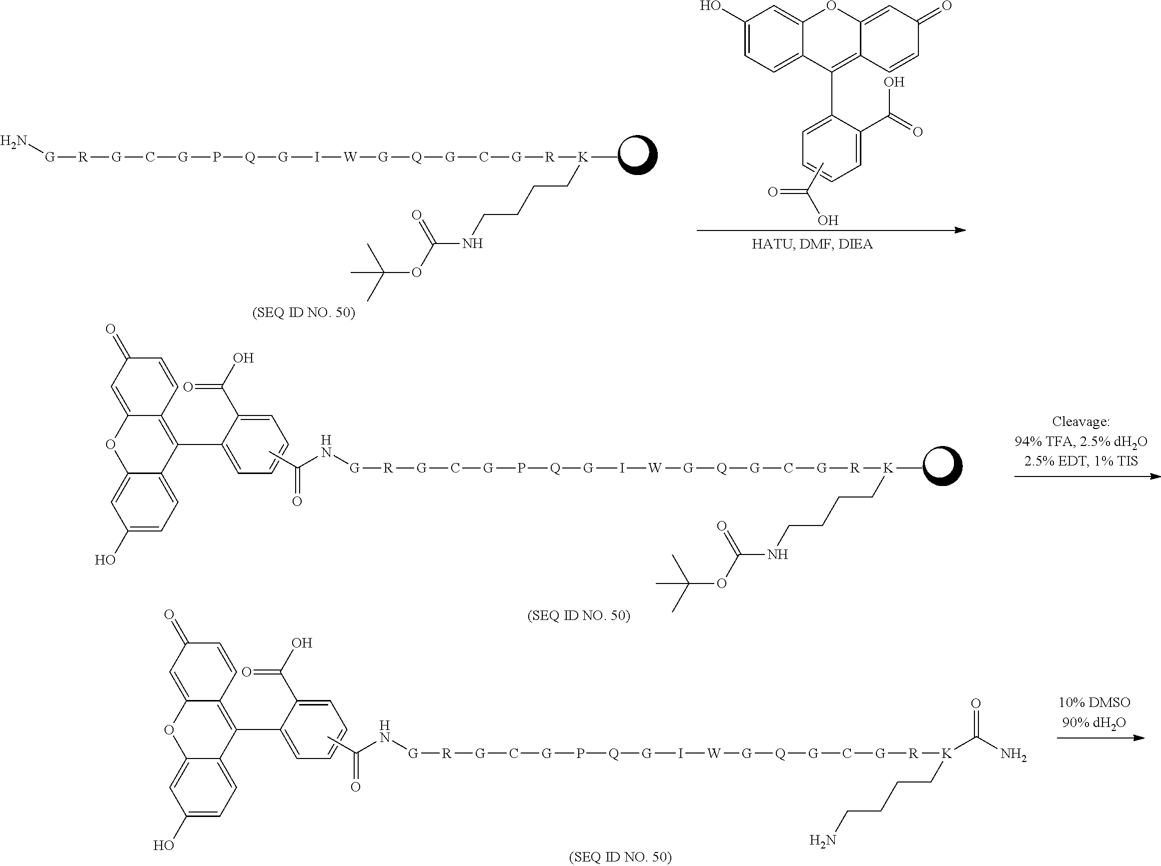

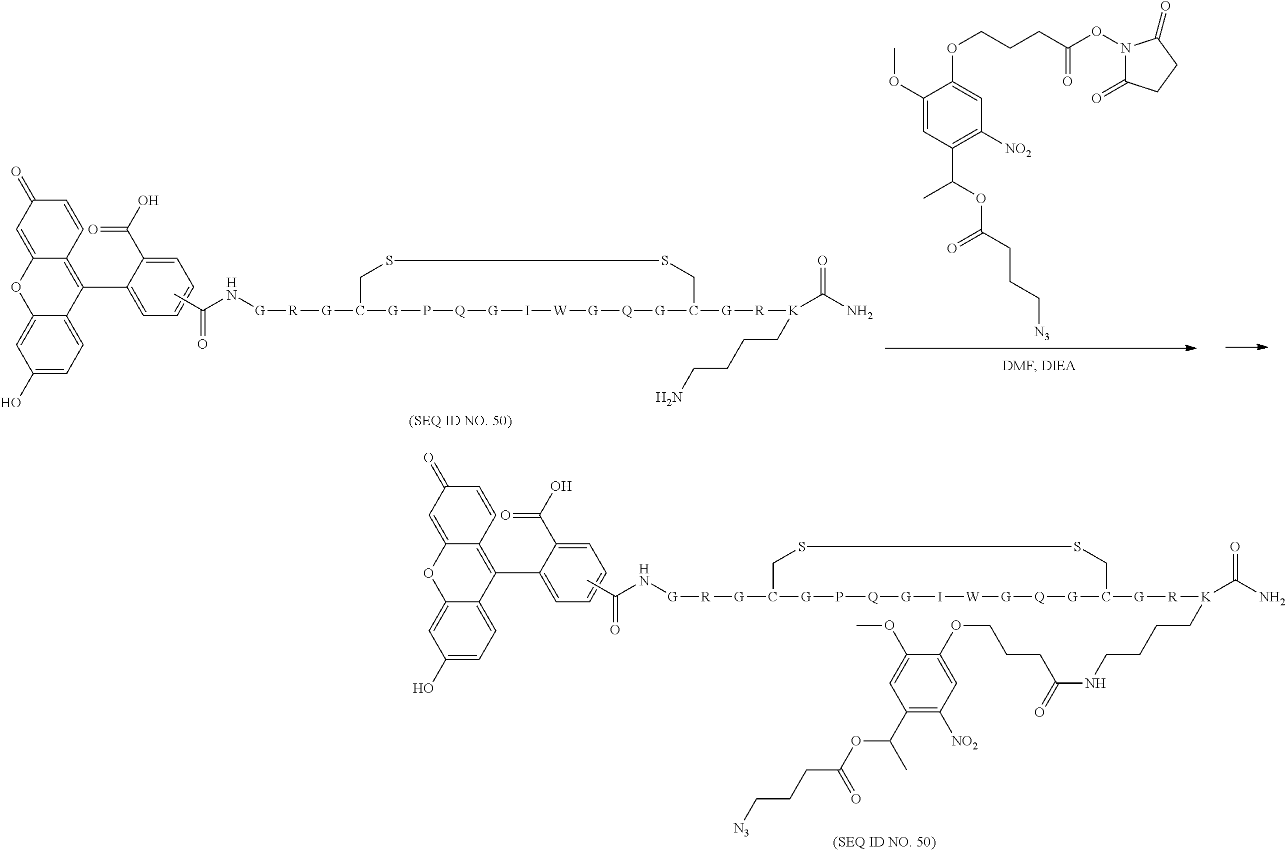

### Synthesis of FAM-GRGCGPOGIWGQGCGRK(PL-N₃)—NH₂ Cyclized) (SEQ ID NO. 50) Pendant

The base peptide H-GRGCGPQGIWGQGCGRK (SEQ ID NO. 50) was synthesized on rink amide resin (ChemPep, 0.8 meg/mg) via standard microwave-assisted Fmoc solid phase methodology and HBTU activation on a scale of 0.25 mmol (CEM Liberty 1). Microwave assisted coupling of 5,6-Carboxyfluorescein (4×, 377 mg, Fisher) was conducted at 60° C. and 25 W for 30 minutes on resin with HATU (3.9×, 371 mg) dissolved in minimal DMF and activated with DIEA (8×, 258.3 mg). Resin was treated with trifluoroacetic acid/1,2-ethanedithiol/water/triisopropylsilane (94:2.5:2.5:1) for 2 hr, and the crude peptide was precipitated in and washed (2×) with ice-cold diethyl ether. The crude peptide was purified using RP-HPLC using a 55 min linear gradient (5-100% of acetonitrile and 0.1% trifluoroacetic acid) and lyophilized to give the intermediate FAM-GRGCGPQGIWGQGCGRK-NH₂ (SEQ ID NO. 50) as a fluffy, yellow solid. The purified peptide (<0.5 mM) was dissolved in a dH₂O/DMSO (90:10) solution (50 mL) and rocked for 24 hours. Product was concentrated, lyophilized and subsequently purified using RP-HPLC using a 55 min linear gradient (5-100% of acetonitrile and 0.1% trifluoroacetic acid) and lyophilized to give the intermediate (FAM-GRGCGPQGIWGQGCGRK-NH₂(SEQ ID NO. 50) cyclized via cysteine-cysteine disulfide bridge) as a fluffy, yellow solid. OSu-PL-N₃ (30.35 mg, 0.06 mmol, 1.3×) was dissolved in minimal DMF and activated with DIEA (23.8 mg, 0.184 mmol, 4×) and added to peptide to react overnight. The peptide was purified using RP-HPLC using a 55 min linear gradient (5-100% of acetonitrile and 0.1% trifluoroacetic acid) and lyophilized to give the product (FAM-GRGCGPQGIWGQGCGRK(PL-N₃)—NH₂ (SEQ ID NO. 50) cyclized via cysteine disulfide bridge) as a fluffy, yellow solid (5.7 mg, 2.6 μmol) with an overall yield of 1.04%. Peptide purity was confirmed with analytical RP-HPLC and matrix-assisted laser desorption-ionization time-of-flight mass spectrometry using α-cyano-4-hydroxycinnamic acid matrix: MALDI-TOF: calculated for C₁₀₈H₁₄₂N₃₂O₃₂S₂⁺ [M+¹H]⁺, 2464.6; found 2465.0.

### Synthesis of Doxorubicin Coupled Logic-Releasable Pendant

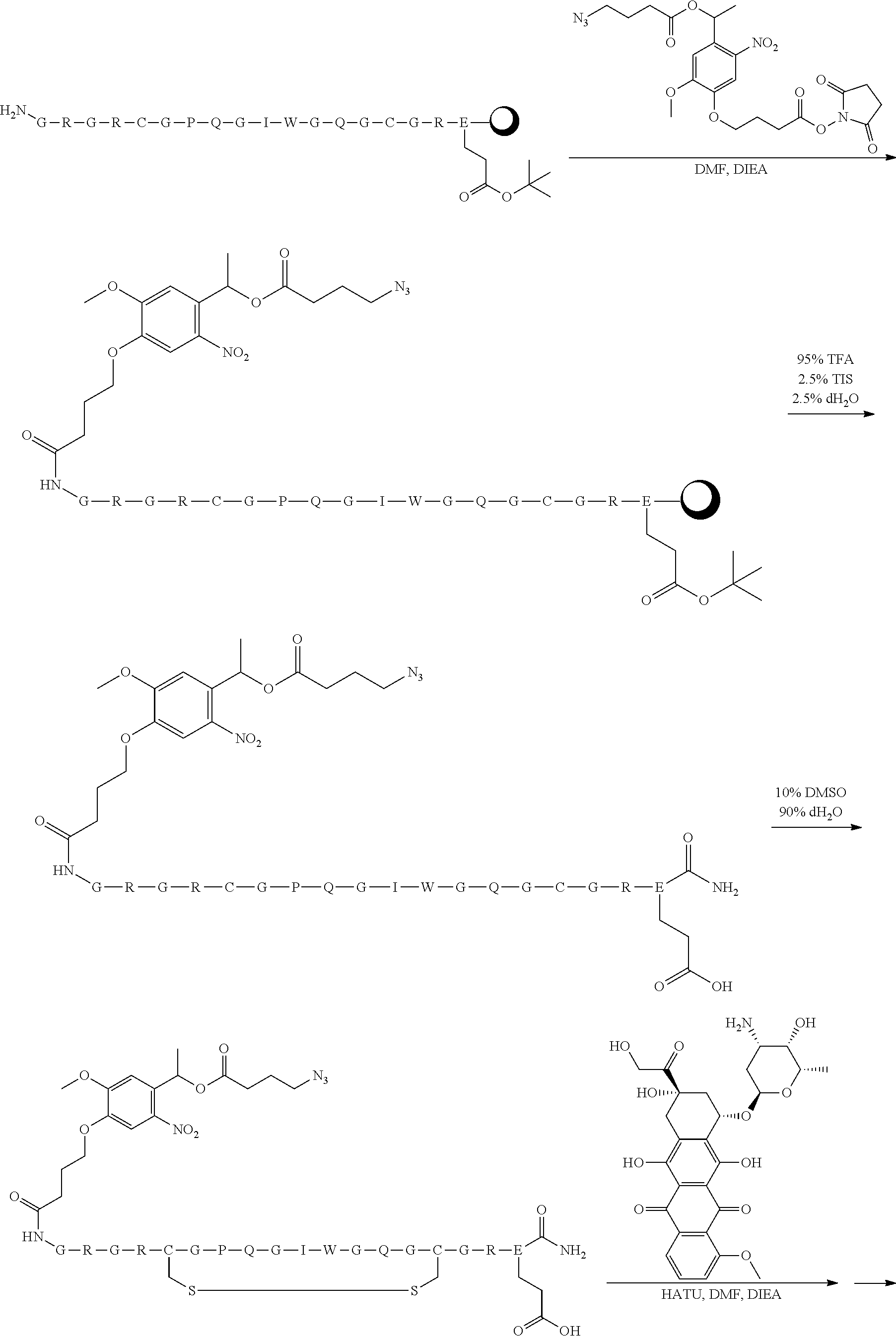

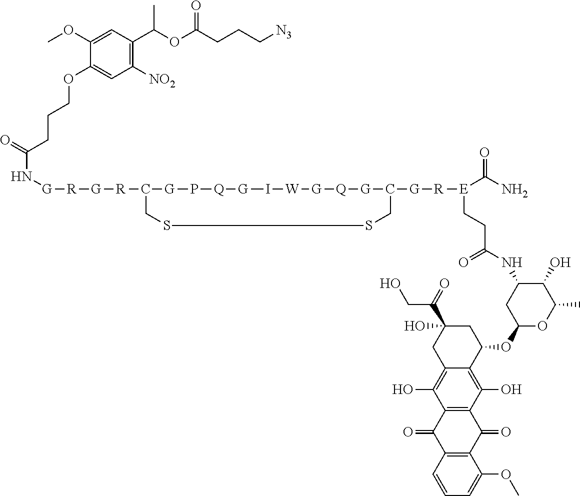

### Synthesis of N₃-PL-GRGRCGPQGIWGQGCGRE(DOX)—NH₂ Cyclized) (SEQ ID NO. 51) Pendant

The base peptide H-GRGRCGPQGIWGQGCGRE (SEQ ID NO. 51) was synthesized on rink amide resin (ChemPep, 0.8 meg/mg) via standard microwave-assisted Fmoc solid phase methodology and HBTU activation on a scale of 0.5 mmol (CEM Liberty 1). OSu-PL-N₃ (95 mg, 0.19 mmol, 0.4×) was dissolved in minimal DMF and activated with DIEA (258.5 mg, 330.5 μL, 2 mmol, 4×) and added to peptide to react overnight. The photolabile coupling was further accomplished with addition of photolabile azide acid (613.71 mg, 1.5 mmol, 3×) in the presence of HATU (551.33 mg, 1.45 mmol, 2.9×), which were codissolved in DMF (˜5 mL), pre-reacted for 5 mins with DIEA (387.7 mg, 495.8 μL, 3.0 mmol, 6×) and allowed to react at room temperature, protected from light for 2 hrs. Resin was treated with trifluoroacetic acid/water/triisopropylsilane (95:2.5:2.5) for 2 hr, and the crude peptide was precipitated in and washed (2×) with ice-cold diethyl ether. The crude peptide was purified using RP-HPLC using a 55 min linear gradient (5-100% of acetonitrile and 0.1% trifluoroacetic acid) and lyophilized to give the intermediate (N₃-PL-GRGRCGPQGIWGQGCGRE-NH₂) (SEQ ID NO. 51) as a fluffy, cream-colored solid. The purified peptide (<0.5 mM) was dissolved in a dH₂O/DMSO (90:10) solution (30 mL) and rocked for 24 hours. Product was concentrated, lyophilized and subsequently purified using RP-HPLC using a 55 min linear gradient (5-100% of acetonitrile and 0.1% trifluoroacetic acid) and lyophilized to give the intermediate (N₃-PL-GRGRCGPQGIWGQGCGRE-NH₂ (SEQ ID NO. 51) cyclized via cysteine-cysteine disulfide bridge) as a fluffy, cream-colored solid. HATU (2.9×) was added to the peptide (1×), dissolved in DMF (˜3 mL), then pre-reacted with DIEA (258.5 mg, 330.5 μL, 2 mmol, 4×) for 2 mins before adding Doxorubicin HCl (1×, Fisher) and allowed to react overnight, protected from light, at room temperature. The peptide was purified using RP-HPLC using a 55 min linear gradient (5-100% of acetonitrile and 0.1% trifluoroacetic acid) and lyophilized to give the product

(N₃-PL-GRGRCGPQGIWGQGCGRE(DOX)—NH₂ (SEQ ID NO. 51) cyclized via cysteine-cysteine disulfide bridge) as a fluffy, red solid. Peptide purity was confirmed with analytical RP-HPLC and matrix-assisted laser desorption-ionization time-of-flight mass spectrometry using α-cyano-4-hydroxycinnamic acid matrix: MALDI-TOF: calculated for C₁₁₉H₁₆₆N₃₆O₃₉S₂⁺ [M+¹H]⁺, 2788.9.

### Synthesis of Pendant Conjugated Hydrogels

The peptide pendants were aliquoted by dissolving them in DMSO to a concentration of 250 μM. PEG_{20 k}-tetra-BCN and PEG₃₅₀₀-diazide was aliquoted in phosphate buffered saline (PBS, 1×, Life Technologies) at concentrations of 20 mM and 40 mM respectively. Gels were formed by mixing PEG_{20 k}-tetra-BCN (56.25 μL, 20 mM) with PBS (22.5 μL, 1×) and the respective pendant (15 μL, 250 μM), then centrifuged and pre-reacted for 1 hr protected from light. PEG₃₅₀₀-diazide (56.25 μL, 40 mM) was then added, and the resulting solution was rapidly mixed, centrifuged, and then divided into 6 clear 0.5 mL centrifuge vials, centrifuged, and allowed to react for 1 hr to form 25 μL hydrogels. The gels were then swelled overnight in PBS (225 μL, 1×). The supernatant was removed 2 more times and replaced with fresh PBS (225 μL, 1×) to remove any unreacted pendant.

### Photopatterning and Release of FAM-Photolabile-AND-Reducible Pendant

Azide functionalized glass slides were synthesized as reported in previous works. Four 25 μL gels were synthesized as described in Gels were formed by mixing PEG_{20 k}-tetra-BCN (37.5 μL, 20 mM) with PBS (11.93 μL, 1×) and the respective pendant (13.07 μL, 765 μM), then centrifuged and pre-reacted for 1 hr protected from light. PEG₃₅₀₀-diazide (37.5 μL, 40 mM) was then added, and the resulting solution was rapidly mixed, centrifuged, and then pipetted (25 μL) onto the azide functionalized glass slide. The gels were allowed to gel to completion for 1 hr, then covered with a RainX treated glass slide, and PBS (1×) was added to swell and rinse the gel overnight at room temperature, protected from light. The following morning, two of the gels (gel 3 and 4) was exposed to 365 nm light at 20 mW/cm² for 10 minutes with a hatched square (100 μm) chrome photomask. Following the light treatment, gel 3 was returned to PBS (1×) and protected from light. Gels 2 and 4 were placed in a solution of Dithiothreitol (DTT, 1 mM) for 4 hours. Gels 2 and 4 were then transferred to PBS (1×) and protected from light for 30 mins. Gel 1 remained in PBS and protected from light the entire time. The resulting fluorescence of these gels were imaged using a confocal scanning microscope at 488 nm. Gels 1 (control), 2 (DTT only), and 3 (light only) displayed no visible loss in fluorescence. Gel 4 (light and DTT) showed release in only the exposed areas directly related to the photomask (FIG. 17).

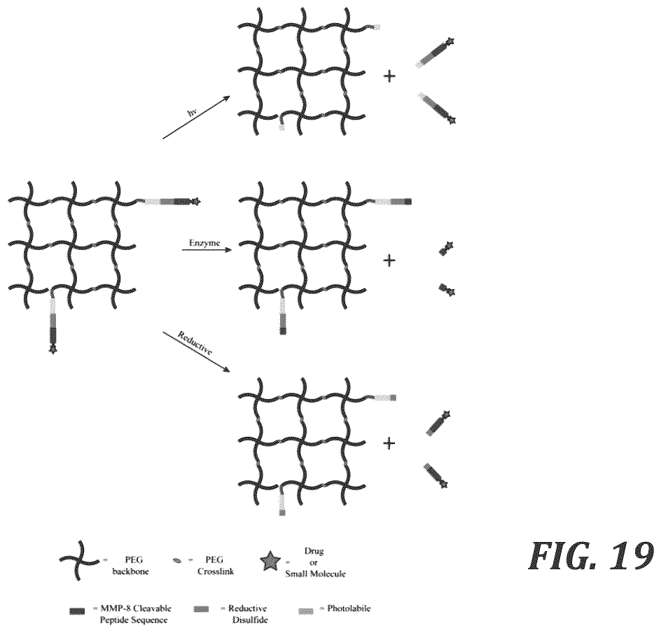

Schematic representations of a release of a fluorescent reporter from a model biomaterial including-AND/OR-responsive multifunctional linker; or of a release of a therapeutic agent from a model biomaterial including-OR-responsive multifunctional linker are also shown in FIGS. 18 and 19.

### Example 3. Independent Logic-Based Delivery of Site-Specifically-Modified Proteins from Gels Through Engineered Biomacromolecular Architecture

A robust synthetic strategy to enable the user-programmable release of site-specially-modified proteins from gels is presented. By tethering proteins of interest to hydrogel networks through modular degradable linkers of defined molecular architecture, Boolean YES/OR/AND logic-based control is obtained over protein release in response to complex sets of inputs. As this approach enables biomacromolecular delivery only when user-specified combinations of external cues are present, sequential and independent release of multiple singly-modified proteins is obtained.

Initial demonstrations of on-demand protein release were performed using poly(ethylene glycol) (PEG)-based hydrogels. PEG is an inert hydrophilic polymer that exhibits exceptional resistance to protein adsorption, important for minimizing fouling upon controlled protein release. Step-growth gels with ideal network structure were formed via a strain-promoted azide-alkyne cycloaddition (SPAAC) reaction between a four-arm PEG tetrabicyclononyne (PEG-tetraBCN, Mₙ˜20,000 Da) and a homobifunctional linear PEG diazide (N₃-PEG-N₃, Mₙ˜3,500 Da). With SPAAC representing the most common of the emerging class of bioorthogonal click reactions, SPAAC-based gels are readily formed in the presence of living cells, complex medium, and full-length proteins with excellent reaction selectivity. By including azide-functionalized proteins in the gel formulation, these biomolecules are tethered uniformly throughout the network at user-specified concentrations with minimal impact on material physical properties.

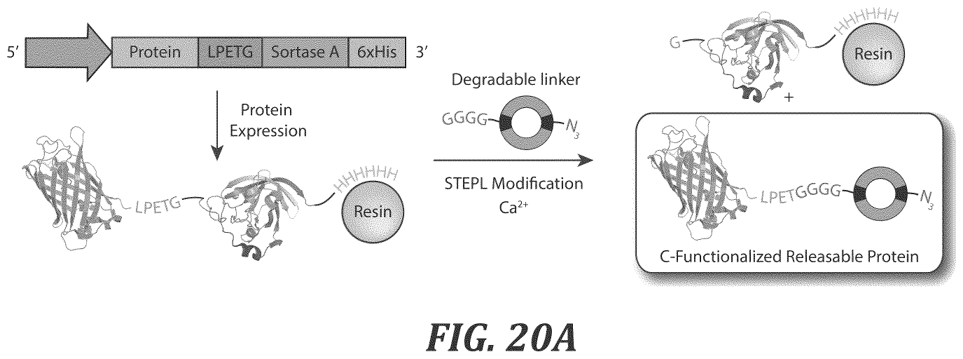

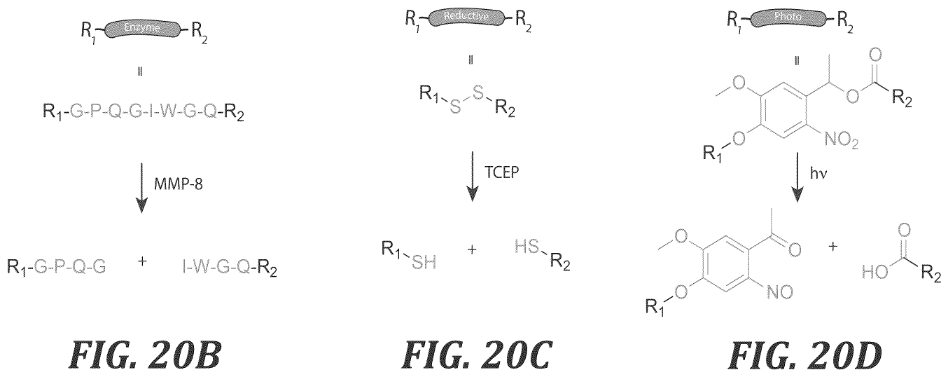

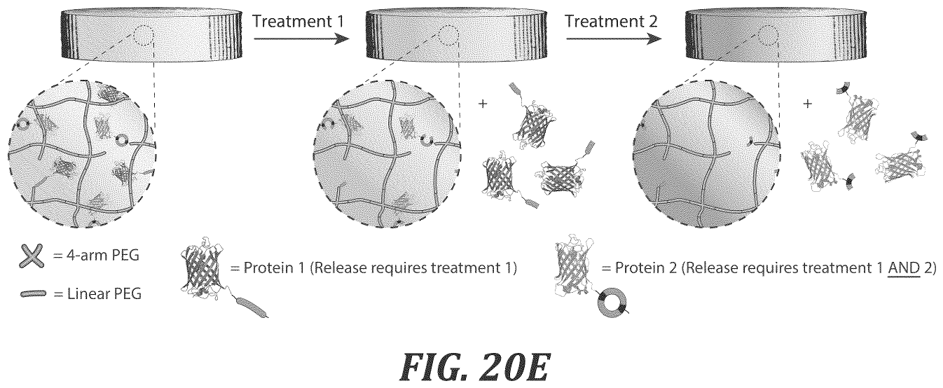

To introduce azide functionality site-specifically onto proteins of interest, thereby preserving bioactivity and yielding a homogeneous working population, a sortase-mediated transpeptidation reaction was used. Staphylococcus aureus sortase A is a calcium-assisted transpeptidase that recognizes the C-terminal sorting signal “LPXTG”, forming an acyl-enzyme intermediate with the protein while simultaneously displacing the C-terminal portion of the sorting signal's threonine residue. The thioester of the acyl-enzyme intermediate can be nucleophilically displaced with a polyglycine probe, thereby conjugating the probe onto the C-terminus while regenerating the sortase A enzyme. Critically, the polyglycine compound may contain non-natural functionality, providing a route to “sortag” bioorthogonal moieties including azides onto the C-terminus of proteins of interest. To facilitate one-step protein biofunctionalization and purification, sortagging through the Sortase-Tag Enhanced Protein Ligation (STEPL) technique (FIG. 20A) was implemented. In STEPL, the protein of interest, sorting sequence LPETG (SEQ ID NO. 4), (GGSGGSGGSGGSGGS (SEQ ID NO. 7) flexible linker, sortase A, and a HisHisHisHisHisHis-Tag (SEQ ID NO. 6) are fused into a single protein construct which is expressed recombinantly in E. coli. The flexible GGSGGSGGSGGSGGS (SEQ ID NO. 7) linker allows the sortase domain to recognize the LPETG (SEQ ID NO. 4) sequence and for intramolecular sortagging to occur. The sortase-LPETG (SEQ ID NO. 4) intermediate is displaced by the addition of calcium and a customizable probe with an N-terminal polyglycine moiety, ligating the protein of interest to the peptide and separating it from the remaining HisHisHisHisHisHis-Tag (SEQ ID NO. 6)-functionalized sortase A. This step can be performed during chromatographic HisHisHisHisHisHis-Tag (SEQ ID NO. 6) purification of the protein, where sortase A remains bound to the Ni-NTA column, allowing for site-specific labeling and purification of proteins in a single step.

Taking advantage of its unique ability to install non-natural functionalities onto the C-terminus of recombinant proteins of interest, STEPL was used to introduce both an azide necessary for hydrogel tethering as well as one of several modular degradable sequences to be used for triggered protein release. Building on Example 1, here, the release of site-specifically modified proteins through the inclusion of linkers that would degrade in response to pre-defined input combinations following Boolean YES/OR/AND logic is presented. YES-based release can achieved by simply including a degradable moiety between the protein and the azide; OR response can be engineered by including two degradable moieties in series between the protein and the azide; AND response can be obtained by including two degradable moieties in parallel, here in the form of a cyclic stapled peptide between the protein and the azide, where degradation of both groups would be required to cleave the protein from the gel.

To assess whether logic-based protein release could be achieved and to test the modularity of this approach, three distinct degradable moieties were used: 1) the matrix metalloproteinase (MMP)-sensitive peptide sequence GPQG↓IWGQ (SEQ ID NO. 1) that is enzymatically cleaved between the glycine and isoleucine residues by MMPs including MMP-8 (FIG. 20 B); 2) disulfide linkages that can be reductively cleaved in the presence of tris(2-carboxyethyl)phosphine (TCEP) or other reducing agents (FIG. 20C); 3) an ortho-nitrobenzyl ester moiety, which undergoes irreversible photocleavage upon UV light exposure (λ=365 nm) (FIG. 20D). As each degradable moiety was selected to be susceptible to a different class of biologically-relevant environmental stimuli (i.e., enzyme, chemical environment, light), we expected that chemical cleavage reactions would proceed orthogonally to one another (FIG. 20E).

Having chosen MMP enzyme—(E), reductive—(R), and light—(P) sensitive moieties to represent our three distinct degradation chemistries, we constructed a total of nine degradable linkers for C-terminal protein modification by STEPL: three YES logic gated peptides (GGGG-E-N₃ (SEQ ID NO. 12), GGGG-R—N₃ (SEQ ID NO. 8), and GGGG-P—N₃ (SEQ ID NO. 9)) that degrade when presented with only one predefined cue; three OR logic gated peptides (GGGG-E∨R—N₃ (SEQ ID NO. 10), GGGG-R∨P—N₃ (SEQ ID NO. 11), and GGGG-E∨P—N₃ (SEQ ID NO. 12)) that are sensitive to two different specified cues; three AND logic gated stapled peptides (GGGG-E∧R—N₃ (SEQ ID NO. 13), GGGG-R∧P—N₃ (SEQ ID NO. 15) bound to (SEQ ID NO. 14) through oNB, GGGG-E∧P—N₃ (SEQ ID NO. 18)) that require two cue-induced cleavage reactions for peptide gate degradation (see methods below). For each peptide, the degradable component was flanked by an N-terminal polyglycine (GGGG) (SEQ ID NO. 3) moiety required for sortagging and a C-terminal azide (N₃) for gel tethering. All peptide linkers were synthesized, purified by HPLC, and characterized by MALDI-TOF mass spectrometry prior to use, and represented the exhaustive set of single- and double-input degradable linkers spanning the E, R, and P chemistries.

With the polyglycine-containing peptides in hand, STEPL was used to install Boolean logic-based degradable linkers onto the C-terminus of several recombinant proteins of interest (see methods below). Though this method could be used to modify virtually any protein species engineered to contain the sorting signal, initial efforts were focused on Enhanced Green Fluorescent Protein (EGFP), whose fluorescence are convenient for visualizing and quantify protein release from hydrogels. Each of the nine degradable polyglycine probes were sortagged onto EGFP, yielding EGFP-E-N₃, EGFP-R—N₃, EGFP-P—N₃, EGFP-E∨R—N₃, EGFP-R∨P—N₃, EGFP-E∨P—N₃, EGFP-E∧R—N₃, EGFP-R∧P—N₃, and EGFP-E∧P—N₃. All proteins were isolated in excellent purity with quantitative terminal functionalization, as confirmed by whole protein mass spectrometry.

To test protein release response to different combinations of environmental stimuli, each of the nine EGFP variants were individual tethered (100 μM) into PEG-based gels (8 experimental conditions in triplicate=24 gels per EGFP variant). Release of each EGFP variant was determined for 8 possible treatment conditions: control (N, no treatment), enzyme treatment (E); reductive treatment (R); light treatment (P); reductive and enzyme (RE); enzyme and light (EP); reductive and light (RP); reductive, enzyme, and light (REP). Supernatant fluorescence was used to quantify EGFP release for each treatment condition.

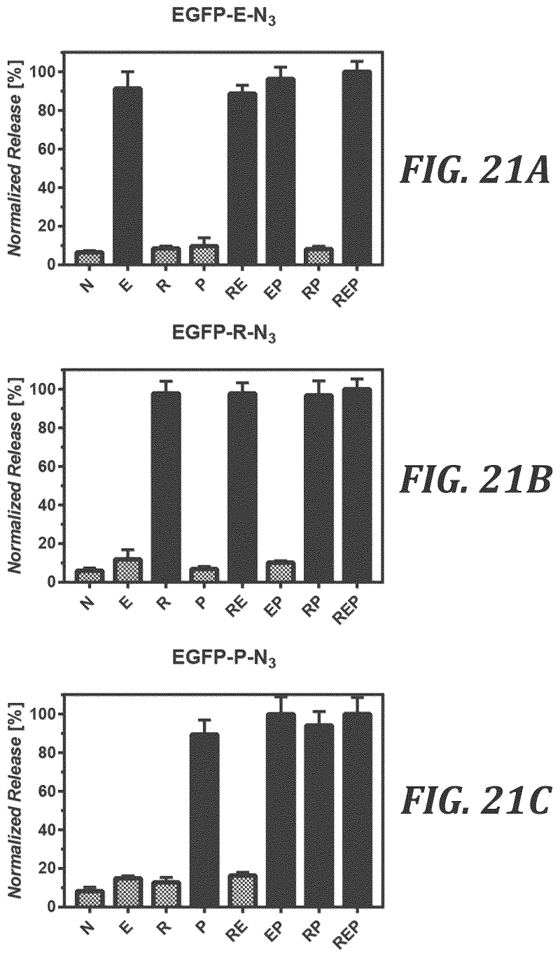

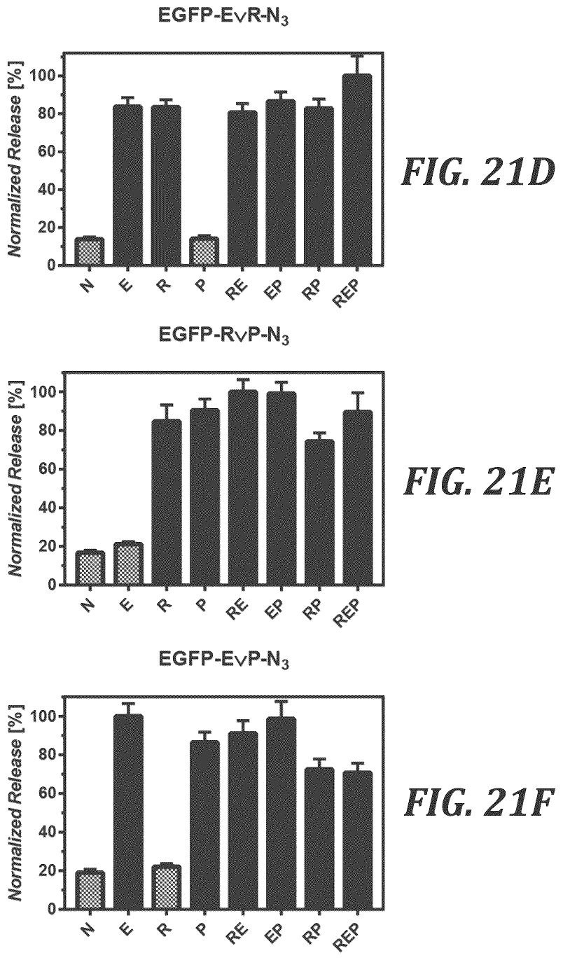

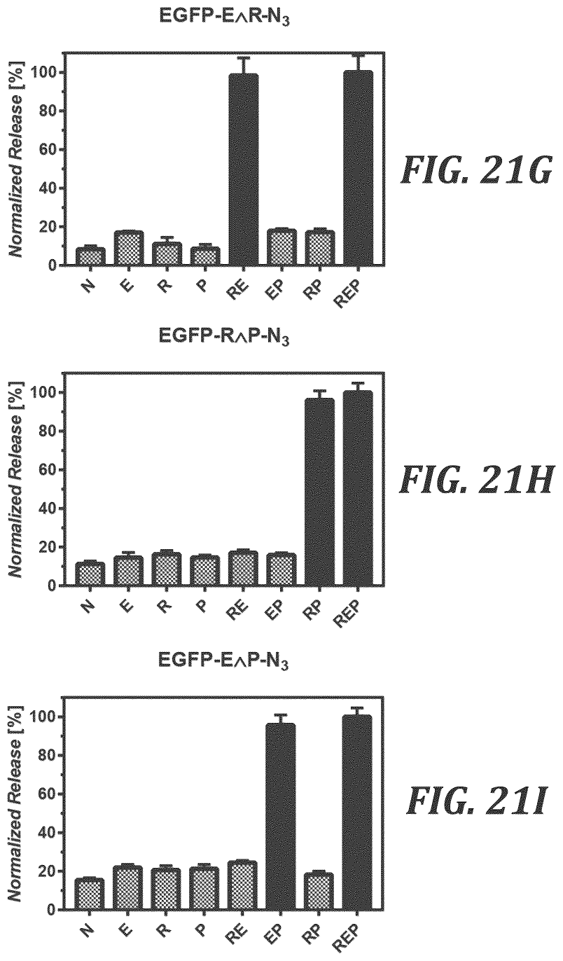

The YES logic-based systems (EGFP-E-N₃, EGFP-R—N₃, EGFP-P—N₃ released protein as expected, exhibiting a ˜10-fold increase in release for treatments that included the relevant environmental stimuli over those that did not (FIGS. 21A-21C). This high level of selectivity demonstrates that the three chosen inputs did indeed offer chemically orthogonal response. The OR logic-based systems (EGFP-E∨R—N₃, EGFP-R∨P—N₃, EGFP-E∨P—N₃ also behaved as expected, where increased protein release was observed in the presence of either programmed cue (FIGS. 21E-21F). Though still high, the selectivity of the OR-responsive systems decreased to >5-fold, consistent with there now being two degradable linkages that could potentially undergo non-specific cleavage. The AND logic-based platforms (EGFP-E∧R—N₃, EGFP-R∧P—N₃, and EGFP-E∧P—N₃) offered protein release only when both requisite environmental cues were present, again with a >5-fold release selectivity (FIGS. 21G-21I). To our knowledge, these AND-based materials represent the very first systems where more than one input is required to release a protein payload from a hydrogel material. The modularity of the approach, where the same protein can be readily sortagged, can yield varied release responses to many different sets of environmental signals.

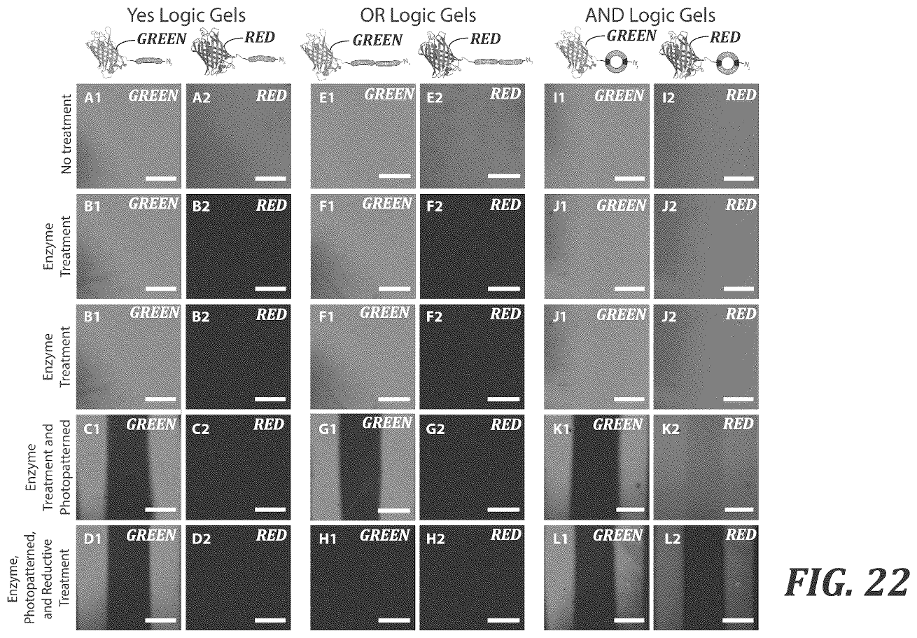

Given the high level of control the approach offers for the release of single proteins in response to different combinations of environmental cues, whether an expanded strategy could be used to permit independent and differentially-triggered release of many proteins from a common gel material was then investigated, such as systems that would simultaneously deliver EGFP as well as the red fluorescent protein mCherry (also synthesized and modified by STEPL) from the same material via a different logic-programmed delivery mechanism (FIG. 22). Gels uniformly decorated with both tethered proteins (50 μM each) were exposed to MMP enzyme treatment, followed by masked UV light exposure (400 μm line patterns), and then reductive treatment. Gels were imaged by fluorescent confocal microscopy before and after each treatment step to visualize any remaining tethered protein.

To demonstrate independent protein release using the YES-logical systems, EGFP-P—N₃ and mCherry-E-N₃ were conjugated into the same hydrogel (FIG. 22 A1, A2).

After MMP enzyme treatment, EGFP remained tethered to the gel (FIG. 22 B1) while mCherry was fully released (FIG. 22 B2). Subsequent treatment with masked light resulted in EGFP release in UV-exposed gel regions (FIG. 22 C1). EGFP remained bound to the gel in the patterned orientation even after reductive conditions were applied (FIG. 22 D1). For OR logic-based gels, EGFP-P∨R—N₃ and mCherry-E∨R—N₃ were conjugated into the same hydrogel (FIG. 22 E1, E2). mCherry was fully released with enzyme treatment (FIG. 22 F2) while EGFP remained tethered to the gel (FIG. 22 F1). Treatment with masked UV light resulted in EGFP being released from exposed regions (FIG. 22 G1). Upon subsequent reductive treatment, complete release of the patterned EGFP was observed (FIG. 22 H1). To demonstrate that different AND logic-based linkers could be used to delivery more than one protein from the same material, EGFP-E∧P—N₃ and mCherry-R∧P—N₃ were tethered to a common gel (FIG. 22 I1, I2). No protein release was observed following enzymatic treatment (FIG. 22 J1, J2). Masked light treatment resulted in EGFP release from the UV exposed portions of the material (FIG. 22 K1), while mCherry remained tethered throughout the network. When treated with reductive conditions, the EGFP pattern remained intact (FIG. 22 L1) while the previously-photopatterned regions of mCherry were reductively “developed” (FIG. 22 L2). In all cases, triggered protein release matches expected responses. These experiments highlight our unique ability to program the independently-triggered delivery of multiple proteins from the same material.

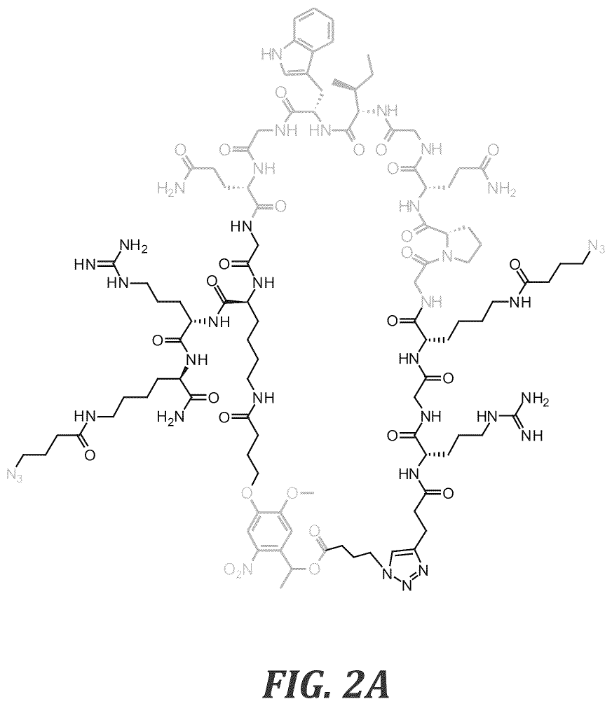

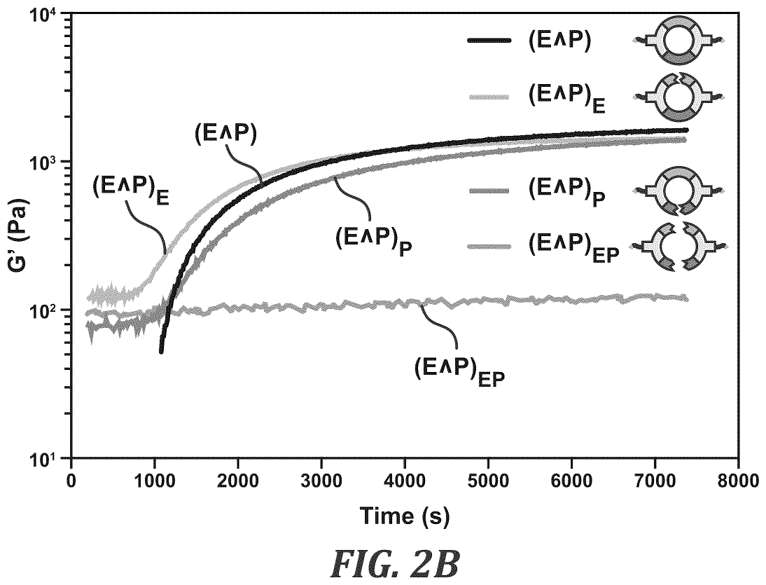

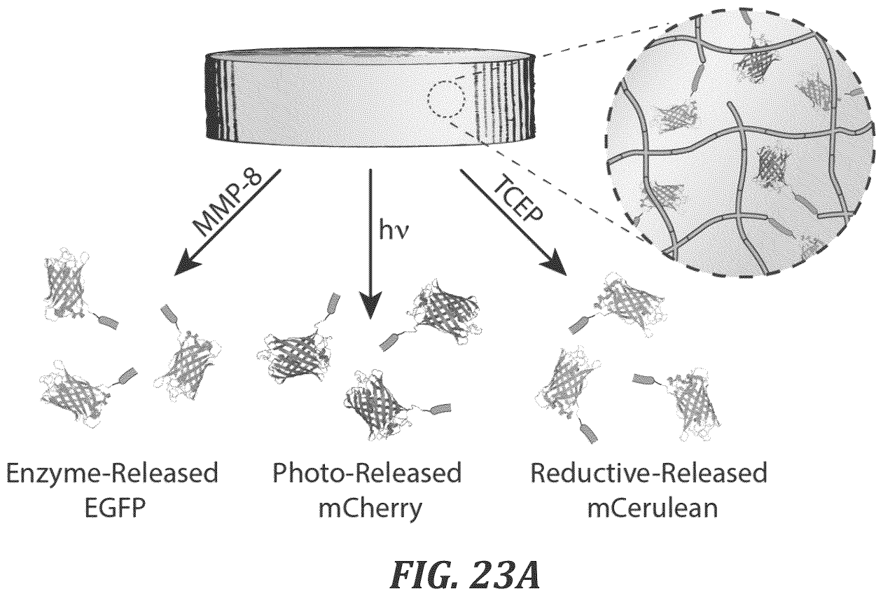

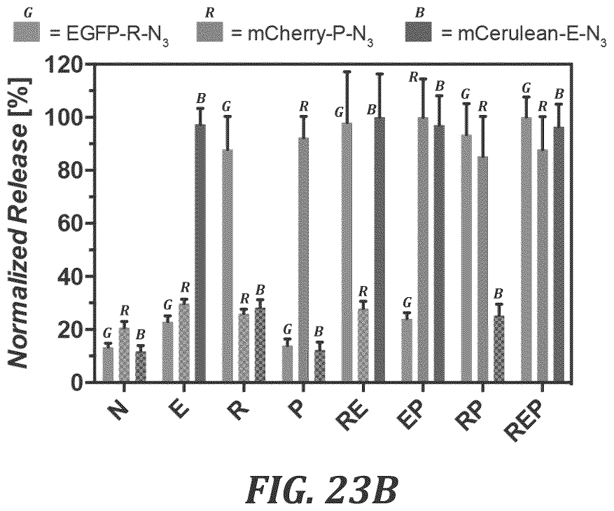

We next sought to further push the boundaries of controlled protein delivery, working with gels tethered with three distinct fluorescent proteins through different YES logic-based degradable linkers. EGFP-R—N₃, mCherry-P—N₃, and a blue fluorescent protein mCerulean-E-N₃ (prepared by STEPL) were included (33 μM each) in a common PEG hydrogel formulation. The use of multiple fluorescent proteins allowed us to quantify and resolve the logic-based release behavior of all the YES logic pendants from a single hydrogel upon different environmental cues (FIGS. 23A-23B). Protein release proceeded as designed: EGFP release was observed for treatments that contained a reductive step (i.e., R, RE, RP, REP); mCherry was released only in conditions involving light treatment (i.e., P, EP, RP, REP); mCerulean was released in those involving MMP (i.e., E, RE, EP, REP). In comparing these experiments with the YES-logic response for just EGFP (FIG. 2A-c), the reduced selectivity was attributed to the lower amount of each individual protein initially tethered to the gel. Otherwise, the release characteristics of each protein are identical to those observed in the single-protein systems, where degradation reaction orthogonality enables independent control of many proteins from the same gel. This represents the only approach to date that enables independent programmability over more than one protein's triggered release from the same homogeneous material.

In this work, a robust strategy for the synthesis of site-specifically modified proteins that can be tethered to and released from hydrogel biomaterials in response to user-programmable external cues was described. Exploiting a unique sortase-mediated transpeptidation reaction, the C-termini of a variety of proteins was quantitatively functionalized with degradable units spanning three different biologically-relevant input classes (i.e., enzyme, light, reductive environments), and Boolean YES/OR/AND logic-based triggered protein release was demonstrated successfully exhaustively for systems requiring one or two of these inputs. Systems where more than one external cue is required to release a protein payload from a material have been demonstrated, as well as the independently-triggered release of multiple proteins from the same homogeneous material. Given their synthetic modularity, these systems can be useful in controlled therapeutic delivery for disease treatment and tissue regeneration.

### Methods

### Synthesis of Logic-Based Peptides for Sortagging

Peptides were synthesized through a mixture of standard Fmoc-based solid-phase and solution-phase synthetic methodologies. Stapled peptides were prepared upon disulfide formation between two intramolecular cysteine residues, or by copper(I)-catalyzed azide-alkyne cycloaddition. Peptides were purified by semi-preparative reversed-phase high performance liquid chromatography (HPLC). Purity was confirmed by analytical RP-HPLC and matrix-assisted laser desorption, ionization time of flight (MALDI-TOF) mass spectrometry.

STEPL-Based Sortagging to Yield C-Terminally Label Proteins

A protein of interest (POI), fused with 6×His-tagged Sortase, was grown and expressed in BL21(DE3) E. coli. Cells were lysed via sonication. 6×His-tagged Sortase-POI fusion protein was immobilized on Ni-NTA. To promote the STEPL reaction, sortaggable peptide (10×) and calcium chloride (0.1 mM) was added to the Ni-NTA resin, and was reacted at 37° C. for 4 hours with gently agitation. The flow through containing the sortagged protein was collected. Excess peptide was removed by centrifugal membrane filtration.

### Protein Release Studies

Hydrogels were formulated from PEG-tetraBCN (2 mM), N₃-PEG-N₃ (4 mM), and azide-functionalized proteins (0.1 mM) in MMP buffer (Tris 50 mM, NaCl 200 mM, CaCl₂) 5 mM, ZnCl₂ 1 μM, pH 7.5). Proteins were pre-reacted with PEG-tetraBCN for 4 hours prior to N₃-PEG-N₃ crosslinker addition. Gels were allowed to form for one hour, after which wash steps were performed to remove any unconjugated protein. Reductive treatment was performed with TCEP-HCl (2 μL, 100 mM in MMP buffer). Excess TCEP was quenched with hydroxyethyl disulfide (5 μL, 100 mM in MMP buffer). Enzyme treatment was performed with recombinantly expressed MMP-8 (2.5 μL, 0.4 mg/mL in MMP buffer). Light exposure was performed with UV light (λ=365 nm, 20 mW cm⁻², 10 minutes). All treatments were performed at 4° C. Supernatant fluorescence was used to quantify amounts of release EGFP (λ_{excitation}=475 nm, λₑₘᵢₛₛᵢₒₙ=510 nm), mCherry (λ_{excitation}=575 nm, λₑₘᵢₛₛᵢₒₙ=610 nm), and mCerrulean (λ_{excitation}=433 nm, λₑₘᵢₛₛᵢₒₙ=475 nm).

### General Synthetic Information

Chemical reagents and solvents were purchased from either Sigma-Aldrich or Fisher Scientific and used without further purification. Distilled water (dH₂O) was obtained from a U.S. Filter Corporation Reverse Osmosis system equipped with a desalination membrane. All chemical reactions were performed under inert nitrogen atmosphere in flame-dried glassware and were stirred with Teflon-coated magnetic stir bars. Solvent was removed under reduced pressure with a Buchi Rotovap R-3 by using either V-700 vacuum pump or Welch 1400 high vacuum pump. All peptides were synthesized using Fmoc-based, microwave-assisted, solid-phase peptide synthesis methodology on a CEM Liberty 1. All peptides were purified by semi-preparative reverse-phase high pressure liquid chromatography (RP-HPLC) performed on Dionex Ultimate 3000 equipped with RS multiple variable wavelength detector, automated fraction collector, and C18 column. Peptide characterization was performed by using Matrix-assisted laser desorption/ionization time of flight (MALDI-TOF) on Bruker AutoFlex II. Lyophilization was performed on a Labconco FreeZone 2.5 Plus freeze-dryer equipped with Labconco rotary vane 117 vacuum pump. Lumen Dynamics OmniCure S1500 Spot UV curing system was used for photochemical cleavage reactions, where light intensity was determined using a Cole-Palmer radiometer (Series 9811-50, λ=365 nm). All cell cultures were maintained in Thermo-Fisher Scientific MaxQ 4000 Benchtop Orbital Shaker-Incubator. Cells were lysed using Fisher Scientific Sonic Dismembrator (Model 505). Protein concentrations were measured on Thermo-Scientific NanoDrop 2000 Spectrophotometer. Protein mass characterization was performed on SYNAPT G2-Si Mass Spectrometer equipped with a liquid chromatography column (LC-MS). Fluorescence measurement data was acquired from SpectraMax M5 spectrometer. Fluorescence microscopy was performed on Leica SP8× confocal microscope. The synthesis of 2,5-dioxopyrrolidin-1-yl 4-azidobutanoate (N₃—OSu), 2,5-dioxopyrrolidin-1-yl 4-(4-(1-((4-azidobutanoyl)oxy)ethyl)-2-methoxy-5-nitrophenoxy) butanoate (N₃-oNB—OSu), 4-arm-PEG_{20 kDa}-tetrabicyclononyne (PEG_{20K}-tetraBCN), and PEG_{3.5 kDa}-diazide were performed as reported in literature. Fmoc-Lys(N₃)—OH was synthesized according literature procedures.

### Synthesis of Enzymatically-Degradable, Sortaggable Peptide (GGGG-E-N₃) (SEQ ID NO. 12)

H-GGGGRGPQGIWGQGRK(N₃)—NH₂ (SEQ ID NO. 12) was synthesized on rink amide resin (0.25 mmol scale) via standard Fmoc-based, microwave-assisted, solid-phase peptide synthesis methodologies; Fmoc-Lys(N₃)—OH was utilized to introduce azide functionality at the C-terminus. The peptide was deprotected and cleaved from resin by treatment with trifluoroacetic acid (TFA)/triisopropylsilane (TIS)/dH₂O (95:2.5:2.5, 15 mL) for 2 hours. Following cleavage, the peptide was precipitated in and washed with ice-cold diethyl ether (2×), purified by RP-HPLC operating with 55 min gradient (5-100%) of acetonitrile in water containing TFA (0.1%), and lyophilized to obtain the final peptide (H-GGGGRGPQGIWGQGRK(N₃)—NH₂ (SEQ ID NO. 12, denoted GGGG-E-N₃) as a white solid (97.6 mg, 0.0613 mmol, 24.5% yield). Peptide purity was confirmed by MALDI-TOF mass spectroscopy: calculated M+¹H]⁺, 1592.70, observed 1592.46.

### Synthesis of Reduction-Degradable, Sortaggable Peptide (GGGG-R—N₃ (SEQ ID NO. 8))

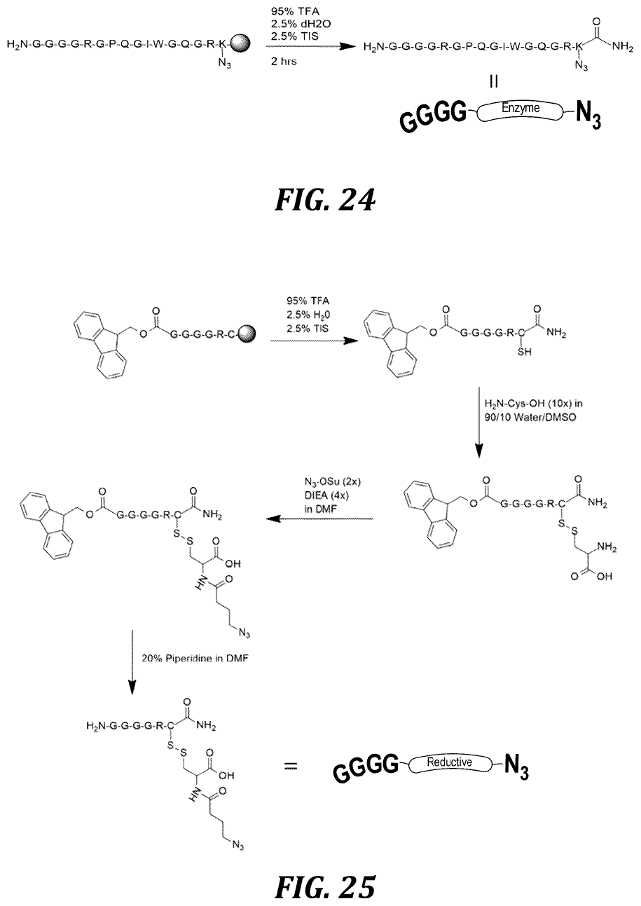

Fmoc-GGGGRC (SEQ ID NO. 8) was synthesized on rink amide resin (0.25 mmol scale) via standard Fmoc-based, microwave-assisted, solid-phase peptide synthesis methodologies (FIG. 24). The peptide was deprotected and cleaved from resin by treatment with TFA/TIS/dH₂O (95:2.5:2.5, 15 mL) for 2 hours. Following cleavage, the peptide was precipitated in and washed with ice-cold diethyl ether (2×), purified by RP-HPLC operating with 43.4 min gradient (20-100%) of acetonitrile in water containing TFA (0.1%), and lyophilized to obtain the intermediate peptide Fmoc-GGGGRC-NH₂ (SEQ ID NO. 8) as a white solid. The intermediate peptide (50 mg, 0.069 mmol, 27.6% yield) and H-Cys-OH (84 mg, 0.69 mmol, 10×) were dissolved dH₂O/DMSO (9:1) and reacted at room temperature for 48 hrs. The solution was concentrated by rotary evaporation, dissolved in dH₂O, purified by RP-HPLC with 43.4 min gradient (20-100%) of acetonitrile in water containing TFA (0.1%), and lyophilized to give the intermediate peptide (Fmoc-GGGGRC-(H—C—OH)—NH₂ (SEQ ID NO. 8) with cysteines linked via disulfide bond) as a white solid. The intermediate peptide (8.2 mg, 0.0097 mmol, 14% yield) was reacted overnight with N₃—OSu (4.38 mg, 0.0194 mmol, 2×) and DIEA (5.01 mg, 0.039 mmol, 4×) in minimal DMF to introduce azide functionality onto the peptide. The reaction mixture was purified by RP-HPLC with 43.4 min gradient (20-100%) of acetonitrile in water containing TFA (0.1%), and the product fraction was lyophilized to obtain the intermediate peptide, Fmoc-GGGGRC(HN(CO(CH₂)₃—N₃)Cys)-NH₂ (SEQ ID NO. 8) with cysteines linked via disulfide bond (9 mg, 0.0094 mmol, 97% yield). The N-terminal Fmoc group was cleaved by incubating the peptide in piperidine (20%) in DMF (9 mL) for 10 mins. The deprotection reaction mixture was concentrated via rotary evaporation, purified by RP-HPLC operating with 55 min gradient (5-100%) of acetonitrile in water containing TFA (0.1%), and lyophilized to obtain the final product (H-GGGGRC((CO(CH₂)₃—N₃)Cys)-NH₂ (SEQ ID NO. 8) with cysteines linked via disulfide bond, denoted GGGG-R—N₃) as a white solid (1.5 mg, 0.002 mmol, 21.3% yield). Peptide purity was confirmed by MALDI-TOF mass spectroscopy: calculated [M+¹H]⁺, 734.97, observed 735.33.

### Synthesis of Photo-Degradable, Sortaggable Peptide (GGGG-P—N₃ (SEQ ID NO. 9))

Fmoc-GGGGRK(mtt) (SEQ ID NO. 9) was synthesized on rink amide resin (0.25 mmol scale) via standard Fmoc-based, microwave-assisted, solid-phase peptide synthesis methodologies (FIG. 25). The acid-labile N-methyltrityl (mtt) moiety protecting the 6-amino group of the lysine side chain was removed by treatment with DCM/TIS/TFA (97:2:1, 9×15 mL, 10 min each). The resin-bound peptide was reacted overnight with N₃-oNB—OSu (165 mg, 0.325 mmol, 1.3×) and DIEA (129.25 mg, 1 mmol, 4×) in minimal DMF to introduce oNB and azido functionality onto the ε-amino group of the lysine side chain. Fmoc deprotection was achieved on resin by treatment with piperidine (20%) and 1-hydroxybenzotriazole (HOBt, 0.1 M) in DMF (2×15 mL, 10 mins each). The peptide was deprotected and cleaved from resin by treatment with TFA/TIS/dH₂O (95:2.5:2.5, 15 mL) for 2 hours. Following cleavage, the peptide was precipitated in and washed with ice-cold diethyl ether (2×), purified by RP-HPLC operating with 55 min gradient (5-100%) of acetonitrile in water containing TFA (0.1%), and lyophilized to obtain the final product (H-GGGGRK(oNB-N₃)—NH₂ (SEQ ID NO. 9), denoted GGGG-P—N₃) as a yellow solid (41.1 mg, 0.45 mmol, 17.83% yield). Peptide purity was confirmed by MALDI-TOF mass spectroscopy: calculated [M+¹H]⁺, 921.96, observed 921.47.

### Synthesis of Enzyme-OR-Reductive-Degradable, Sortaggable Peptide (GGGG-E∨R—N₃ (SEQ ID NO. 10))

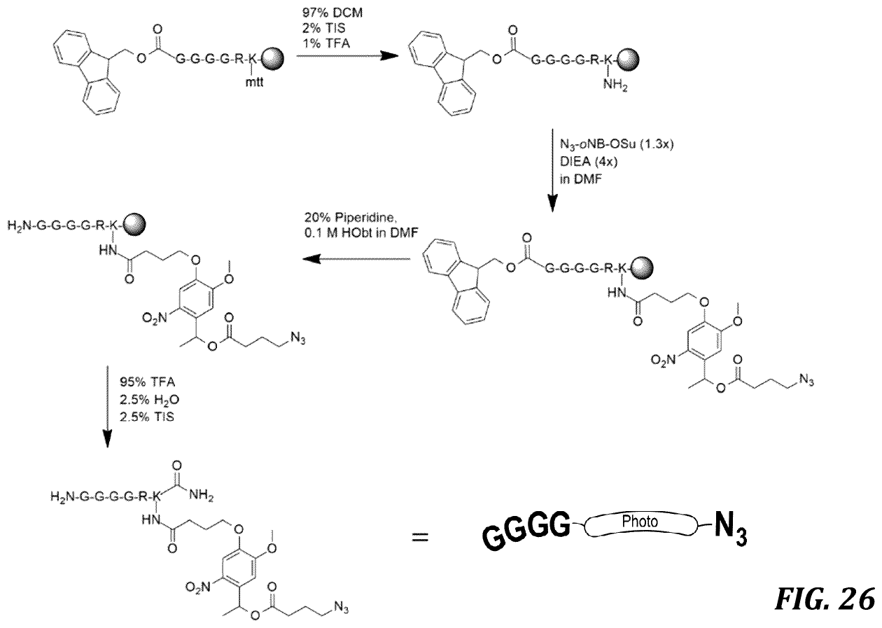

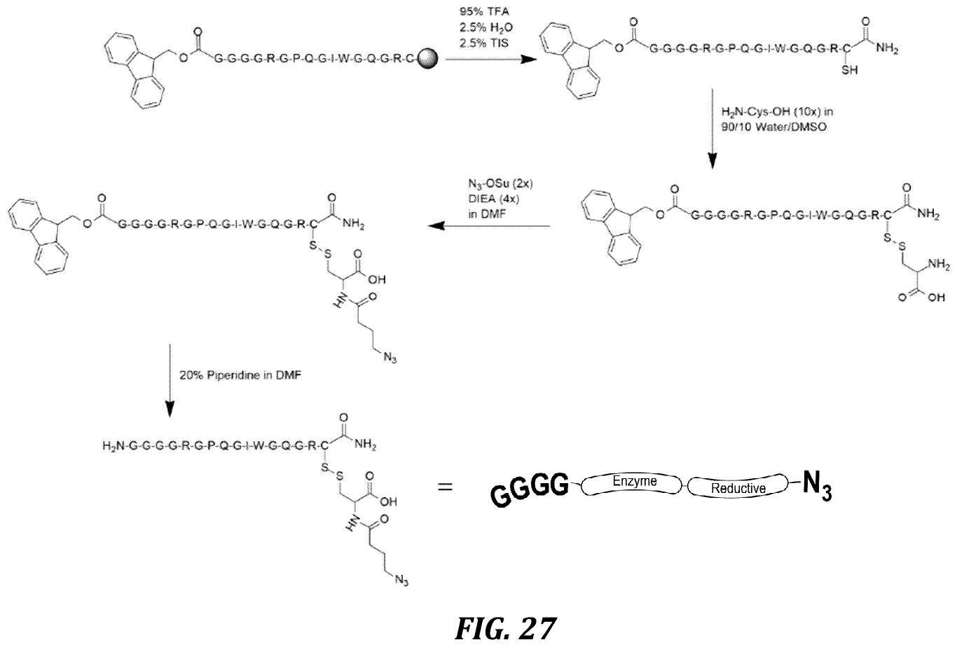

Fmoc-GGGGRGPQGIWGQGRC (SEQ ID NO. 10) was synthesized on rink amide resin (0.25 mmol scale) via standard Fmoc-based, microwave-assisted, solid-phase peptide synthesis methodologies (FIG. 26). The peptide was deprotected and cleaved from resin by treatment with TFA/TIS/dH₂O (95:2.5:2.5, 15 mL) for 2 hours. Following cleavage, the peptide was precipitated in and washed with ice-cold diethyl ether (2×), purified by RP-HPLC operating with 55 min gradient (5-100%) of acetonitrile in water containing TFA (0.1%), and lyophilized to obtain the intermediate peptide as a white solid (52 mg, 0.0295 mmol, 11.8% yield). The intermediate peptide and H₂N-Cys-OH (35.71 mg, 0.295 mmol, 10×) were dissolved in dH₂O/DMSO (9:1, 10 mL) and reacted at room temperature for 48 hrs. The solution was concentrated by rotary evaporation, dissolved in dH₂O, purified by RP-HPLC with 43.4 min gradient (20-100%) of acetonitrile in water containing TFA (0.1%), and lyophilized to give the intermediate peptide (Fmoc-GGGGRGPQGIWGQGRC(NH₂—C—OH)—NH₂ (SEQ ID NO. 10) with cysteines linked via disulfide bond) as a white solid (38 mg, 0.0202 mmol, 68.5% yield). The intermediate peptide was reacted overnight with N₃—OSu (9.1294 mg, 0.0403 mmol, 2×) and DIEA (10.42 mg, 0.087 mmol, 4×) in minimal DMF to introduce azide functionality onto the peptide. The reaction mixture was purified by RP-HPLC with 43.4 min gradient (20-100%) of acetonitrile in water containing TFA (0.1%), and the product fraction was lyophilized to obtain the intermediate peptide (Fmoc-GGGGRGPQGIWGQGRC (CO(CH₂)₃N₃)Cys)-NH₂ (SEQ ID NO. 10) with cysteines linked via disulfide bond) as a white solid (25 mg, 0.0125 mmol, 61.8% yield). The N-terminal Fmoc group was cleaved by incubating the peptide in piperidine (20%) in DMF (25 mL) for 10 mins. The deprotection reaction mixture was concentrated via rotary evaporation, purified by RP-HPLC operating with 55 min gradient (5-100%) of acetonitrile in water containing TFA (0.1%), and lyophilized to obtain the final product (H-GGGGRGPQGIWGQGRC(HN(CO(CH₂)₃N₃)Cys)-NH₂(SEQ ID NO. 10) with cysteines linked via disulfide bond, denoted GGGG-E∨R—N₃) as a white solid (14.5 mg, 0.0082 mmol, 65% yield). Peptide purity was confirmed by MALDI-TOF mass spectroscopy: calculated [M+¹H]⁺, 1771.8; observed 1771.6.

### Synthesis of Reductive-OR-Photo-Degradable, Sortaggable Peptide (GGGG-R∨P—N₃ (SEQ ID NO. 11))

H-GRC-NH₂ was synthesized on rink amide resin (0.25 mmol scale) via standard Fmoc-based, microwave-assisted, solid-phase peptide synthesis methodologies (FIG. 27). The resin-bound peptide was reacted overnight with N₃-oNB—OSu (165 mg, 0.325 mmol, 1.3×) and DIEA (129.25 mg, 1 mmol, 4×) in minimal DMF to introduce oNB and azido functionality onto the peptide N-terminus. The N-terminal azide was reduced to an amine by Staudinger reduction; the resin-bound peptide was washed THF/dH₂O (90:10, 3×20 mL) and reacted overnight with 5 wt % triphenylphosphine in THF/dH₂O (90/10, 30 mL). The peptide Fmoc-GGGGRG (SEQ ID NO. 11) was appended to the amine via standard microwave-assisted solid-phase peptide synthesis methodology. The peptide was deprotected and cleaved from resin by treatment with TFA/TIS/dH₂O (95:2.5:2.5, 15 mL) for 2 hours. Following cleavage, the peptide was precipitated in and washed with ice-cold diethyl ether (2×), purified by RP-HPLC operating with 43.4 min gradient (20-100%) of acetonitrile in water containing TFA (0.1%), and lyophilized to obtain the intermediate peptide (Fmoc-GGGGRG-oNB-GRC-NH₂) (SEQ ID NO. 11) as a yellow solid (75 mg, 0.055 mmol, 22% yield). The intermediate peptide and H-Cys-OH (66.64 mg, 0.55 mmol, 10×) were dissolved in dH₂O/DMSO (9:1, 10 mL) and reacted at room temperature for 48 hrs. The solution was concentrated by rotary evaporation, dissolved in dH₂O, purified by RP-HPLC with 43.4 min gradient (20-100%) of acetonitrile in water containing TFA (0.1%), and lyophilized to give the intermediate peptide (Fmoc-GGGGRG-oNB-GRC(HN(CO(CH₂)₃N₃)—C—OH)—NH₂ (SEQ ID NO. 11) with cysteines linked via disulfide bond) as a yellow solid (43.33 mg, 0.0292 mmol, 53.1% yield). The intermediate peptide was reacted overnight with N₃—OSu (13.22 mg, 0.584 mmol, 2×) and DIEA (15.13 mg, 0.117 mmol, 4×) in minimal DMF to introduce azide functionality onto the peptide. The reaction mixture was purified by RP-HPLC with 43.4 min gradient (20-100%) of acetonitrile in water containing TFA (0.1%), and the product fraction was lyophilized to obtain the intermediate peptide (Fmoc-GGGGRG-oNB-GRC(HN(CO(CH₂)₃N₃)—C—OH)—NH₂ (SEQ ID NO. 11) with cysteines linked via disulfide bond) as a yellow solid (38.6 mg, 0.0242 mmol, 82.9% yield). The N-terminal Fmoc group was cleaved by incubating the peptide in piperidine (20%) in DMF (40 mL) for 10 mins. The deprotection reaction mixture was concentrated via rotary evaporation, purified by RP-HPLC operating with 55 min gradient (5-100%) of acetonitrile in water containing TFA (0.1%), and lyophilized to obtain the final product (H-GGGGRG-oNB-GRC(HN(CO(CH₂)₃N₃)—C—OH)—NH₂ (SEQ ID NO. 11) with cysteines linked via disulfide bond, denoted GGGG-R∨P—N₃ (SEQ ID NO. 11)) as a yellow solid (7.86 mg, 0.0057 mmol, 20% yield). Peptide purity was confirmed by MALDI-TOF mass spectroscopy: calculated [M+¹H]⁺, 1371.65; observed 1371.52.

### Synthesis of Enzyme-OR-Photo-Degradable, Sortaggable Peptide (GGGG-E∨P—N₃) (SEQ ID NO. 12)

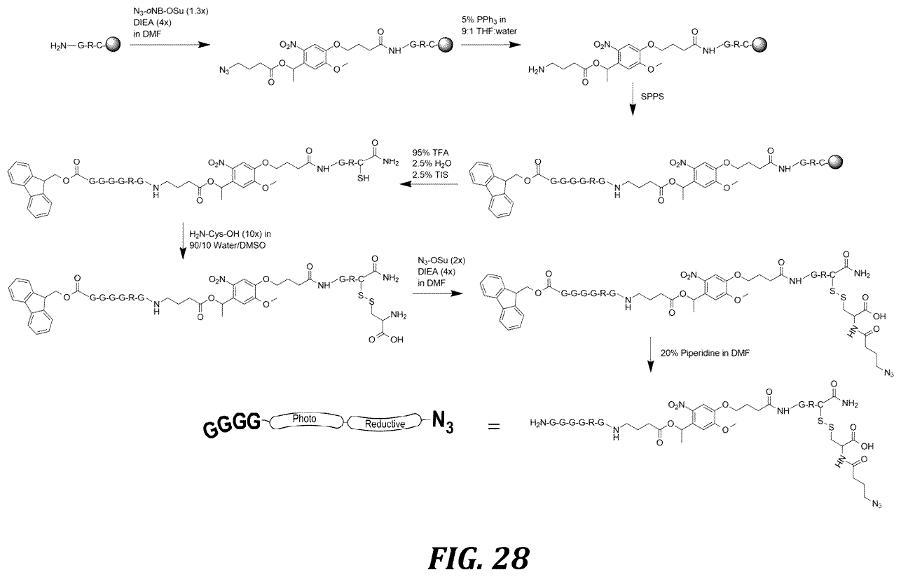

Fmoc-GGGGRGPQGIWGQGRK(mtt) (SEQ ID NO. 12) was synthesized on rink amide resin (0.25 mmol scale) via standard Fmoc-based, microwave-assisted, solid-phase peptide synthesis methodologies (FIG. 28). The highly acid labile N-methyltrityl (mtt) protection group on ε-amino group of the Lysine side chain was removed via treatment with Dichloromethane/triisopropylsilane/trifluoroacetic acid (97:2:1, 9×15 mL, 10 min each). The resin-bound peptide was reacted overnight with N₃-oNB—OSu (165 mg, 0.325 mmol, 1.3×) and DIEA (129.25 mg, 1 mmol, 4×) in minimal DMF to introduce oNB and azido functionality onto the ε-amino group of the Lysine side chain. The Fmoc deprotection was achieved on resin by treatment with a solution of piperidine (20%) and HOBt (0.1 M) in DMF (2×15 mL) for 10 mins. The peptide was deprotected and cleaved from resin by treatment with TFA/TIS/dH₂O (95:2.5:2.5, 15 mL) for 2 hours. Following cleavage, the peptide was precipitated in and washed with ice-cold diethyl ether (2×), purified by RP-HPLC operating with 55 min gradient (5-100%) of acetonitrile in water containing TFA (0.1%), and lyophilized to obtain the final product (H-GGGGRGPQGIWGQGRK(oNB-N₃)—NH₂ (SEQ ID NO. 12), denoted GGGG-E∨P—N₃) as a yellow solid (31.5 mg, 0.016 mmol, 6.4% yield). Peptide purity was confirmed by MALDI-TOF mass spectroscopy: calculated [M+¹H]⁺, 1958.2, observed 1958.5.

### Synthesis of Enzyme-AND-Reductive-Degradable, Sortaggable Peptide (GGGG-E∧R—N₃) (SEQ ID NO. 13)

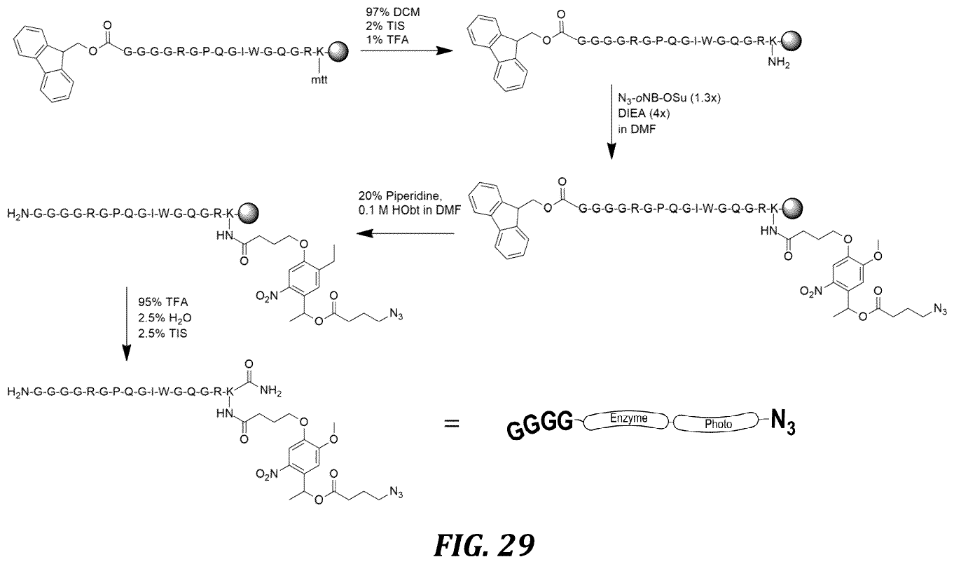

Fmoc-GGGGRGCGPQGIWGQGQGCGRK (SEQ ID NO. 13) was synthesized on rink amide resin (0.25 mmol scale) via standard Fmoc-based, microwave-assisted, solid-phase peptide synthesis methodologies (FIG. 29). The peptide was deprotected and cleaved from resin by treatment with TFA/TIS/dH₂O (95:2.5:2.5, 15 mL) for 2 hours. Following cleavage, the peptide was precipitated in and washed with ice-cold diethyl ether (2×), purified by RP-HPLC operating with 43.4 min gradient (20-100%) of acetonitrile in water containing TFA (0.1%), and lyophilized to obtain the intermediate peptide (Fmoc-GGGGRGCGPQGIWGQGQGCGRK-NH₂) (SEQ ID NO. 13) as a white solid (126.5 mg, 0.059 mmol, 23.9% yield). The peptide was stapled via formation of an intramolecular disulfide bridge between the cysteine residues of the peptide; the intermediate peptide (1 mM) was dissolved in a dH₂O/DMSO (90:10, 63 mL) solution and reacted at room temperature with no agitation for 48 hours. The stapled peptide was concentrated by rotary evaporation, dissolved in dH₂O, purified by RP-HPLC with 43.4 min gradient (20-100%) of acetonitrile in water containing TFA (0.1%), and lyophilized to give the intermediate peptide (Fmoc-GGGGRGCGPQGIWGQGQGCGRK-NH₂ (SEQ ID NO. 13), stapled intramolecularly via cysteine-cysteine disulfide bond) as a white solid (47.6 mg, 0.0226 mmol, 37.7% yield). The intermediate peptide was reacted overnight with N₃—OSu (10.11 mg, 0.044 mmol, 2×) and DIEA (11.55 mg, 0.089 mmol, 4×) in minimal DMF to introduce azide functionality onto the peptide. The reaction mixture was purified by RP-HPLC operating with 43.4 min gradient (20-100%) of acetonitrile in water containing TFA (0.1%), and lyophilized to obtain the intermediate peptide (Fmoc-GGGGRGCGPQGIWGQGQGCGRK(CO(CH₂)₃N₃)—NH₂ (SEQ ID NO. 13), stapled intramolecularly via cysteine-cysteine disulfide bond) as a white solid (34.4 mg, 0.0155 mmol, 68.6%). The N-terminal Fmoc group was cleaved by incubating the peptide in piperidine (20%) in DMF (30 mL) for 10 mins. The deprotection reaction mixture was concentrated via rotary evaporation, purified by RP-HPLC operating with 55 min gradient (5-100%) of acetonitrile in water containing TFA (0.1%), and lyophilized to obtain the final product (H-GGGGRGCGPQGIWGQGQGCGRK(CO(CH₂)₃N₃)—NH₂ (SEQ ID NO. 13), stapled intramolecularly via cysteine-cysteine disulfide bond, denoted GGGG-E∧R—N₃) as white a solid (8.3 mg, 0.0042 mmol, 27% yield). Peptide purity was confirmed by MALDI-TOF mass spectroscopy: calculated [M+¹H]⁺, 1997.13; observed 1996.9.

### Synthesis of Reductive-AND-Photo-Degradable, Sortaggable Peptide (GGGG-R∧P—N₃) (SEQ ID NO. 15 bound to (SEQ ID NO. 14) through oNB)

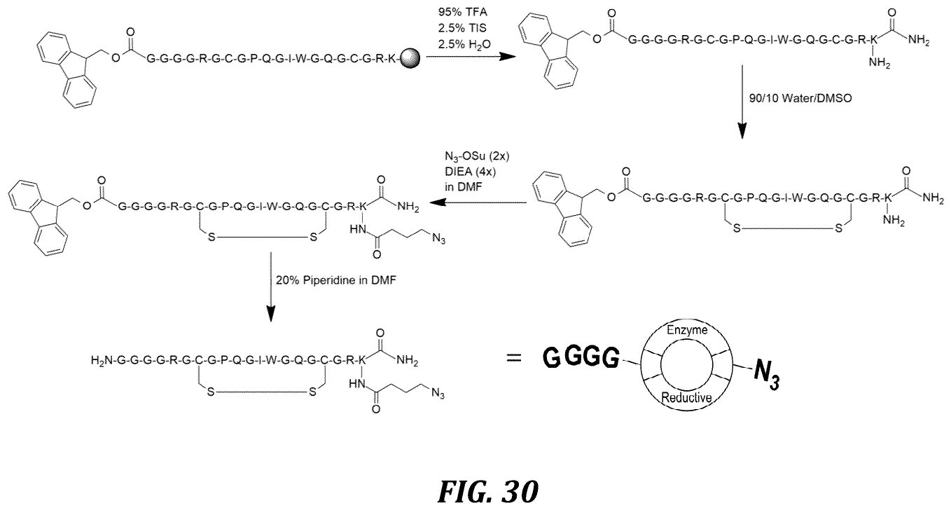

H-GGCGRK(boc) (SEQ ID NO. 14) was synthesized on rink amide resin (0.25 mmol scale) via standard Fmoc-based, microwave-assisted, solid-phase peptide synthesis methodologies (FIG. 30). The resin-bound peptide was reacted overnight with N₃-oNB—OSu (165 mg, 0.325 mmol, 1.3×) and DIEA (129.25 mg, 1 mmol, 4×) in minimal DMF to introduce oNB and azido functionality onto the N-terminus. The azide was reduced to an amine by Staudinger reduction; the resin-bound peptide was washed THF/dH₂O (90:10, 3×20 mL) and reacted overnight with 5 wt % triphenylphosphine in THF/dH₂O (90/10, 30 mL). Fmoc-GGGGRGCGG-OH (SEQ ID NO. 15) was appended to the amine of (SEQ ID NO. 14) via standard microwave-assisted solid-phase peptide synthesis methodology. The peptide was deprotected and cleaved from resin by treatment with TFA/TIS/dH₂O (95:2.5:2.5, 15 mL) for 2 hours. Following cleavage, the peptide was precipitated in and washed with ice-cold diethyl ether (2×), purified by RP-HPLC operating with 43.4 min gradient (20-100%) of acetonitrile in water containing TFA (0.1%), and lyophilized to obtain the intermediate peptide (Fmoc-GGGGRGCG-oNB-GGCGRK-NH₂) ((SEQ ID NO. 15) bound to (SEQ ID NO. 14) through oNB), as a yellow solid (100 mg, 0.055 mmol, 22% yield). The peptide was stapled via formation of an intramolecular disulfide bridge between the cysteine residues of (SEQ ID NO. 15) and (SEQ ID NO. 14). This intermediate peptide (1 mM) was dissolved in a dH₂O/DMSO (90:10, 55 mL) solution and reacted at room temperature with no agitation for 48 hours. The stapled peptide was concentrated by rotary evaporation, dissolved in dH₂O, purified by RP-HPLC with 43.4 min gradient (20-100%) of acetonitrile in water containing TFA (0.1%), and lyophilized to give the intermediate, (SEQ ID NO. 15) bound to (SEQ ID NO. 14) through oNB), stapled intramolecularly via cysteine-cysteine disulfide bond as a yellow solid (60 mg, 0.0329 mmol, 59.8% yield). The intermediate was reacted overnight with N₃—OSu (10.5 mg, 0.464 mmol, 2×) and DIEA (11.98 mg, 0.093 mmol, 4×) in minimal DMF to introduce azide functionality. The reaction mixture was purified by RP-HPLC operating with 43.4 min gradient (20-100%) of acetonitrile in water containing TFA (0.1%), and lyophilized to obtain the intermediate azide Fmoc-GGGGRGCG-oNB-GGCGRK(CO(CH₂)₃N₃)—NH₂ ((SEQ ID NO. 15) bound to (SEQ ID NO. 14) through oNB) and stapled intramolecularly via cysteine-cysteine disulfide bond) as a yellow solid (44 mg, 0.0227 mmol, 50%). The N-terminal Fmoc group was cleaved by incubating in piperidine (20%) in DMF (10 mL) for 10 mins. The deprotection reaction mixture was concentrated via rotary evaporation, purified by RP-HPLC operating with 55 min gradient (5-100%) of acetonitrile in water containing TFA (0.1%), and lyophilized to obtain the final product (H-GGGGRGCGG-oNB-GGCGRK(CO(CH₂)₃N₃)—NH₂ ((SEQ ID NO. 15) bound to (SEQ ID NO. 14) through oNB and, stapled intramolecularly via cysteine-cysteine disulfide bond, denoted GGGG-R∧P—N₃) as a yellow solid (6.3 mg, 0.0037 mmol, 16.3% yield). Product purity was confirmed by MALDI-TOF mass spectroscopy: calculated [M+¹H]⁺, 1709.9; observed 1709.6

### Synthesis of Enzyme-AND-Photo-Degradable, Sortaggable Peptide (GGGG-E∧P—N₃) (SEQ ID NO. 18)

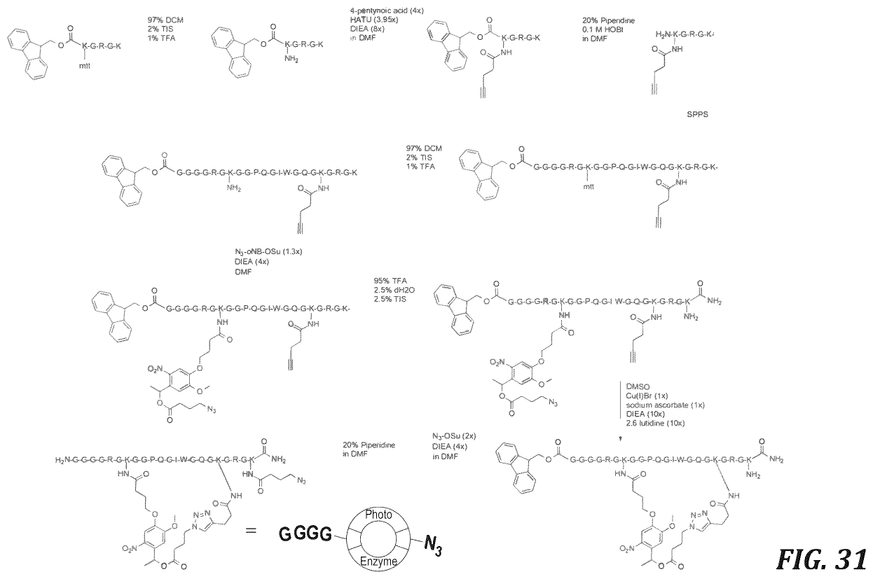

Fmoc-K(mtt)GRGK(boc) (SEQ ID NO. 16) was synthesized on rink amide resin (0.25 mmol scale) via standard Fmoc-based, microwave-assisted, solid-phase peptide synthesis methodologies (FIG. 31). The highly acid labile N-methyltrityl (mtt) protection group on ε-amino group of the Lysine side chain was removed via treatment with Dichloromethane/triisoproylsilane/trifluoroacetic acid (97:2:1, 9×15 mL, 10 min each). HATU coupling was used to functionalize ε-amino group of the Lysine side chain with an alkyne; 4-pentynoic acid (98.1 mg, 1 mmol, 4×) was pre-activated upon reaction with HATU (0.376 g, 0.99 mmol, 3.95×) and DIEA (260.7 mg, 2 mmol, 8×) in minimal DMF for 5 minutes and then reacted with the resin for 90 minutes. The Fmoc protecting group on the N-terminus of the peptide was cleaved on resin by treatment with a solution of piperidine (20%) and HOBt (0.1 M) in DMF (2×15 mL, 10 mins each). The peptide Fmoc-GGGGRGK(mtt)GGPQGIWGQG (SEQ ID NO. 17) was appended to the N-terminus of (SEQ ID NO. 16) via standard microwave-assisted solid-phase peptide synthesis methodology to produce Fmoc-GGGGRGK(mtt)GGPQGIWGQGK(CO(CH₂)₂CCH)GRGK-Resin (SEQ ID NO. 18). The N-methyltrityl (mtt) protection group on 8-amino group of the Lysine side chain was removed via treatment with Dichloromethane/triisoproylsilane/trifluoroacetic acid (97:2:1, 9×15 mL, 10 min each). The resin-bound peptide was reacted overnight with N₃-oNB—OSu (165 mg, 0.325 mmol, 1.3×) and DIEA (129.25 mg, 1 mmol, 4×) in minimal DMF to introduce oNB and azido functionality onto the ε-amino group of the Lysine side chain. The peptide was deprotected and cleaved from resin by treatment with TFA/TIS/dH₂O (95:2.5:2.5, 15 mL) for 2 hours. Following cleavage, the peptide was precipitated in and washed with ice-cold diethyl ether (2×), purified by RP-HPLC operating with 43.4 min gradient (20-100%) of acetonitrile in water containing TFA (0.1%), and lyophilized to obtain the intermediate peptide (Fmoc-GGGGRGK(oNB-N₃)GGPQGIWGQGK(CO(CH₂)₂CCH)GRGK-NH₂) (SEQ ID NO. 18) as a yellow solid (85 mg, 0.0316 mmol, 12.7% yield). The alkyne and azide functionalities present on the peptide side chains were stapled together via CuAAC (copper(I)-catalyzed azide-alkyne cycloaddition) click reaction; the linear peptide (1 mM) was dissolved in nitrogen-purged DMSO (32 mL) containing cooper(I) bromide (4.53 mg, 0.032 mmol, 1 eq), sodium ascorbate (6.19 mg, 0.032 mmol, 1 eq) in water (316 μL), lutidine (32.23 mg, 0.316 mmol, 10 eq), and DIEA (40.84 mg, 0.316 mmol, 10 eq); this mixture was allowed to react under nitrogen at room temperature overnight, concentrated via rotary evaporation, passed through ion exchange column (Dowex, M4195 resin, 5 g), and lyophilized. The stapled product was purified by RP-HPLC operating with 43.4 min gradient (20-100%) of acetonitrile in water containing TFA (0.1%), and lyophilized to obtain the intermediate peptide (Fmoc-GGGGRGK(oNB-N₃)GGPQGIWGQGK(CO(CH₂)₂CCH)GRGK-NH₂ (SEQ ID NO. 18), stapled intramolecularly via triazole linkage between the alkyne and oNB-N₃ side chains) as a yellow solid (7.8 mg, 2.9 μmol, 9.18% yield). The intermediate peptide was reacted overnight with N₃—OSu (1.31 mg, 0.0058, 2×) and DIEA (1.498 mg, 0.0116 mmol, 4×) in minimal DMF to introduce azide functionality onto the peptide. The reaction mixture was purified by RP-HPLC operating with 43.4 min gradient (20-100%) of acetonitrile in water containing TFA (0.1%), and lyophilized to obtain the intermediate peptide (Fmoc-GGGGRGK(oNB)GGPQGIWGQGK(CO(CH₂)₂)GRGK(CO(CH₂)₃—N₃)—NH₂ (SEQ ID NO. 18), stapled intramolecularly via triazole linkage between the alkyne and oNB-N₃ side chains as a yellow solid (8 mg, 2.85 μmol, 98% yield). The N-terminal Fmoc group was cleaved by incubating the peptide in piperidine (20%) in DMF (8 mL) for 10 mins. The deprotection reaction mixture was concentrated via rotary evaporation, purified by RP-HPLC operating with 55 min gradient (5-100%) of acetonitrile in water containing TFA (0.1%), and lyophilized to obtain the final product (H-GGGGRGK(oNB)GGPQGIWGQGK(CO(CH₂)₂)GRGK(CO(CH₂)₃—N₃)—NH₂ (SEQ ID NO. 18) stapled intramolecularly via triazole linkage between the alkyne and oNB-N₃ side chains, denoted GGGG-E∧P—N₃ (SEQ ID NO. 18) as a solid (3.6 mg, 1.4 μmol, 49% yield). Peptide purity was confirmed by MALDI-TOF mass spectroscopy: calculated [M+¹H]⁺, 2577.7; observed 2577.5.

### Protein Expression and Protein-Peptide Conjugation.

### Protein Expression and Ni-NTA Immobilization

The pSTEPL-POI construct was transformed into BL21 (DE3) line. A starter culture (20 mL) was grown overnight in LB containing Ampicillin (0.1 mg/mL). The starter culture was used to inoculate LB (480 mL) containing Ampicillin (0.1 mg/mL). The culture was grown to an OD600 of 0.4-0.6 before induction with Isopropyl β-D-1-thiogalactopyranoside (0.5 mM, IPTG). The culture was grown overnight at 25° C. The cells were centrifuged (4000×g, 10 mins) to obtain a pellet. The cells were re-suspended in STEPL Lysis Buffer (20 mM Tris-base, 50 mM NaCl, 10 mM imidazole, pH 7.5) containing phenylmethanesulfonyl fluoride (1 mM, PMSF) as a protease inhibitor. The cells were lysed using sonication; the cell suspension was kept on ice and subjected to short sonication burst cycles (1 sec pulse, 2 sec pause) with total sonication time 6 minutes. After sonication, the lysate was clarified by centrifugation (4000×g, 10 minutes) and loaded onto Ni-NTA resin. The resin was washed with STEPL wash buffer (20 mM Tris-base, 50 mM NaCl, 20 mM imidazole, pH 7.5) to remove non-specifically bound proteins.

Similar constructs for mCherry and mCerulean expression were prepared by standard molecular cloning techniques.

### POI-Peptide Conjugation by STEPL Reaction

Ni-NTA column was loaded with EGFP as described above. Sortaggable peptide was dissolved in STEPL buffer (5 mL) containing calcium chloride (0.1 mM, Ca⁺⁺) and added to the column. To promote STEPL reaction, the column was reacted at 37° C. for 4 hours with gently agitation, resulting to the modification and subsequent displacement of POI with sortaggable peptide. After the STEPL reaction, the column flow through, containing the protein-peptide construct, was collected. Centrifugal membrane filter (Amicon Ultra-4, MW cutoff: 10 kDa) were used to simultaneously concentrate and further purify the protein-peptide construct.

### Synthesis of Enzyme-Responsive EGFP Pendant (EGFP-E-N₃)

EGFP was expressed as a STEPL construct and loaded on the Ni-NTA column (Method S10.1). Sortaggable peptide GGGG-E-N₃ (SEQ ID NO. 57) was conjugated onto EGFP. Protein purity was confirmed by LC-MS: calculated [M], 29179.7; observed 29179.5.

### Synthesis of Reductive-Responsive EGFP Pendant (EGFP-R—N₃)

EGFP was expressed as a STEPL construct and loaded on the Ni-NTA column. Sortaggable peptide GGGG-R—N₃ (SEQ ID NO. 8) (Method S2) was conjugated onto EGFP. Protein purity was confirmed by LC-MS: calculated [M], 28321.97; observed 28322.

### Synthesis of Photo-Responsive EGFP Pendant (EGFP-P—N₃)

EGFP was expressed as a STEPL construct and loaded on the Ni-NTA column. Sortaggable peptide GGGG-P—N₃ (SEQ ID NO. 9) was conjugated onto EGFP. Protein purity was confirmed by LC-MS: calculated [M], 28508.96; observed 28509.

### Synthesis of Enzyme-OR-Reductive-Responsive EGFP Pendant (EGFP-E∨R—N₃)

EGFP was expressed as a STEPL construct and loaded on the Ni-NTA column. Sortaggable peptide GGGG-E∨R—N₃ (SEQ ID NO. 10) was conjugated onto EGFP. Protein purity was confirmed by LC-MS: calculated [M], 29358.8; observed 29358.5.

### Synthesis of Photo-OR-Reductive-Responsive EGFP Pendant (EGFP-R∨P—N₃)

EGFP was expressed as a STEPL construct and loaded on the Ni-NTA column. Sortaggable peptide GGGG-R∨P—N₃ (SEQ ID NO. 11) was conjugated onto EGFP. Protein purity was confirmed by LC-MS: calculated [M], 28958.65; observed 28958.

### Synthesis of Enzyme-OR-Photo-Responsive EGFP Pendant (EGFP-E∨P—N₃)

EGFP was expressed as a STEPL construct and loaded on the Ni-NTA column. Sortaggable peptide GGGG-E∨P—N₃ (SEQ ID NO. 12) was conjugated onto EGFP. Protein purity was confirmed by LC-MS: calculated [M], 29545.2; observed 29545.5.

### Synthesis of Enzyme-AND-Reductive-Responsive-EGFP Pendant (EGFP-E∧R—N₃)

EGFP was expressed as a STEPL construct and loaded on the Ni-NTA column. Sortaggable peptide GGGG-E∧R—N₃ (SEQ ID NO. 13) was conjugated onto EGFP. Protein purity was confirmed by LC-MS: calculated [M], 29584.13; observed 29583.5.

### Synthesis of Photo-AND-Reductive-Responsive-EGFP Pendant (EGFP-R∧P—N₃)

EGFP was expressed as a STEPL construct and loaded on the Ni-NTA column. Sortaggable peptide GGGG-R∧P—N₃ (SEQ ID NO. 14) and (SEQ ID NO. 15) was conjugated onto EGFP. Protein purity was confirmed by LC-MS: calculated [M], 29296.9; observed 29296.5.

### Synthesis of Enzyme-AND-Photo-Responsive EGFP Pendant (EGFP-E∧P—N₃)

EGFP was expressed as a STEPL construct and loaded on the Ni-NTA column. Sortaggable peptide GGGG-E∧P—N₃ (SEQ ID NO. 18) was conjugated onto EGFP. Protein purity was confirmed by LC-MS: calculated [M], 30165.7; observed 30165.

### Synthesis of Enzyme-Responsive mCherry Pendant (mCherry-E-N₃)

mCherry was expressed as a STEPL construct and loaded on the Ni-NTA column. Sortaggable peptide GGGG-Enzyme-N₃ (SEQ ID NO. 12) was conjugated onto mCherry.

Protein purity was confirmed by LC-MS: calculated [M], 28959.9; observed 28958.5.

### Synthesis of Enzyme-OR-Reductive-Responsive-mCherry Pendant (mCherry-E∨R—N₃)

EGFP was expressed and loaded on the Ni-NTA column. Sortaggable peptide was conjugated onto mCherry. Protein purity was confirmed by LC-MS: calculated [M], 29140.9; observed 29137.5.

### Synthesis of Reductive-AND-Photo-Responsive-mCherry Pendant (mCherry-R∧P—N₃)

mCherry was expressed as a STEPL construct and loaded on the Ni-NTA column. Sortaggable peptide GGGG-R∧P—N₃ (SEQ ID NO. 15) and (SEQ ID NO. 14) was conjugated onto mCherry. Protein purity was confirmed by LC-MS: calculated [M], 29079.0; observed 29075.5.

### Protein Release Study Methodologies

Hydrogels were formulated from PEG_{20 k}-tetraBCN (2 mM), PEG_{3.5 k}-diazide crosslinker (4 mM) and peptide-protein construct (0.1 mM) in MMP buffer (total: 40 μL). PEG_{20 k}-tetraBCN and peptide-protein construct were pre-reacted for 4 hours prior to PEG_{3.5 k}-diazide crosslinker addition. This solution was vortexed, centrifuged, transferred to microcentrifuge tubes (4×10 μL), centrifuged and allowed to gel for 1 hour. For each study, a total of 24 hydrogels were synthesized (8 gel treatment conditions, each in triplicate).

Samples were thoroughly washed in MMP buffer at 4° C. to remove any unconjugated protein prior to beginning the release study.

Samples receiving reductive input were treated with TCEP-HCl (2 μL, 100 mM in MMP buffer) and incubated overnight at 4° C. Following the reductive input, these samples were further treated with hydroxyethyl disulfide (5 μL, 100 mM in MMP buffer) and incubated at 4° C. for 5 hours.

Samples receiving the enzyme input were treated with recombinantly-expressed MMP-8 (2.5 μL, 0.4 mg/mL in MMP buffer) and all samples were incubated overnight at 4° C.

Samples receiving light input were treated with light (λ=365 nm, 20 mW cm² incident light, 10 minutes exposure) and incubated overnight at 4° C.

After incubation, supernatant was removed from each sample and transferred to a 96-well plate. The fluorescence of these solutions and washed were measured. Measured fluorescence was related to protein release concentration.

### Calibration of Protein Concentration vs Fluorescence

### EGFP Fluorescence Calibration

The EGFP calibration curve was obtained for peptide-protein conjugate (EGFP-E-N₃, Method 10.3) dissolved in MMP Buffer (Tris 50 mM, NaCl 200 mM, CaCl₂) 5 mM, ZnCl₂ 1 μM, pH 7.5). The concentration range (maximum concentration: 11.25 μM; minimum concentration: 1.25 μM) was divided into 9 equally spaced intervals corresponding to a total 10 points, for which fluorescence was measured in triplicates (λ_{excitation}=475 nm, λₑₘᵢₛₛᵢₒₙ=510 nm).

mCherry Fluorescence Calibration

The mCherry calibration curve was obtained for peptide-protein conjugate (mCherry-P—N₃) dissolved in MMP Buffer. The concentration range (maximum concentration: 3.75 μM; minimum concentration: 0.375 μM) was divided into 9 equally spaced intervals corresponding to a total 10 points, for which fluorescence was measured in triplicates (λ_{excitation}=575 nm, λₑₘᵢₛₛᵢₒₙ=610 nm).

mCerulean Fluorescence Calibration

The mCerulean calibration curve was obtained for peptide-protein conjugate (mCerulean-E-N₃) dissolved in MMP Buffer. The concentration range (maximum concentration: 3 μM; minimum concentration: 0.375 μM) was divided into 7 equally spaced intervals corresponding to a total 8 points, for which fluorescence was measured in triplicates (λ_{excitation}=433 nm, λₑₘᵢₛₛᵢₒₙ=475 nm).

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of selectively releasing a payload from a material having a molecular-logic gate configuration with at least two cleavable moieties, the method comprising a step of applying stimuli sufficient to cleave the at least two cleavable moieties so as to release the payload.
